(19) United States
(12) Patent Application Publication
(10) Pub. No.: US 2006/0270591 A1
(43) Pub. Date: Nov. 30, 2006

(54) Androgen receptor coregulators

(76) Inventor: Chawnshang Chang, Pittford, NY (US)

Correspondence Address: NEEDLE & ROSENBERG, P.C., SUITE 1000, 999 PEACHTREE STREET, ATLANTA, GA, 30309-3915, US

(21) Appl. No.: 10/517,155

(22) PCT Filed: Jun. 6, 2003
(86) PCT No.: PCT/US03/17937

§ 371 Date: Jan. 10, 2005

Related U.S. Application Data
(60) Provisional application No. 60/387,087, filed on Jun. 6, 2002.

(51) Int. Cl. A61K 38/17; C07H 21/04; C12P 21/06; A01K 67/00; C07K 14/705; C07K 14/72

(52) U.S. Cl. 514/12; 800/8; 435/69.1; 435/320.1; 435/325; 530/350; 536/23.5

(57) ABSTRACT

Disclosed are compositions and methods related to androgen receptor coregulators.

1. This application claims the benefit of U.S. Provisional Application 60/387,087 filed on Jun. 6, 2003, and herein incorporated by reference in its entireity. Related application 60/093,239 filed Jul. 17, 1998, 60/100,243, filed Sep. 14, 1998, and Ser. No. 09/354,221, filed Jul. 15, 1999 are all herein incorporated by reference in their entireties.

This work was supported by NIH Grants CA55639 and CA71570 (C.C), NIH grant CA71570, and CA71570.

### I. BACKGROUND OF THE INVENTION

2. Androgens constitute a class of hormones that control the development and proper function of mammalian male reproductive systems, including the prostate and epididymis. Androgens also affect the physiology of many non-reproductive systems, including muscle, skin, pituitary, lymphocytes, hair growth, nd brain. Androgens exert their effect by altering the level of gene expression of specific genes in a process that is mediated by binding of androgen to an androgen receptor. The androgen receptor, which is a member of the stroid receptor super family, and plays an important role in male sexual differentiation and in prostate cell proliferation.

3. Binding of androgen by the androgen receptor allows the androgen receptor to interact with androgen responsive element (AREs), DNA sequences found in genes whose expression is regulated by androgen.

4. Androgen-mediated regulation of gene expression is a complicated process that may involve ultiple co-activators (Adler et al., Proc. National Acad. Sci. USA 89:6319-6325, 1992). A fundamental question in the field of steroid hormone biology is how specific androben-activated transcription can be achieved in vivo when several different receptors recognize the same DNA sequence. For example, the androgen receptor (AR), the glucocorticoid receptor (GR), and the progesterone eceptor (PR) all recognize the same sequence but activate different transcription activities. Coactivators which interact wuth a subset of these different receptors is one way to obtain differential gene regulation.

5. Prostate cancer is the most common malignant neoplasm in aging males in the United States. Standard treatment includes the surgical or chemical castration of the patient in combination with the administration of anti-androgens such as 17˜estradiol (Glass et al. (2000) Genes & Development. 14, 121-41) or hydroxyflutamide (HF). However, most prostate cancers treated with androgen ablation and anti-androgens progress from an androgen-dependant to an androgen-independent state, causing a high incidence of relapse within 18 months (Crawford, Br. J. Urolog˜70: suppl. 1, 1992).

6. AIB1 was identified as estrogen receptor coactivator that is expressed at higher levels in ovarian cancer cell lines and breast cancer cells than in noncancerous cells (Anzick, et al. Science 277:965-968, 1997). This result suggests that steroid hormone receptor cofactors may play an important role in the progression of certain diseases, such as hormone responsive tumors.

7. The identification, isolation, and characterization of genes that encode factors involved in the regulation of gene expression by androgen receptors will facilitate the development of screening assays to evaluate the potential efficacy of drugs in the treatment of prostate cancers. Also disclosed are co-reglators of AR which can increase and/or decrease the transcription activity.

### II. SUMMARY OF THE INVENTION

8. In accordance with the purposes of this invention, as embodied and broadly described herein, this invention, in one aspect, relates to androgen receptor.

9. Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### III. BRIEF DESCRIPTION OF THE DRAWINGS

10. The accompanying drawings, which are incorporated in and constitute a part of this specification, illustrate several embodiments of the invention and together with the description, serve to explain the principles of the invention.

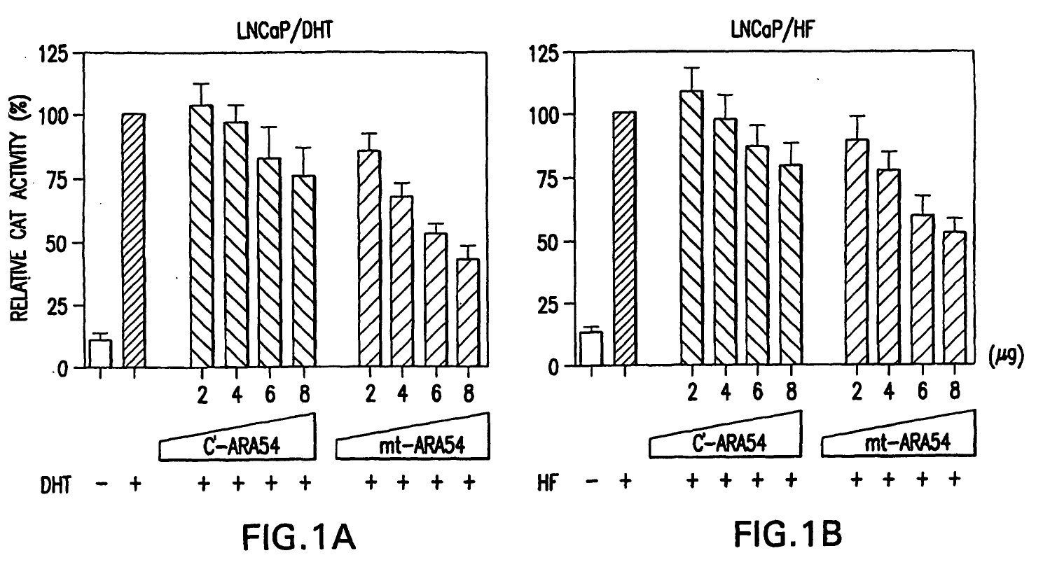

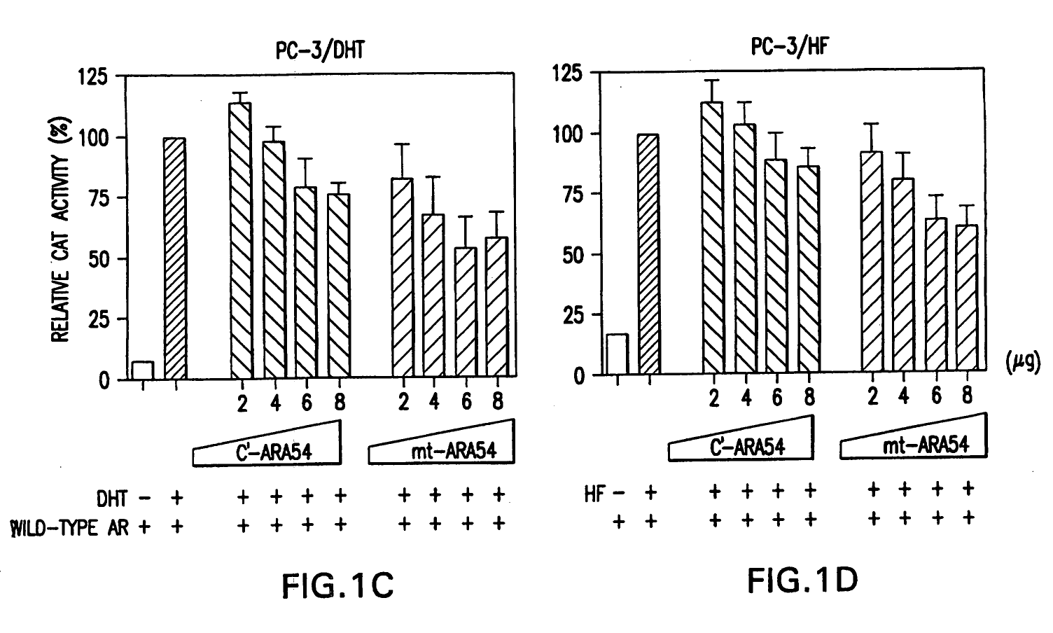

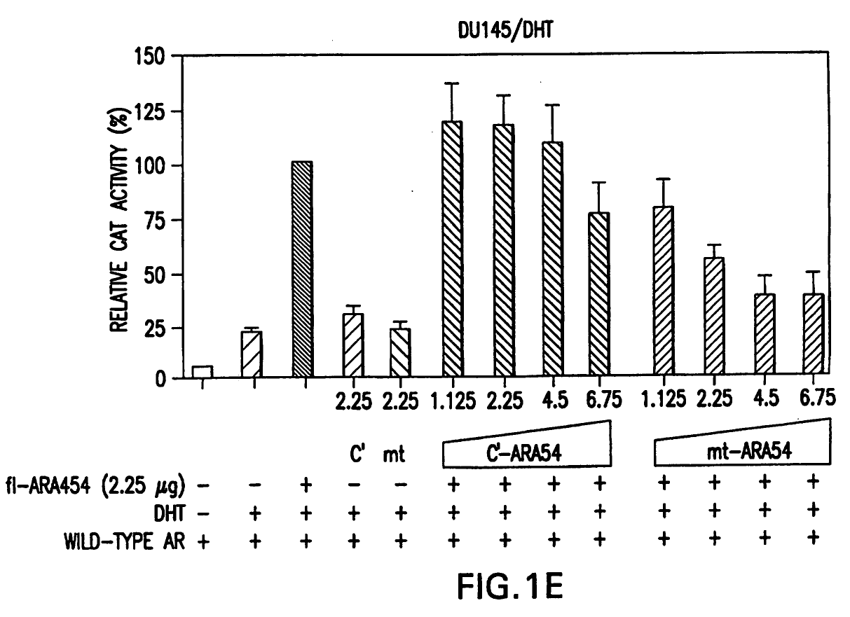

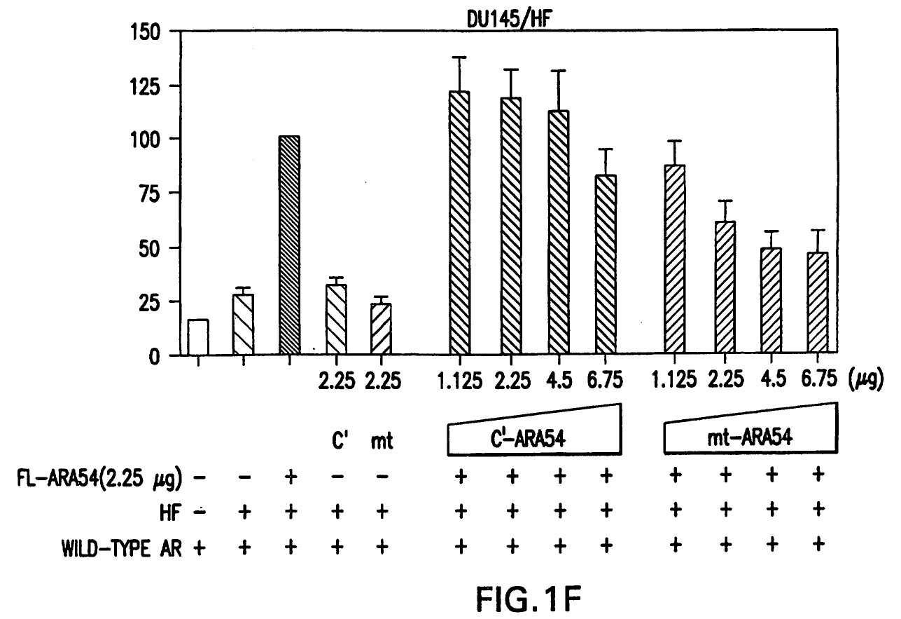

11. FIG. 1. shows the dominant-negative effects of C′-ARA54 and mt-ARA54 on AR transcription activity in human prostate cancer cell lines. LNCaP (A, B), PC-3 (C, D), or DU145 (E, F) cells were transfected with mouse mammary tumor virus (MMTV)-CAT plasmid (2.5 μg) and increasing amounts of pSG5-C′-ARA54 or pSG5-mt-ARA54 as indicated. The wild-type AR expression plasmid pSG5-AR was cotransfected in PC-3 and DU145 cells (1.0 μg for PC-3 and 0.75 μg for DU145). DU145 cells were also transfected with 2.25 μg of pSG5-fl-ARA54. The total amount of DNA was adjusted to 11.5-13.25 μg with pSG5 for each transfection. Twenty-four h after transfection, cells were cultured for an additional 24 h in the presence or absence of 1 nM DHT (A, C, E) or 1 μM HF (B, D, F). The CAT activity is presented relative to that of lane 2 (vector alone with DHT or HF) in each panel (black bars; set as 100%). Values represent the mean±SD of at least three determinations.

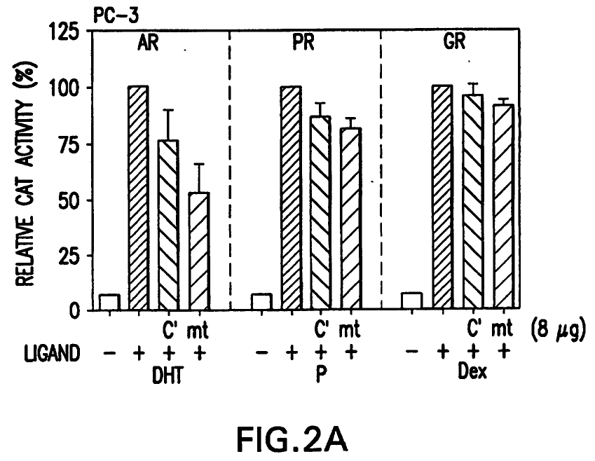

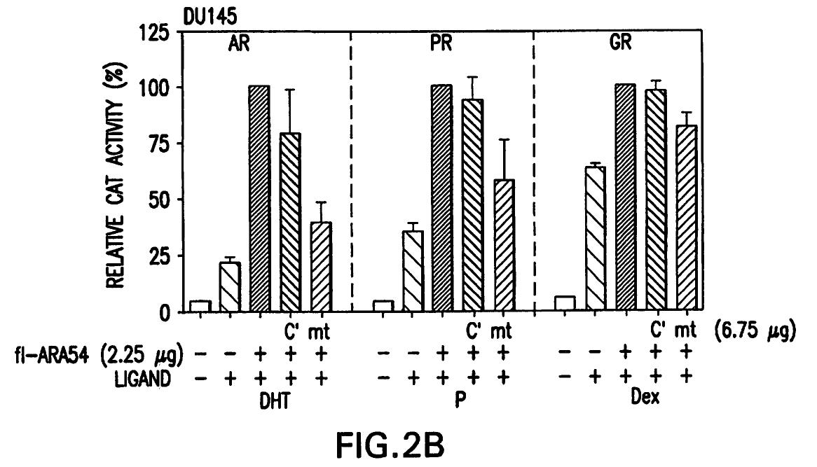

12. FIG. 2 shows the dominant-negative effects of C′-ARA54 and mt-ARA54 on the transcription activity of AR, PR, and GR. PC-3 (A) or DU145 (B) cells were transfected with MMTV-CAT (2.5 μg), steroid receptor expression plasmid (AR, PR, or GR; 1.0 μg for PC-3 and 0.75 μg for DU145), and pSG5-C′-ARA54 (C′) or pSG5-mt-ARA54 (mt) (8.0 μg for PC-3 and 6.75 μg for DU145), with (for DU145) or without (for PC-3) pSG5-fl-ARA54 (2.25 μg). The total amount of DNA was adjusted to 12.5-13.25 μg with pSG5 for each transfection. Twenty-four h after transfection, cells were cultured for an additional 24 h in the presence or absence of 1 nM DHT, 10 nM P, or 10 nM Dex as indicated. The CAT activity is presented relative to that of vector alone with cognate ligand in each panel (black bars; set as 100%). Values represent the mean±SD of at least three determinations.

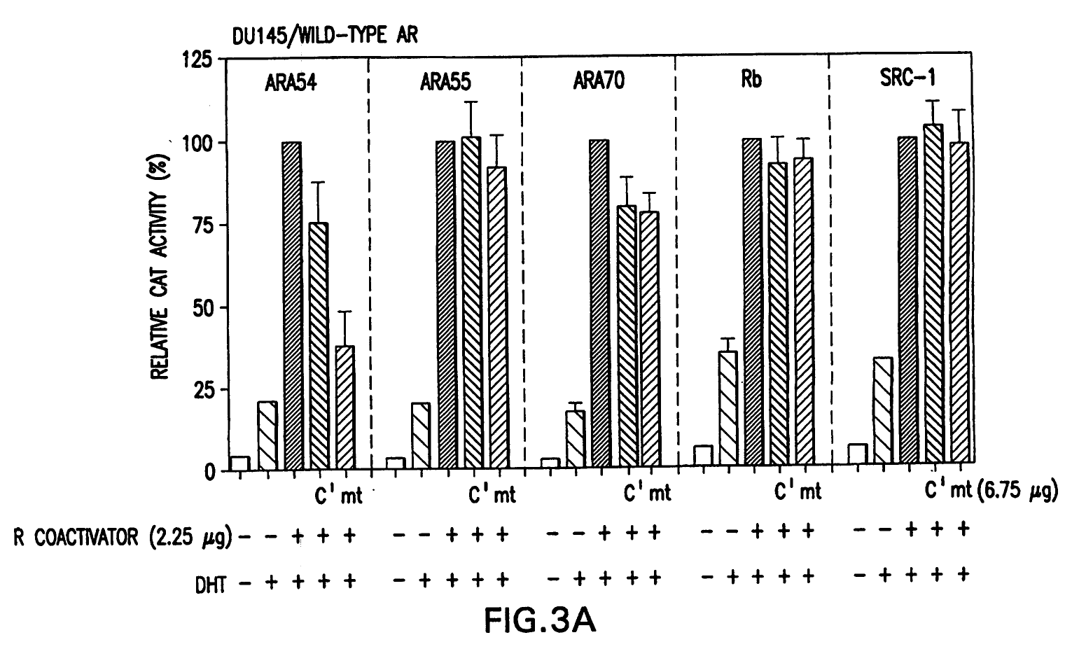

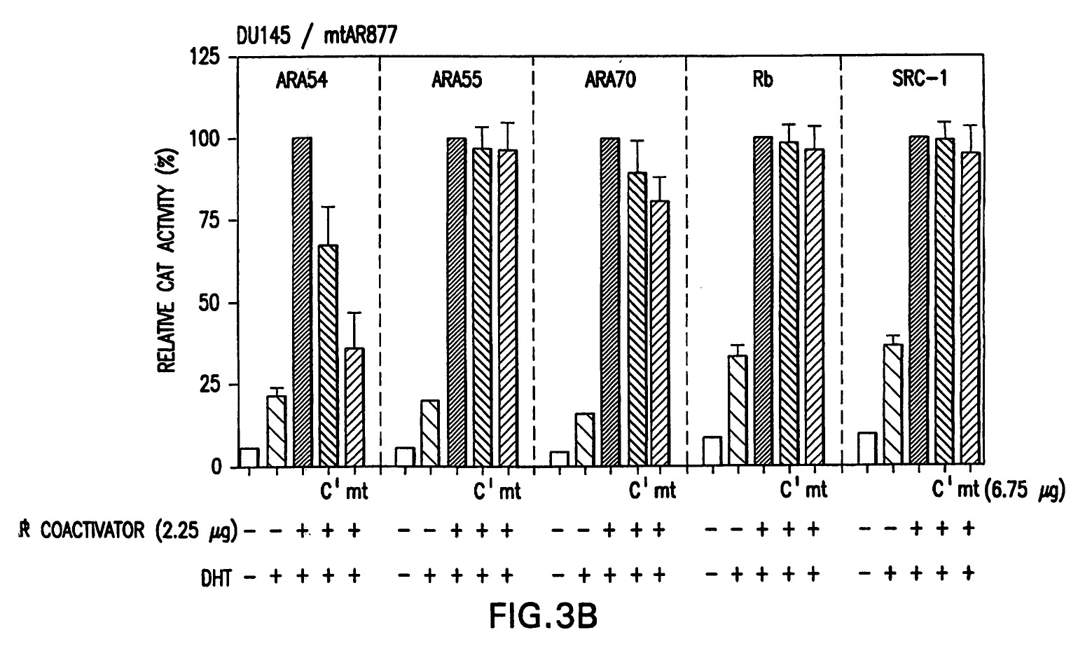

13. FIG. 3 shows the effects of C′-ARA54 and mt-ARA54 on AR transcription activity in the presence of different AR coactivators. DU145 cells were transfected with 2.5 μg of MMTV-CAT, 0.75 μg of AR expression plasmid (wild-type (A) and mtAR877 (B)), 2.25 μg of different AR coactivators (ARA54, ARA55, SRC-1, ARA70, Rb, or SRC-1), and 6.75 μg of pSG5-C′-ARA54 (C′) or pSG5-mt-ARA54 (mt). The total amount of DNA was adjusted to 13.25 μg with pSG5 for each transfection. Twenty-four h after transfection, cells were cultured for an additional 24 h in the presence or absence of 1 nM DHT as indicated. The CAT activity is presented relative to that of vector alone with DHT in each panel (black bars; set as 100%). Values represent the mean±SD of at least three determinations.

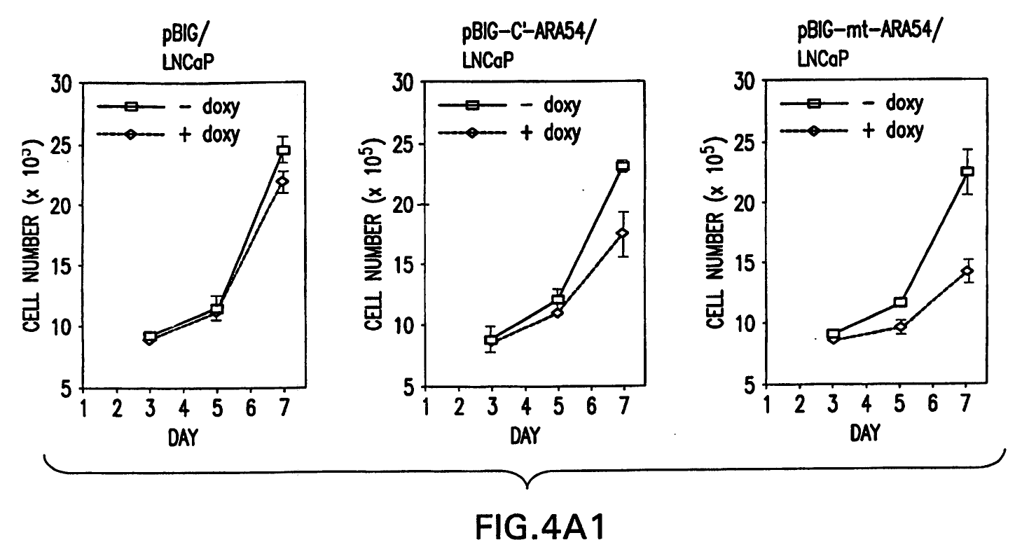

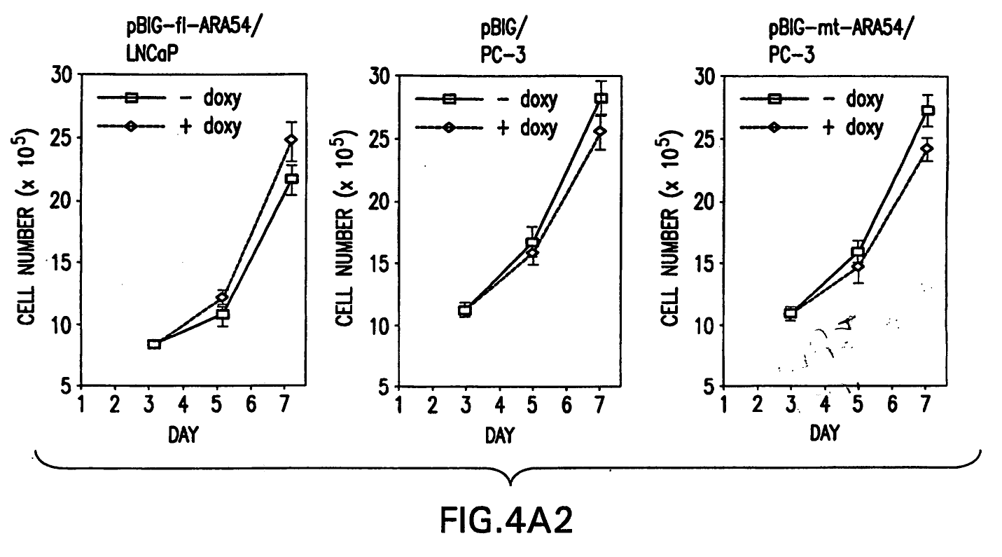

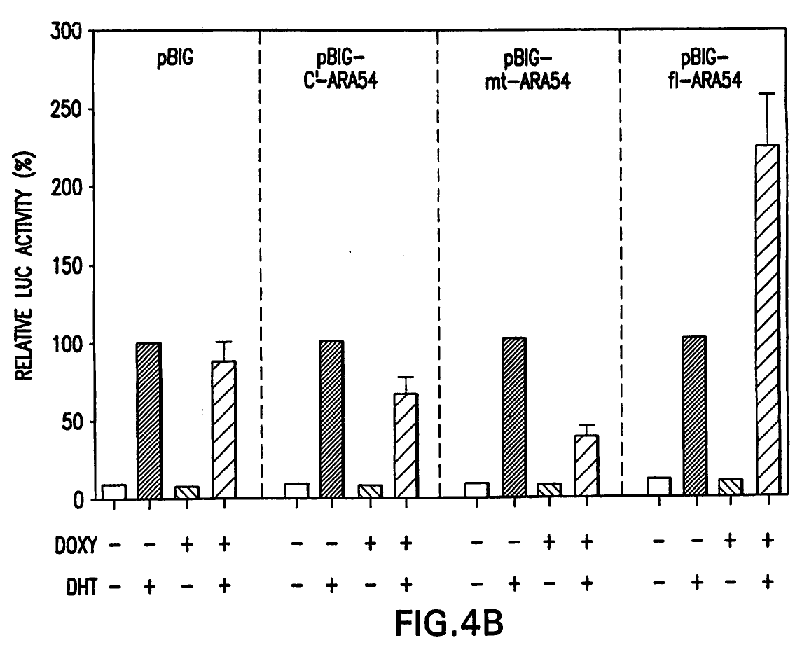

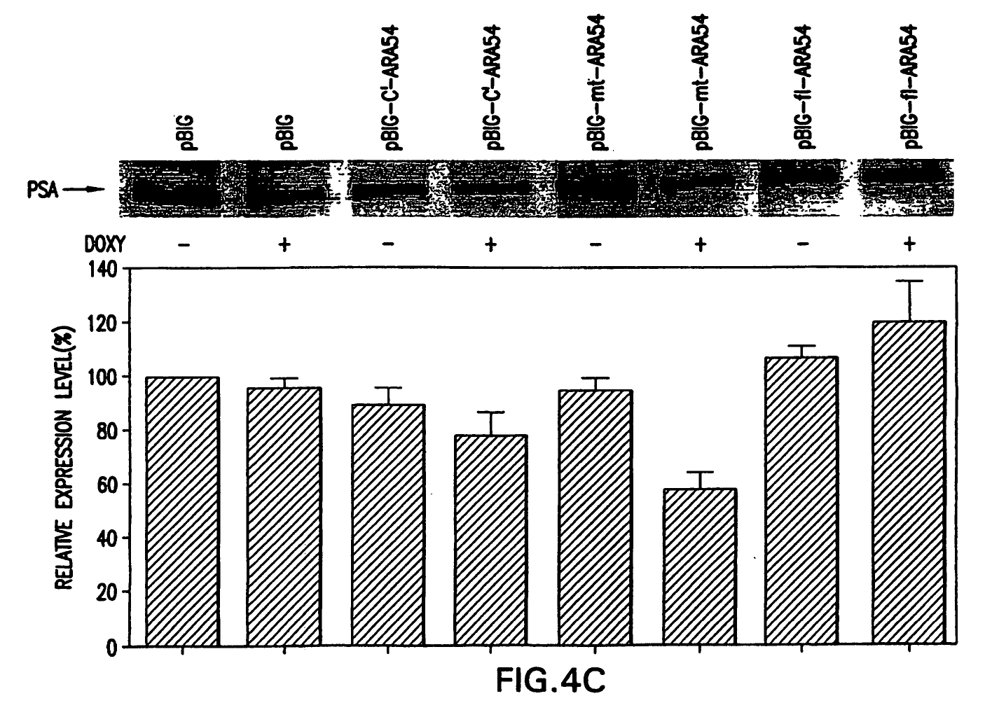

14. FIG. 4 shows the effects of the mutant ARA54 in the LNCaP cells stably transfected with pBIG2i-C′-ARA54 or pBIG2i-mt-ARA54 under tetracycline inducible system. (A) The effects of C′-ARA54 and mt-ARA54 on cell proliferation. LNCaP cells stably transfected with pBIG2i (vector alone), pBIG2i-C′-ARA54, pBIG2i-mt-ARA54, or pPIG2i-fl-ARA54 and PC-3 cells stably transfected with pBIG2i (vector alone) or pPIG2i-fl-ARA54 were cultured in the presence or absence of 2 μg/ml doxy with 1 nM DHT. Total cell number was counted by hemocytometer. Values represent the mean±SD of at least three determinations. (B) The effects of C′-ARA54 and mt-ARA54 on AR transcription activity. LNCaP cells stably transfected with pBIG2i (vector alone), pBIG2i-C′-ARA54, pBIG2i-mt-ARA54, or pBIG2i-fl-ARA54 were transiently transfected with MMTV-Luc. After transfection, cells were cultured in the presence or absence of 2 μg/ml doxy and 1 nM DHT as indicated. The Luc activity is presented relative to that in absence of doxy and presence of DHT in each panel (black bars; set as 100%). Values represent the mean±SD of at least three determinations. (C) The effects of C′-ARA54 and mt-ARA54 on PSA expression. Cell extracts from LNCaP cells stably transfected with pBIG2i (vector alone), pBIG2i-C′-ARA54, or pBIG2i-dn-mt-ARA54 cultured for 48 h, with 1 nM DHT in the presence or absence of 2 μg/ml doxy as indicated, were analyzed on Western blots using an antibody to the PSA. The 33-kDa of protein was detected as indicated and quantitated by Collage Image Analysis software (Fotodyne). The normalized expression level in the first lane (vector alone without doxy treatment) was set as 100%. Values represent the mean±SD of three separate experiments.

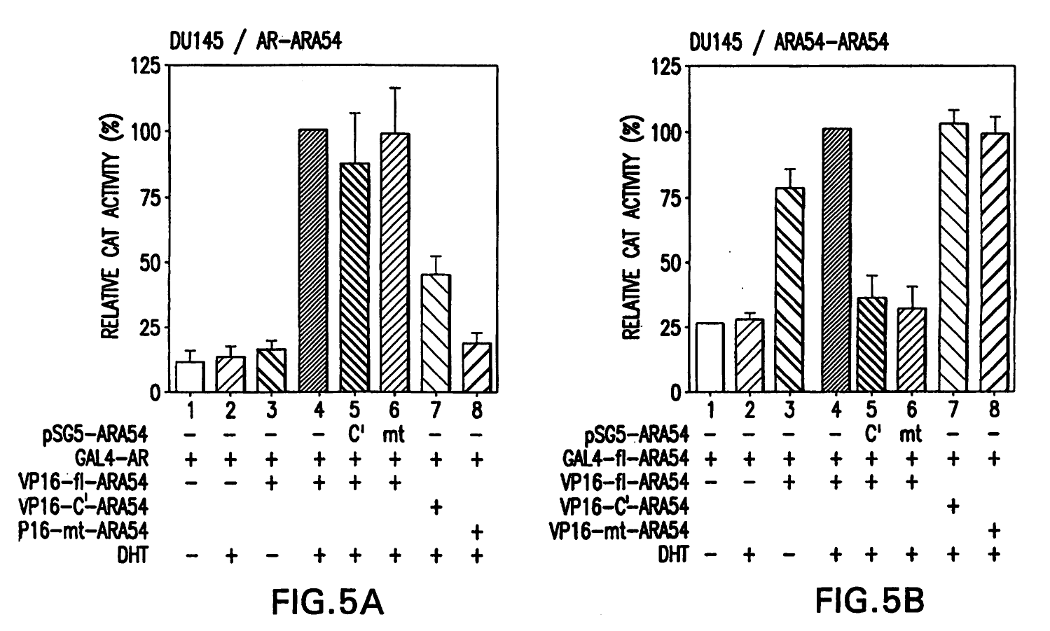

15. FIG. 5 shows the effects of C′-ARA54 and mt-ARA54 on AR-ARA54 and ARA54-ARA54 interactions. DU145 cells were transfected with 2.5 μg of GAL4-hybrid expression plasmid (pGAL0-AR (A) or pCMX-GAL4 DBD-fl-ARA54 (B)), 2.5 μg of VP16-hybrid expression plasmid (pCMX-VP16-fl-ARA54), and 2.5 μg of pG5-CAT, with or without 2.5 μg of pSG5-C′-ARA54 (C′) or pSG5-mt-ARA54 (mt). pCMX-VP16-C′-ARA54 and pCMX-VP16-mt-ARA54 were also cotransfected to test the interactions with AR (A) and fl-ARA54 (B). The total amount of DNA was adjusted to 11.0 μg with pSG5 and/or pVP16 for each transfection. The CAT activity was determined and each CAT activity is presented relative to that of lane 4 in each panel (black bars; set as 100%). Values represent the mean±SD of at least three determinations.

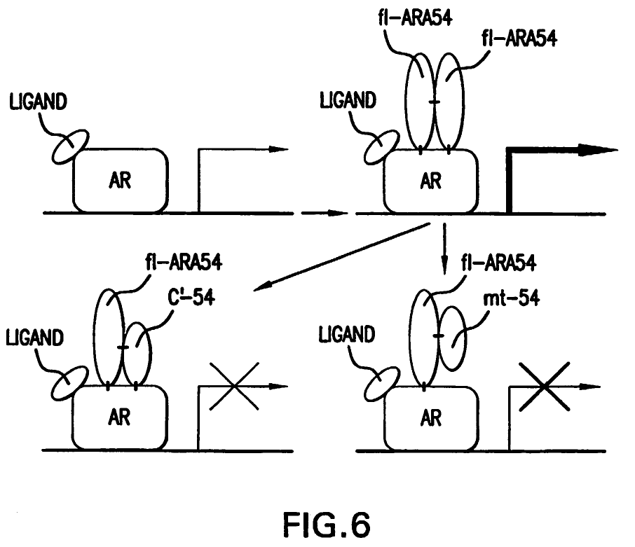

16. FIG. 6 shows a model for suppression of AR activity by C′-ARA54 and mt-ARA54. Fine and bold lines indicate the strength of transcription or inhibition.

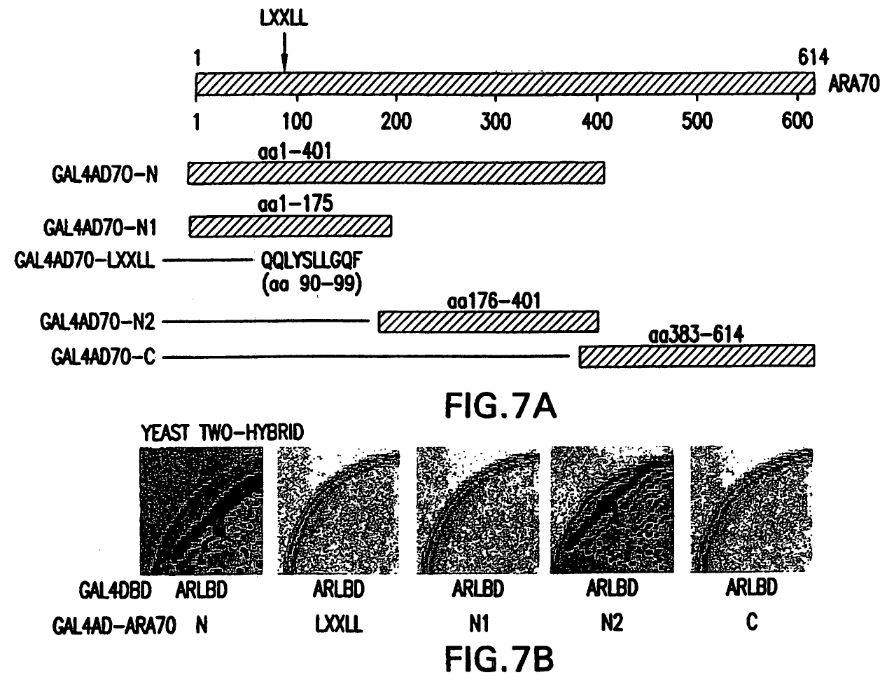

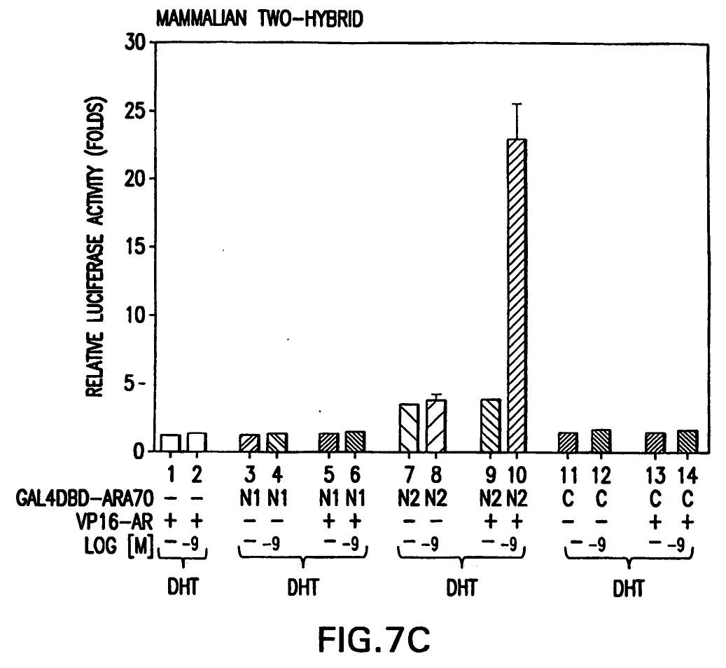

17. FIG. 7 shows the mapping the domains of ARA70 responsible for AR interaction. (A) Schematic diagram of the four GAL4AD-ARA70 fusion constructs, GALAD70-N: aa 1-401, GALAD70-N1: aa 1-175, GALAD70-N2: aa 176-401, GALAD70-LXXLL: aa 90-99 and GALAD70-C: aa 383-614, which were used to map the domains of ARA70 responsible for AR interaction. ARA70 residues are marked relative to translation initiation site. (B) The domains of ARA70 responsible for AR interaction by yeast two-hybrid assay. The interaction of different domains/motifs of ARA70 with wtAR assayed by plate nutritional selection in the yeast Y190 strain. GAL4AR, a fusion protein with the GAL4 DBD and an AR peptide containing part of the DBD, the whole hinge region, and the LBD (aa 595 to 918) was used as bait to test the interaction with different parts of ARA70. The interaction was tested by plate nutritional selection: the AR and ARA70 co-transformed yeast cells were selected for growth on plates with 20 mM 3-aminotriazole and 10 nm DHT but without histidine, leucine, or tryptophan. The colonies formed on plates with AR and ARA70-N, AR and ARA70-N2, but not on AR and ARA70-N1. Data were reproducible in two independent transformations. (C) The domains of ARA70 responsible for AR interaction by mammalian two-hybrid assay. DU145 cells in 60-mm dishes were transiently co-transfected with 3 μg of reporter plasmid pG5-Luc and 3 μg of GAL4 DBD fused ARA70 constructs, with or without 3 μg of VP16 fused AR, for 24 hours. Ten nM DHT was added for another 24 hours, and then the cells were harvested for the luciferase assay. Data represent the mean±S.D. of three independent experiments.

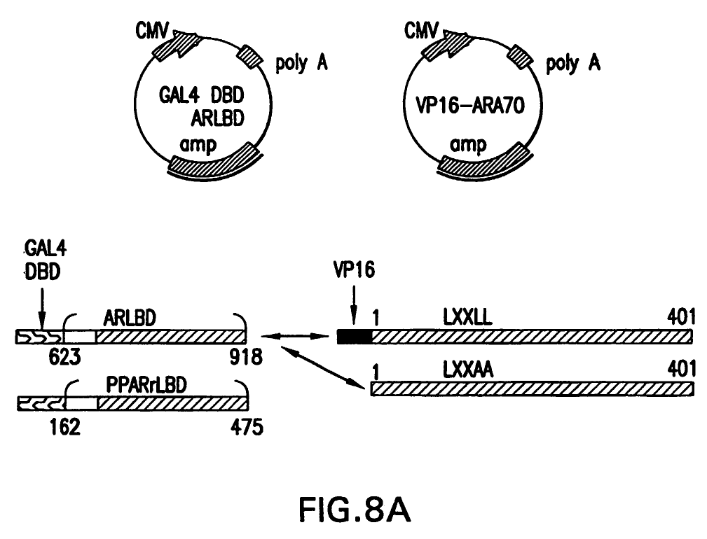

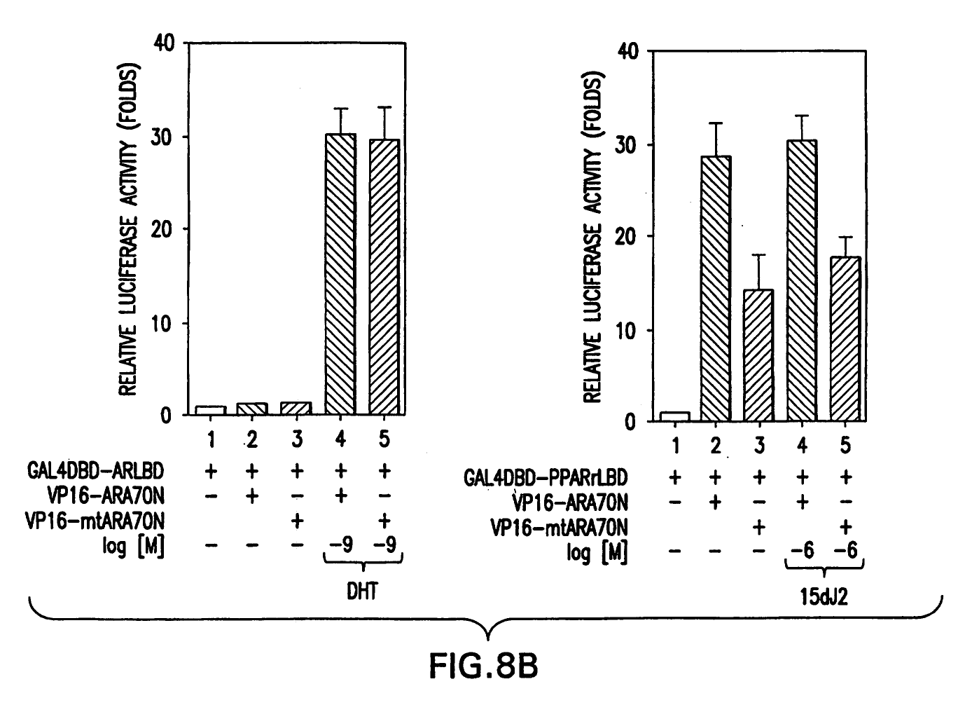

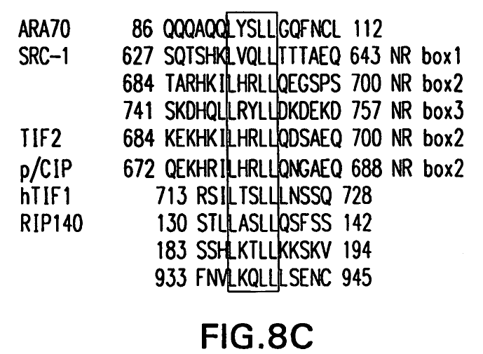

18. FIG. 8 shows the importance of ARA70 LXXLL motif for interaction with AR and PPARr. (A) Schematic diagram of GAL4 DBD fused AR and PPARγ, and VP16 fused wtARA70 and mtARA70 constructs generated by site-directed mutagenesis. (B) DU145 cells in 60-mm dishes were transiently co-transfected with 3 μg of reporter plasmid pG5-Luc and 3 μg of GAL4 DBD fused nuclear receptor constructs, with or without 3 μg of VP16 fused wtARA70 or mutant LXXAA, for 24 hours. Ten nM DHT or 1 uM 15dJ2 was added for another 24 hours, and then the cells were harvested for the luciferase assay. Data represent the mean±S.D. of three independent experiments. (C) Comparison of the consensus LXXLL motifs of ARA70 and other coregulators.

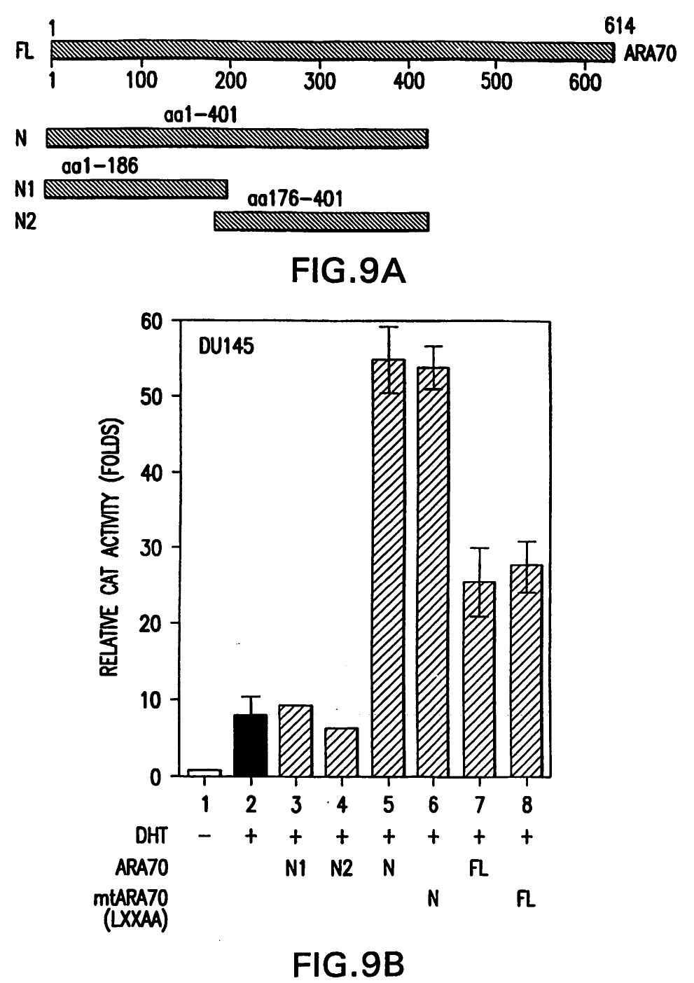

19. FIG. 9 shows the characterization of the influence of different ARA70 domains on AR-mediated transactivation in prostate cancer cells. (A) Schematic diagram of different pSG5-ARA70 constructs. (B) DU145 cells, transiently co-transfected with wtAR and different ARA70 constructs (lanes 3-8), were treated with 1 nM DHT for 24 hours. The cells were then harvested and whole cell extracts were used for the CAT assay.

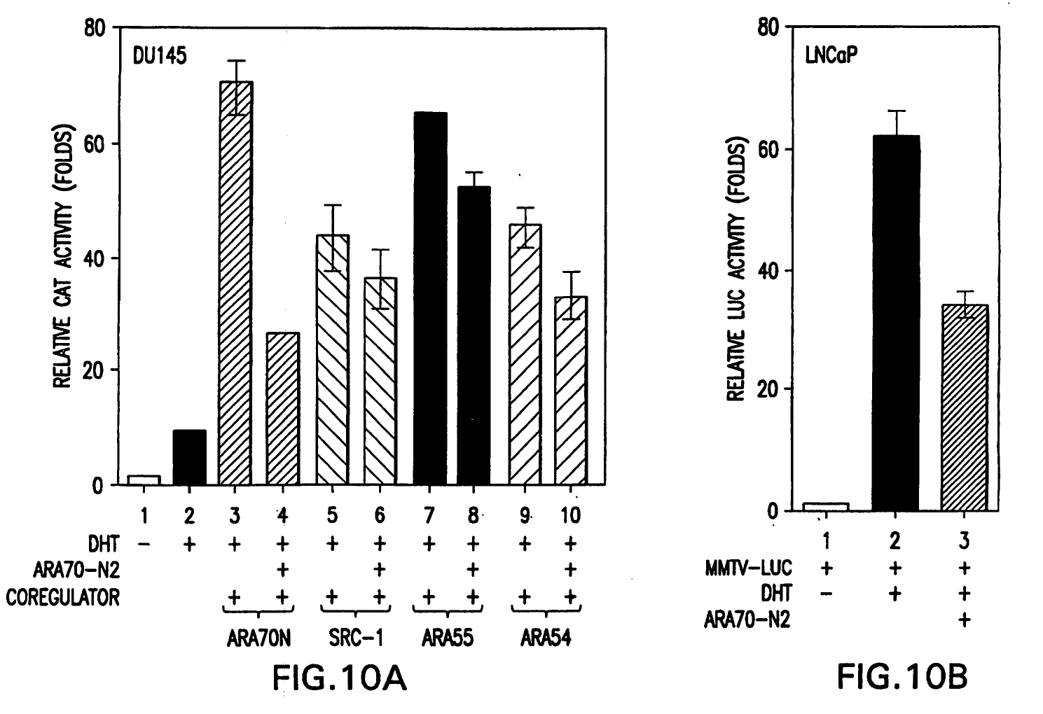

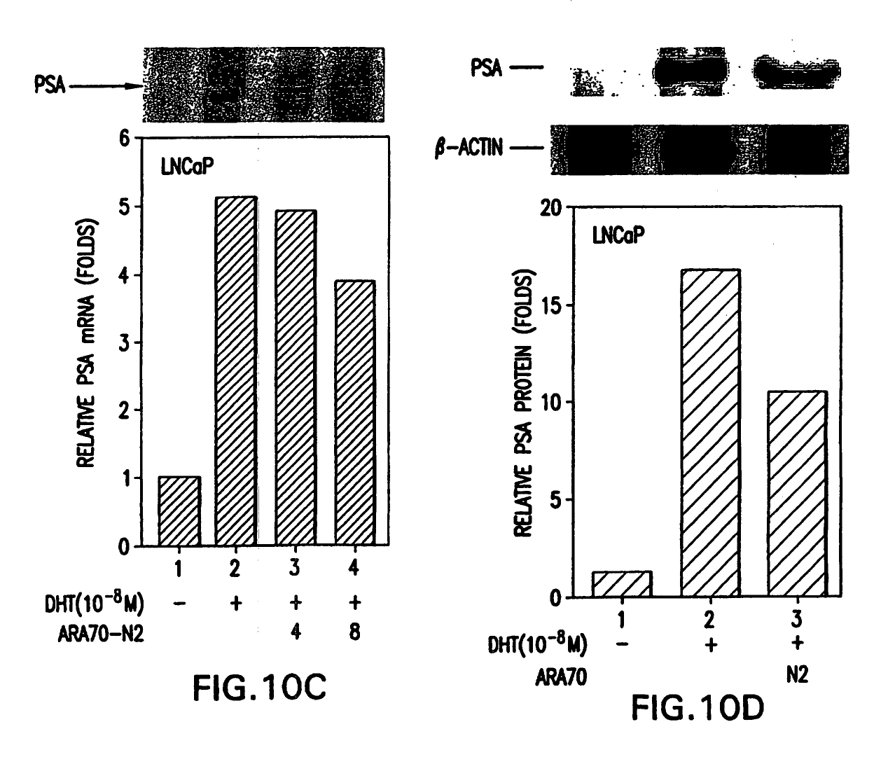

20. FIG. 10 shows the ARA70-N2 serves as a dominant-negative repressor of AR activity. (A) ARA70-N2 can serve as a dominant-negative to inhibit coregulator enhanced AR activity in DU145 cells. The pCMV-β-gal construct was used as an internal control, and the relative CAT activity was normalized by the β-gal activity. Data represent the mean±S.D. of four independent experiments. (B) ARA70-N2 can serve as a dominant-negative repressor to compete with the function of endogenous coregulators and inhibit AR transactivation in LNCaP cells. (C) ARA70-N2 can serve as a dominant-negative repressor to inhibit the expression of PSA mRNA in LNCaP cells. Human prostate cancer LNCaP cells were transfected with 4 and 8 μg of ARA70-N2 for 3 hours. One nM of DHT was then added for 24 hours before the cells were harvested for PSA northern blot analysis. The blot containing 20 μg total RNA in each lane was hybridized with a PSA specific cDNA probe. The 28S RNA was stained for equal RNA loading (data not shown). (D) ARA70-N2 can inhibit PSA protein expression in a dominant-negative manner. 4×10⁶ LNCaP cells were plated on 100-mm dishes 24 hours before transfection. 16 μg of plasmid DNA, as indicated in figure, was transfected into cells for 3 hours using Superfect (Qiagen). One nM of DHT or mock was added for another 24 hours, and then the cells were harvested for PSA western blot analysis. The blot containing 70 μg total cell lysate in each lane was hybridized with a PSA specific antibody. The same membrane was hybridized with a specific antibody for β-actin for equal protein loading.

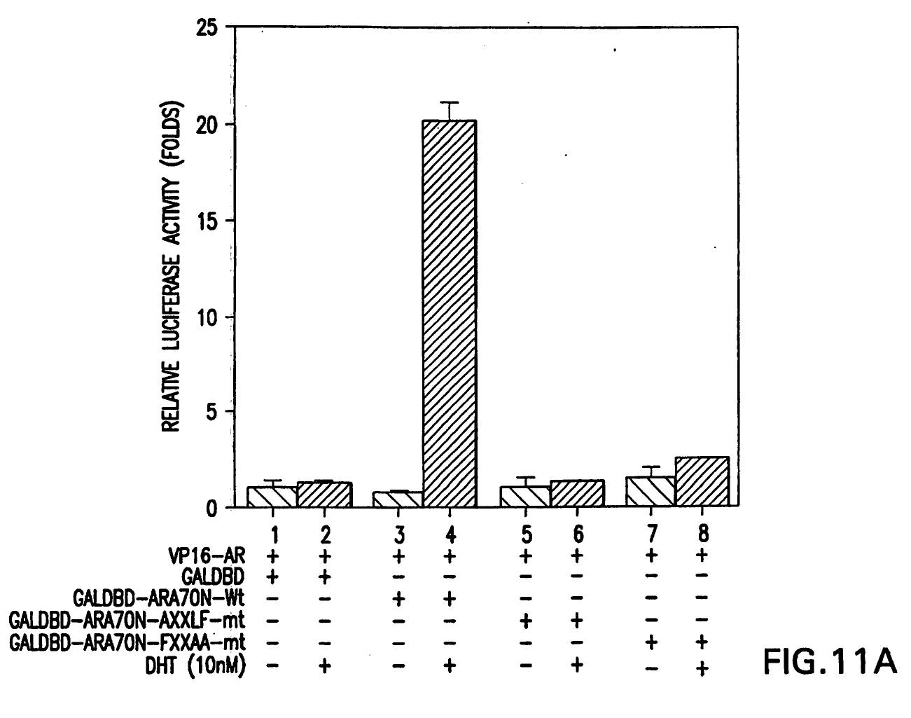

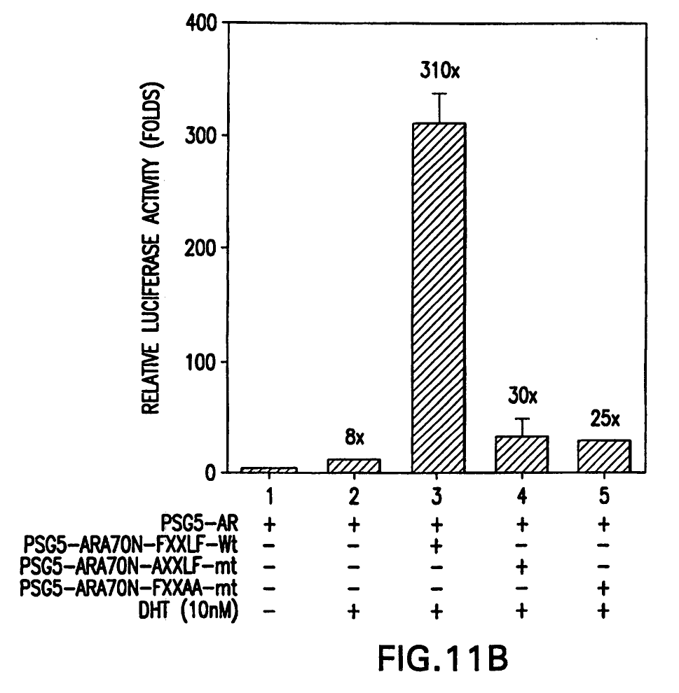

21. FIG. 11 shows the effect of wild type and mutant FXXLF motifs ARA70N on AR interaction in COS-1 cell line. Total 1 μg plasmid which contains 350 ng VP16-AR, 300 ng reporter pG5-LUC, and 0.5 ng SV40-Renila Luciferase was transfected to COS-1 cells without (lane 1, 3, 5, and 7) or with (lane 2, 4, 6, and 8) 10 nM testosterone. Further adding GAL-DBD (lane 1 and 2) or GAL-DBD-ARA70N with wild type (lane 3 and 4) or mutant (lane 5-8) FXXLF motifs to the cells. (B) Effect of wild type and mutant FXXLF motifs ARA70N on AR transactivation in COS-1 cells. Total 1 μg plasmid was transfected with fixed 40 ng pSG5-AR and 200 ng reporter plasmid MMTV-LUC to the cells cultured in a 24 wells plate without or with 10 nM testosterone. 0.5 ng SV40-Renila Luciferase was used as an internal control. Relative luciferase activity was calculated by dual luciferase system.

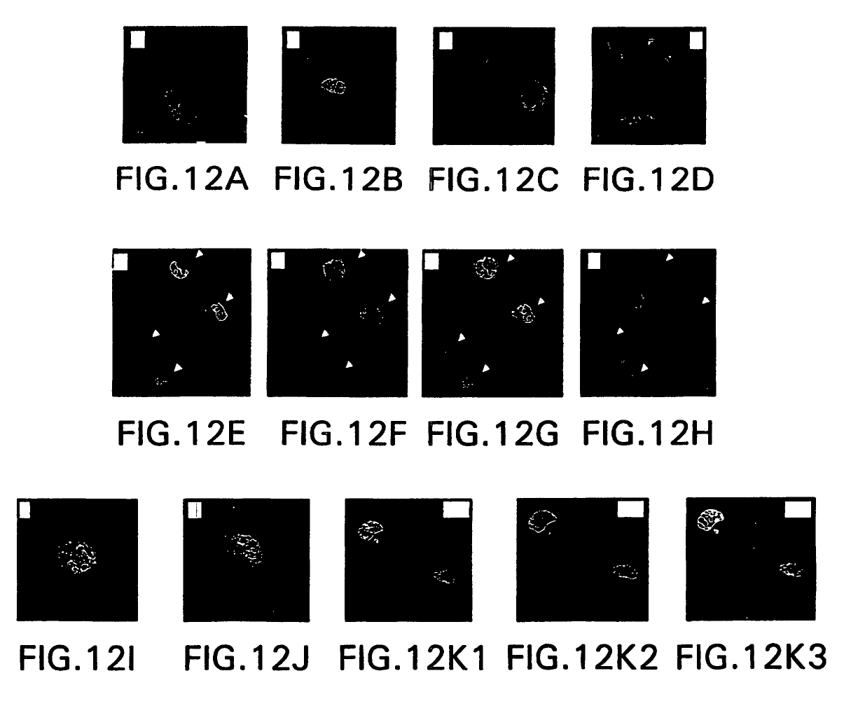

22. FIG. 12 shows the immunocytofluorescence detection of the AR and ARA70 in COS-1 cells. COS-1 cells were seeded on two-well Lab tek II chamber slides (Nalge) 24 hours before transfection. Two micrograms of DNA per 10⁵ cell was transfected with the AR, with or without ARA70, using FuGENE6 transfection reagent (Boehringer-Manheim). Twenty-four hours after transfection, the cells were treated with 10 nM DHT or ethanol. Immunostaining was performed by incubation with the rabbit anti-AR polyclonal antibody (NH27) or mouse anti-ARA70 monoclonal antibody (CC70), followed by incubation with either fluorescence-conjugated goat anti-rabbit or anti-mouse antibodies (ICN). The red signal represents the AR and the green signal represents ARA70. Blue DAPI staining was used to show the location of nuclei. (A) AR staining without DHT. (B) AR staining with DHT treatment. (C) ARA70-FL staining without DHT treatment. (D) ARA70-FL staining with DHT treatment. (E-H) The co-transfection of the AR and ARA70-FL with staining for both proteins in the same field. The cells expressing the AR only are indicated with yellow arrows, and the cells expressing the AR and ARA70-FL are indicated with white arrows: (E) staining for AR-Texas red, (F) staining for ARA70-FITC, (G) overlay (H) DAPI staining represents total cell nuclei in this field. (I-K) Enhancing the nuclear translocation of ARA70-N (aa 1-401) in the presence of androgen and the AR. FITC staining represents ARA70-N. Only FITC staining is shown for 1 and J. (I) 10 nM DHT treatment in the absence of the AR, (J) coexpression with the AR in the absence of ligand, (K) coexpression with the AR in the presence of 10 nM DHT. In the same field: K-1 indicates ARA70 staining; K-2 indicates the AR staining; K-3 indicates the overlay of both fluorochromes. Color pictures were produced by confocal microscopy.

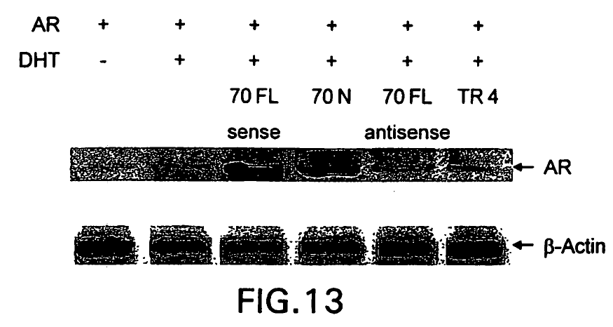

23. FIG. 13 shows the ARA70, but not antisense ARA70 and TR4, enhances the amount of AR protein. COS-1 cells were transfected with 5 μg of AR and 5 μg of empty vector, or 5 μg of ARA70-FL, or 5 μg of TR4. Nuclear extracts were prepared and 30 μg of nuclear extract was applied for western blotting with polyclonal anti-AR antibody (NH27).

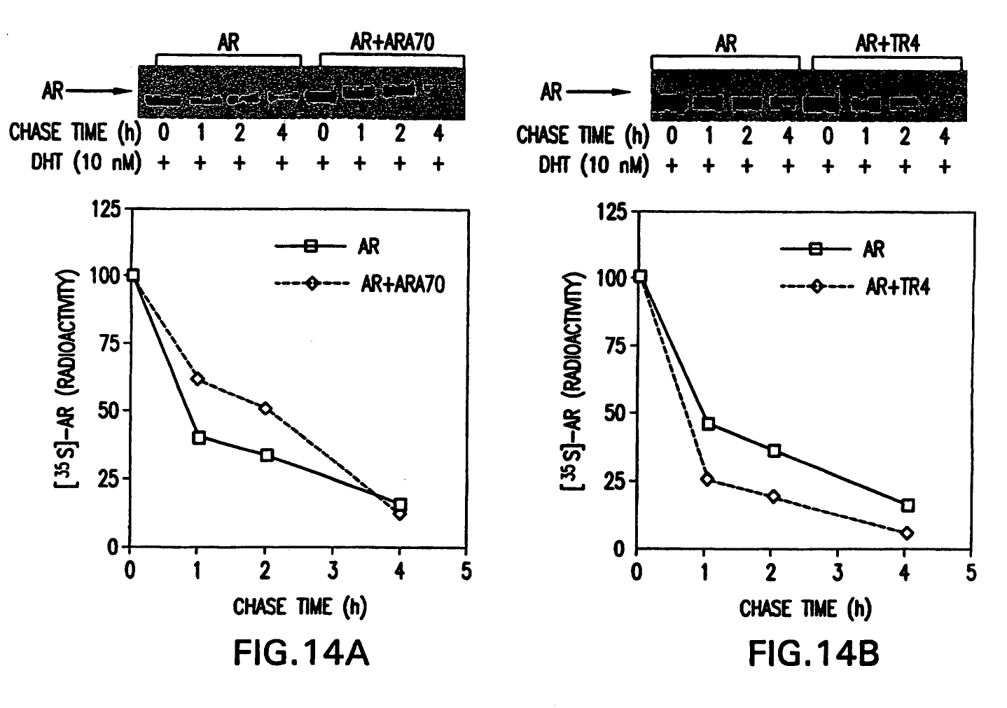

24. FIG. 14 shows the ARA70 enhances the metabolic stability of the AR. COS-1 cells were incubated as indicated and subjected to pulse-chase metabolic labeling of AR with [³⁵S] methionine/cysteine for 30 minutes. After changing the medium, the cells were harvested at the times indicated in the figure. Whole cell extracts were prepared by RIPA buffer and immunoprecipitated with a polyclonal anti-AR antibody (NH27). The cells were transfected with 5 μg of AR and 5 μg of vector, or 5 μg of ARA70-FL or 5 μg of TR4. In addition to the AR, ARA70, or TR4, the cells were co-transfected with 40 ng of Renilla luciferase expression construct as a transfection control. The specificity of the immunoprecipitation was confirmed using preimmune serum as well as protein A-Separose beads alone (data not shown). The AR signals were normalized with internal control Renilla luciferase activity.

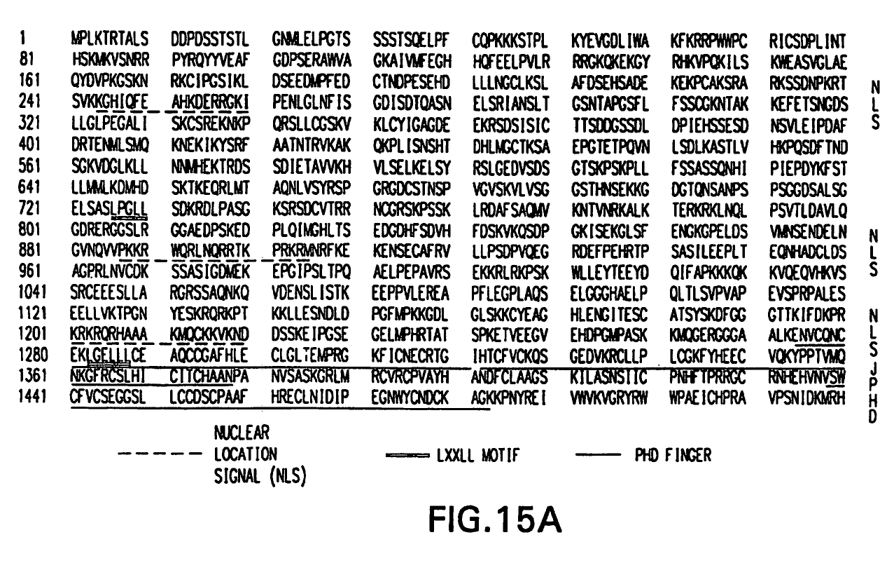

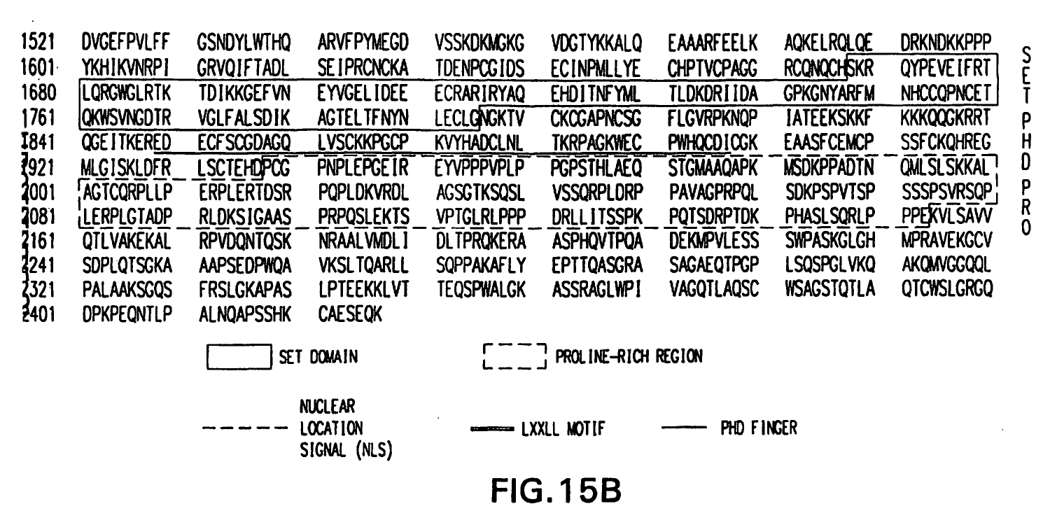

25. FIG. 15 shows the amino acid alignment of human ARA267. The open reading frame of ARA267 encodes 2427 amino acids. Some potential functional domains were boxed or underlined. Based on database search, ARA267 contains one Cysteine-rich region (aa 1277-1342), one SET domain (aa 1668-1795), two LXXLL motifs (aa 726-730 and aa 1283-1287), three NLS (aa 243-260, aa 888-905, and aa 1202-1219), and four PHD fingers (aa 1274-1320, aa 1321-1377, aa 1438-1482, and aa 1849-1896) as indicated.

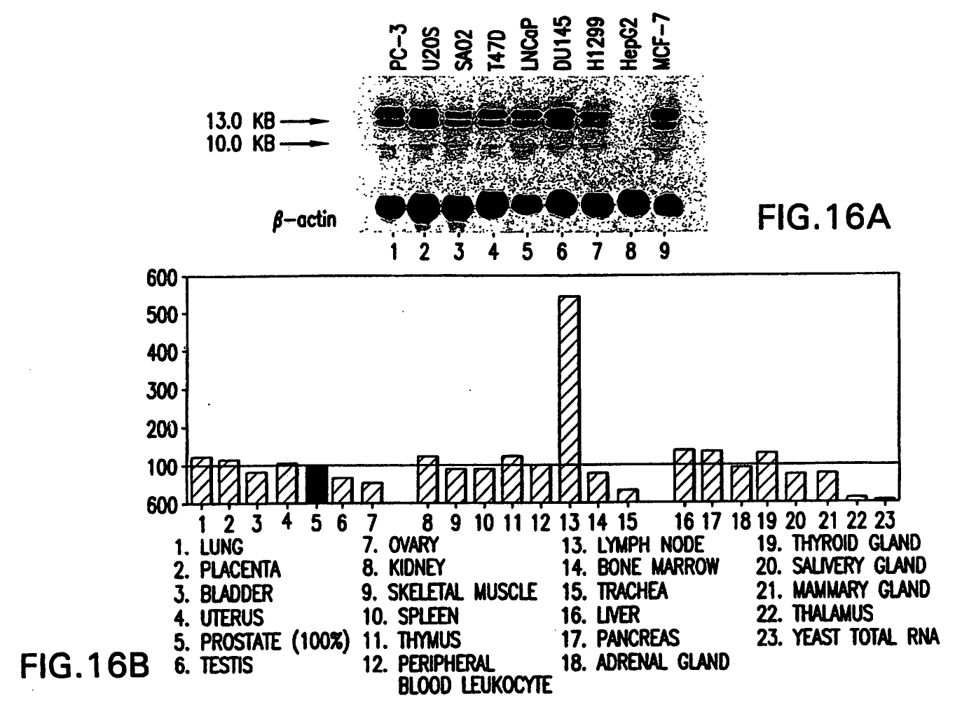

26. FIG. 16 shows the tissue distribution of ARA267 by Northern blot and dot blot. (A) Northern blot analysis indicated that ARA267 is expressed as a mRNA of 13.0 Kb and 10.0 Kb in many cell lines including, PC-3, U2OS, SAO2, T47D, LNCaP, DU145, H11299, and MCF-7 (lanes 1-7 and 9), but is absent in HepG2 cell line (lane 8). (B) Multiple tissues dot blots were used to determine the expression of ARA267 in different tissues, including prostate, testis, adrenal gland, liver, ovary, thymus, etc. The relative expression of ARA267 was indicated, using prostate as 100%. In lung, placenta, uterus, kidney, thymus, lymph node, liver, pancreas, and thyroid gland tissues (lanes 1, 2, 4, 8, 11, 13, 16, 17, and 19) the ARA267 expression is greater than 100% and the rest are lower than 100% (lanes 3, 6, 7, 9, 10, 12, 14, 15, 18, 20, 21, 22, and 23).

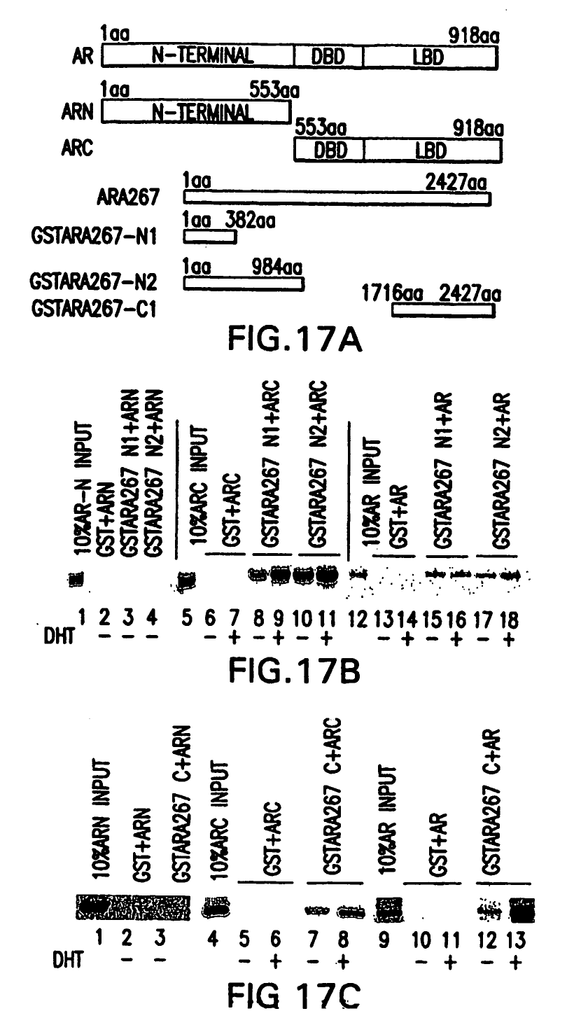

27. FIG. 17 shows the interaction between ARA267 and AR. (A) Maps of the domains of AR used for ARA267 interaction and three recombinant GST-ARA267 fusion proteins, GST-ARA267N1, GST-ARA267N2, and GST-ARA267C. (B) All GST fusion proteins were generated in Escherichia coli as described. 5 μl of in vitro translated [³⁵S]-methionine-labeled AR-N (aa 36-553), AR-C (aa 553-918), and AR full-length was used to perform the GST pull-down assay. 10% TNT expressed AR-N, AR-C, and AR full-length ³⁵S-methionine-labeled products were loaded as controls (lanes 1, 5, and 12). GST only was the control in the absence and presence of DHT, (lanes 2, 6, and 13) and (lanes 7 and 14) respectively. Both GST-ARA267N1 and GST-ARA267N2 can not pull-down AR-N (lanes 3, 4), but can pull-down AR-C and AR full-length in presence and absence of 1 μM DHT (lanes 8-11) and (lanes 15-18), respectively. (C) GST-ARA267C 10% TNT expression of AR-N, AR-C, and AR full-length [³⁵S]-methionine-labeled products were used as controls (lanes 1, 4. and 9). GST only also used in (lanes 2, 5, 6, 10 and 11) and GST-ARA267C can not pull-down AR N-terminal (lane 3) but can pull-down both AR-C and AR full-length in presence and absence of 1 μM DHT (lanes 7 and 8) and (lanes 12 and 13) respectively.

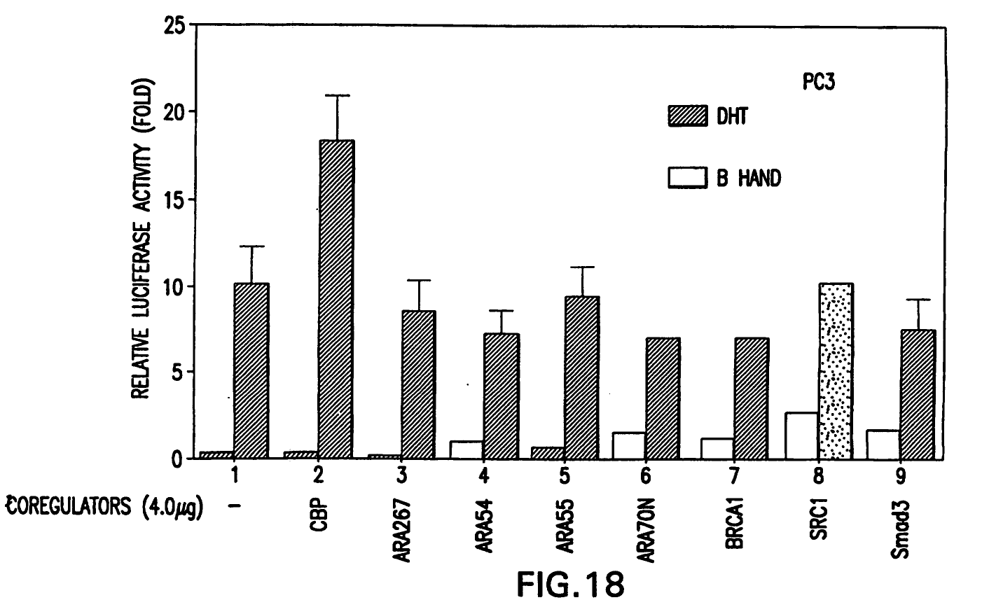

28. FIG. 18 shows ARA267 does not affect the interaction between N-terminal and C-terminal of AR. PC-3 cells in 60-mm dishes were transiently transfected with 3 μg of the report gene plasmaid pG5-LUC), 2 μg each of Gal4 DBD fused AR C-terminal and VP16 fused AR N-terminal, and 10 ng SV40-PRL plasmid. Cells also were transfected without or with 4 μg pSG5ARA267 (lanes 1, 3 respectively) and other AR coregulaters in absence and presence of DHT as indicated. The luciferase activity of the interaction between Gal4ARC and VP16ARN in the absence of coregulateor and DHT was standardized to one fold. All values represent the mean+/−SD of three independent experiments.

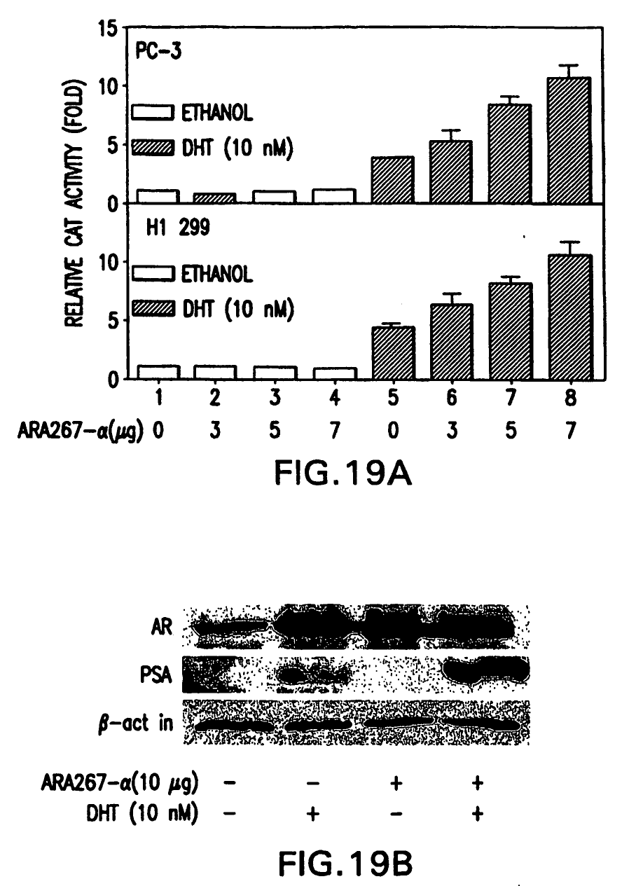

29. FIG. 19 shows the effects of full-length ARA267 on AR transactivation. (A) PC-3 and H1299 cells in 60-mm dishes were transiently co-transfected with 3 μg of MMTV-CAT reporter gene, 1 μg of AR expression vector (pSG5AR), and increasing amounts of full-length ARA267 as indicated, using the calcium phosphate precipitation method. The total amount of plasmid was adjusted by pSG5 vector to 11 μg for each transfection. Cells transfected without pSG5-ARA267 (lanes 1 and 5) and with increasing concentrations: 3, 5, and 7 μg of pSG5-ARA267 (lanes 2-4 and 6-8) in the absence (open bars) and presence (closed bars) of DHT indicated that ARA267 enhanced AR transcription activity in a ligand dependent manner. The CAT activity of without ARA267 and DHT was set as one fold. All values represent the mean+/−SD of three independent experiments. (B) The endogenous PSA expression was further induced by ARA267 in presence of 10 nM DHT. LNCaP cells were transfected with ARA267 and parental vector as indicated in 10 cm dishes by Superfect. After 2 hours of transfection, the medium was changed, and ethonal and 10 nM DHT were applied for another 36 hours. In each experiment, 50 μg of whole-cell extract was applied for the Western blotting.

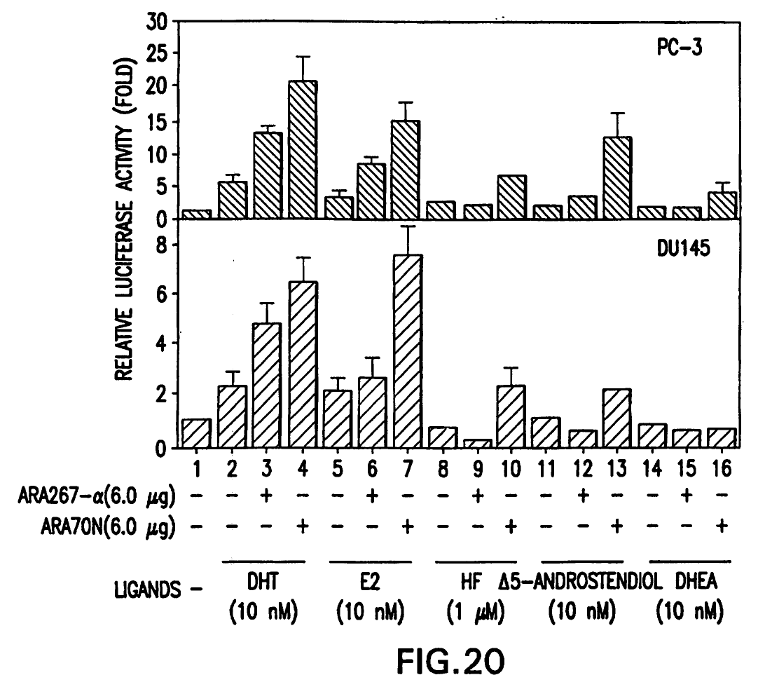

30. FIG. 20 shows ARA267 effect on AR transactivation with different ligands. PC3 and DU145 cells were transiently co-transfected with 3 μg of MMTV-LUC reporter gene, 1 μg of pSG5-AR and 6 μg ARA267, 6 μg ARA70N as indicated then treated without or with different ligands 10 nM DHT, E2, Adiol, DHEA and 1 mM HF. After 24 hours, luciferase assay was performed. The luciferase activity of AR without coregulator and ligands was set as one fold. (the first bar). All values represent the mean+/−SD of three independent experiments

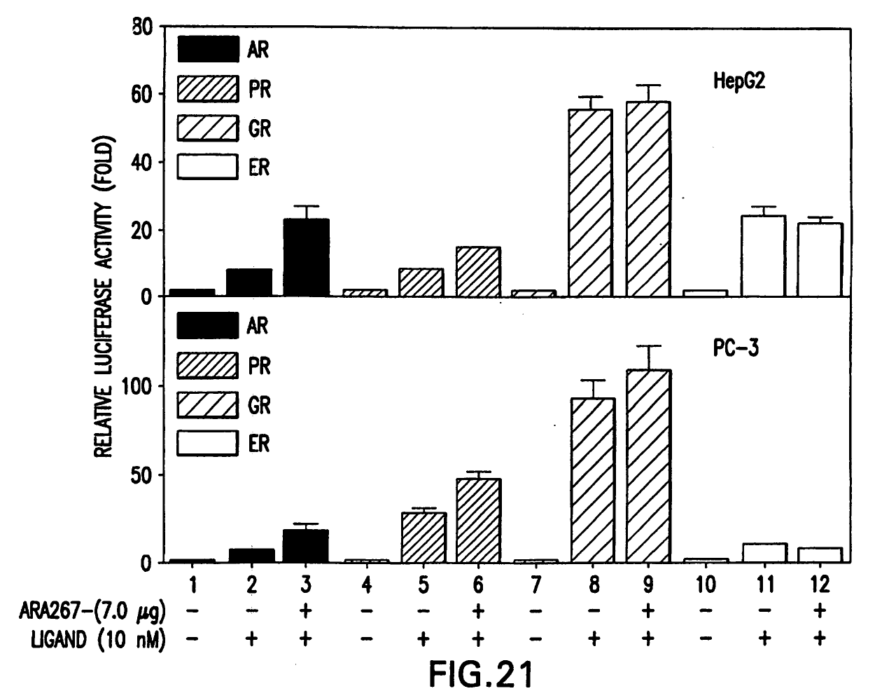

31. FIG. 21 shows Full-length ARA267 effect on AR and other steriod receptor transcription. HepG2 cells (an ARA267 negative cell line) and PC3 cells were co-transfected with 1.0 μg various nuclear receptor gene plasmids, 3 μg reporter gene plasmids (MMTV-luciferase plasmid for AR, PR, and GR, Lanes 1-3, 4-6, 7-9 and ERE-luciferase plasmid for ER lanes 10-12), 10 ng of SV40-pRL and 7 μg pSG5-ARA267 plasmids in the absence and presence of 10⁻⁸ M various ligands DHT, progestrone, DEX 17β-estradiol (E2), respectively as indicated. The luciferase activity of each receptor without ARA267 and ligands was set as one fold. All values represent the mean SD of three independent experiments.

32. FIG. 22 shows that ARA267 additionally enhances AR transactivation with other AR coregulators. PC3 cells were cotransfected with 2 μg of pG5-LUC, 10 ng SV40-pR1, 0.5 μg pSG5-AR and ARA267, ARA24, PCAF alone or togather with different dosage as indicated in the presence and absence of 10 nM DHT. The luciferase activity of AR without ARA267 and ligand was set as one fold. All values represent the mean+/−SD of three independent experiments.

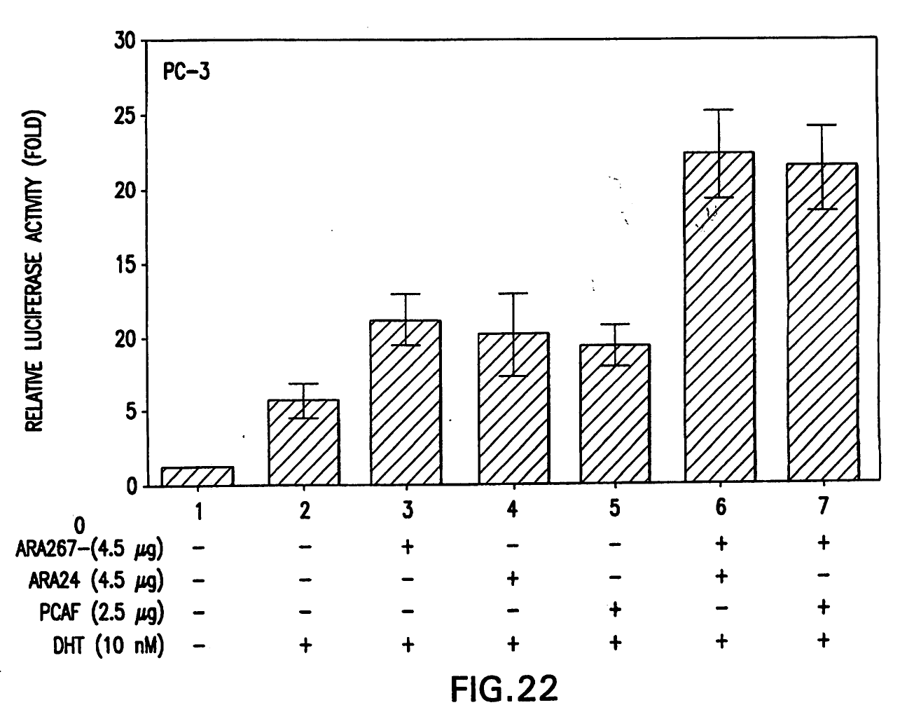

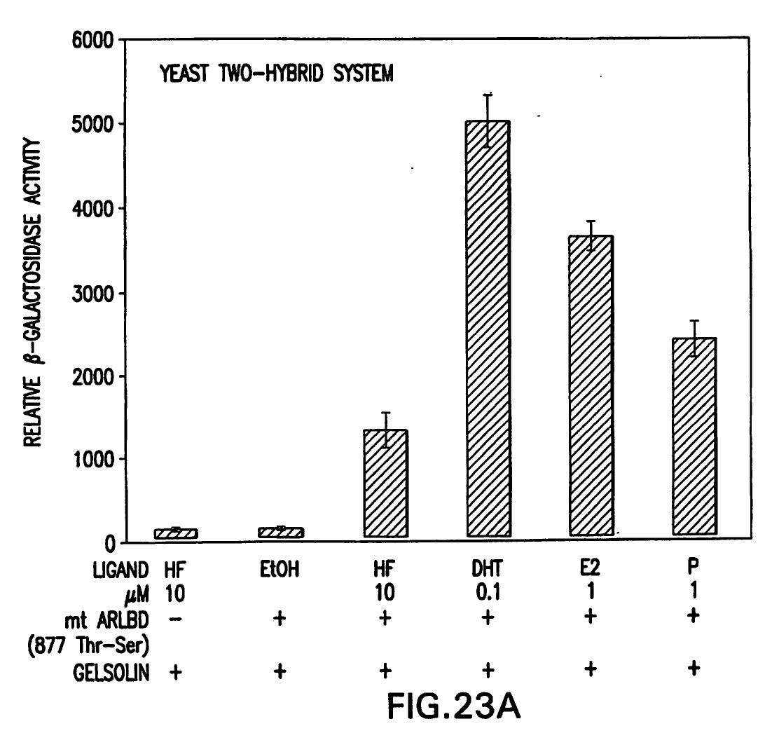

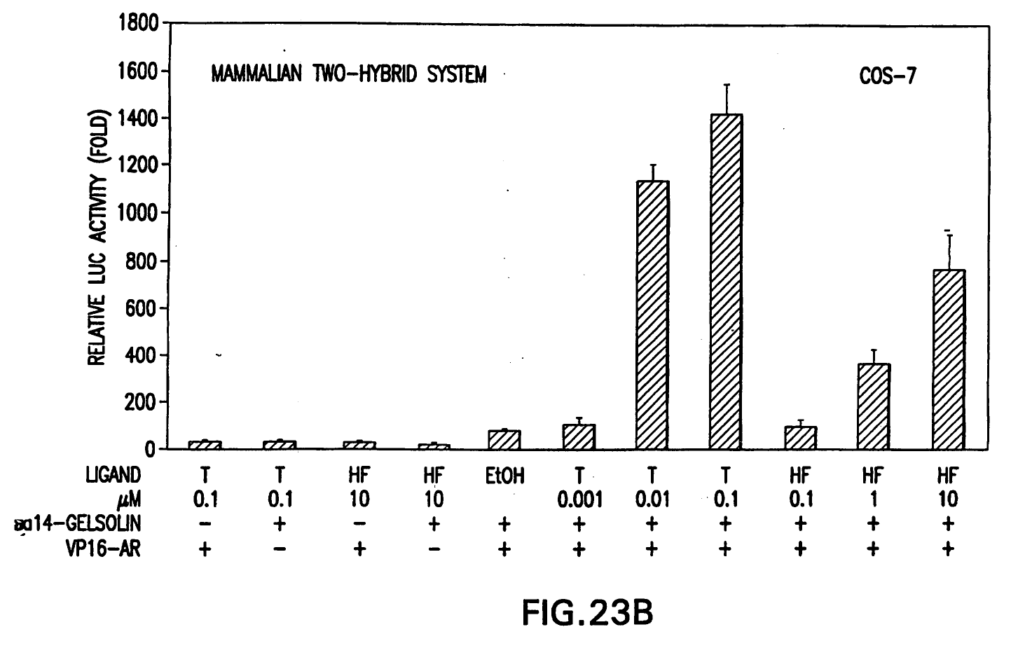

33. FIG. 23 shows that AR interacts with gelsolin in two-hybrid assays. (A) Y190 yeast cells were transformed with Gal4 DBD fused with the C-terminus (aa 595-918) of mtARt877s and Gal4AD fused with gelsolin (aa 281-731). Transformants were selected by their growth in the presence of DHT, HF, P, E2, or EtOH vehicle, and assayed for liquid β-gal activity as described previously (4). (B) COS-7 cells were transfected with expression vectors for C-terminus (aa 281-731) of gelsolin fused with Gal4, AR (aa 36-918) fused with VP16, pG5-LUC reporter and internal control pRL-CMV reporter. Relative LUC activity was determined as Gal4-LUC activity relative to control LUC activity.

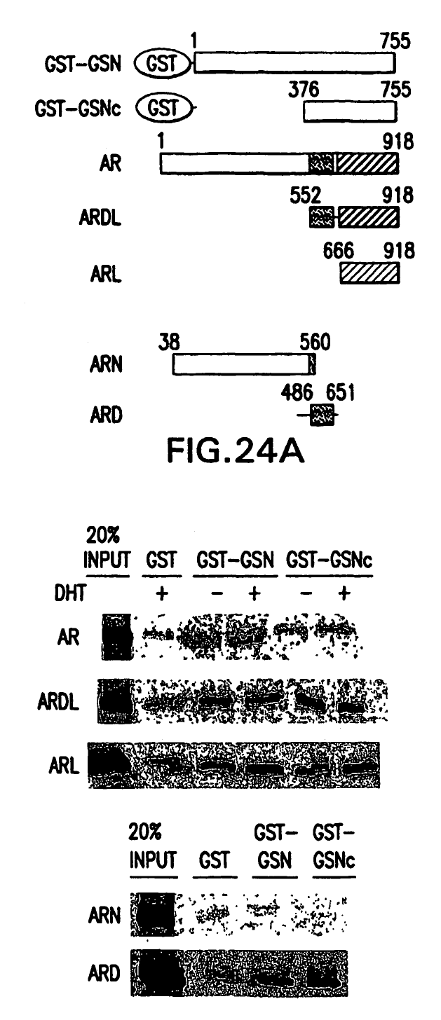

34. FIG. 24 shows that the interaction domain between gelsolin and AR. (A) The diagram of GST-GSN fusion proteins and AR functional domain used in GST-pull down assay (B) GST fusion proteins were expressed and purified by GSH-conjugated beads. AR fragments in vitro translated and labeled by ³⁵S-methionine were incubated with GST proteins. Protein complexes pulled down by GST proteins were separated on SDS-PAGE and visualized by Phosphorimager.

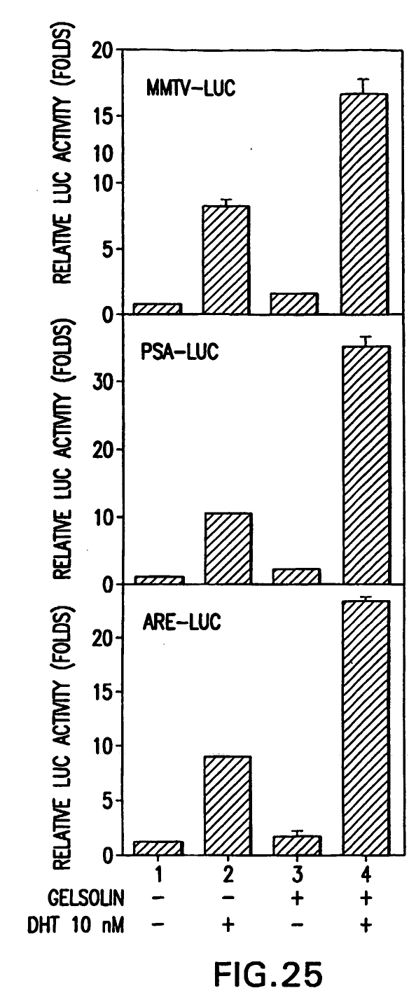

35. FIG. 25 shows that gelsolin overexpression enhances AR transcription activity. DU145 cells were co-transfected with pSG5-AR, pSG5-gelsolin, pRL-SV40, and reporter gene as indicated by using SuperFect. Cells were treated with EtOH or DHT and then lysed for LUC activity assay. The Firefly LUC activity from AR reporter gene was normalized by Renilla LUC activity. After measuring the LUC activity, values relative to lane 1 were calculated. Results are the mean±S.D. of three independent experiments.

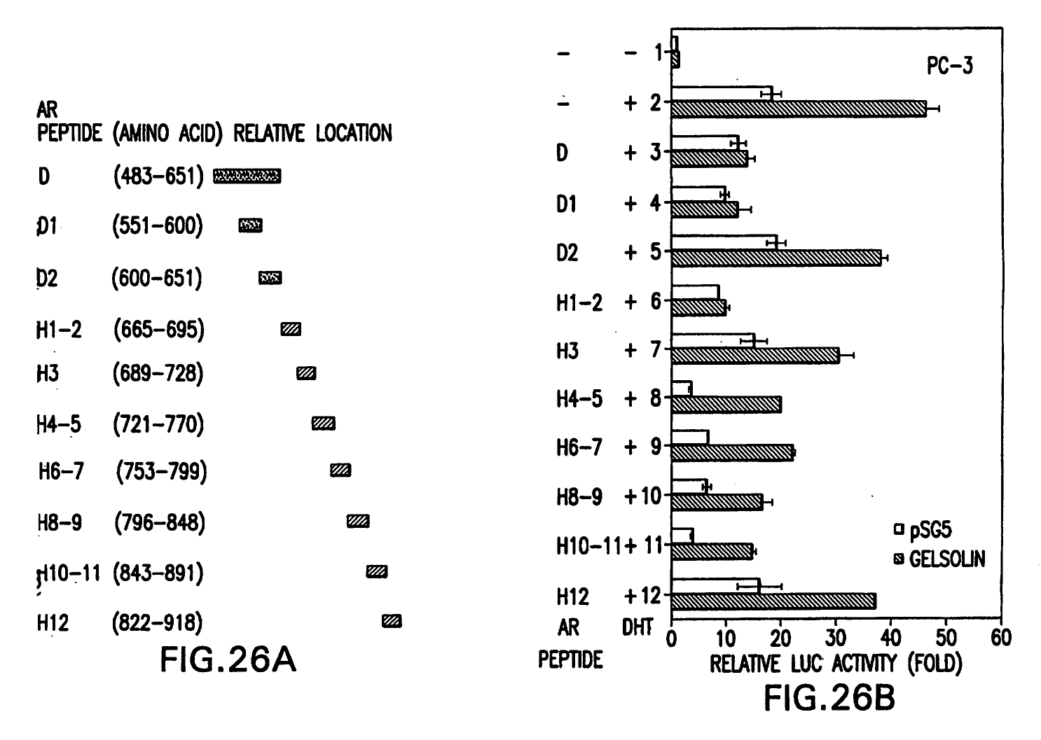

36. FIG. 26 shows that overexpression of AR peptides interrupts gelsolin enhancing AR activity. (A) The design of AR peptides, the amino acids and relative location they represent. (B) PC-3 cells were co-transfected with AR, pSG5 (O) or pSG5-gelsolin (v), MMTV-LUC. pRL-SV40, and flag-AR peptides expression plasmids by using SuperFect. Cells were treated with EtOH or DHT and then lysed for LUC activity assay as described in FIG. 4.

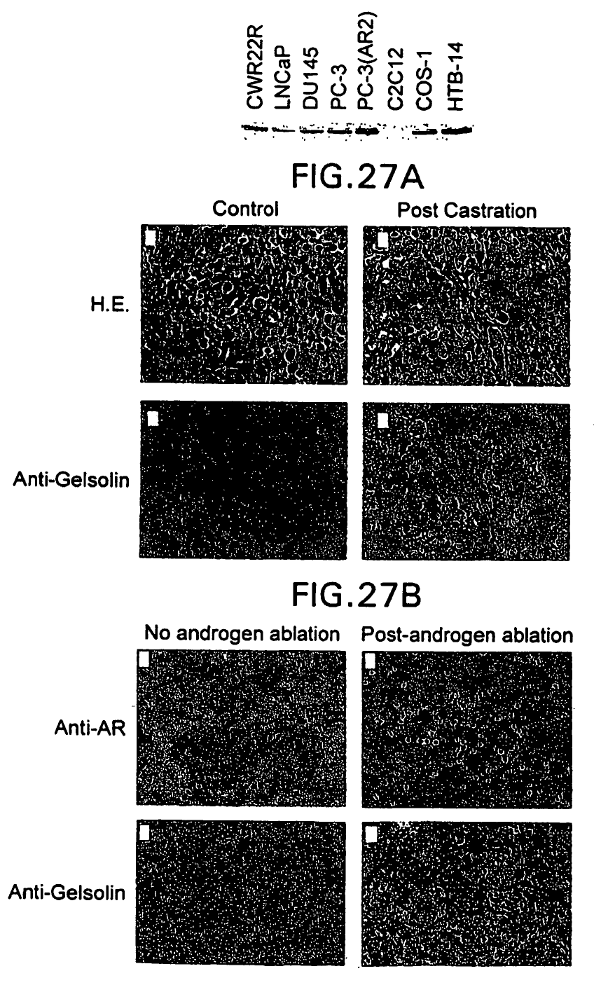

37. FIG. 27 shows that gelsolin expression is increased in prostate cancer after androgen ablation. (A), Western blot analysis for gelsolin in human prostate cancer cell lines, CWR22, LNCaP, PC3, DU145, and other cell lines, C2C12, COS-1, HTB-14. (B), LNCaP xenografts in nude mice after castration (b, d) versus sham operation (a, c). HE, hemotoxylin and eosin staining (a, b). Immunohistochemical staining of gelsolin (c, d). Note more intensive immunostaining in d versus in c. (C) Human prostate cancer specimens treated with (b, d) or without (a, c) androgen ablation therapy. Immunohistochemical staining of AR (a, b) and gelsolin (c, d). Note more intensive immunostaining in d versus in c.

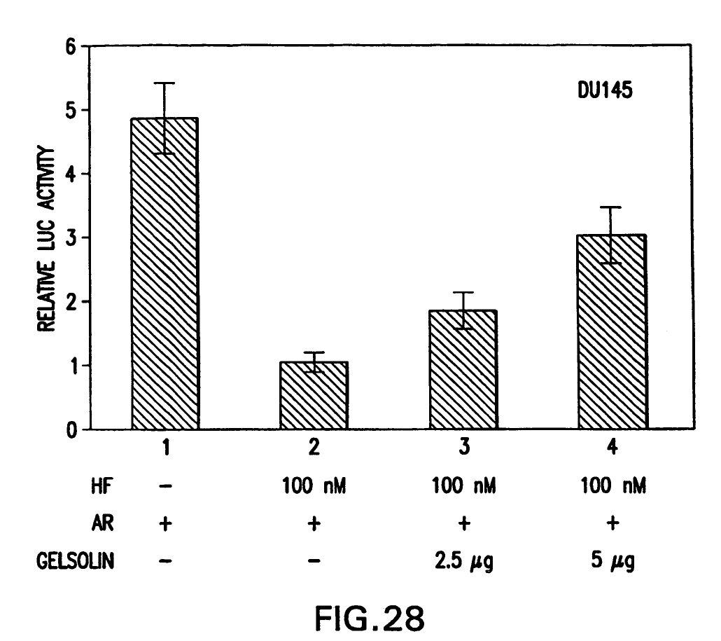

38. FIG. 28 shows that gelsolin promote the androgenic activity of HF. The cells were transfected with expression vectors for either empty vector pSG5 or pSG5 plus increasing amount of full-length gelsolin as indicated. EtOH or HF was added in the normal serum supplemented medium. Relative LUC activities were calculated using the activity of AR in the absence of gelsolin and the presence of HF as 1.

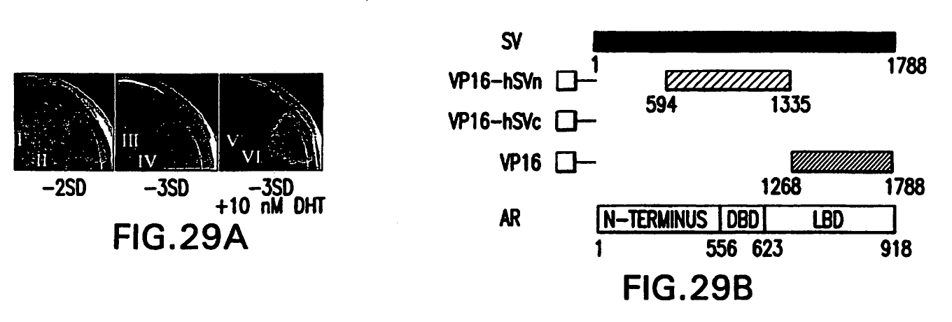

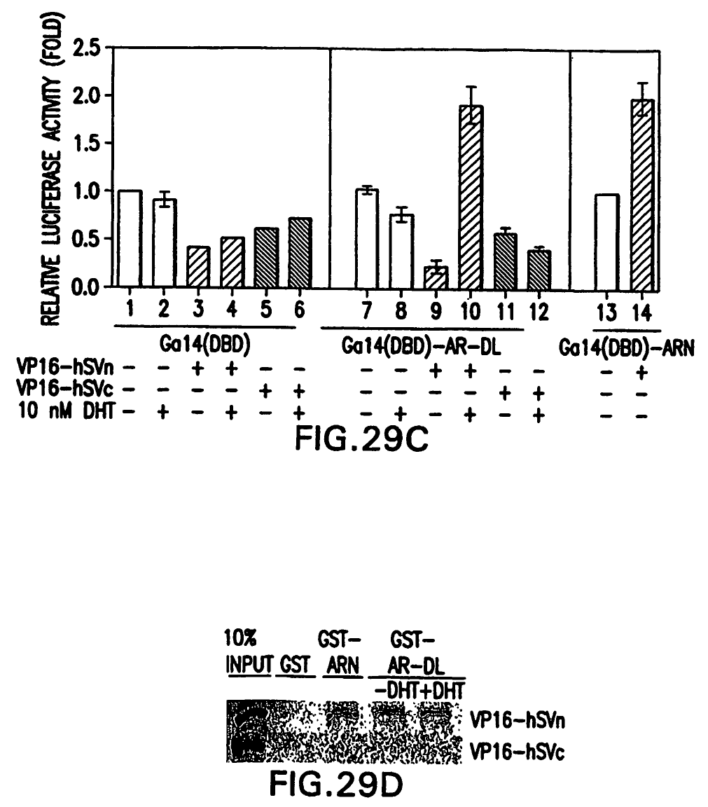

39. FIG. 29 shows that supervillin fragments interact with AR in yeast two-hybrid, mammalian two-hybrid and GST pull-down assays. (A) Yeast two-hybrid assay demonstrated the interaction between AR and SV. Yeast strain Y190 was co-transformed with pAS-AR and pACTII or pACTII-SV(595-1788). After transformation, yeast were plated on −2SD nutrition selection plates and cultured in 30° C. incubator for 3 days. Colonies were selected and plated on −2SD, −3SD, and −3SD+10 nM DHT nutrition selection plates. I, III, V are the yeast transformed with pAS-AR and pACTII; II, IV, VI are the yeast transformed with pAS-AR-DL and pACTII-SV(595-1788). The growth of yeast was observed after 3 days culture in 30° C. incubator. (B) Diagram of VP16-hSV constructs and AR functional domains. (C) Plasmids expressing Gal4(DBD), Gal4(DBD)-AR-DL or Gal4(DBD)-ARN were co-transfected with VP16-SVn or VP16-SVc expression plasmids into COS-1 cells. Gal4 response element controlled luciferase reporter gene, G5-Luc, was used to detect the interaction and pRL-SV40 was used for internal control. After 16 h transfection, 10 nM DHT or EtOH were added for another 16 h. Cells were harvested and assayed for luciferase activity. The activities relative to VP16 alone without ligand were calculated. Results are the mean±S.D. of three independent experiments. (D) GST protein and two GST fusion proteins containing AR N-terminus (GST-ARN) and AR DBD plus LBD (GST-AR-DL) were expressed in bacteria and purified by GSH-beads. SV fragments were expressed by in vitro translation and labeled by ³⁵S-methionine. After incubation of SV fragment and GST-AR with EtOH or 1 μM DHT, pulled down proteins were loaded on gel and detected by PhosphorImager.

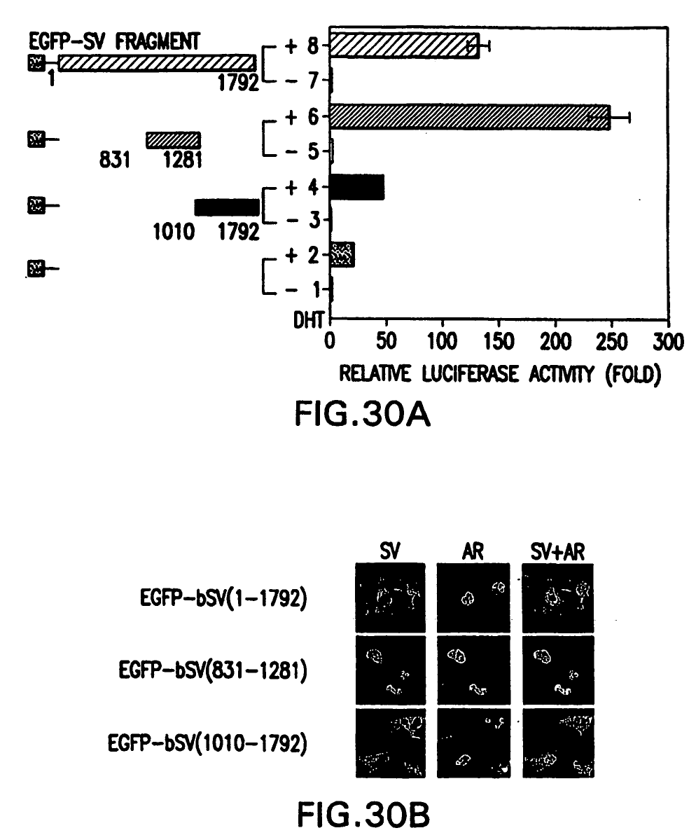

40. FIG. 30 shows the functional domain and cellular localization of SV fragment with AR. (A) 1.5 μg plasmids expressing EGFP only or EGFP-bSV fragments were co-expressed with 30 ng pCMV-AR, 0.5 μg MMTV-Luc and 1 ng pRL-SV40 into COS-1 cell. Cells were treated with EtOH or 10 nM DHT as indicated for 20 h. The Firefly luciferase activity from AR reporter gene, MMTV-Luc, was normalized by Renilla luciferase activity. After measuring the luciferase activity, values relative to lane 1 were calculated. Results are the mean±S.D. of three independent experiments. (B) EGFP-bSV fragments were co-expressed in COS-1 cell line with AR. After transfection and treatment with 10 nM DHT for 16 h, cells were stained with AR antibody (NH27), followed by Texas-red conjugated secondary antibody, and analyzed under confocal microscope. Signals of single focal plane are scanned and computerized to images. Merged images are shown as indicated in labels.

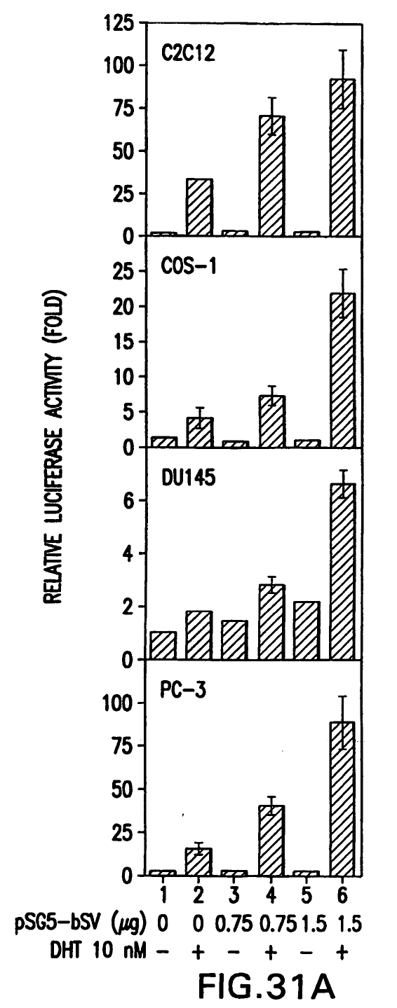

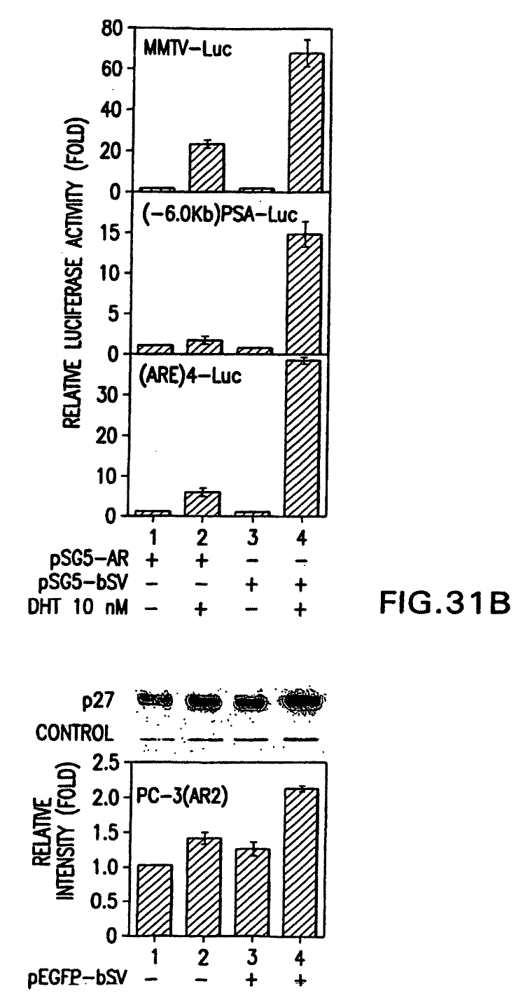

41. FIG. 31 shows SV enhanced AR transcription activity. (A) C2C12, COS-1, DU145 and PC-3 cell lines were co-transfected with 30 ng pSG5-AR, 0.5 μg MMTV-Luc, 1 ng pRL-SV40, various amounts of pSG5-bSV as indicated, and adjusted to total amount of 2 μg DNA with pSG5. The assay method was the same as FIG. 2. After measuring the luciferase activity, values relative to lane 1 were calculated. Results are the mean±S.D. of three independent experiments. (B) PC-3 was co-transfected with 30 ng pSG5-AR, 1.5 μg pSG5-bSV, 1 ng pRL-SV40, and 0.5 μg reporter gene as indicated by using SuperFect. After 20 h, cells were treated with EtOH or 10 nM DHT for another 24 h and then lysed for luciferase activity assay. (C) PC-3(AR2) cell line was transfected with EGFP or EGFP-bSV expressing vector using SuperFect. After 20 h, cells were treated with EtOH or 10 nM DHT for another 30 h. Proteins extracted from cells were loaded on 15% SDS-PAGE and analyzed by western blotting. The intensity of each p27 band was quantified and normalized with control protein which is a non-specific band pick up by the antibody in the same blot. The relative intensities to lane 1 were calculated.

42. FIG. 32 shows that SV interacted with other steroid receptors and enhanced their function. (A) The interaction of SV with AR, GR, PPAR-γ and ER-α is tested in mammalian two-hybrid assay. One μg plasmids expressing Gal4(DBD)-AR, GR, PPAR-γ or ER-α was co-transfected with 4 μg plasmids expressing VP16 or VP16-SVn to COS-1 cells. 10 nM DHT, 10 nM dexamethasone, 1 μM 15-deoxy-Δ12,14-prostaglandin J2, and 10 nM 17β-estradiol were applied to AR, GR, PPAR-γ. and ER-α, respectively. The assay method was the same as described in FIG. 29. Relative activities of ligand treatment to EtOH treatment are shown. (B) The coactivation function of SV in different receptors was assayed using reporter gene study. MMTV-Luc, PPRE-Luc, ERE-Luc are the reporter genes for AR, GR, PPAR-γ, and ER-α respectively. Lanes 1, 5, 9 and 13 are regarded as 1 fold in each panel.

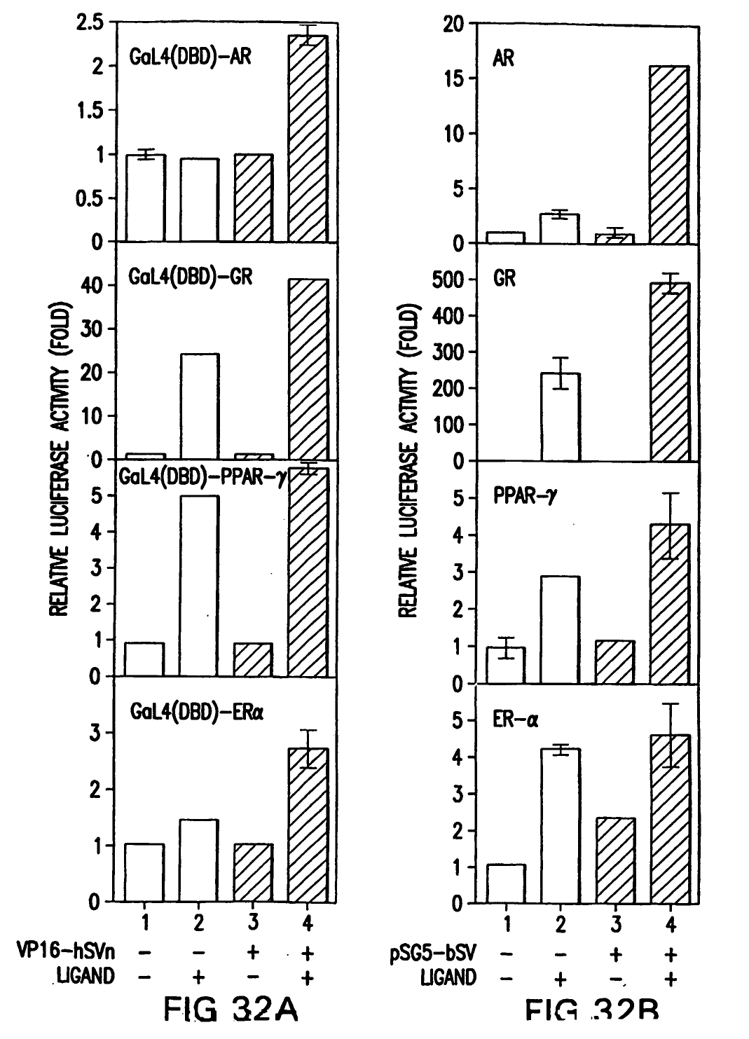

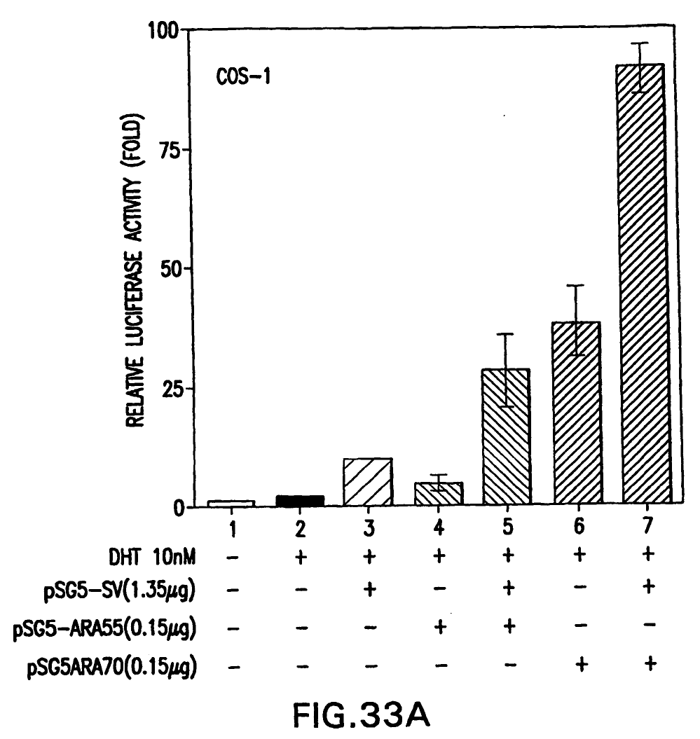

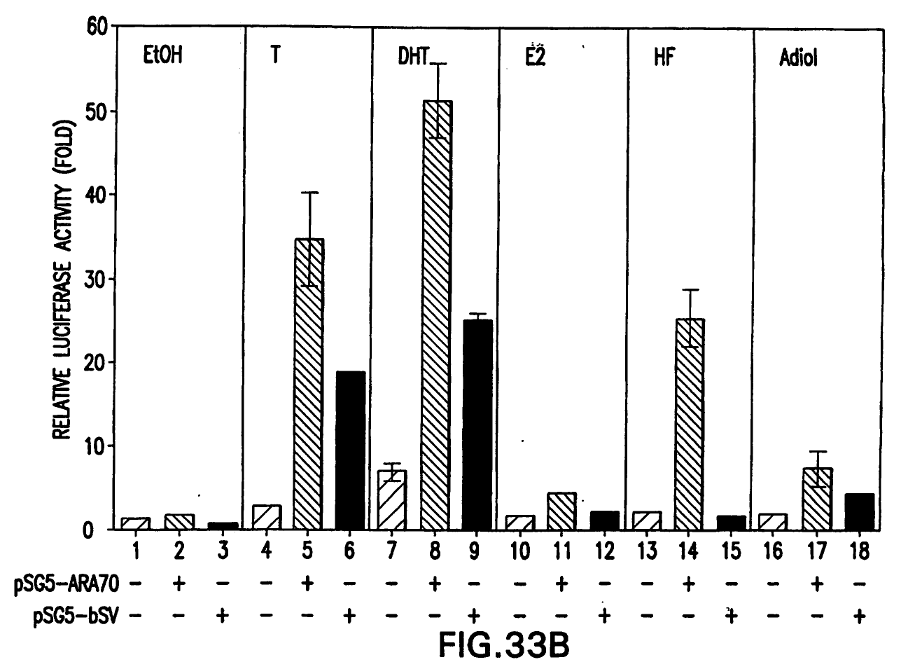

43. FIG. 33 shows that SV cooperates with other ARAs and affects various steroids induced AR transactivation. (A) COS-1 cells were co-transfected with 0.5 μg MMTV-Luc, 1 ng pRL-SV40, pSG5-AR (30 ng) and combination of 1.4 μg pSG5-bSV, 0.1 μg ARA55 or 0.1 μg ARA70N as described in the figure. The total amount of DNA was adjusted to 2 μg with pSG5. The assay was carried out as in FIG. 30. (B) COS-1 cells were transfected with 0.5 μg MMTV-Luc, 1 ng pRL-SV40, 30 ng pSG5-AR with 1.5 μg pSG5, 0.1 μg pSG5-ARA70N or 1.5 μg pSG5-bSV. The total amount of DNA was adjusted to 2 μg with pSG5. After 16 h transfection, cells were treated with vehicle (EtOH) or steroids (10 nM T, DHT, E2, HF, or Adiol) for 20 h as indicated. The assay was carried out as described in FIG. 30.

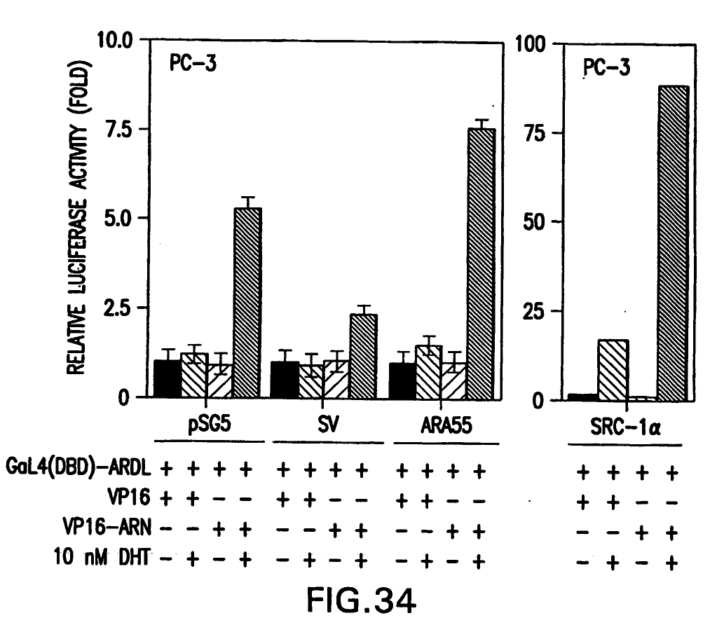

44. FIG. 34 shows that AR N—C interaction is reduced by bSV. PC-3 cells were transfected with 30 ng plasmids expressing Gal4(DBD)-AR-DL, VP16 or VP16-ARN combined with 1.5 μg pSG5, pSG5-bSV, -ARA55, or -SRC-1α as indicated. The reporter plasmid pG5-Luc (0.5 μg) and control plasmid pRL-Luc (1 ng) were transfected to every sample. The assay was carried out as described in FIG. 29C.

### IV. DETAILED DESCRIPTION

45. The present invention may be understood more readily by reference to the following detailed description of preferred embodiments of the invention and the Examples included therein and to the Figures and their previous and following description.

46. Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

47. The abbreviations used are: AR, androgen receptor; SR, steroid receptor; DHT, 5α-dihydrotestosterone; HF, hydroxyflutamide; Adiol, Δ5-androstendiol: E2, 17β-estradiol; DEX, dexamethasone; DHEA, dehydoepiandrosterone; DBD, DNA-binding domain; LBD, Ligand-binding domain; PSA, prostate-specific antigen; ARA; androgen-receptor associated protein; CAT, chloramphenical acetyltransferase; LUC, luciferase; GST, glutathione S-transferase; MMTV, mouse mammary tumor virus; C′-ARA54, C-terminal region of ARA54; fl-ARA54, full-length ARA54; dn-mt-ARA54, dominant-negative mutant ARA54; DHT, 5α-dihydrotestosterone; P, progesterone; Dex, dexamethasone; AD, activation domain; SD, synthetic dropout; DMEM, Dulbecco's minimum essential medium; FCS, fetal calf serum; CAT, chloramphenicol acetyltransferase; Luc, luciferase; PSA, prostate-specific antigen; GR, glucocorticoid receptor; PR, progesterone receptor; doxy, doxycycline; MMTV, mouse mammary tumor virus.

48. As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

49. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed.

50. Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon. Furthermore, references are typically cited along with a letter, such as (Chang et al. (1995) Critical Reviews in Eukaryotic Gene Expression 5, 97-125). This letter refers to particular reference list disclosed herein, designated with the letter. Furthermore, should a letter not be associated with a reference number, it will be clear to the skilled artisan, from the context and the potential references, which reference is being relied upon.

51. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

52. In this specification and in the claims which follow, reference will be made to a number of terms which shall be defined to have the following meanings:

53. “Optional” or “optionally” means that the subsequently described event or circumstance may or may not occur, and that the description includes instances where said event or circumstance occurs and instances where it does not.

54. “Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

55. “Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

### B. COMPOSITIONS AND METHODS

56. The Androgen receptor (AR) is a member of the steroid receptor superfamily that binds to the androgen response element to regulate target gene transcription. AR may need to interact with some selected coregulators for the maximal or proper androgen function. Disclosed herein is the isolation of AR coregulators,

57. Disclosed are compositions comprising AR, ARA54, ARA55, SRC-1, ARA70, RB, ARA24, ARA160, ARA267, gelsolin, and/or supervillin, or fragment thereof, wherein the composition interacts with AR, such that AR transcription activity is regulated relative to transcription activity in the absence of the composition.

58. Also disclosed are compositions wherein they possess the disclosed activities and wherein the composition comprises AR, ARA54, ARA55, SRC-1, ARA70, RB, ARA24, ARA160, ARA267, gelsolin, and/or supervillin proteins, or fragments thereof, and wherein the proteins or fragments thereof have at least 80%, 85%, 90%, or 95% identity to the sequences of these proteins disclosed herein.

59. Disclosed are compositions comprising an androgen receptor coactivator, wherein the coactivator has been mutated forming a mutated coactivator.

60. Disclosed are compositions, wherein the mutated coactivator retains the ability to dimerize, wherein the mutated coactivator is a dominant negative coactivator, wherein the androgen receptor coactivator is selected from the group consisting of AR, ARA54, ARA55, SRC-1, ARA70, RB, ARA24, ARA160, ARA267, gelsolin, and/or supervillin proteins, or fragments thereof

61. Disclosed are compositions comprising AR, ARA54, ARA55, SRC-1, ARA70, RB, ARA24, ARA160, ARA267, gelsolin, and/or supervillin proteins, or fragments thereof, wherein any variations in the proteins or fragments thereof are conserved variants.

62. Disclosed are methods of regulating transcription activity of AR comprising administering any of the disclosed compositions herein, such as AR, ARA54, ARA55, SRC-1, ARA70, RB, ARA24, ARA160, ARA267, gelsolin, and/or supervillin proteins, or fragments thereof.

63. Disclosed are methods wherein the regulation of AR transcription activity decreases or increases the transcription activity of AR by 10%, 25%, 50%, or 90%.

64. Disclosed are methods of regulating AR transcription activity comprising administering a composition that binds AR as disclosed herein, such as AR, ARA54, ARA55, SRC-1, ARA70, RB, ARA24, ARA160, ARA267, gelsolin, and/or supervillin proteins, or fragments thereof, or a molecule that competitively competes with AR, ARA54, ARA55, SRC-1, ARA70, RB, ARA24, ARA160, ARA267, gelsolin, and/or supervillin proteins, or fragments thereof, for AR binding.

65. Disclosed are methods of identifying a regulator of an interaction between AR and AR, ARA54, ARA55, SRC-1, ARA70, RB, ARA24, ARA160, ARA267, gelsolin, and/or supervillin proteins, or fragments thereof, comprising incubating a library of molecules with AR or an AR fragment forming a mixture, and identifying the molecules that disrupt the interaction between AR and AR, ARA54, ARA55, SRC-1, ARA70, RB, ARA24, ARA160, ARA267, gelsolin, and/or supervillin proteins, or fragments thereof, wherein the interaction disrupted comprises an interaction between the AR-ARA54, ARA55, SRC-1, ARA70, RB, ARA24, ARA160, ARA267, gelsolin, and/or supervillin proteins, or fragments thereof, binding site.

66. Disclosed are methods wherein the step of isolating comprises incubating the mixture with molecule comprising AR, ARA54, ARA55, SRC-1, ARA70, RB, ARA24, ARA160, ARA267, gelsolin, and/or supervillin proteins, or fragments thereof.

67. Disclosed are methods of identifying a regulator of an interaction between AR and AR, ARA54, ARA55, SRC-1, ARA70, RB, ARA24, ARA160, ARA267, gelsolin, and/or supervillin proteins, or fragments thereof, comprising incubating a library of molecules with AR, ARA54, ARA55, SRC-1, ARA70, RB, ARA24, ARA160, ARA267, gelsolin, and/or supervillin proteins, or fragments thereof, forming a mixture, and identifying the molecules that disrupt the interaction between AR and ARA54, ARA55, SRC-1, ARA70, RB, ARA24, ARA160, ARA267, gelsolin, and/or supervillin proteins, or fragments thereof, wherein the interaction disrupted comprises an interaction between the AR-ARA54, ARA55, SRC-1, ARA70, RB, ARA24, ARA160, ARA267, gelsolin, and/or supervillin, or fragments thereof, binding site.

68. Disclosed are methods wherein the step of isolating comprises incubating the mixture with molecule comprising AR or fragment thereof.

69. Disclosed are compositions comprising a fragment of ER, wherein the composition interacts with AR, such that AR transcription activity is decreased relative to transcription activity in the absence of the composition, wherein the fragment comprises a polypeptide having at least 80%, 85%, 90%, or 95% identity to the sequence set forth in herein of AR, ARA54, ARA55, SRC-1, ARA70, RB, ARA24, ARA160, ARA267, gelsolin, and/or supervillin proteins, or fragments thereof.

70. Disclosed are methods of identifying compounds, wherein the identified compound binds AR, ARA54, ARA55, SRC-1, ARA70, RB, ARA24, ARA160, ARA267, gelsolin, and/or supervillin, or fragments thereof, with a kd less than or equal to 10⁻⁵ M, 10⁻⁶ M, 10⁻⁷ M, 10⁻⁸ M, 10⁻⁹ M, or 10⁻¹⁰ M, 10⁻¹¹ M, or 10⁻¹² M.

71. Disclosed are methods of regulating AR transcription activity comprising administering a composition that binds AR, ARA54, ARA55, SRC-1, ARA70, RB, ARA24, ARA160, ARA267, gelsolin, and/or supervillin, or fragments thereof, wherein the composition is AR, ARA54, ARA55, SRC-1, ARA70, RB, ARA24, ARA160, ARA267, gelsolin, and/or supervillin, or fragments thereof, or a molecule that competitively competes with AR, ARA54, ARA55, SRC-1, ARA70, RB, ARA24, ARA160, ARA267, gelsolin, and/or supervillin, or fragments thereof, for AR binding.

72. Disclosed are methods of regulating AR transcription activity comprising administering a composition, wherein the composition regulates AR transcription activity, wherein the composition is defined as a composition capable of being identified by administering the composition to a system, wherein the system supports AR transcription activity, assaying the effect of the composition on the amount of transcription activity in the system, and selecting a composition which regulates the amount of AR transcription activity present in the system relative to the system without the addition of the composition.

73. Also disclosed are methods of regulating AR transcription activity comprising administering a composition that binds AR, wherein the composition is ARA54, ARA55, SRC-1, ARA70, RB, ARA24, ARA160, ARA267, gelsolin, and/or supervillin, or fragment thereof, or a molecule that competitively competes with ARA54, ARA55, SRC-1, ARA70, RB, ARA24, ARA160, ARA267, gelsolin, and/or supervillin, or fragment thereof, for AR binding.

74. Disclosed are methods of making a composition capable of regulating AR transcription activity comprising admixing a compound with a pharmaceutically acceptable carrier, wherein the compound is identified by administering the compound to a system, wherein the system supports AR transcription activity, assaying the effect of the compound on the amount of AR transcription activity in the system, and selecting a compound which regulates the amount of AR transcription activity in the system relative to the system without the addition of the compound.

75. Disclosed are methods of manufacturing a regulator of AR transcription activity comprising, a) administering a composition to a system, wherein the system supports AR transcription activity, b) assaying the effect of the composition on the amount of AR transcription activity in the system, c) selecting a composition which regulates the amount of AR transcription activity present in the system relative to the system with the addition of the composition, and d) synthesizing the composition.

76. Also disclosed are methods comprising the step of admixing the composition with a pharmaceutical carrier.

77. Disclosed are cells further comprising a regulator of a AR transcription activity.

78. Disclosed are systems where the systems also include ARA54, ARA55, SRC-1, ARA70, RB, ARA24, ARA160, ARA267, gelsolin, or supervillin, or fragment thereof, in any combination with the AR transactivation in the system.

79. It is understood that the systems include cells that are expressing the disclosed proteins, such as AR, ARA54, ARA55, SRC-1, ARA70, RB, ARA24, ARA160, ARA267, gelsolin, or supervillin, or fragment thereof, in any combination.

80. Disclosed are compositions comprising an isolated mutant of an ARA54 peptide comprising a peptide having at least 80% identity to SEQ ID NO:1, wherein the peptide prevents homodimerization of ARA54. Further disclosed are compositions, wherein the mutant ARA further comprises a substitution at position 472 of SEQ ID NO:1, wherein the mutant ARA comprises a lysine substitution at position 472 of SEQ ID NO:1.

81. Disclosed are nucleic acids encoding the disclosed mutant Andorgen receptor interacting proteins, and nucleic acids wherein the nucleic acid further comprises a promoter sequence operably linked to the sequence encoding the mutant ARA.

82. Disclosed are cells comprising the disclosed nucleic acids and/or disclosed peptides.

83. Also disclosed are animals comprising the disclosed nucleic acids, peptides, and/or cells.

84. Disclosed are methods of inhibiting androgen receptor transactivation comprising administering the disclosed compositions.

85. Disclosed are methods of identifying a molecule that modulates the activity of androgen receptor comprising administering the molecule to a system comprising androgen receptor and the disclosed compositions, assaying the activity of androgen receptor, and selecting molecules that modulate the activity of androgen receptor.

86. Disclosed are methods, wherein the system further comprises one or more in any combination of ARA54, ARA55, SRC-1, ARA24, Rb, ARA70, RB, ARA24, ARA267, gelsolin, or supervillin, or variant comprising androgen receptor modulating activity, in any combination.

87. Disclosed are methods of identifying a dominant negative inhibitor of androgen receptor comprising administering a mutagen to a nucleic acid encoding an ARA interacting protein forming a nucleic acid encoding a mutated ARA interacting protein, performing a screening system, wherein the system comprises the mutated ARA interacting protein and androgen receptor, assaying the activity of the androgen receptor, and identifying those mutated ARA interacting proteins that reduce androgen receptor activity. Also disclosed are methods, wherein the mutagen comprises hydroxylamine.

88. Disclosed are compositions comprising an ARA267 peptide comprising a peptide having at least 80% identity to SEQ ID NO:34, wherein the peptide enhances androgen receptor transactivation of androgen receptor. Further disclosed are compositions, wherein the mutant ARA wherein the mutant ARA further comprises an LXXLL motif, wherein the mutant ARA wherein the mutant ARA further comprises a SET motif, wherein the mutant ARA wherein the mutant ARA further comprises a proline rich region, wherein the mutant ARA wherein the mutant ARA further comprises a Ring finger motif, and/or wherein the mutant ARA wherein the mutant ARA further comprises a Zinc finger motif.

89. Also disclosed are compositions comprising an ARA267 peptide comprising amino acids 1668-1795 of SEQ ID NO: 34, amino acids 726-730 of SEQ ID NO:34, and amino acids 1283-1287 of SEQ ID NO:34, amino acids 1324-1369 of SEQ ID NO:34 and amino acids 1884-1909 of SEQ ID NO:34.

90. Disclosed are compositions comprising an isolated mutant of an ARA70 peptide comprising a peptide having at least 80% identity to SEQ ID NO:26, wherein the peptide prevents androgen receptor transactivation of androgen receptor. Further disclosed are compositions, wherein the mutant ARA wherein the mutant ARA70 does not contain an LXXLL motif, compositions comprising an isolated mutant of an ARA70 peptide comprising a peptide having at least 80% identity to amino acids 176-401 of SEQ ID NO ID NO:26, wherein the peptide prevents androgen receptor transactivation of androgen receptor, and/or composition comprising an isolated mutant of an ARA70 peptide comprising a peptide having at least 80% identity to amino acids 176-401 of SEQ ID NO:26 and comprising an FXXLF domain, wherein the mutant ARA70 enhances androgen transactivation.

91. Disclosed are compositions comprising FXXLF, wherein the peptide interacts with androgen receptor, and wherein the peptide is not ARA54, ARA55, SRC-1, SRC-1, ARA24, Rb, ARA70, RB, ARA24, ARA267, gelsolin, and supervillin.

92. Also disclosed are compositions comprising FXXLF, wherein the peptide interacts with androgen receptor, and wherein the peptide is less than or equal to the size of ARA54, ARA55, SRC-1, SRC-1, ARA24, Rb, ARA70, RB, ARA24, ARA267, gelsolin, and supervillin.

93. Also disclosed are methods of inhibiting androgen receptor activity comprising, administering a molecule that blocks an interaction between the androgen receptor and gelsolin. Further disclosed are methods, wherein the molecule is a peptide, wherein the peptide comprises a region of androgen receptor, wherein the peptide comprises amino acids 551-600 of SEQ ID NO:44, and/or wherein the peptide comprises amino acids 655-695 of SEQ ID NO:44.

94. Disclosed are methods of identifying an androgen receptor activity inhibiting molecule, comprising administering a molecule or set of molecules to a system, wherein the system comprises androgen receptor and gelsolin, and assaying whether the molecule reduces the interaction between androgen receptor and gelsolin. Further disclosed are methods, wherein the system further comprises an androgen receptor ligand, and/or wherein the ligand is DHT.

95. Also disclosed are methods of identifying an mutant androgen receptor activity inhibiting molecule, comprising administering a molecule or set of molecules to a system, wherein the system comprises the mutant androgen receptor and gelsolin, and assaying whether the molecule reduces the interaction between the mutant androgen receptor and gelsolin. Further disclosed are methods, wherein the system further comprises a mutant androgen receptor ligand, and/or wherein the ligand is HF.

96. Disclosed are methods of making a composition, the method comprising synthesizing a molecule, wherein the molecule inhibits androgen receptor activity, and wherein the molecule inhibits an interaction between androgen receptor and gelsolin.

97. Disclosed are systems comprising ARA267 or a peptide or protein comprising FXXLF. Further disclosed are systems, wherein the ARA267 has at least 80% identity to the sequence set forth in SEQ ID NO:34, wherein the system further comprises a cell, wherein the system further comprises a androgen receptor, and/or wherein the system further comprises one or more in any combination of ARA54, ARA55, SRC-1, ARA24, Rb, ARA70, ARA267, gelsolin, or supervillin, or fragment or variant thereof.

98. Disclosed are methods of inhibiting androgen receptor activity comprising, administering a molecule that blocks an interaction between the androgen receptor and Supervillin. Further disclosed are methods, wherein the supervillin comprises amino acids 558-1788 of SEQ ID NO:38, and/or wherein the peptide comprises amino acids 594-1335 of SEQ ID NO:38.

99. Disclosed are methods of inhibiting activity of a mutant androgen receptor comprising, administering a molecule that blocks an interaction between the mutant androgen receptor and supervillin. Further disclosed are methods, wherein the molecule is a peptide, and/or wherein the peptide comprises a region of androgen receptor.

100. Disclosed are methods of identifying an androgen receptor activity inhibiting molecule, comprising administering a molecule or set of molecules to a system, wherein the system comprises androgen receptor and supervillin, and assaying whether the molecule reduces the interaction between androgen receptor and supervillin. Disclosed are methods, wherein the system further comprises an androgen receptor ligand, and/or wherein the ligand is DHT.

101. Also disclosed are methods of making a composition, the method comprising synthesizing a molecule, wherein the molecule inhibits androgen receptor activity, and wherein the molecule inhibits an interaction between androgen receptor and supervillin.

### C. COMPOSITIONS

102. Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular AR, ARA54, ARA55, SRC-1, ARA70, RB, ARA24, ARA160, ARA267, gelsolin, and/or supervillin, or fragment thereof, is disclosed and discussed and a number of modifications that can be made to a number of molecules including the AR, ARA54, ARA55, SRC-1, ARA70, RB, ARA24, ARA160, ARA267, gelsolin, and/or supervillin, or fragment thereof, are discussed, specifically contemplated is each and every combination and permutation of AR, ARA54, ARA55, SRC-1, ARA70, RB, ARA24, ARA160, ARA267, gelsolin, and/or supervillin, or fragment thereof, and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

103. Disclosed are isolated polynucleotides that encode co-regulators for human androgen receptor. The polynucleotides comprise sequences that encodes AR, ARA54, ARA55, SRC-1, ARA24, Rb, ARA70, ARA267, gelsolin, and/or supervillin, or fragment thereof.

104. Also disclosed are genetic constructs comprising a promoter functional in a prokaryotic or eukaryotic cell operably connected to the disclosed polynucleotides, where the polynucleotide is for example, AR, ARA54, ARA55, SRC-1, ARA24, Rb, ARA70, ARA267, gelsolin, and/or supervillin, or fragment thereof.

105. Also disclosed are methods for screening candidate pharmaceutical molecules for the ability to promote or inhibit the interaction of ARs and AREs to modulate androgenic activity comprising the steps of: (a) providing a genetic construct as disclosed herein, (b) cotransforming a suitable eukaryotic cell with the construct of step a), and a construct comprising at least a portion of an expressible androgen receptor sequence; (c) culturing the cells in the presence of a candidate pharmaceutical molecule; and (d) assaying the transcription activity induced by the androgen receptor.

106. Also disclosed are genetic constructs capable of expressing a factor involved in co-activation of the human androgen receptor.

107. Also disclosed are methods for evaluating the ability of candidate pharmaceutical molecules to modulate the effect of androgen receptor coactivators on gene expression.

108. Transactivation of genes by the androgen receptor is a system that involves many different coactivators. It is not currently known just how many factors are involved in androgen receptor-mediated regulation of gene expression. The identification and/or characterization of many androgen receptor coregulators is reported herein. Inclusion of one or more of these coregulators in an assay for androgenic and antiandrogenic activity is expected to increase the sensitivity of the assay. A preliminary assessment of the efficacy of a potential therapeutic agent can be made by evaluating the effect of the agent on the ability of the coactivator to enhance transactivation by the androgen receptor.

109. One aspect of the present invention is an isolated polynucleotide that encodes a co-activator for human androgen receptor, the polynucleotide comprising a sequence that encodes AR, ARA54, ARA55, SRC-1, ARA70, RB, ARA24, ARA160, ARA267, gelsolin, and/or supervillin, or fragment thereof.

110. Another aspect of the present invention is a genetic construct comprising a promoter functional in a prokaryotic or eukaryotic cell operably connected to a polynucleotide that encodes AR, ARA54, ARA55, SRC-1, ARA70, RB, ARA24, ARA160, ARA267, gelsolin, and/or supervillin, or fragment thereof.

111. The present invention includes a method for screening candidate pharmaceutical molecules for the ability to promote or inhibit the ARs and AREs to result in modulation of androgenic effect comprising the steps of (a) providing a genetic construct comprising a promoter functional in a eukaryotic cell operably connected to a polynucleotide comprising a sequence that encodes AR, ARA54, ARA55, SRC-1, ARA70, RB, ARA24, ARA160, ARA267, gelsolin, and/or supervillin, or fragment thereof; (b) cotransforming a suitable eukaryotic cell with the construct of step a, and a construct comprising at least a portion of an expressible androgen receptor sequence; (c) culturing the cells in the presence of a candidate pharmaceutical molecule; and (d) assaying the transcription activity induced by the androgen receptor gene.

112. In certain cases, progression of prostate cancer from androgen dependent- to androgen independent-stage may be caused by a mutation in the LBD that alters the ligand specificity of the mAR (Taplan et al., New Engl. J. Med. 332:1393-1398 (1995); Gaddipati et al., Cancer Res. 54:2861-2864 (1994)). We examined whether differential steroid specificity of wild type (wt) AR and mAR involves the use of different androgen receptor-associated (ARA) proteins or coactivators by these receptors.

113. As described in the examples, a yeast two-hybrid system with mART887S as bait was used to screen the human prostate cDNA library. The sequences of two clones encoding a putative coactivators (designated ARA54 and ARA55) are shown in SEQ ID NO:1 and SEQ ID NO:3, respectively. The putative amino acid sequences of ARA54 and ARA55 are shown in SEQ ID NO:2 and SEQ ID NO:4, respectively. Also provided are the DNA and amino acid sequences of ARA24 (SEQ ID NO:5 and SEQ ID NO:6, respectively) and Rb (SEQ ID NO:7 and SEQ ID NO:8, respectively). These coactivators were further characterized as detailed below. It is expected that some minor variations from SEQ ID NOs:1-8, as well as any sequences disclosed herein can be associated with nucleotide additions, deletions, and mutations, whether naturally occurring or introduced in vitro, will not affect coactivation by the expression product or polypeptide.

114. It is understood that the disclosed compositions, including AR, ARA54, ARA55, SRC-1, ARA70, RB, ARA24, ARA160, ARA267, gelsolin, and/or supervillin, or fragment thereof, can be transfected into any type of cell either alone or in any combination. Disclosed herein are the advantages of having more than one co-regulator expressed in cells in any of the disclosed assays and methods disclosed herein, because of the fact that the disclosed co-regulators can act together, to enhance and/or reduce transciption activity of AR. It is also understood that the various ligands for AR can also be included alone or in any combination with any of the cells or coregulators and andrgoen receptors disclosed herein.

115. In the examples, various eukaryotic cell types, including yeast, prostate cells having mutant AR and cells lacking AR, were used to evaluate the ability of the putative androgen coactivators to enhance transactivation by AR. It is expected that in the method of the present invention, any eukaryotic cell could be employed in an assay for AR activity.

116. Changes in the level of transactivation by AR can be assessed by any means, including measuring changes in the level of mRNA for a gene under the control of AR, or by quantitating the amount of a particular protein expressed using an antibody specific for a protein, the expression of which is under the control of AR. Most conveniently, transactivation by AR can be assessed by means of a reporter gene.

117. As used herein, a reporter gene is a gene under the control of an androgen receptor, the gene encoding a protein susceptible to quantitation by a colormetric or fluorescent assay. In the examples below, a chloramphenicol acetyltransferase or a luciferase gene were used as reporter genes. The gene may either be resident in a chromosome of the host cell, or may be introduced into the host cell by cotransfection with the coactivator gene.

1. AR

118. The Andorgen receptor (AR) is a ligand-dependent transcription factor that belongs to the steroid receptor (SR) superfamily (Chang et al. (1988) Science 240, 324-326; Chang et al. (1989) Proc. Natl. Acad. Sci. USA 85, 7211-7215).

119.). Although several studies have revealed how hormone-bound SRs can recognize and interact with hormone-response elements (HREs) (3B-5B), the mechanism of how SRs activate target gene expression is not fully understood. After AR binds to androgens, it dissociates from chaperone proteins with subsequent processes, including nuclear translocation, dimer formation, and DNA response element binding, that result in its target genes regulation (Chang et al. (1995) Crit. Rev. Eukaryot. Gene Expr. 5, 97-125).

120. There is a substantial amount of evidence to indicate that steroid hormone receptors function as a tripartite system, involving the receptor, its ligands, and its coregulator proteins (Katzenellenbogen et al. (1996) Mol. Endocrinol. 10, 119-131; Torchia et al. (1998) Curr. Opin. Cell Biol. 10, 373-383; McKenna et al. (1999) J. Steroid Biochem. Mol. Biol. 69, 3-12; Yeh et al. (1998) Proc. Natl. Acad. Sci. U.S.A. 95, 5524-5532; Miyamoto et al. (1998) Proc. Natl. Acad. Sci. U.S.A. 95, 7379-7384). The androgen receptor (AR)¹, a member of this receptor superfamily, is a ligand-dependent transcription factor that mediates the biological effects of androgens in a variety of target tissues, including the prostate. AR involvement is also associated with a number of pathological conditions, notably prostate cancer (. Chang et al. (1988) Science 240, 324-326; Evans, R. M. (1988) Science 240, 889-895; Montie, J. E., and Pienta, K. J. (1994) Urology 43, 892-899; Ruijter et al. (1999) Endocr. Rev. 20, 2245). Examples of a number of steroid receptor coactivators, include SRC-1 (Onate et al. (1995) Science 270, 1354-1357), GRIP1/TIF2 (Hong et al. (1996) Proc. Natl. Acad. Sci. U.S.A. 93, 4948-4952; Voegel et al. (1996) EMBO J. 15, 3667-3675) pCIP/ACTR/AIB1/RAC3/TRAM-1 (Torchia et al. (1997) Nature 387, 677-684; Chen et al. (1997) Cell 90, 569-580; Anzick et al. (1997) Science 277, 965-968; Li et al. (1997) Proc. Natl. Acad. Sci. U.S.A. 94, 8479-8484).

121. TIF1 (Le Douarin et al. (1995) EMBO J. 14, 2020-2033), RIP140 (Cavailles et al. (1995) EMBO J. 14, 3741-3751), TAFII30 (Verrier et al. (1997) Mol. Endocrinol. 11, 1009-1019), PGC-1 (Puigserver et al. (1998). A cold-inducible coactivator of nuclear receptors linked to adaptive thermogenesis. Cell 92, 829-839), SNURF (Moilanen et al. (1998) Mol. Cell. Biol. 18, 5128-5139), and others (Torchia et al. (1998) Curr. Opin. Cell Biol. 10, 373-383; McKenna et al. (1999) J. Steroid Biochem. Mol. Biol. 69, 3-12; Di Croce et al. (1999) EMBO J. 18, 6201-6210; Hsiao et al. J Biol Chem 274, 20229-20234. (1999); Kang et al. J Biol Chem 274, 8570-8576. (1999); Fujimoto et al. J Biol Chem 274, 8316-8321. (1999); Yeh et al. Proc Natl Acad Sci USA 93, 5517-5521. (1996); Hsiao, P. W. & Chang, C. J Biol Chem 274, 22373-22379. (1999); Wang. et al. J Biol Chem 276, 40417-40423. (2001); Yeh et al. Biochem Biophys Res Commun 248, 361-367. (1998); Ding et al. Mol Endocrinol 12, 302-313. (1998); Berrevoets et al. Mol Endocrinol 12, 1172-1183. (1998); Tan et al. Endocrinology 141, 3440-3450. (2000)), have been identified as being able to modulate steroid receptor transactivation. Several coregulators, AR-associated (ARA) proteins that enhance AR transcription activation by interacting with AR in a ligand-dependent manner, have also been isolated and characterized (Yeh, S, and Chang, C, (1996) Proc. Natl. Acad. Sci. U.S.A. 93, 5517-5521; Yeh et al. (1998) Biochem. Biophys. Res. Commun. 248, 361-367; Fujimoto et al. (1999) J. Biol. Chem. 274, 8316-8321; Kang et al. (1999) J. Biol. Chem. 274, 8570-8576;

122. Hsiao et al. (1999) J. Biol. Chem. 274, 20229-20234; Hsiao, P.-W., and Chang, C. (1999) J. Biol. Chem. 274, 22373-22379; Yeh et al. (1999) Endocrine 11, 195-202).

123. One of the AR coregulators, ARA54, can enhance transactivation of wild-type AR and a mutant AR, derived from LNCaP prostate cancer cells, in prostate cancer cells by 2-6 fold in the presence of androgens or the antiandrogen hydroxyflutamide (HF) (Kang et al. (1999) J. Biol. Chem. 274, 8570-8576; Yeh et al. (1999) Endocrine 11, 195-202).

124. Prostate cancer is the second leading cause of death in American men (Wingo et al. (1995) CA Cancer J Clin 45, 8-30. (1995). Androgens and AR have been well documented to correlate with prostate cancer growth (Prins et al. J Urol 159, 641-649. (1998). Androgen ablation therapy with chemical/surgical castration in combination with antiandrogens (flutamide or casodex) remains as mainstream therapy to treat the metastatic prostate cancer (Eisenberger et al. N Engl J Med 339, 1036-1042. (1998); Crawford et al. N Engl J Med 321, 419-424. (1989)). However, most prostate cancers undergoing such androgen ablation treatment develop “flutamide withdrawal syndrome”, in which patients show worse clinical performance but improve after flutamide withdrawal (Scher et al. J Clin Oncol 11, 1566-1572. (1993); Kelly et al. Urol Clin North Am 24, 421-431. (1997)). Furthermore, tumor may progress from an androgen-dependent to an androgen-independent state (Dreicer, R. Cleve Clin J Med 67, 720-722, 725-726. (2000). Some patients with androgen-dependent disease develop a withdrawal syndrome that is associated with an agonist effect of antiandrogens resulting in antiandrogen treatment promoting prostate cancer progression (Kelly et al. (1997) Urol. Clin. North Am. 24, 421-431). Previous studies are consistent with AR coactivators promoting the agonist activity of antiandrogens through the interaction with AR (Miyamoto et al. (1998) Proc. Natl. Acad. Sci. U.S.A. 95, 7379-7384; Yeh et al. (1999) Endocrine 11, 195-202; Yeh et al. (1996) Lancet 349, 852-853). The interruption of this AR-coregulator interaction may therefore provide a target for the development of novel treatment strategies for advanced prostate cancer. Several mechanisms have been proposed as following. First, the mutant AR with broaden ligands specificity has been detected in prostate tumors and results in non-androgen steroids and hydroxyflutamide (HF) responsive AR (Taplin et al. N Engl J Med 332, 1393-1398. (1995); Fenton et al. Clin Cancer Res 3, 1383-1388. (1997)).

125. Second, the cross talk between AR and Her-2/neu pathway suggests growth factors stimulated signals can activate AR (Yeh et al. Proc Natl Acad Sci USA 96, 5458-5463. (1999)). The androgen receptor (AR) is aligand inducible transcription regulator that can activate or repress its target genes by binding to its hormone response elements (HRE) as a homodimer. The AR consists of four major functional domains including a ligand binding domain (LBD), and two activation functions (AF) residing in the N-terminal (AF-1) and the C-terminal end of the LBD (AF-2) respectively.

126. By forming a homodimer and taking into account of the ligand and coregulators, the androgen receptors interact and regulate the transcription of numerous target genes (1 ng, 1992; Schulman, 1995; Beatp, 1996; Yeh, 1996; Glass, 1997, Shibata, 1997). Androgen is the strongest ligand of the androgen receptor. However, it is not the only ligand. Estradiol has been found to activate androgen receptor transactivation through the interaction with androgen receptor (Yeh, 1998). Besides, androgen and androgen receptor do not only act in male. The increasing evidence has displayed that the androgen and androgen receptor (AR) may also play important role in female physiological processes, including the process of folliculogenesis, the bone metabolism and the maitainence of brain functions (Miller, 2001).

127. Androgen is the most conspicuous amount of steroid hormone in ovary (Risch H A, 1998). The concentrations of testosterone and estradiol in the late-follicular phase when estrogens are at their peak are 0.06-0.10 mg/day and 0.04-0.08 mg.day respectively (Risch H A, 1998). The ratio of androgens versus estrogens in the ovarian veins of postmenopausal women is 15 to 1 (Risch, 1998; Doldi N, 1998). Androgen receptor is expressed dominantly in granulosa cells of ovary (Hiller S G, 1992; Hild-Petito S, 1991). With the overproduction of ovarian androgen, women with polycystic ovarian syndrome suffered from impairment of ovulatory function which is characterized with the increasing number of small antral follicles, but arrest in grafian follicles development (Kase, 1963; Futterweit W, 1986; Pache T D, 1991; Spinder T, 1989; Spinder T, 1989; Hughesdon P E, 1982). This symptom has suggested that AR may play a proliferative role in early folliculogenesis but turn to inhibitory effect in late folliculogenesis. The recent studies conducted in animals have supported this hypothesis (Harlow C R, 1988; Hilllier S, 1988; Weil S, 1998; Vendola K, 1998; Weil S, 1999; Vendola K, 1999). Administration of hihydroxytestosterone (DHT) in rheusus monkeys has increased the number of primary, preantral and small antral follicles. Since DHT is the metabolite of testosterone and cannot be aromatized, the result suggested the proliferative effect was through AR system (Vendola K, 1999).

2. Estrogen Receptor

128. Estrogen receptors (ERs), including ERα and ERβ, belong to nuclear hormone receptor superfamily and mediate estrogen actions in regulation of cell growth and differentiation, particularly in mammary glands and uterus in females (see reviews in (Kang et al. (1999) J. Biol. Chem. 274, 8570-8576; Hsiao et al. (1999) J. Biol. Chem. 274, 20229-20234)).

129. The proliferation of mammary glands is mainly dependent on estrogen stimulation; however, the proliferating epithelial cells detected in terminal end buds (TEBs) at the tip of elongating ducts in mammary glands are usually ER-negative (Hsiao, P.-W., and Chang, C. (1999) J. Biol. Chem. 274, 22373-22379; Yeh et al. (1999) Endocrine 11, 195-202; Greenlee et al. (2001) CA Cancer J. Clin. 51, 15-36).

130. Despite the unclear role of ER in this process, in mice with a homozygous disruption of ER genes, the mammary glands remain undeveloped as demonstrated by the lack of TEBs and alveolar structures, even though the serum estrogen levels are 10 times higher than those in wild-type mice (Kelly et al. (1997) Urol. Clin. North Am. 24, 421-431; Yeh et al. (1996) Lancet 349, 852-853).

131. This indicates a role of ER in the growth of mammary glands. Also, the fact that more than two thirds of breast cancers from patients are ER-positive and benefit from antiestrogen or ovariectomy therapies, strengthens the ER involvement in stimulation of cell growth in mammary glands in response to estrogen (Taplin et al. (1995) N. Engl. J. Med. 332, 1393-1398).

132. Estrogen receptors (ER) that play many essential roles for the growth in female reproductive tissues are encoded by two distinct genes, ERα and ERβ (Sadovsky et al. (1995) Mol. Cell. Biol. 15, 1554-1563). It has been demonstrated that ERα and ERβ can form heterodimers, and ERα was able to directly bind to TR, RAR, RXR (Baniahmad et al. (1993) Proc. Natl. Acad. Sci. USA 90, 8832-8836), short heterodimer partner (SHP) (McEwan, I. J., and Gustafsson, J. (1997) Proc. Natl. Acad. Sci. USA 94, 8485-8490; Lee, D. K., Duan, H. O., and Chang, C. (2000) J. Biol. Chem. 275, 9308-9313), and ERβcx (17B). ERα-TR and ERα-RXR heterocomplexes moderately enhance ER-mediated transcription in transient transfection experiments with CV-1 cells. In contrast, RAR repressed ER-mediated transactivation (Baniahmad et al. (1993) Proc. Natl. Acad. Sci. USA 90, 8832-8836). The SHP inhibits ER transcription activity by preventing coactivator binding to ER (16B) and ERβcx inhibits ER transactivation by preventing ER binding to DNA (Pugh, B. F., and Tjian, R. (1990) Cell 61, 1187-1197). Here we demonstrate that TR4 also inhibits ER transcription activity in lung cancer H1299 cells and in breast cancer MCF-7 cells. Further studies indicate that TR4 can suppress ER function via protein-protein interaction that results in the interruption of ER-ER homodimerization and in preventing ER binding to its estrogen response element (ERE). The analysis of ERα KO mice indicated that ERα may play important in vivo functions, such as the growth of the adult female reproductive tract and mammary gland, the regulation of gonadotropin gene transcription, mammary neoplasia induction, and sexual behaviors. Surprisingly, ERα also play important roles in spermatogenesis and sperm function (see review).

3. Interactions with AR

133. Disclosed herein AR can interact with a number of proteins. These interactions can alter AR transcription activation activity as well as altering the transcription activation activity of the disclosed proteins. Disclosed herein AR interacts with AR, ARA54, ARA55, SRC-1, ARA70, RB, ARA24, ARA160, ARA267, gelsolin, or supervillin, or fragment thereof.

a) Interaction Between AR and AR, ARA54, ARA55, SRC-1, ARA70, RB, ARA24, ARA160, ARA267, Gelsolin, or Supervillin, or Fragment Thereof.

134. Disclosed are methods to screen for drugs for AR-related diseases by testing a compound's effect on AR transcription level. If a compound can increase or decrease the level of AR in a cell, then it can be selected for further testing for treatment of AR-related diseases. The screening method can measure AR level directly. It can also measure AR level indirectly, for example, through any reporter system that measures the increase or decrease of AR transactivation. Examples of such reporter systems are described below.

135. A compound that is identified or designed as a result of any of the disclosed methods can be obtained (or synthesized) and tested for its biological activity, e.g., inhibition of AR transcription activity.

136. Disclosed are methods for regulating transcription activity of AR, comprising incubating a regulator of heterodimerization between AR or fragment thereof and ARA54, ARA55, SRC-1, ARA70, RB, ARA24, ARA160, ARA267, gelsolin, and/or supervillin, or fragment thereof, for example.

137. Disclosed are methods of treating a subject comprising administering to the subject a regulator of transcription activity of AR, wherein the regulator reduces the heterodimerzation between AR or fragment thereof and ARA54, ARA55, SRC-1, ARA70, RB, ARA24, ARA160, ARA267, gelsolin, and/or supervillin, or fragment thereof, and wherein the subject is in need of such treatment.

4. Coregulators of AR

138. Recent progression in SR studies indicate that, in addition to contacting the basal transcription machinery directly, SRs may inhibit or enhance transcription by recruiting an array of coregulators. (Yeh et al. Proc. Natl. Acad. Sci. U.S.A. (1996) 93, 5517-5521). Several coregulators that are associated with AR have been identified, such as ARA70, ARA55, ARA54, ARA24, ARA160, Rb, BRCA1, Smad3, AIB1 and SRC1 (Yeh et al. Proc. Natl. Acad. Sci. U.S.A. (1996) 93, 5517-5521; Fujimoto et al. (1999) J. Biol. Chem. 274, 8316-8321; Kang et al. (1999) 274, 8570-8576; Hsiao et al. (1999) J. Biol. Chem. 274, 20229-20234; Hsiao et al. (1999) J. Biol. Chem. 274, 22373-22379; Yeh et al. Biochem. Biophys. Res Commun. (1998) 248, 361-367; Yeh et al. (2000) Proc. Natl. Acad. Sci. U.S.A. 97, 11256-11261; Kang et al. (2001) Proc. Natl. Acad. Sci. U.S.A. 98, 3018-3023; Yeh et al. Proc. Natl. Acad. Sci. U.S.A. (1998) 95, 5527-5532; Yeh et al. (1999) Endocrine 11, 195-202).

139. All of these coregulators can interact with either the C-terminal or N-terminal of AR and enhance AR transactivation (Yeh et al. (1999) Endocrine 11, 195-202). The overexpression of AIB1 has been linked to the risk of breast and ovarian cancer (Anzick et al. (1997) Science 277, 965-968). Variable polyQ lengths within AR and AIB1 were also linked closely to the risk of prostate cancer (Hsing et al. (2000) Cancer. Res. 60, 5111-5116) and ARA24 was associated with the variable polyQ lengths in AR N-terminal domain that may have some roles in the Kennedy's Neuron disease (Hsiao et al. (1999) J. Biol. Chem. 274, 20229-20234). Furthermore, both ARA55 and Smad3 have been suggested to function as bridges for the cross-talk between TGFβ signaling and androgen/AR action (Fujimoto et al. (1999) J. Biol. Chem. 274, 8316-8321; Kang et al. (2001) Proc. Natl. Acad. Sci. U.S.A. 98, 3018-3023).

a) Rb

140. Androgen receptor mutations do not account for all cases of androgen-independent tumors, because some androgen-independent tumors retain wild-type AR. A significant percentage of androgen-insensitive tumors have been correlated with reduced expression of retinoblastoma protein (Rb) (Bookstein, et al., Science 247:712-715, (1990)), expression a truncated Rb protein (Bookstein, et al. Proc. Natl. Acad. Sci. USA 87:7762-7766 (1990)), or a missing Rb allele (Brooks, et al. Prostate 26:35-39, (1995)). The prostate cancer cell line DU145 has an abnormal short mRNA transcript of Rb exon 21 (Sarkar, et al. Prostate 21:145-152 (1992)) and transfecton of the wild-type Rb gene into DU145 cells was shown to repress the malignant phenotype (Bookstein, et al. Proc. Natl. Acad. Sci. USA 87: 7762-7766 (1990)).

141. Rb functions in the control of cell proliferation and differentiation (Weinberg, R. A., Cell 81:323-330 (1995) i Kranenburg et al., FEBS Lett. 367:103-106 (1995)). In resting cells, hypophophorylated Rb prevents inappropriate entry of cells into the cell division cycle.

142. Phosphorylation of Rb by cyclin-dependent kinases relieves Rb-mediated growth suppression, and allows for cell proliferation (Dowdy et al., Cell 73:499-511 (1993) i Chen et al., Cell 58:1193-1198 (1989)). Conversely, dephosphorylation of Rb during G1 progression induces growth arrest or cell differentiation (Chen et al. (1989) i Mihara et al., Science 246:1300-1303 (1989)). In dividing cells, Rb is dephosphorylated during mitotic exit and G1 entry (Ludlow et al., Mol. Cell. Biol. 13:367-372 (1993)). This dephosphorylation activates Rb for the ensuing G1 phase of the cell cycle, during which Rb exerts it growth suppressive effects.

143. Disclosed herein Rb can induce transcription activity of wtAR or ˜s877t in the presence of DHT, E2, or HF, and rnARe708k in the presence of DHT. We also discovered that Rb and ARA70 transciptional activity act synergistically to enhance transciptional activity of ARs. The sequence of the cloned Rb gene and the deduced amino acid sequence of the ORF are shown in SEQ ID NO:7 and SEQ ID NO:8, respectively. An Rb polypeptide is a polypeptide that is substantially homologous to SEQ ID NO:8, that interacts with the N-terminal domain of AR, and which acts synergistically with ARA70 in enhancing transactivation by AR.

b) ARA24

144. As described in the examples, experiments undertaken to identify potential coactivators that interact with the AR poly-Q region led to the isolation of a clone encoding a coactivator, designated ARA24, that interacts with the poly-Q region. The sequences of the ARA24 clone and its putative translation product is shown in SEQ ID NO:5 and SEQ ID NO:6.

145. The ARA24 clone has an ORF that is identical to the published ORF for human Ran, an abundant, ras-like small GTPase (Beddow et al. Proc. Natl. Acad. Sci. USA 92:3328-3332, 1995). Overexpression of ARA24 in the presence of DHT does enhance transcription activation by AR over that observed in cells transfected with AR alone. Moreover, expression of antisense ARA24 (ARA24 as) does reduce DHT-induced transcription activation.

146. Disclosed are ARA24 polypeptides that interact with the poly-Q region of an AR as disclosed herein. An ARA24 polypeptide is further characterized by its ability to increase transactivation when overexpressed in eukaryotic cells; having some endogenous ARA24, but expression of an ARA24 antisense RNA reduces AR receptor transactivation.

c) ARA55

147. Among several AR coregulators, ARA70 and ARA55 can enhance the androgenic effect of HF, the active metabolite of flutamide^{26D}. ARA55 has higher expression in prostate cancer compared to normal prostate^{6D}. TIF2 and SRC-1 are highly expressed in most recurrent prostate tumor after androgen ablation therapy^{27D}. The increasing expression of TIF-2 and SRC-1 after androgen deprivation has been proposed to play a role in tumor progression, but they weakly promote the androgenic effect of HF.

148. The polynucleotide sequence of ARA55 (SEQ ID NO:3) exhibits high homology to the C-terminus of mouse hic5 (hydrogen peroxide inducible clone) (Pugh, B., Curro Opin. Cell Biol. 8:303-311 (1996)), and like hic5, ARA55 expression is induced by TGFb. Cotransfection assays of transcription activation, which are described in detail below, revealed that ARA55 is able to bind to both wtAR and mART887S in a ligand-dependent manner to enhance AR transcription activities. ARA55 enhanced transcription activation by wtAR in the presence of 10⁻⁹ M DHT or T, but not 10⁻⁹ M E2 or HF. In contrast, ARA55 can enhance transcription activation by mART887S in the presence of DHT, testosterone (T), E2, or HF. ARA55 did not enhance transcription activation of mARe708k in the presence of E2, but can enhance transcription in the presence of DHT or T.

149. The C-terminal domain of ARA55 (amino acids 251-444 of SEQ ID NO:3) is sufficient for binding to ARs, but does not enhance transcription activation by ARs.

150. The invention is not limited to the particular ARA55 polypeptide disclosed in SEQ ID NO:4. It is expected that any ARA55 polypeptide could be used in the practice of the present invention. By “an ARA55 polypeptide” it meant a polypeptide that is capable of enhancing transactivation of wtAR” the mutant receptor mARt877a, in the presence of DHT, E2, or HF or intact receptor mARe708k in the presence of DHT or T. Such polypeptides include allelic variants and the corresponding genes from other mammalian species as well as truncations.

151. The AR N-terminal domain comprises a polymorphic poly-glutamine (Q) stretch and a polymorphic poly-glycine (G) stretch that account for variability in the length of human AR cDNA observed. The length of the poly-Q region (normally 11-33 residues in length) is inversely correlated with the risk of prostate cancer, and directly correlated with the SBMA, or Kennedy's disease (La Spada et al., Nature (London) 352:77-79 (1991>. The incidence of higher grade, distant metastatic, and fatal prostate cancer is higher in men having shorter AR poly-Q stretches.

d) ARA54 and Mutant ARA54s

152. ARA54 is a 54 kDa protein that interacts with AR in an androgen-dependent manner. Coexpression of ARA54 and AR in a mammalian two-hybrid system demonstrated that reporter gene activity was enhanced in an androgen-dependent manner. ARA54 functions as a coactivator relatively specific for AR-mediated transcription. However, ARA54 may also function as a general coactivator of the transcription activity for other steroid receptors through their cognate ligands and response elements. ARA54 was found to enhance the transcription activity of AR and PR up to 6 fold and 3-5 fold, respectively. In contrast, ARA54 has only marginal effects (less than 2 fold) on glucocorticoid receptor (GR) and estrogen receptor (ER) in DU145 cells.

153. Coexpression of ARA54 with known AR coactivators SRC-1 or ARA70 revealed that each of these coactivators may contribute individually to achieve maximal AR-mediated transcription activity. Moreover, when ARA54 was expressed simultaneously with SRC-1 or ARA70, the increase in AR-mediated transactivation was additive but not synergistic relative to that observed in the presence of each coactivator alone.

154. The C-terminal domain of ARA54 (a.a. 361-471 of SEQ ID NO:1) serves as a dominant negative inhibitor of AR-mediated gene expression of target genes. Coexpression of exogenous full-length ARA54 can reduce this squelching effect in a dose-dependent manner. ARA54 enhanced transactivation of wtAR in the presence of DHT (10⁻¹⁰ to 10⁻⁸ M) by about 3-5 fold. However, transactivation of wtAR was enhanced only marginally with E2 (10⁻⁹-10⁻⁷ M) or HF (10⁻⁷-10⁻⁵ M) as the ligand. The ability of ARA54 to enhance transactivation by two mutant receptors (rnARt877a and mARe708k) that exhibit differential sensitivities to E2 and HF (Yeh et al., Proc. Natl. Acad. Sci. USA, in press (1998)) was also examined. The mutant mARt 877a, which is found in many prostate tumors (23), was activated by E2 (10⁻⁹-10⁻⁷ M) and HF (10⁻⁷-10⁻⁵ M), and ARA54 could further enhance E2- or HF-mediated AR transactivation. In contrast, the mutant mARe708k, first identified in a yeast genetic screening (Wang, C., Ph.D. Thesis of University of Wisconsin-Madison (1997)), exhibited ligand specificity and response to ARE54 comparable to that of wtAR.

155. It is expected that any polypeptide having substantial homology to ARA54 that still actuates˜the same biological effect can function as “an ARA54 polypeptide.” With the sequence information disclosed herein, one skilled in the art can obtain any ARA54 polypeptide using standard molecular biological techniques. An ARA54 polypeptide is a polypeptide that is capable of enhancing transactivation of AR in an androgen-dependent manner, enhancing E2 or HF transactivation by the mutant receptor mARt877a, and reducing inhibition of AR-mediated gene expression caused by overexpression of the C-terminal domain of ARA54 (a.a. 361-471 of SEQ ID NO:1). The sequence information presented in this application can be used to identify, clone or sequence allelic variations in the ARA54 genes as well as the counterpart genes from other mammalian species. it is also contemplate that truncations of the native coding region can be made to express smaller polypeptides that will retain the same biological activity.

156. The ligand-bound androgen receptor (AR) regulates target genes via a mechanism involving coregulators, such as ARA54. Using in vitro mutagenesis and a yeast two-hybrid screening assay, a mutant ARA54 (mt-ARA54) carrying a point mutation at amino acid 472 changing a glutamic acid to lysine, which acts as a dominant-negative inhibitor of AR transactivation, was isolated. In transient transfection assays of prostate cancer cell lines, the mt-ARA54 suppressed endogenous mutated AR- and exogenous wild-type AR-mediated transactivation in LNCaP and PC-3 cells, respectively. In DU145 cells, the mt-ARA54 suppressed exogenous ARA54-, but not other coregulators-, such as ARA55- or SRC-1-, enhanced AR transactivation. In the LNCaP cells stably transfected with the plasmids encoding the mt-ARA54 under the doxycycline inducible system, overexpression of the mt-ARA54 inhibited cell growth and endogenous expression of prostate-specific antigen. Mammalian two-hybrid assays further demonstrated that the mt-ARA54 can disrupt the interaction between wild-type ARA54 molecules, suggesting ARA54 dimerization or oligomerization may play an essential role in the enhancement of AR transactivation. Together, these results demonstrate that a dominant-negative AR coregulator can suppress AR transactivation and cell proliferation in prostate cancer cells, and interruption of the AR coregulator function could lead to down-regulation of AR activity.

157. The C-terminal region (amino acids 361-474) of ARA54 (C′-ARA54), which was originally isolated from a human prostate cDNA library, interacted with AR (Kang et al. (1999) J. Biol. Chem. 274, 8570-8576). Full-length ARA54 (fl-ARA54), but not C′-ARA54, enhanced AR transactivation (Kang et al. (1999) J. Biol. Chem. 274, 8570-8576; Yeh et al. (1999) Endocrine 11, 195-202). Disclosed are compositions and methods that can suppress AR transactivation induced by fl-ARA54 in prostate cancer cells. Mutant ARA54, which has lost the ability to bind to AR, is disclosed herein to act as a dominant-negative inhibitor of AR transcription. Using a chemical mutagenesis method to create a mutated C′-ARA54 library for two-hybrid screening in yeast, a mutant ARA54 (mt-ARA54), C-terminal fragment of ARA54 with a point mutation, which functions in a dominant-negative manner was isolated. This dominant-negative clone disrupts the ability of wild-type ARA54 to interact with itself, indicating that ARA54 dimerization or oligomerization can play an important role in the enhancement of AR transactivation. The hydroxylamine-mediated mutagenesis screening technique disclosed herein can be used to isolate additional dominant-negative coregulators that are able to inhibit a broad spectrum of receptor-coregulator interactions. Such dominant-negative coregulators could be used in gene therapy as part of a therapeutic option in the treatment of prostate cancer.

e) ARA 70

158. ARA70 is a ligand-enhanced AR coregulator (Dynlacht et al. (1991) Cell 66, 563-576). The androgenic activity of antiandrogens or 17β-estradiol (Glass et al. (2000) Genes & Development. 14, 121-41) can also be enhanced in the presence of ARA70 (Yeh et al. (1998) Proc Natl Acad Sci USA 95, 5527-5532; Miyamoto et al. (1998) Proc Natl Acad Sci USA 95, 7379-7384; Yeh et al. (1999) Proc Natl Acad Sci USA 96, 5458-5463.), consistent with previous observations that the AR can be activated by non-androgen agonists (Kemppainen et al. (1992) J. Biol. Chem. 267, 968-974; Kokontis et al. (1991) Receptor 1, 271-279Truica; Truica et al. (2000) Cancer Res. 1, 4709-4713).

159. Another study also indicated that the expression of ARA70 could be induced in the absence of androgen in the human prostate cancer xenograft, CWR22 (43B). Furthermore, resveratrol, a growth inhibitor for prostate cancer LNCaP cells, could repress the expression of ARA70 and AR transactivation (Mitchell et al. (1999) Cancer Res. 59, 5892-5895).

160. Disclosed herein are the receptor interaction domain (RID) of ARA70, ARA70-N2, which excludes the putative LXXLL signature motif ARA70-N2 can function as a dominant negative repressor to inhibit AR-induced transactivation by ARE-containing reporter gene assay or prostate specific antigen (PSA) mRNA expression (45). Also disclosed is that full length ARA70 is located in the cytosol. Also disclosed ARA70 can stabilize and/or increase the synthesis of AR protein, potentially enhancing AR transactivation. Thus, ARA70 is a cytosolic AR coregulator that may enhance AR transactivation by either stabilizing newly synthesized AR protein or promoting AR nuclear translocation.

161. The p160 coregulators such as SRC-1, and many other SR associated proteins capable of interacting with liganded SRs, share a common motif containing a core consensus sequence, LXXLL. These motifs are sufficient for ligand-dependent interaction with SRs, and were predicted to assume a helical conformation (Anzick et al. (1997) Science 277, 965-968); Heery et al. (1997) Nature 387, 733-736).

162. SRC-1, TIF2/GRIP1, and p/CIP/AIB1/ACTR all contain three LXXLL motifs in a conserved central sequence which has been defined as the SR interaction domain. In addition, SRC-1 has a single splicing variant that has an additional carboxyl-terminal LXXLL-containing motif (Hsiao et al. (1999) J. Biol. Chem. 274, 20229-20234; Anzick et al. (1997) Science 277, 965-968). Our conclusion that ARA70-N2, lacking the LXXLL motif, interacts with the AR contradicts the generally accepted concept that the LXXLL domain within SR coregulators plays an essential role in the interaction with SRs (Heery et al. (1997) Nature 387, 733-736).

f) ARA 267

163. Disclosed herein is the cloning and characterization of ARA267, a novel AR-associated protein that contains a Su(var)3-9, Enhancer-of-zeste, and Trithorax (SET) domain.

164. For example, disclosed is ARA267, with a calculated molecular weight of 267 kD, named as ARA267. ARA267 contains 2427 amino acids, including 1 SET domain, 2 LXXLL motifs, 3 nuclear translocation signal sequences, and 4 PHD finger domains. Northern blot analyses reveal that ARA267 is expressed predominantly in the lymph node as a 13 kb and 10 kb transcript. HepG2 is the only cell line tested that does not express ARA267. Yeast two-hybrid and glutathione S-transferase (GST) pull-down assays show that both the N-terminus and C-terminus of ARA267 interact with AR DNA-binding domain and ligand-binding domain. Unlike other coregulator, such as CBP, which enhance the interaction between the N-terminus and C-terminus of AR, we found that ARA267 has little influence on the interaction between N-terminus and C-terminus of AR. Luciferase and CAT assays show that ARA267 can enhance AR transactivation in a dihydrotestosterone-dependant manner in PC-3 and H11299 cells. ARA267 can also enhance AR transactivation with other coregulators, such as ARA24 or PCAF, a histone acetylase, in an additive manner. Together, our data demonstrate that ARA267 is a new AR coregulator containing the SET domain with an exceptionally larger molecular weight that can enhance AR transactivation in prostate cancer cells.

165. ARA267 is a AR coregulator that contains the SET domain, an evolutionarily conserved sequence that has 130 amino acid motif named from three originally identified proteins: Su(var)3-9, Enhancer-of-zeste, and Trithorax (Jenuwein et al. (1998) Cell Mol Life Sci. 54, 80-93; Firestein et al. (2000) Mol Cell Biol, 20, 4900-4909).

166. These 3 proteins are members of the polycomb group (Pc-G) and Trithoraz group (Tri-G) proteins, that play important roles in the homeotic gene expression in Drosophila (Gould, A. (1997) Curr Opin Genet Dev 7(4), 488-494). Evidence indicates that human homologues of these genes, such as ALR, huASH, or ALL-1 (Prasad et al. (1997) Oncogene 15, 549-560; Nakamura et al. (2000) Proc Natl Acad Sci USA 97, 7284-7289; Gu et al. (1992) Cell 71, 701-708) can also play important roles in the regulation of transcription activation or repression via direct modulation of the chromatin structure (Gould, A. (1997) Curr Opin Genet Dev 7(4), 488-494), which can result in cell growth control or disease progression (Firestein et al. (2000) Mol Cell Biol, 20, 4900-4909; Cardoso et al. (1998) Hum Mol Genet 7, 679-684; Cui, X. et al. (1998) Nat Genet 18,331-337). The SET domains can self interact (Rozovskaia et al. (2000) Oncogene 20, 351-357).

167. One of the most distinct features of SR coregulators is the presence of LXXLL motif, which plays an important role in the interaction between coregulators and receptors for the enhancement of SR transactivation. By mutating LXXLL to LXXAA, Heery et al. found that SRC1 failed to function as a steroid receptor coregulator (Heery et al. 1997 Nature 387, 733-736). Similar results also occurred with the TIFII coregulators (Leers et al. (1998) Mol Cell Biol 18, 6001-6013) ARA267 contains 2 LXXLL motifs consistent with ARA267 enhancement of AR transactivation.

168. In addition to the SET domain and LXXLL motifs, ARA 267 also contains 3 NLS domains that have been shown to play essential roles for the translocation of proteins from cytoplasm to nucleus (Dingwall et al. (1991) Trends Biochem Sci 16, 478-481). Furthermore, ARA267 has 4 PHD fingers that may play important roles in the chromatin-mediated transcription regulation. As these PHD fingers overlap with the Cysteine-rich region, the zinc-finger, and the ring finger, consistent with ARA267 being able to bind to DNA via these regions. Other proteins with Cysteine-rich regions, such as the members of the Trithorax or Polycomb groups are well known for their roles in the chromatin-mediated transcription regulation (Aasland et al. (1995) Trends Biochem Sci 20, 56-59). Some PHD finger proteins have been linked to the chromatin remodeling via histone acetylation (Loewith et al. (2000) Mol Cell Biol 20, 3807-3816). Other SR coregulators, such as TIF1α and CBP/p300 also contain PHD finger motifs and have been demonstrated to play important roles in the SR-mediated gene transcription. The domains of ARA267 are consistent with AR-mediated gene transcription via SET domain or PHD fingers.

169. AR transactivation can be enhanced by 10 nM E2 in the presence of selected coregulators, such as ARA70 (Yeh et al. Proc. Natl. Acad. Sci. U.S.A. (1998) 95, 5527-5532). Han et al. (Han et al. (2001) J Biol Chem 276, 11204-11213), Weigel et al. (Agoulnik et al. (2000) Abstract (#302) in Keystone Steroid Symposium, Colorado), Truica et al. (Han et al. (2001) J Biol Chem 276, 11204-11213) also reported that E2 could enhance AR transactivation in the presence of ARA70, SRC1, or β-Catenin respectively. Results shown in FIG. 20 confirmed these studies. ARA70N can enhance AR transactivation in the presence of 10 nM E2. In contrast ARA267 only has maginal effect on the enhancement of AR transactivation in the presence of 10 nM E2. These data therefore suggest that different coregulators may have distinct mechanism to enhance AR transactivation in the presence of various ligands.

170. Results from FIG. 21 indicate that in the HepG2 and PC3 cells, ARA267 has marginal enhancement effect on the transactivation of other steroid receptors, such as PR, ER and GR. As any given steroid receptor's maximal function could be the combination of the availability of the receptors and their relative abundance compared to many other general transcription factors and coregulators, which could differ in various cell lines (Yeh et al. (1999) Endocrine 11, 195-202), it is consistent that in other cells the ARA267 has different preferential coactivations and may be able to greatly increase the enhancement of other steroid receptor transactivation.

171. ARA267 acts as an AR coregulator to increase AR transactivation.

g) Gelsolin

172. Disclosed herein gelsolin as an antiandrogen, hydroxyflutamide, potentiated androgen receptor coregulator. Hydroxyflutamide, as well as testosterone, can promote the interaction between AR and gelsolin in a dose dependent manner. Gelsolin interacts with AR DNA-binding domain and ligand-binding domain via its C-terminal. Functional analysis further demonstrates that two regions within androgen receptor can block the coactivator activity of gelsolin. The expression of gelsolin is enhanced in LNCaP xenograft and human prostate tumor after androgen ablation treatment. This induction of gelsolin enhances the androgenic activity of hydroxyflutamide and reduces its capacity to suppress AR activity. Together, these data indicate gelsolin is involved in flutamide withdrawal syndrome. Blockage of the interaction between androgen receptor and gelsolin can be used in the treatment of prostate cancer.

173. Disclosed herein gelsolin is a HF responsive AR coregulator and provides models the prostate tumor progression in flutamide withdrawal syndrome. Gelsolin is an actin severing protein well characterized in its function for cytoskeleton reorganization, cell morphology and motility (Kwiatkowski et al. Curr Opin Cell Biol 11, 103-108. (1999); Sun et al. J Biol Chem 274, 33179-33182. (1999)). Since gelsolin is identified as a substrate for capspase-3, its dual roles in promoting apoptosis and protecting cell from apoptosis are reported Koya et al. J Biol Chem 275, 15343-15349. (2000); Fujita et al. Ann NY Acad Sci 886, 217-220 (1999)). Several reports have indicated gelsolin expresses differentially in various cancers, including prostate cancer (Dhanasekaran et al. Nature 412, 822-826. (2001); Lee et al. Prostate 40, 14-19. (1999)).

174. Disclosed herein gelsolin enhances the androgenic activity of HF and the increased expression of gelsolin after androgen ablation treatment.

175. Gelsolin is a multifunction actin-binding protein that has been implicated in cell motility, signalling, apoptosis, and carcinogenesis (Kwiatkowski et al. Curr Opin Cell Biol 11, 103-108. (1999); Sun et al. J Biol Chem 274, 33179-33182. (1999).

176. Disclosed herein gelsolin is an AR coregulator. Other actin-binding proteins, such as filamin (Ozanne et al. Mol Endocrinol 14, 1618-1626. (2000)), and supervillin have also been characterized to function as AR coregulators and modulate AR activity. Early reports have linked actin-associated proteins to the signal transduction pathway in the nucleus (Prendergast et al. Embo J 10, 757-766. (1991); Wulfkuhle et al. J Cell Sci 112, 2125-2136. (1999)).

177. While some reports showed the nuclear localization of gelsolin in differential endothelial cells (Salazar et al. Exp Cell Res 249, 22-32. (1999)), immunostaining data suggested gelsolin was located mainly in the cytosol. As gelsolin lacks the nuclear localization signal, it is possible that gelsolin could be co-translocated into nucleus with binding to other proteins. This is in agreement with the results disclosed herein that gelsolin and AR overexpressed in COS-1 cells revealed that gelsolin was present in the nucleus temporarily after T treatment. Therefore, it is likely that gelsolin interacts with AR at the time of its nuclear localization to facilitate the nuclear translocation of AR.

178. Disclosed herein gelsolin functions as a coregulator of HF activated AR and participates in the development of the “flutamide withdrawal syndrome” because the expression of gelsolin increases after androgen ablation. Disclosed herein, surgical/chemical castration to reduce the androgen concentration increases the gelsolin expression in prostate cancer cells (FIG. 27B, C). This increased gelsolin can then enhance the HF bound AR activity (FIG. 28) to increase tumor growth and the expression of prostate-specific antigen (PSA) which is an androgen regulated clinical marker for prostate cancer. Blockage of the HF-induced interaction between AR and gelsolin can be used for advanced prostate cancer and prostate cancer therapy.

179. Disclosed herein peptides D1 (aa 551-600) and H1-2 (aa 655-695) located within AR DBD and LBD block gelsolin-induced AR activity and these and other homologs can be used in prostate cancer therapy. These two peptides and homlogs can also interfere with functions of other AR coregulators.

180. Gelsolin expression is down-regulated in several cancers, such as prostate, breast, lung, and bladder cancer (Dhanasekaran et al. Nature 412, 822-826. (2001); Asch et al. Cancer Res 56, 4841-4845. (1996); Dosaka-Akita et al. Cancer Res 58, 322-327. (1998); Tanaka et al. Cancer Res 55, 3228-3232. (1995)), therefore it is regarded as a tumor suppressor. However, higher expression of gelsolin was reported to be associated with higher risk of recurrence in lung cancer (Shieh et al. Cancer 85, 47-57. (1999)) and may represent a sensitive and specific marker for renal cystadenomas and carcinoma (Onda et al. J Clin Invest 104, 687-695. (1999)).

h) Supervillin

181. Activation of androgen receptor (AR) via androgen in muscle cells has been closely linked to their growth and differentiation. Disclosed herein is the cloning and characterization of supervillin (SV), a 205 kDa actin binding protein, as an AR coregulator from the skeletal muscle cDNA library. Mammalian two-hybrid and GST pull-down assays indicate a domain within SV (amino acid position 594-1268) can interact with AR N-terminus as well as DNA binding domain-ligand binding domain in a ligand-enhanced manner. Subcellular colocalization studies using fluorescence staining indicates SV can colocalize with AR in the presence of Sc-dihydrotestosterone in COS-1 cells. The functional reporter assays showed full-length SV as well as the SV peptide (amino acid position 831-1281) within the interaction domain can enhance AR transactivation. Furthermore, SV can enhance the endogenous AR target gene, p27^{KIP1}, expression in prostate PC-3(AR2) cells. SV preferentially enhanced AR rather than other tested nuclear receptors and could be induced by natural androgens better than other steroids. SV can also cooperate with other AR coregulators, such as ARA55 or ARA70, to further enhance AR transactivation. Unlike SRC-1 that can enhance the interaction between AR N-terminus and AR C-terminus, SV shows a suppressive effect on N—C interactions.

182. Since the expression of coregulators varies among different cell types, AR functions depend on the availability of expressed coregulators in the same cell. While it is well documented that SRC-1 can enhance estrogen receptor (ER) transactivation in many reporter assays, immunohistochemistry studies, however, demonstrated that SRC-1 and ER are not located in the same subset of epithelial cells within the adult mammary gland (7E). This finding excludes any possibility for SRC-1 to bind to ER and modulate ER function in those cells. Moreover, FHL2 and ARIP3 are two AR coregulators reported to express mostly in myocardium and testes, respectively (Muller et al. (2000) EMBO J. 19, 359-69; Kotaja et al. (2000) Mol. Endocrinol. 14, 1986-2000).

183. Skeletal muscle has been reported to be an AR target organ (Mooradian et al. (1987) Endocr. Rev. 8, 1-28; Doumit et al. (1996) Endocrinology 137, 1385-94). To understand how T induces AR function in skeletal muscle, yeast two-hybrid screen was done to identify T responsive AR interacting proteins from skeletal muscle cDNA library. One of the clones identified from this screening encodes the partial sequence of supervillin (SV).

184. SV is an actin binding protein first identified from blood cells. In addition to blood cells, it also expresses in muscle enriched tissues, especially skeletal muscles, and several cancer cell lines (Pope et al. (1998) Genomics 52, 342-51). The roles of SV in muscle and cancer are still under investigation. Although its carboxyl terminal shows high homology to gelsolin and villin (Pestonjamasp et al. (1997) J. Cell Biol. 139, 1255-69), functional domain studies determined that the amino terminus of SV represents the strong actin binding activity (Wulfkuhle et al. (1999) J. Cell Sci. 112, 2125-36). The nuclear localization signal located in the middle of this protein is functional and may contribute to its nuclear translocation (Wulfkuhle et al. (1999) J. Cell Sci. 112, 2125-36). However, the functions of SV in the cytoskeleton network and the nucleus remain unclear. Early studies also found that SV is a T down-regulated gene in dermal pappiloma cells, which may contribute to male baldness syndrome (Pan et al. (1999) Endocrine 11, 321-7). Recently, the use of systematic RNA mediated interference in C. elegans has demonstrated the SV homologue plays a role in sex determination (Fraser et al. (2000) Nature 408, 325-30). Disclosed herein SV is an AR interacting protein and demonstrate that SV can function as an AR coregulator by enhancing AR transactivation.

185. Disclosed herein SV is an AR coregulator to enhance transactivation from skeletal muscle. SV binds to actin and increases the amount of F-actin and vinculin when overexpressed (Wulfkuhle et al. (1999) J. Cell Sci. 112, 2125-36). These suggest it functions in the cell adhesion and motility. On the other hand, actin itself was proposed to be the key regulator of serum response factor that could modulate gene expression by functioning as a suppressor to sequester the coregulators of serum response factor (Sotiropoulos et al. (1999) Cell 98, 159-69).

186. Among identified AR coregulators, ARA24 and ARA160 interact with ARN (Hsiao et al. (1999) J. Biol. Chem. 274, 22373-9; Hsiao et al. (1999) J. Biol. Chem. 274, 20229-34), ubc-9 and SNURF interact with AR DBD (Poukka et al. (1999) J. Biol. Chem. 274, 19441-6; Poukka et al. (2000) J. Cell Sci. 113, 2991-3001), and ARA54, ARA55 and ARA70 interact with AR LBD (Fujimoto et al. (1999) J. Biol. Chem. 274, 8316-21; Kang et al. (1999) J. Biol. Chem. 274, 8570-6; Yeh, S. & Chang, C. (1996) Proc. Natl. Acad. ScL USA 93, 5517-21). SV and some nuclear receptor coregulator members, such as NCoA, can interact with both N-terminal activation function-1 and C-terminal activation function-2 of AR (Bevan et al. (1999) Mol. Cell. Biol. 19, 8383-92; Alen et al. (1999) Mol. Cell. Biol. 19, 6085-97). It has been reported that the LXXLL motif of several coregulators plays essential role for the interaction and coactivation function with most receptors except AR (Heery et al. (1997) Nature 387, 733-6; Leo, C. & Chen, J. D. (2000) Gene 245, 1-11). We found that the SV peptide (a.a. 594-1335), which does not contain the LXXLL motif, can still interact with ARN and ARC. The motifs important for AR N—C interaction have been reported (He et al. (2000) J. Biol. Chem. 275, 22986-94). Those motifs, including FXXLF and WXXLF, that play important roles for the interaction with AR C-terminus, are located in ARN. It is possible that AR N—C interactions may stabilize the dimer of AR and promote its activity. Since SV interacts with both N and C-terminus of AR, it is consistent that SV can play a role in the AR dimerization. However, the results in FIG. 34 indicate SV can suppress AR N—C interaction.

187. The disclosed data showed SV(a.a. 831-1281) has a better enhancing effect on AR transactivation compared to full length SV and SV(a.a. 1010-1792). Immunostaining shows this peptide is mainly in the nucleus and colocalizes with DHT bound AR in contrast to SV(a.a. 1010-1792) which remains in the cytosol. The consequence of these events may then result in the increase of AR transactivation.

188. Due to the differences of transcription-translation efficiency of transfected genes, the amount of amount of transfected plasmid expressing coregulators and steroid receptors can be adjusted to an optimal ratio in order to show maximum coactivator activity. For example, SRC-1 needs a ratio up to 100:1 as compared to steroid receptors to show the significant coactivator activity (McInerney et al. (1996) Proc. Natl. Acad. Sci. USA 93, 10069-73; Takeshita et al. (1997) J. Biol. Chem. 272, 27629-34). In contrast, other coregulators, such as ARA55 or ARA70N may require lower ratios of expression plasmids (coregulator:AR up to 3-5:1) for their maximal coactivator activities. Since different cells have various amounts of endogenous coregulators that may affect the impact of exogenously transfected SV, we expect the amount of transfected SV plasmids for maximum AR activity varies between cells. Similarly, SV does not necessarily always function as a coregulator to preferentially enhance AR transactivation as compared to other steroid receptors. Considering that any given cell may have multiple coregulators interacting with multiple steroid receptors, squelching effects can occur in some cells resulting in less coregulator effect for any particular receptor. Furthermore, under varying physiological environments and clinical situations, cells are exposed to multiple steroid hormones. Compared to ARA70N, SV is generally much weaker in promoting non-androgen steroid-mediated AR transactivation. SV, however, is able to coordinate with other AR coregulators, such as ARA70N and ARA55, to enhance AR transactivation. These results again suggest the final AR activity may be the balance and coordination of multiple coregulators in any given cell. It is well documented that different concentrations of DHT and various amounts of AR within one cell may change the androgen-AR function to either promote cell proliferation or stimulate cell apoptosis. For example, while 0.1 nM DHT can stimulate LNCaP cell proliferation, 10 nM DHT promotes LNCaP cell apoptosis (Langeler et al. (1993) Prostate 23, 213-23; Sonnenschein et al. (1989) Cancer Res. 49, 3474-81). Similarly, 10 nM DHT can also arrest PC-3(AR2) cell growth and promote cells into apoptosis (Yuan et al. (1993) Cancer Res. 53, 1304-11; Heisler et al. (1997) Mol. Cell Endocrinol. 126, 59-73). Androgen can down-regulate the SV gene expression (Wulfkuhle et al. (1999) J. Cell Sci. 112, 2125-36), SV may provide a nice feedback mechanism for cells to determine how AR and SV perform their physiological function in muscle and other cells.

i) Steriod receptors

189. Ligand-unbound SRs have been found in the cytosol associated with heat shock proteins (HSPs), including HSP90, HSP70, and HSP56 (Rajapandi et al. (2000) J. Biol. Chem. 275, 22597-22604; Pratt, W. B., and Toft, D. O. (1997) Endocr. Rev. 18, 306-360; Pratt et al. (1993) J. Steroid Biochem. Mol. Biol. 46, 269-279). Studies of the HSP chaperone machinery in eukaryotes have suggested that HSP family proteins are sufficient to prevent SR misfolding and aggregation and promote refolding of denatured polypeptides (Fliss et al. (1999) J. Biol. Chem. 274, 34045-34052; Chen, S., and Smith, D. F. (1998) J. Biol. Chem. 273, 35194-35200). It has also been reported that HSP90 may enhance the ligand binding capacity of the AR, but not the glucocorticoid receptor (GR) (Fang et al. (1996) J. Biol. Chem. 271, 28697-28702).

190. Recently, it has been reported that several SRs can interact directly with components of the basal transcription machinery, such as TBP (Sadovsky et al. (1995) Mol. Cell. Biol. 15, 1554-1563), TFIIB, TFIIF (Baniahmad et al. (1993) Proc. Natl. Acad. Sci. USA 90, 8832-8836), and TFIIH (McEwan, I. J., and Gustafsson, J. (1997) Proc. Natl. Acad. Sci. USA 94, 8485-8490). In addition, specific sets of proteins are recruited by the SRs as coregulators that may function as bridge factors between the receptors and general transcription factors in the preinitiation complex (Lee, D. K., Duan, H. O., and Chang, C. (2000) J. Biol. Chem. 275, 9308-9313; Pugh, B. F., and Tjian, R. (1990) Cell 61, 1187-1197; Ptashne, M., and Gann, A. A. F. (1990) Nature 346, 329-331).

191. Identifying and understanding the function of individual components of these complexes are crucial in determining how SRs regulate their target genes. Indeed, several coregulators including ARA70 (Dynlacht et al. (1991) Cell 66, 563-576), ARA55 (Yeh, S., and Chang, C. (1996) Proc. Natl. Acad. Sci. USA 93, 5517-5521), ARA54 (Fujimoto et al. (1999) J. Biol. Chem. 274, 8316-8321), ARA 160 (Kang et al. (1999) J. Biol. Chem. 274, 8570-8576), ARA24 (Hsiao, P., and Chang, C. (1999) J. Biol. Chem. 274, 22373-22379), SRC-1 (Hsiao et al. (1999) J. Biol. Chem. 274, 20229-20234), GRIP1/TIF2 (Onate et al. (1995) Science 270, 1354-1357; Hong et al. (1996) Proc Natl Acad Sci USA 93, 4948-4952; RAC3/ACTR/AIB1/PCIP/SRC-3 (Voegel et al. (1996) EMBO J. 15, 3667-3675; Li et al. (1997) Proc Natl Acad Sci USA 94, 8479-8484; Chen et al. (1997) Cell 90, 569-580; Anzick et al. (1997) Science 277, 965-968); CBP/p300 (Torchia et al. (1997) Nature 387, 677-684), and the BRCA1 and Rb tumor suppressors (Smith et al. (1996) Proc Natl Acad Sci USA 93, 8884-8888; Yeh et al. (2000) Proc. Natl. Acad. Sci. USA 97, 11256-11261; Yeh et al. (1998) Biochem. Biophys. Res. Commun. 242,361-367).), have been identified as being able to modulate the transactivation of SRs. Coregulators have also had their transcription activation of SRs linked to chromatin acetylation. Some of these coregulators, such as RAC3/ACTR (. Voegel et al. (1996) EMBO J. 15, 3667-3675; Li et al. (1997) Proc Natl Acad Sci USA 94, 8479-8484; Chen et al. (1997) Cell 90, 569-580; Anzick et al. (1997) Science 277, 965-968).

192. CBP/p300 (34), and SRC-1 (35B), have been found to either have intrinsic histone acetyltransferase (HAT) activity or have the capacity to recruit the p300/CBP-associated factor (P/CAF) that has HAT activity.

5. Molecules that Coregulate AR

a) Functional Nucleic Acids

193. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

194. Functional nucleic acid molecules can interact with any macromolecule, such as DNA, RNA, polypeptides, or carbohydrate chains. Thus, functional nucleic acids can interact with the mRNA of AR, ARA54, ARA55, SRC-1, ARA70, RB, ARA24, ARA160, ARA267, gelsolin, and/or supervillin, or fragment thereof, or the genomic DNA of AR, ARA54, ARA55, SRC-1, ARA70, RB, ARA24, ARA160, ARA267, gelsolin, and/or supervillin, or fragment thereof, or they can interact with the polypeptide AR, ARA54, ARA55, SRC-1, ARA70, RB, ARA24, ARA160, ARA267, gelsolin, and/or supervillin, or fragment thereof. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

195. Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than 10⁻⁶. It is more preferred that antisense molecules bind with a k_{d} less than 10⁻⁸. It is also more preferred that the antisense molecules bind the target moelcule with a k_{d} less than 10⁻¹⁰. It is also preferred that the antisense molecules bind the target molecule with a k_{d} less than 10⁻¹². A representative sample of methods and techniques which aid in the design and use of antisense molecules can be found in the following non-limiting list of United States patents: U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

196. Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶. It is more preferred that the aptamers bind the target molecule with a k_{d} less than 10⁻⁸. It is also more preferred that the aptamers bind the target molecule with a k_{d} less than 10⁻¹⁰. It is also preferred that the aptamers bind the target molecule with a k_{d} less than 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10 fold lower than the k_{d} with a background binding molecule. It is more preferred that the aptamer have a k_{d} with the target molecule at least 100 fold lower than the k_{d} with a background binding molecule. It is more preferred that the aptamer have a k_{d} with the target molecule at least 1000 fold lower than the k_{d} with a background binding molecule. It is preferred that the aptamer have a k_{d} with the target molecule at least 10000 fold lower than the k_{d} with a background binding molecule. It is preferred when doing the comparison for a polypeptide for example, that the background molecule be a different polypeptide. For example, when determining the specificity of TR2, TR4, AR, or ER, or fragments thereof, aptamers, the background protein could be serum albumin. Representative examples of how to make and use aptamers to bind a variety of different target molecules can be found in the following non-limiting list of United States patents: U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

197. Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following United States patents: U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following United States patents: U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following United States patents: U.S. Pat. Nos. 5,595,873 and 5,652,107). There are also a number of ribozymes that are not found in natural systems, but which have been engineered to catalyze specific reactions de novo (for example, but not limited to the following United States patents: U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of United States patents: U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

198. Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶. It is more preferred that the triplex forming molecules bind with a k_{d} less than 10⁻⁸. It is also more preferred that the triplex forming molecules bind the target moelcule with a k_{d} less than 10⁻¹⁰. It is also preferred that the triplex forming molecules bind the target molecule with a k_{d} less than 10⁻¹². Representative examples of how to make and use triplex forming molecules to bind a variety of different target molecules can be found in the following non-limiting list of United States patents: U.S. Pat. Nos. 5,176,996, 5,645,985, 5,650,316, 5,683,874, 5,693,773, 5,834,185, 5,869,246, 5,874,566, and 5,962,426.

199. External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

200. Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al. Proc. Natl. Acad. Sci. (USA) 92:2627-2631 (1995)). Representative examples of how to make and use EGS molecules to facilitate cleavage of a variety of different target molecules be found in the following non-limiting list of United States patents: U.S. Pat. Nos. 5,168,053, 5,624,824, 5,683,873, 5,728,521, 5,869,248, and 5,877,162

b) Antibodies

(1) Antibodies Generally

201. The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with AR, ARA54, ARA55, SRC-1, ARA70, RB, ARA24, ARA160, ARA267, gelsolin, and/or supervillin, or fragment thereof, such that AR, ARA54, ARA55, SRC-1, ARA70, RB, ARA24, ARA160, ARA267, gelsolin, or supervillin, or fragment thereof, are regulated for transactivation activity, such as increasing or decreasing transactivation activity. Antibody also includes, chimeric antibodies and hybrid antibodies, with dual or multiple antigen or epitope specificities, and fragments, such as F(ab′)2, Fab′, Fab and the like, including hybrid fragments, as well as conjugates of antibody fragments and antigen binding proteins (single chain antibodies) as described, for example, in U.S. Pat. No. 4,704,692, the contents of which are hereby incorporated by reference. Antibodies that bind the disclosed regions of AR, ARA54, ARA55, SRC-1, ARA70, RB, ARA24, ARA160, ARA267, gelsolin, and/or supervillin, or fragment thereof, such that AR, ARA54, ARA55, SRC-1, ARA70, RB, ARA24, ARA160, ARA267, gelsolin, or supervillin, or fragment thereof, regulate, such as decrease or increase, their transactivation activity are also disclosed. The antibodies can be tested for their desired activity using the in vitro assays described herein, or by analogous methods, after which their in vivo therapeutic and/or prophylactic activities are tested according to known clinical testing methods. Thus, fragments of the antibodies that retain the ability to bind their specific antigens are provided. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

202. The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

203. The disclosed monoclonal antibodies can be made using any procedure, which produces mono clonal antibodies. For example, monoclonal antibodies of the invention can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro, e.g., using the binding domains of the compositions described, herein, such as the PTAP binding domain, described herein.

204. The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

205. In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

206. The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

207. As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods of the invention serves to lessen the chance that an antibody administered to a human will evoke an undesirable immune response.

(2) Human Antibodies

208. The human antibodies of the invention can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86-95, 1991). Human antibodies of the invention (and fragments thereof) can also be produced using phage display libraries (Hoogenboom et al., J. Mol. Biol., 227:381, 1991; Marks et al., J. Mol. Biol., 222:581, 1991).

209. The human antibodies of the invention can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

(3) Humanized Antibodies

210. Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

211. To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

212. Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

(4) Administration of Antibodies

213. Administration of the antibodies can be done as disclosed herein. Nucleic acid approaches for antibody delivery also exist. The broadly neutralizing anti-AR, ARA54, ARA55, SRC-1, ARA70, RB, ARA24, ARA160, ARA267, gelsolin, or supervillin, or fragment thereof, antibody fragments of the invention can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

c) Compositions Identified by Screening with Disclosed Compositions/Combinatorial Chemistry

(1) Combinatorial Chemistry

214. The disclosed compositions can be used as targets for any combinatorial technique to identify molecules or macromolecular molecules that interact with the disclosed compositions in a desired way. The nucleic acids, peptides, and related molecules disclosed herein, such as AR, ARA54, ARA55, SRC-1, ARA70, RB, ARA24, ARA160, ARA267, gelsolin, and/or supervillin, or fragment thereof, can be used as targets for the combinatorial approaches. Also disclosed are the compositions that are identified through combinatorial techniques or screening techniques in which the compositions disclosed in herein, such as AR, ARA54, ARA55, SRC-1, ARA70, RB, ARA24, ARA160, ARA267, gelsolin, and/or supervillin, or fragment thereof, are used as the target in a combinatorial or screening protocol.

215. It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as AR, ARA54, ARA55, SRC-1, ARA70, RB, ARA24, ARA160, ARA267, gelsolin, and/or supervillin, or fragment thereof, are also disclosed. Thus, the products produced using the combinatorial or screening approaches that involve the disclosed compositions, such as AR, ARA54, ARA55, SRC-1, ARA70, RB, ARA24, ARA160, ARA267, gelsolin, and/or supervillin, or fragment thereof, are also considered herein disclosed.

216. Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

217. There are a number of methods for isolating proteins, which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

218. A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

219. Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain. A peptide of choice, for example a portion of AR, ARA54, ARA55, SRC-1, ARA70, RB, ARA24, ARA160, ARA267, gelsolin, and/or supervillin, or fragment thereof, is attached to a DNA binding domain of a transcription activation protein, such as Gal 4. By performing the Two-hybrid technique on this type of system, molecules that bind the portion of AR, ARA54, ARA55, SRC-1, ARA70, RB, ARA24, ARA160, ARA267, gelsolin, or supervillin, or fragment thereof, can be identified.

220. Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

221. Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

222. Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. No. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

223. Screening molecules similar to AR, ARA54, ARA55, SRC-1, ARA70, RB, ARA24, ARA160, ARA267, gelsolin, and/or supervillin, or fragment thereof, for example, for regulation of AR transactivation activity or AR binding ability, for example, is a method of isolating desired compounds.

224. Molecules isolated which bind AR, ARA54, ARA55, SRC-1, ARA70, RB, ARA24, ARA160, ARA267, gelsolin, and/or supervillin, or fragment thereof, are typically competitive regulators so that the heterodimerzation properties, such as regulation of AR, transactivation activity, possessed between AR and ARA54, ARA55, SRC-1, ARA70, RB, ARA24, ARA160, ARA267, gelsolin, and/or supervillin, or fragment thereof, are disclosed.

225. In another embodiment the regulators are non-competitive regulators, which, for example, cause allosteric rearrangements which prevent AR transcription activity regulated by the heterodimers disclosed herein.

226. As used herein combinatorial methods and libraries included traditional screening methods and libraries as well as methods and libraries used in interative processes.

(2) Computer Assisted Drug Design

227. The disclosed compositions can be used as targets for any molecular modeling technique to identify either the structure of the disclosed compositions or to identify potential or actual molecules, such as small molecules, which interact in a desired way with the disclosed compositions. The nucleic acids, peptides, and related molecules disclosed herein can be used as targets in any molecular modeling program or approach.

228. It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as AR, ARA54, ARA55, SRC-1, ARA70, RB, ARA24, ARA160, ARA267, gelsolin, and/or supervillin, or fragment thereof, are also disclosed. Thus, the products produced using the molecular modeling approaches that involve the disclosed compositions, such as AR, ARA54, ARA55, SRC-1, ARA70, RB, ARA24, ARA160, ARA267, gelsolin, and/or supervillin, or fragment thereof, are also considered herein disclosed.

229. Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

230. Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

231. A number of articles review computer modeling of drugs interactive with specific proteins, such as Rotivinen, et al., 1988 Acta Pliarniaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, OSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

232. Although described above with reference to design and generation of compounds which could alter binding, one could also screen libraries of known compounds, including natural products or synthetic chemicals, and biologically active materials, including proteins, for compounds which alter substrate binding or enzymatic activity.

d) Methods of Identifying Regulators of AR-TR4 Interactions

233. Disclosed are methods of identifying a regulator of an interaction between AR and ARA54, ARA55, SRC-1, ARA70, RB, ARA24, ARA160, ARA267, gelsolin, and/or supervillin, or fragment thereof, comprising incubating a library of molecules with AR forming a mixture, and identifying the molecules that disrupt the interaction between AR and ARA54, ARA55, SRC-1, ARA70, RB, ARA24, ARA 160, ARA267, gelsolin, and/or supervillin, or fragment thereof, wherein the interaction disrupted comprises an interaction between the AR and TR4 binding site.

234. Also disclosed are methods, wherein the step of isolating comprises incubating the mixture with a molecule comprising AR, ARA54, ARA55, SRC-1, ARA70, RB, ARA24, ARA160, ARA267, gelsolin, and/or supervillin, or fragment thereof.

235. Disclosed are methods of identifying a regulator of an interaction between AR and ARA54, ARA55, SRC-1, ARA70, RB, ARA24, ARA160, ARA267, gelsolin, and/or supervillin, or fragment thereof, comprising incubating a library of molecules with TR4 forming a mixture, and identifying the molecules that disrupt the interaction between AR and ARA54, ARA55, SRC-1, ARA70, RB, ARA24, ARA160, ARA267, gelsolin, and/or supervillin, or fragment thereof, wherein the interaction disrupted comprises an interaction between the AR and ARA54, ARA55, SRC-1, ARA70, RB, ARA24, ARA160, ARA267, gelsolin, or supervillin, or fragment thereof, binding site.

236. Also disclosed are the methods, wherein the step of isolating comprises incubating the mixture with molecule comprising AR, ARA54, ARA55, SRC-1, ARA70, RB, ARA24, ARA160, ARA267, gelsolin, and/or supervillin, or fragment thereof.

237. Also disclosed are compositions produced by any of the processes as disclosed herein, as well as compositions capable of being identified by the processes disclosed herein.

238. Disclosed are methods of manufacturing a composition for regulating the interaction between AR and ARA54, ARA55, SRC-1, ARA70, RB, ARA24, ARA160, ARA267, gelsolin, and/or supervillin, or fragment thereof, comprising synthesizing the regulators as disclosed herein.

239. Also disclosed are methods that include mixing a pharmaceutical carrier with the regulators as disclosed herein, and produced by any of the disclosed methods.

240. Disclosed are methods of identifying regulators of AR and ARA54, ARA55, SRC-1, ARA70, RB, ARA24, ARA160, ARA267, gelsolin, and/or supervillin, or fragment thereof, interaction comprising, a) administering a composition to a system, wherein the system supports AR and ARA54, ARA55, SRC-1, ARA70, RB, ARA24, ARA160, ARA267, gelsolin, and/or supervillin, or fragment thereof, interaction, b) assaying the effect of the composition on the amount of AR-AR, ARA54, ARA55, SRC-1, ARA70, RB, ARA24, ARA160, ARA267, gelsolin, and/or supervillin, or fragment thereof, in the system, and c) selecting a composition which causes a decrease in the amount of AR, ARA54, ARA55, SRC-1, ARA70, RB, ARA24, ARA160, ARA267, gelsolin, and/or supervillin, or fragment thereof, present in the system relative to the system without the addition of the composition.

241. Also disclosed are methods of identifying regulators of AR transcription activity comprising, a) administering a composition to a system, wherein the system supports AR transcription activity, b) assaying the effect of the composition on the amount of AR transcription activity in the system, and c) selecting a composition which causes a decrease in the amount of AR transcription activity present in the system relative to the system without the addition of the composition.

6. Aspects Applicable to All Compositions

a) Sequence Similarities

242. It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

243. In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

244. Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

245. The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

246. For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

b) Hybridization/Selective Hybridization

247. The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

248. Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987: 154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

249. Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

250. Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

251. Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

252. It is understood that those of skill in the art understand that if a composition or method meets any one of these criteria for determining hybridization either collectively or singly it is a composition or method that is disclosed herein.

c) Nucleic Acids

253. There are a variety of molecules disclosed herein that are nucleic acid based, including for example the nucleic acids that encode, for example AR, ARA54, ARA55, SRC-1, ARA70, RB, ARA24, ARA160, ARA267, gelsolin, or supervillin, or fragment thereof, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

(1) Nucleotides and Related Molecules

254. A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

255. A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

256. Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

257. It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556),

258. A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

259. A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

(2) Sequences

260. There are a variety of sequences related to the genes of AR, ARA54, ARA55, SRC-1, ARA70, RB, ARA24, ARA160, ARA267, gelsolin, and/or supervillin, or fragment thereof, which can be found at Genbank, at for example, http://www.pubmed.gov and these sequences and others are herein incorporated by reference in their entireties as well as for individual subsequences contained therein.

261. Those of skill in the art understand how to resolve sequence discrepancies and differences and to adjust the compositions and methods relating to a particular sequence to other related sequences (i.e. sequences of AR, ARA54, ARA55, SRC-1, ARA70, RB, ARA24, ARA160, ARA267, gelsolin, and/or supervillin, or fragment thereof). Primers and/or probes can be designed for any AR, ARA54, ARA55, SRC-1, ARA70, RB, ARA24, ARA160, ARA267, gelsolin, and/or supervillin, or fragment thereof sequence given the information disclosed herein and known in the art.

(3) Primers and Probes

262. Disclosed are compositions including primers and probes, which are capable of interacting with the AR, ARA54, ARA55, SRC-1, ARA70, RB, ARA24, ARA160, ARA267, gelsolin, and/or supervillin, or fragment thereof, as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the AR, ARA54, ARA55, SRC-1, ARA70, RB, ARA24, ARA160, ARA267, gelsolin, and/or supervillin, or fragment thereof, and/or fragments thereof, nucleic acid or region of the ARA54, ARA55, SRC-1, ARA70, RB, ARA24, ARA160, ARA267, gelsolin, and/or supervillin, or fragment thereof, nucleic acid or they hybridize with the complement of the ARA54, ARA55, SRC-1, ARA70, RB, ARA24, ARA160, ARA267, gelsolin, and/or supervillin, or fragment thereof, nucleic acid or complement of a region of the ARA54, ARA55, SRC-1, ARA70, RB, ARA24, ARA160, ARA267, gelsolin, and/or supervillin, or fragment thereof, thereof nucleic acid.

d) Delivery of the Compositions to Cells

263. There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991) Such methods are well known in the art and readily adaptable for use with the compositions and methods described herein. In certain cases, the methods will be modifed to specifically function with large DNA molecules. Further, these methods can be used to target certain diseases and cell populations by using the targeting characteristics of the carrier.

(1) Nucleic Acid Based Delivery Systems

264. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

265. As used herein, plasmid or viral vectors are agents that transport the disclosed nucleic acids, such as nucleic acids encoding ARA54, ARA55, SRC-1, ARA70, RB, ARA24, ARA160, ARA267, gelsolin, and/or supervillin, or fragment thereof, into the cell without degradation and include a promoter yielding expression of the gene in the cells into which it is delivered. In some embodiments the vectors are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone, as well as lentiviruses. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

266. Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(a) Retroviral Vectors

267. A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

268. A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

269. Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(b) Adenoviral Vectors

270. The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

271. A viral vector can be one based on an adenovirus which has had the E1 gene removed and these virons are generated in a cell line such as the human 293 cell line. In another preferred embodiment both the E1 and E3 genes are removed from the adenovirus genome.

(c) Adeno-Asscociated Viral Vectors

272. Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

273. In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

274. Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorproated by reference for material related to the AAV vector.

275. The vectors of the present invention thus provide DNA molecules which are capable of integration into a mammalian chromosome without substantial toxicity.

276. The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(d) Large Payload Viral Vectors

277. Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

278. Other useful systems include, for example, replicating and host-restricted non-replicating vaccinia virus vectors.

(2) Non-Nucleic Acid Based Systems

279. The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

280. Thus, the compositions can comprise, in addition to the disclosed compositions or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Feigner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

281. In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

282. The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other speciifc cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

283. Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

284. Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

(3) In Vivo/Ex Vivo

285. As described above, the compositions can be administered in a pharmaceutically acceptable carrier and can be delivered to the subjects cells in vivo and/or ex vivo by a variety of mechanisms well known in the art (e.g., uptake of naked DNA, liposome fusion, intramuscular injection of DNA via a gene gun, endocytosis and the like).

286. If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

e) Expression Systems

287. The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(1) Viral Promoters and Enhancers

288. Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

289. Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

290. The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

291. In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

292. It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

293. Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

(2) Markers

294. The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

295. In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

296. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycoplienolic acid) or hygromycin, respectively. Others include the neomycin analog G418 and puramycin.

f) Peptides

(1) Protein Variants

297. As discussed herein there are numerous variants of the AR, ARA54, ARA55, SRC-1, ARA70, RB, ARA24, ARA160, ARA267, gelsolin, and/or supervillin proteins or fragments thereof that are known and herein contemplated. In addition, to the known functional AR, ARA54, ARA55, SRC-1, ARA70, RB, ARA24, ARA160, ARA267, gelsolin, and/or supervillin proteins, or fragments thereof, species homologs, there are derivatives of the AR, ARA54, ARA55, SRC-1, ARA70, RB, ARA24, ARA 160, ARA267, gelsolin, and/or supervillin proteins, or fragments thereof, which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| alanine | AlaA |
| allosoleucine | AIle |
| arginine | ArgR |
| asparagine | AsnN |
| aspartic acid | AspD |
| cysteine | CysC |
| glutamic acid | GluE |
| glutamine | GlnQ |
| glycine | GlyG |
| histidine | HisH |
| isolelucine | IleI |
| leucine | LeuL |
| lysine | LysK |
| phenylalanine | PheF |
| proline | ProP |
| pyroglutamic acidp | Glu |
| serine | SerS |
| threonine | ThrT |
| tyrosine | TyrY |
| tryptophan | TrpW |
| valine | ValV |

TABLE 2
Amino Acid Substitutions
Original Residue Exemplary Conservative Substitutions,
others are known in the art.

| | |
|---|---|
| Ala | ser |
| Arg | lys, gln |
| Asn | gln; his |
| Asp | glu |
| Cys | ser |
| Gln | asn, lys |
| Glu | asp |
| Gly | pro |
| His | asn; gln |
| Ile | ieu; val |
| Leu | ile, val |
| Lys | arg; gln; |
| Met | Leu; ile |
| Phe | met; leu; tyr |
| Ser | thr |
| Thr | ser |
| Trp | tyr |
| Tyr | trp; phe |
| Val | ile; leu |

299. Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

300. For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gin; Ser, Thr; Lys, Arg; and Phe, Tyr. Such conservatively substituted variations of each explicitly disclosed sequence are included within the mosaic polypeptides provided herein.

301. Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

302. Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W.H. Freeman & Co., San Francisco pp 79-86 [1983]), acetylation of the N-terminal amine and, in some instances, amidation of the C-terminal carboxyl.

303. It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

304. Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

305. The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

306. It is understood that the description of conservative mutations and homology can be combined together in any combination, such as embodiments that have at least 70% homology to a particular sequence wherein the variants are conservative mutations.

307. As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein in the particular organism from which that protein arises is also known and herein disclosed and described.

g) Pharmaceutical Carriers/Delivery of Pharamceutical Products

308. As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

309. The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

310. Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

311. The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

(1) Pharmaceutically Acceptable Carriers

312. The compositions, including antibodies, can be used therapeutically in combination with a pharmaceutically acceptable carrier.

313. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

314. Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

315. Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

316. The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

317. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

318. Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

319. Compositions for oral administration include powders or granules, suspensions or solutions in water or non-aqueous media, capsules, sachets, or tablets. Thickeners, flavorings, diluents, emulsifiers, dispersing aids or binders may be desirable.

320. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

(2) Therapeutic Uses

321. Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

322. Following administration of a disclosed composition, such as an antibody or other molecule, such as a fragment of AR, ARA54, ARA55, SRC-1, ARA70, RB, ARA24, ARA160, ARA267, gelsolin, and/or supervillin, or fragment thereof, for forming or mimicking an interaction between AR and ARA54, ARA55, SRC-1, ARA70, RB, ARA24, ARA160, ARA267, gelsolin, and/or supervillin, or fragment thereof, for example, the efficacy of the therapeutic antibody or fragment can be assessed in various ways well known to the skilled practitioner. For instance, one of ordinary skill in the art will understand that a composition, such as an antibody or fragment, disclosed herein is efficacious in forming or mimicking an AR interaction in a subject by observing, for example, that the composition reduces the amount of AR transcription activity. The AR activity can be measured using assays as disclosed herein. Any change in activity is disclosed, but a 5%, 10%, 15%, 20%, 25%, 30%, 35%, 40%, 45%, 50%, 55%, 60%, 65%, 70%, 75%, 80%, 90%, or a 95% reduction in AR activity are also disclosed.

323. Other molecules that interact with AR to inhibit interactions with AR, ARA54, ARA55, SRC-1, ARA70, RB, ARA24, ARA160, ARA267, gelsolin, and/or supervillin, or fragment thereof, which do not have a specific pharmacuetical function, but which may be used for tracking changes within cellular chromosomes or for the delivery of diagnositc tools for example can be delivered in ways similar to those described for the pharmaceutical products.

324. The disclosed compositions and methods can also be used for example as tools to isolate and test new drug candidates for a variety of AR related diseases.

h) Chips and Micro Arrays

325. Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

326. Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

i) Computer Readable Mediums

327. It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

328. Disclosed are computer readable mediums comprising the sequences and information regarding the sequences set forth herein.

j) Kits

329. Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended.

### D. METHODS OF MAKING THE COMPOSITIONS

330. The compositions disclosed herein and the compositions necessary to perform the disclosed methods can be made using any method known to those of skill in the art for that particular reagent or compound unless otherwise specifically noted.

1. Nucleic Acid Synthesis

331. For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

332. One method of producing the disclosed proteins is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

333. For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

334. Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

3. Process for Making the Compositions

335. Disclosed are processes for making the compositions as well as making the intermediates leading to the compositions. There are a variety of methods that can be used for making these compositions, such as synthetic chemical methods and standard molecular biology methods. It is understood that the methods of making these and the other disclosed compositions are specifically disclosed.

336. Disclosed are cells produced by the process of transforming the cell with any of the disclosed nucleic acids. Disclosed are cells produced by the process of transforming the cell with any of the non-naturally occurring disclosed nucleic acids.

337. Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

338. Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate including a human, ape, monkey, orangutang, or chimpanzee.

339. Also disclosed are animals produced by the process of adding to the animal any of the cells disclosed herein.

### E. METHODS OF USING THE COMPOSITIONS

1. Methods of Using the Compositions as Research Tools

340. The compositions can be used for example as targets in combinatorial chemistry protocols or other screening protocols to isolate molecules that possess desired functional properties related to AR interactions. For example, AR, ARA54, ARA55, SRC-1, ARA70, RB, ARA24, ARA160, ARA267, gelsolin, and/or supervillin, or fragments thereof, and their interaction domains can be used in procedures that will allow the isolation of molecules or small molecules that mimic their binding properties. For example, disclosed herein AR and ARA54, ARA55, SRC-1, ARA70, RB, ARA24, ARA160, ARA267, gelsolin, and/or supervillin, or fragments thereof, interact. Libraries of molecules can be screened for interaction with AR that mimic the AR-ARA54, ARA55, SRC-1, ARA70, RB, ARA24, ARA160, ARA267, gelsolin, and/or supervillin, or fragments thereof, interaction by incubating the potential AR binding molecules with AR and then isolating those that are specifically competed off with AR, ARA54, ARA55, SRC-1, ARA70, RB, ARA24, ARA160, ARA267, gelsolin, and/or supervillin, or fragments thereof. There are many variations to this general protocol.

341. The disclosed compositions can also be used diagnostic tools related to diseases such as AR related diseases.

342. The disclosed compositions can be used as discussed herein as either reagents in micro arrays or as reagents to probe or analyze existing microarrays. The disclosed compositions can be used in any known method for isolating or identifying single nucleotide polymorphisms. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

2. Method of Treating Cancer

343. The disclosed compositions can be used to treat any disease where uncontrolled cellular proliferation occurs such as cancers. Disclosed are methods for regulating cancers related to AR, such as prostate cancer.

3. Methods of Gene Modification and Gene Disruption

344. The disclosed compositions and methods can be used for targeted gene disruption and modification in any animal that can undergo these events. Gene modification and gene disruption refer to the methods, techniques, and compositions that surround the selective removal or alteration of a gene or stretch of chromosome in an animal, such as a mammal, in a way that propagates the modification through the germ line of the mammal. In general, a cell is transformed with a vector which is designed to homologously recombine with a region of a particular chromosome contained within the cell, as for example, described herein. This homologous recombination event can produce a chromosome which has exogenous DNA introduced, for example in frame, with the surrounding DNA. This type of protocol allows for very specific mutations, such as point mutations, to be introduced into the genome contained within the cell. Methods for performing this type of homologous recombination are disclosed herein.

345. One of the preferred characteristics of performing homologous recombination in mammalian cells is that the cells should be able to be cultured, because the desired recombination event occur at a low frequency.

346. Once the cell is produced through the methods described herein, an animal can be produced from this cell through either stem cell technology or cloning technology. For example, if the cell into which the nucleic acid was transfected was a stem cell for the organism, then this cell, after transfection and culturing, can be used to produce an organism which will contain the gene modification or disruption in germ line cells, which can then in turn be used to produce another animal that possesses the gene modification or disruption in all of its cells. In other methods for production of an animal containing the gene modification or disruption in all of its cells, cloning technologies can be used. These technologies generally take the nucleus of the transfected cell and either through fusion or replacement fuse the transfected nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is transfected and has a gene modification or disruption event take place, and then cells derived from this cell can be used to clone a whole animal.

4. Method of Treating Cancer

347. The disclosed compositions can be used to treat any disease where uncontrolled cellular proliferation occurs such as cancers. Disclosed are methods for regulating cancers related to AR, such as prostate cancer.

### F. EXAMPLES

348. The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Androgen Receptor Coactivators

a) Plasmid Construction

349. A human prostate library in pACT2 yeast expression vector (a gift from Dr. S. Elledge) consists of the GAL4 activation domain (GAL4AD, a.a. 768-881) fused with human prostate cDNA. pSG5 wtAR was constructed as described previously (Ye: and Chang, Proc. Natl. Acad. Sci QSA 93:5517-5521, 1996). pGALO-AR (wild-type) was obtained from D. Chen (University of Massachusetts). pGALO contains the GAL4 DN binding domain (DBD).

350. For construction of pAS2-wtAR or -mAR, the C-terminal fragments (aa 595-918) from wtAR, mARt877s (Dr. S. P. Balk, Beth Israel Hospital, Boston, Mass.), or mARe708k (H. Shim, Hyogo Medical College, Japan) were inserted in pAS2 yeast expression vector (Clontech). Another AR mutant (mARv888m), derived from androgen insensitive syndrome patient, was constructed as previously described (Mowszowicz, et al. Endocrine 1:203-209, 1993). pGAL4-VP16 was used to construct a fusion of ARA70. pGAL4-VP16 contains the GAL4 DBD linked to the acidic activation domain of VP16. pCMX-Gal-N-RB and pCMX-VP16-AR were constructed by inserting fragments Rb (aa 370-928) and AR (aa 590-918) into pCMX-gal-N and pCMX-VP16, respectively. The sequence of construction junction was verified by sequencing. pYX-ARA24/Ran was constructed by placing the ARA24 gene under the control of the gal-1 promoter of yeast expression plasmid pYX243 (Ingenus). A cDNA fragment encoding the AR poly-Q stretch and its flanking regions (AR a.a. 11-208) was ligated to a PAS2 yeast expression plasmid for use as bait in the two hybrid assay. AR cDNAs of different poly-Q lengths that span the same AR poly-Q region as our bait plasmid were constructed in pAS2 in the same way, for yeast two-hybrid liquid culture ˜-gal assay. These AR bait plasmids with poly-Q lengths of 1, 25, 49 were all transformed into yeast Y190 and found to not be autonomously active. pCMV-antisense ARA24/Ran (ARA24 as) expression plasmid was constructed by inserting a 334-bp Bgl II fragment of ARA24/Ran, which spans 5′-untranslated region and the translation start codon of ARA24/Ran (nucleotides 1-334 of SEQ ID NO:5), into pCMV vector in the antisense orientation. The MMTV-CAT and MMTV-Luc reporter genes were used for the AR transactivation assay. pSG5-AR and- pSV-˜gal are under the regulation of SV40 promoter and —-globulin gene intron-1 enhancer. p6R-ARQ1, p6R-ARQ25, p6R-ARQ49 were kindly provided by Dr. Roger L. Meisfield (Chamberlain, et al. Nucleic Acids Res. 22:3181-3186, 1994) pSG5-GAL4 DBD-ARA24 was generated by inserting the coding sequence of Gal4 DBD-ARA24 hybrid protein into pSG5 vector. pVP16-ARN-Q1, pVP16-ARN-Q25, pVP16-ARN-Q25, pVP16-ARN-Q35, pVP16-ARN-Q49 were generated by inserting each poly-Q AR N-terminal domain (a.a. 34-555) into pVP16 vector (Clontech) to be expressed as a VP16AD hybrid protein. GALOAR plasmid, which contains GAL4 DBD fused to E region of human AR, was a gift from Dr. D. Chen. The pSG5-CAT reporter plasmid (Clontech) contains five GAL4 binding sites upstream of the E1 b TATA box, linked to the CAT gene. pSG5-AR and pSG5-ARA70 were constructed as previously described (Yeh and Chang, Proc. Natl. Acado sci USA 93:5517-5521, 1996). Two mutants of the AR gene (mAR877 derived from prostate cancer, codon 877 mutation Thr to Alai and mAR708 derived from partial androgen insensitive syndrome (PIAS), codon 708 mutation Glu to Lys), were provided by S. Balk (Beth Israel Hospital, Boston) and H. Shima (Hyogo Medical College, Japan), respectively. Clones used in the two-hybrid system to evaluate the role of Rb in AR transactivation were made by ligating an Rb fragment (aa 371-928) to the DBD of GAL4. Similarly, near full-length (aa 36-918) AR (nAR) and AR-LBD (aa 590-918) fragments ligated to transcription activator VP16.

b) Screening of Prostate cDNA Library for Yeast Two-Hybrid Screens for ARAs Associated with the Ligand Binding Domain

351. To identify ARA coactivators interact with the LBD, a pACT2-prostate cDNA library was cotransformed into Y190 yeast cells with a plasmid of pAS2 mAR(mART877S) which contains GAL4 DBD(aa 1-147) fused with the C-terminal domain of this mAR. Transformants were selected for growth on SD plates with 3-aminotriazole (25 mM) and DHT (100 nM) lacking histidine, leucine and tryptophan (−3SD plates). Colonies were also filter-assayed for β-galactosidase activity. Plasmid DNA from positive cDNA clones were found to interact with mtARt877s but not GAL4TR4 was isolated from yeast, amplified in E. coli, and the inserts confirmed by DNA sequencing.

352. To identify clones that interact with the poly-Q region of the N-terminal domain, the AR poly-Q stretch (aa

353. 11-208) was inserted into the pAS2 yeast expression plasmid and cotransformed into Y190 yeast cells with a human brain cDNA library fused to the Gal4 activation domain. Transformants were selected for growth on SD plates lacking histidine, leucine and tryptophan and supplemented with 3-aminotriazole (40 roM).

c) Amplification and Characterization of ARA Clones

354. Full length DNA sequences comprising two coactivators, designated ARA54 (SEQ ID NO:1) and ARA55 (SEQ ID NO:3), that were found to interact with rnARt877s were isolated by 5′RACE PCR using Marathon cDNA Amplification Kit (Clontech) according to the manufacturer's protocol.

355. The missing 5′ coding region of the ARA54 gene was isolated from H1299 cells using the gene-specific antisense primer shown in SEQ ID NO:9 and following PCR reaction conditions: 94° C. for 1 min, 5 cycles of 94° C. for 5 sec-72° C. for 3 min, 5 cycles of 94° C. for 5 sec-70° C. for 3 min, then 25 cycles of 94° C. for 5 sec-68° C. for 3 min. The PCR product was subcloned into pT7-Blue vector (Novagen) and sequenced.

356. ARA55 was amplified by PCR from the HeLa cell line using an ARA55-specific antisense primer (SEQ ID NO:10) and the PCR reaction conditions described for isolation of ARA54.

357. Using the 5′-RACE-PCR method, we were able to isolate a 1721 bp DNA fragment (SEQ ID NO:1) from the H1299 cell line with an open reading frame that encodes a novel protein 474 amino acids in length (SEQ ID NO:2). The in-vitro translation product is a polypeptide with an apparent molecular mass of 54.2 kDA, consistent with the calculated molecular weight (53.8 kDa). The middle portion of ARA54 (a.a. 220-265 of SEQ ID NO:2) contains a cysteine-rich region that may form a zinc finger motif called the RING finger, defined as CX2CX9-27CXHX2CX2CX6-17CX2C (SEQ ID NO: 11), a domain conserved among several human transcription factor or proto-oncogeny proteins, including BRCA1, RING1, PML and MEL-18 (Miki et al., Science 266:66-71 (1994); Borden et al., EMBO J. 14:1532-1541 (1995); Lovering et al., Proc. Natl. Acad. Sci. USA 90:2112-2116 (1993); Blake et al., Oncogene 6: 653-657 (1991); Ishida et al, Gene 129:249-255 (1993)). In addition, ARA54 also contains a second cysteine-rich motif which has a B box like structure located at 43 amino acids downstream from the RING finger motif. However, ARA54 differs from members of the RING finger-B-box family in that it lacks a predicted coiled-coil domain immediately C-terminal to the B box domain, which is highly conserved in the RING finger-B-box family.

358. The full-length human ARA55 has an open reading frame that encodes a 444 aa polypeptide (SEQ ID NO:4) with a predicted molecular weight of 55 kD that ARA55 shares 91% homology with mouse hic5. Human ARA55 has four LIM motifs in the C-terminal region. An LIM motif is a cysteine-rich zinc-binding motif with consensus sequence: CX2CX 16-23HX2CX2CX2CX16-21CX2(C,H,D) (SEQ ID NO:12) (Sadler, et al., J. Cell Biol. 119:1573-1587 (1992)). Although the function of the LIM motif has not been fully defined, some data suggest that it may play a role in protein-protein interaction (Schmeichel & Beckerle, Cell 79:211-219, 1994). Among all identified SR associated proteins, only ARA55 and thyroid hormone interacting protein 6 (Trip 6) (Lee, et al. Mol. Endocrinol. 9:243-254 (1995)) have LIM motifs.

359. A clone that showed strong interaction with the poly-Q bait was identified and subsequently subjected to sequence analysis. This clone contains 1566 bp insert (SEQ ID NO:5) with an open reading frame encoding a 216 aa polypeptide (SEQ ID NO:6) with a calculated molecular weight of 24 kDa. GenBank sequence comparison showed that this clone has the same open reading frame sequence as RanjTC4, an abundant ras-like small GTPase involved in nucleocytoplasmic transport that is found in a wide variety of cell types (Beddow et al., Proc. Natl. Acad. Sci. U.S.A. 92:3328-3332, (1995). Accordingly, the factor was designated ARA24/Ran. The cDNA sequence of the ARA24 clone (SEQ ID NO:5) (GenBank accession number AF052578) is longer than that of the published ORF for human Ran, in that it includes 24 and 891 bp of 5′- and 31-untranslated regions, respectively.

d) Northern Blotting

360. The total RNA (25˜g) was fractionated on a 1% formaldehyde-MOPS agarose gel, transferred onto a Hybond-N nylon membrane (Amersham) and prehybridized. A probe corresponding to the 900 bp C-terminus of ARA55 or an ARA54-specific sequence was 32P-labeled in vitro using Random Primed DNA Labeling Kit (Boehringer-Mannheim) according to the manufacture's protocol and hybridized overnight. After washing, the blot was exposed and quantified by Molecular Dynamics PhosphorImager. β-actin was used to monitor the amount of total RNA in each lane.

361. Northern blot analysis indicated the presence of a 2 kb ARA55 transcript in Hela and prostate PC3 cells. The transcript was not detected in other tested cell lines, including HepG2, H1299, MCF7, CHO, PC12, P19, and DU145 cells. The ARA54 transcript was found in H1299 cells, as well as in prostate cancer cell lines PC3 and LNCaP.

e) Co-Immunoprecipitation of AR and ARAs

362. Lysates from in-vitro translated full-length of AR and ARA54 were incubated with or without 10⁻⁸ M DHT in the modified RIPA buffer (50 mM Tris-HCL pH 7.4, 150 mM NaCl, 5 mM EDTA, 0.1% NP40, 1 mM PMSF, aprotinin, leupeptin, pepstatin, 0.25% Na-deoxycholate, 0.25% gelatin) and rocked at 4° C. for 2 hr. The mixture was incubated with rabbit anti-His-tag polyclonal antibodies for another 2 hr and protein A/G PLUS-Agarose (Santa Cruz) were added and incubated at 4° C. for additional 2 hr. The conjugated beads were washed 4 times with RIPA buffer, boiled in SDS sample buffer and analyzed by 8% SDS/PAGE and visualized by STORM 840 (Molecular Dynamics). ARA54 and AR were found in a complex when immunoprecipitated in the presence of 10⁻⁸ M DHT, but not in the absence of DHT. This result suggests that ARA54 interacts with AR in an androgen-dependent manner.

363. Interaction between recombinant full-length human AR and ARA24/Ran proteins further examined by co-immunoprecipitation, followed by SDS-PAGE and western blotting. Results of the co-immunoprecipitation assay indicate that ARA24/Ran interacts directly with AR. The phosphorylation state of bound guanine nucleotide to the small GTPases does not affect this interaction.

f) AR pull-down assay using GST-Rb

364. Full-length Rb fused to glutathione-S-transferase (ST-Rbl-92S) was expressed and purified from E. coli. strain B121pLys as described recently (Zarkowska & Mittnacht, J. Biol. Chem. 272:12738-12746, 1997). approximately 2 J.Lg of His-tag column purified baculovirus AR was mixed with GST-loaded glutathione-Sepharose beads in 1 ml of NET-N (20 roM Tris-HCL (pH 8.0, 100 roM NaCl, 1 roM EDTA, 0.5% (v/v) Noniodet P-40) and incubated with gentle rocking for 3 hr at 4° C. Following low-speed centrifugation to pellet the beads, the clarified supernatant was mixed with GST-Rb-loaded glutathione-Sepharose beads in the presence or absence of 10 nM DHT and incubated for an additional 3 hr with gentle rocking at 4° C. The pelleted beads were washed 5 times with NET-N, mixed with SDS-sample buffer, boiled, and the proteins separated by electrophoresis on a 7.5% polyacrylamide gel. A Western blot of the gel was incubated with anti-AR polyclonal antibody NH27 and developed with alkaline phosphatase-conjugated secondary antibodies.

365. AR was coprecipitated with GST-Rb, but not GST alone, indicating that AR and Rb are associated in a complex together.

g) Transfection Studies

366. Human prostate cancer DU145 or PC3 cells, or human lung carcinoma cells NCI H1299 were grown in Dulbecco's minimal essential medium (DMEM) containing penicillin (25 U/ml), streptomycin (25˜g/ml), and 5% fetal calf serum (FCS). One hour before transfection, the medium was changed to DMEM with 5% charcoal-stripped FCS. Phenol red-free and serum-free media were used on the experiments employing E2 or TGF-β, respectively. A β-galactosidase expression plasmid, pCMV-β-gal, was used as an internal control for transfection efficiency.

367. Cells were transfected using the calcium phosphate technique (Yeh, et al. Molec. Endocrinol. 8:77-88, 1994). The medium was changed 24 hr posttransfection and the cells treated with either steroid hormones or hydroxyflutamide, and cultured for an additional 24 hr. Cells were harvested and assayed for CAT activity after the cell lysates were normalized by using β-galactosidaseas an internal control. Chloramphenicol acetyltransferase (CA) activity was visualized by PhosphorImager (Molecular Dynamics) and quantitated by ImageQuant software (Molecular Dynamics).

h) Mammalian Two-Hybrid Assay

368. The mammalian two-hybrid system employed was essentially the protocol of Clontech (California), with the following modifications. In order to obtain better expression, the GAL4 DBD (a.a. 1-147) was fused to pSGS under the control of an SV40 promoter, and named pGALO.

369. The hinge and LBD of wtAR were then inserted into pGALO. Similarly, the VPI6 activation domain was fused to pCMX under the control of a CMV promoter, and designated pCMX-VP16 (provided by Dr. R. M. Evan).

370. The DHT-dependent interaction between AR and ARA54 was confirmed in prostate DU145 cells using two-hybrid system with CAT reporter gene assay. Transient transfection of either ARA54 or wtAR alone showed negligible transcription activity. However, coexpression of AR with ARA54 in the presence of 10⁻⁸ M DHT significantly induced CAT activity.

371. ARA54 functions as a coactivator relatively specific for AR-mediated transcription. ARA54 induces the transcription activity of AR and PR by up to 6 fold and 3-5 fold, respectively. In contrast, ARA54 showed only marginal effects (less than 2 fold) on GR and ER in DU145 cells. These data suggest that ARA54 is less specific to AR as relative to ARA70, which shows higher specificity to AR.

372. Coexpression of ARA54 with SRC-1 or ARA70 was found to enhance AR transcription activity additively rather than synergistically. These results indicate that these cofactors may contribute individually to the proper or maximal AR-mediated transcription activity.

373. Since the C-terminal region of ARA54(a.a. 361-471 of SEQ ID NO:2) isolated from prostate cDNA library has shown to be sufficient to interact with AR in yeast two-hybrid assays, it was investigated whether it could squelch the effect of ARA54 on AR-activated transcription in H1299 cells, which contain endogenous ARA54. The C-terminal region of ARA54 inhibits AR-mediated transcription by up to 70%; coexpression of exogenous full-length ARA54 reverses this squelching effect in a dose-dependent manner. These results demonstrate that the C-terminal domain of ARA54 can serve as a dominant negative inhibitor, and that ARA54 is required for the proper or maximal AR transactivation in human H1299 cells.

374. Examination of the effect of ARA54 on the transcription activities of wtAR and mtARs in the presence of DHT, E2 and HF revealed differential ligand specificity. Translational activation of wtAR occurred in the presence of DHT (10⁻¹⁰ to 10⁻⁸ M); coexpression of ARA54 enhanced transactivation by another 3-5 fold. However, wtAR responded only marginally to E2 (10⁻⁹-10⁻⁷ M) or HF (10⁻⁷-10⁻⁵ M) in the presence or absence of ARA54. As expected, the positive control, ARA70, is able to enhance the AR transcription activity in the presence of 10⁻⁹-10⁻⁷ M E2 and 10⁻⁷-10⁻⁵ M HF, that matches well with previous reports (Yeh, PNAS, Miyamoto, PNAS).

375. The AR mutants Art877a, which is found in many prostate tumors (23), and Are708k, found in a yeast genetic screening (24) and a patient with partial androgen insensitivity, exhibited differential specificity for lignands. In the absence of ARA54, Art877a responded to E2 (10⁻⁹-10⁻⁷ M) and HF (10⁻⁷-10⁻⁵ M), and ARA54 could further enhance E2- or HF-mediated AR transactivation. These results suggested that mtARs might also require cofactors for the proper or maximal DHT-, E2-, or HF-mediated AR transcription activity. The DHT response of mARe708k was only a slightly less sensitive than that of wtAR or mARt877s, whereas E2 and HF exhibited no agonistic activity toward ARe708k. Together, these results imply that the change of residue 708 on AR might be critical for the interaction of the antiandrogen-ARe708k-ARA54 complex, and that both AR structure and coactivators may playa role in determining ligand specificity.

376. CAT activity in DU145 cells cotransfected with a plasmid encoding the hormone binding domain of wtAR fused to the GAL4 DBD(GAL4AR) and a plasmid encoding full-length ARA55 fused to the activation domain of VP16 (VP16-ARA55) was significantly induced by the cotransfection of VP16-ARA55 and GAL4AR in the presence of 10 nM DHT, but not induced by E2 or HF. Combination of GAL4 empty vector and VP16-ARA55 did not show any CAT activity. Combination of GAL4AR and VP16 vector showed negligible CAT activity. These results indicate that ARA55 interacts with AR in an androgen-dependent manner.

377. Transient transfection assays were conducted to investigate the role of ARA55 in the transactivation activity of AR. DU145 cells were cotransfected with MMTV-CAT reporter, increasing amounts of ARA55 and wtAR under eukaryotic promoter control. Ligand-free AR has minimal MMTV-CAT reporter activity in the presence or absence of ARA55. ARA55 alone also has only minimal reporter activity Addition of 10 nM DHT resulted in 4.3 fold increase of AR transcription activity and ARA55 further increased this induction by 5.3 fold (from 4.3 fold to 22.8 fold) in a dose-dependent manner. The induced activity reached a plateau at the ratio of AR:ARA55 to 1:4.5. Similar results were obtained using PC3 cells with DU145 cells, or using a CAT reporter gene under the control of a 2.8 kb promoter region of a PSA gene. The C-terminus of ARA55 (ARA55251-444) (a.a. 251-444 of SEQ ID NO:4) did not enhance CAT activity. Cotransfection of PC3 cells, which contain endogenous ARA55, with ARA55251-444, AR and MMTV-CAT reporter in the presence of 10 nM DHT demonstrated dramatically reduced AR transcription activity relative to cells transfected with AR and MMTV-CAT alone. These results demonstrate that ARA55 is required for the proper or maximal AR transcription activity in PC3 cellsJ and that the C-terminus of ARA55 can serve as a dominant negative inhibitor.

378. The effect of ARA55 on mARt877s and mARe708k in the presence of DHT and its antagonists, E2, and HF. The mARt877s receptor is found in LNCaP cells and/or advanced prostate cancers and has a point mutation at codon 877 (Thr to Ser) (Gaddipati et al., Cancer Res. 54:2861-2864 (1994); Veldscholte et al., Biochem. Biophys. Commun. 173:534-540 (1990)). The mARe708k receptor, has a point mutation at codon 708 (Glu to Lys), was isolated by a yeast genetic screening and exhibits reduced sensitivity to HF and E2 relative to wtAR (Wang, C., PhD thesis of University of Wisconsin-Madison (1997)). The transcription activities of wtAR, mARt877s, and mARe708k are induced by DHT (10⁻¹¹ to 10⁻⁸ M). ARA55 enhanced the transactivation of all three receptors by 4-8 fold. In the presence of E2 or HF, wtAR responded marginally only at higher concentrations (10⁻⁷ M for E2 and 10⁻⁵ M for HF). Cotransfection of wtAR with ARA55 at a 1:4.5 ratio, however, increases AR transcription activity at 10⁻⁸-10⁻⁷ M for E2 or 10⁻⁶ to 10⁻⁵ M for HF. Compared to wtAR, the LNCaP mAR responded much better to E2 and HF and ARA55 significantly enhanced its transcription activity. ARA55 may be needed for the proper or maximal DHT-, E2-, or HF-mediated AR transcription activity.

379. The effect of ARA55 on transcription activation by GR, PR, and ER was tested in DU145 cells. ARA55 is relatively specific to AR, although it may also enhance GR and PR to a lesser degree, and has only a marginal effect on ER. ARA70 shows much higher specificity to AR than ARA55, relative to the other tested steroid receptors. Although ARA55 enhances AR-mediated transcription to a greater degree than GR-, PR-, or ER-mediated transcription, it appears to be less specific than ARA70.

380. Because the amino acid sequence of ARA55 has very high homology to mouse hic5, and early studies hic5 suggested this mouse gene expression can be induced by the negative TGF-β (Shibanuma et al., J. Biol. Chem. 269:26767-26774 (1994)), it was tested to see whether ARA55 could serve as a bridge between TGF˜ and AR steroid hormone system. Northern blot analysis indicated that TGF-β treatment (5 ng/ml) could induce ARA55 mRNA by 2-fold in PC3 cells. In the same cells, TGF-β treatment increased AR transcription activity by 70%. This induction is weak relative to the affect achieved upon transfection of PC3 cells with exogenous ARA55 (70% vs. 4 fold). This may be related to the differences in the ratios of AR and ARA55. The best ratio of AR:ARA55 for maximal AR transcription activity is 1:4.5. Whether other mechanisms may also be involve in this TGF-β induced AR transcription activity will be an interesting question to investigate. The unexpected discovery that TGF-β may increase AR transcription activity via induction of ARA55 in prostate may represent the first evidence to link a negative regulatory protein function in a positive manner, by inducing the transcription activity of AR, the major promoter for the prostate tumor growth.

381. The ability of ARA55 to induce transcription activity of both wtAR and mARt877s in the presence of DHT, E2, and HF suggests an important role for ARA55 in the progression of prostate cancer and the development of resistance to hormonal therapy. Evaluation of molecules that interfere with the function of ARA55 may aid in the identification of potential chemotherapeutic pharmaceuticals.

382. Human small lung carcinoma H 1299 cell line, which has no endogenous AR protein, were transfected with AR and ARA24/Ran. Because ARA24/Ran is one of the most abundant and ubiquitously expressed proteins in various cells, both sense and antisense ARA24/Ran mammalian expression plasmids were tested. Overexpression of sense ARA24/Ran did not significantly enhance the AR transactivation, a result that is not surprising, in view of the abundance of endogenous ARA24/RAN. However, expression of antisense ARA24/Ran (ARA24 as) markedly decreased DHT-induced CAT activity in a dose dependent manner. Furthermore, increasing the DHT concentration from 0.1 nM to 10 nM DHT resulted in strong induction of AR transactivation and decreased the inhibitory effect of ARA24as effect, indicating that increased DHT concentration can antagonize the negative effect of ARA24as.

383. The affinity between ARA24/Ran and AR is inversely related to the length of AR poly-Q stretch. AR transactivation decreases with increasing AR poly-Q length. Reciprocal two-hybrid assays with exchanged fusion partners, Gal4 DBD-ARA24/Ran and VP16AD-ARNs (a.a. 34-555 with poly-Q lengths of 1, 25, 35, 49 residues) were conducted using mammalian CHO cells. These results consistently show that the affinity between ARA24/Ran and AR poly-Q region is inversely correlated with AR poly-Q length in both yeast and mammalian CHO cells.

384. The regulation of AR transactivation by ARA24/Ran correlates with their affinity. These results suggest that ARA24/Ran could achieve differential transactivation of AR, with ARs having different poly-Q length could exist in a single cell or cell system. ARA24 as was again used in the ARE-Luc transfection assays to address the role of AR poly-Q length in the regulation o£ AR by ARA24/Ran. ARs of poly-Q lengths 1, 25, and 49 residues, and increasing amounts (1, 2, and 4 μg) of ARA24 as expression vectors were co-transfected with equal amounts of reporter plasmid (pMMTV-Luc) in CHO cells. Although the basal reporter activity is slightly affected by increasing amounts of antisense ARA24/Ran, ARA24 as showed a more significant decrease of AR transactivation. As AR poly-Q length increased, the ARA24 as effect on AR transactivation decreased. These results suggest that the affinity of ARA24/Ran for AR and the effect of decreasing ARA24/Ran on AR transactivation faded over the expansion of AR poly-Q length.

385. Coexpression of Rb and AR expression plasmids in DU145 cells using the mammalian two-hybrid system resulted in a 3 fold increase in CAT activity by cotransfection of near full length AR (nAR, amino acids 36-918) and Rb. Cells cotransfected with nAR and PR-LBD or Rb and ARA70 did not show increased CAT activity. Surprisingly, addition of 10 nM DHT made very little difference in the interaction between Rb and nAR. The inability of Rb to interact with AR-LBD suggest that interaction site of AR is located in N-terminal domain (aa 36 to 590). Together, the data suggest the interaction between Rb and AR is unique in the following ways: first, the interaction is androgen-independent and binding is specific but relatively weak as compared to other AR associated protein, such as ARA70 (3 fold vs. 12 fold induced CAT activity in mammalian two-hybrid assay, data not shown). Second, unlike most identified steroid receptor associated proteins that bind to C-terminal domain of steroid receptor, Rb binds to N-terminal domain of AR. Third, no interaction occurred between Rb and ARA70, two AR associated proteins in DU145 cells DU145 cells containing mutated Rb (Singh et al., Nature 374: 562-565 (1995)) were cultured with charcoal-stripped FCS in the presence or absence of 1 nM DHT. No AR transcription activity was observed in DU145 cells transiently transfected with wild type AR and Rb at the ratio of 1:3 in the absence of DHT. When However, AR transcription activity could be induced 5-fold when wild type AR was expressed in the presence of 1 nM DHT. Cotransfection of Rb with AR can further enhance the AR transcription activity from 5-fold to 21-fold in the presence of 1 nM DHT. As a control, cotransfection of ARA70, the first identified AR coactivator, can further enhance in DU145 cells transcription activity from 5-fold to 36-fold. In DU145 cells transfected with Rb, ARA70, and AR, the induction of AR transcription activity was synergistically increased from 5-fold to 64-fold. Upon transfection of wild type AR without Rb or ARA70, only marginal induction (less than 2-fold) was detected in the presence of 10 nM E2 or 1 nM HF. In contrast, cotransfection of the wild type AR with Rb or ARA70 can enhance the AR transcription activity to 12-fold (E2) or 3-4 fold (HF), and cotransfection of Rb and ARA70 with AR can further enhance the AR transcription activity to 36-fold (E2 or 12-fold (HF). We then extended these findings to two different AR mutants: mARt877s from a prostate cancer patient and mARe708k from a partial-androgen-insensitive patient. Similar inductions were obtained when wild type AR was replaced by mARt877s. In contrast, while similar induction was also detected in the presence of 1 nM DHT when ˜e replace wild type AR with mARe708k, there was almost no induction by cotransfection of meAR708k with Rb and/or ARA70 in the presence of 10 nM E2 or 1 ˜M HF. These results indicated that Rb and ARA70 can synergistically induce the transcription activity of wild type AR and mAR877 in the presence of 1 nM DHT, 10 nM E2 or 1 ˜M HF.

386. However, Rb and ARA70 synergistically induce the transcription activity of mAR708 only in the presence of 1 nM DHT, but not 10 nM E2 or 1 ˜M HF. The fact that Rb and ARA70 can induce transcription activity of both wild type AR and mutated AR that occur in many prostate tumors may also argue strongly the importance of Rb and ARA70 in normal prostate as well as prostate tumor. Also, the differential induction of DHT vs. E2/HF may suggest the position of 708 in AR may play vital role for the recognition of androgen vs anti-androgens to AR.

387. The effect of Rb and ARA70 on the transcription activity of other steroid receptors through their cognate DNA response elements [MMTV-CAT for AR, glucocorticoid receptor (GR), and progesterone receptor (PR); ERE-CAT for estrogen receptor (ER)] was also examined. Although Rb and ARA70 ccan synergistically induce AR transcription activity up to 64-fold, Rb and ARA70 can only have marginal induction on the transcription activity of GR, PR, and ER in DU145 cells. These results suggest that Rb and ARA70 are more specific coactivators for AR in prostate DU145 cells. However, it cannot be ruled out that possibly the assay conditions in prostate DU145 cells are particularly favorable for Rb and ARA70 to function as coactivators for AR only, and Rb and ARA70 may function as stronger coactivators for ER, PR, and GR in other cells or conditions. Failure of Rb to induce transactivation by mutant AR888, which is unable to bind androgen, suggests that while interaction between Rb and AR is androgen-independent, the AR-Rb (and AR-ARA70) complexes require a ligand for the transactivation activity.

388. The activity of Rb in cell cycle control is related essentially to its ability to bind to several proteins, thus modulating their activity. To date, many cellular proteins have been reported which bind to Rb (Weinberg, R. A., Cell 81:323-330 (1995)). These include a number of transcription factors, a putative regulator of ras, a nuclear structural protein, a protein phosphatase, and several protcin kinases.

389. Much attention has been given to the functional interaction between Rb and transcription factors. To date, several of these factors have been shown to form complexes with Rb in cells. Such complex formation and subsequent function studies have revealed that the modulating activity of Rb can take the form of repression of transcription as with E2F {Weintraub et al., Nature 375:812-815 (1995)), or activation as with NF-IL6 {Chen et al., Proc. Natl. Acad. Sci. USA 93:465-469 (1996)) and the hBrm/BRGI complex {Singh et al., (1995)). Disclosed herein Rb can bind to AR and induce the AR transcription activity.

390. A relationship between Rb expression and response to endocrine therapy of human breast tumor has been suggested {Anderson et al., J. Pathology 180:65-70 (1996)). Other studies indicate that Rb gene alterations can occur in all grades and stages of prostate cancer, in localized as well as metastatic disease {Brooks et al., Prostate 26:35-39 (1995)). How Rb function may be linked to androgen-dependent status In prostate tumor progression remains unclear. One possible explanation is that Rb alteration may be a necessary event in prostate carcinogenesis for a subset of prostatic neoplasms, which may be also true for the AR expression in prostate tumors.

### 2. Example 2

### A Dominant-Negative Mutant of Androgen Receptor Coregulator ARA54 Inhibits Androgen Receptor-Mediated Prostate Cancer Growth

a) Materials and Methods

(1) Chemicals and Plasmids

391. 5α-Dihydrotestosterone (DHT), progesterone (P), and dexamethasone (Dex) were obtained from Sigma, and HF was from Schering. pAS2-AR containing the C-terminus of the ligand binding domain (LBD) from wild-type AR fused to the GAL4 DNA binding domain (DBD) was constructed as previously described (Fujimoto et al. (1999) J. Biol. Chem. 274, 8316-8321). pACT2-C′-ARA54 fused with the GAL4 activation domain (AD) was the clone originally identified from prostate cDNA library (26). pSG5-AR, pSG5-C′-ARA54, pSG5-fl-ARA54, pSG5-ARA55, pSG5-ARA70, and pSG5-SRC-1 were constructed as previously described (Yeh et al. (1998) Proc. Natl. Acad. Sci. U.S.A. 95, 5524-5532; Yeh, S, and Chang, C, (1996) Proc. Natl. Acad. Sci. U.S.A. 93, 5517-5521; Fujimoto et al. (1999) J. Biol. Chem. 274, 8316-8321; Kang et al. (1999) J. Biol. Chem. 274, 8570-8576). pSV-mutant AR877 (33) and pSG5-Rb were provided by Drs. S. Balk and W. Kaelin, Jr., respectively. pGAL0-AR containing the AR LBD fused with the GAL4 DBD and pCMX-VP16-fl-ARA54 fused to the AD of VP16 were constructed as previously described (Kang et al. (1999) J. Biol. Chem. 274, 8570-8576; Yeh et al. (1999) Endocrine 11, 195-202). pCMX-GAL4 DBD-fl-ARA54 was constructed by inserting the EcoRI/SacI fragment of ARA54 in frame to the GAL4 DBD. pCMX-VP16-C′-ARA54 and pCMX-VP16-mt-ARA54 were constructed using the C′-ARA54 and mt-ARA54 BamHI fragments.

(2) Mutated Library Construction

392. An ARA54 mutated library was generated by incubating 100 μg of pACT2-C′-ARA54 with 1 M hydroxylamine (Sigma) at 70 C for 1 h, followed by DNA extraction.

(3) Yeast Two-Hybrid Screening

393. Plasmids with pAS2-AR and the mutated ARA54 library were sequentially transformed into the yeast strain, Y190, harboring reporter genes (i.e. lacZ and His3), according to the CLONTECH Yeast Protocols Handbook. The transformed yeast cells were plated with 100 nM DHT on synthetic dropout (SD) plates lacking tryptophan and leucine. Colonies were filter-assayed for β-galactosidase activity, and white colonies that indicated no interaction between the AR bait and mutant ARA54 were selected. The mutant ARA54 plasmid DNAs were isolated from the yeast cells that have spontaneously lost the cycloheximide-bearing plasmid (pAS2-AR) by plating the selected white colonies on SD (-leucine) in the presence of 10 μg/ml cycloheximide (Sigma). The mutant ARA54 clones were then subcloned into the pSG5 mammalian expression vector (Stratagene).

(4) Cell Culture, Transient Transfections, and Reporter Gene Assays

394. The human prostate cancer cell lines, LNCaP, PC-3, and DU145, were maintained in Dulbecco's minimum essential medium (DMEM) containing 5% fetal calf serum (FCS). Transfections using the calcium phosphate precipitation method and chloramphenicol acetyltransferase (CAT) and luciferase (Luc) assays were performed as previously described (Miyamoto et al. (1998) Proc. Natl. Acad. Sci. U.S.A. 95, 7379-7384; Yeh et al. (1999) Endocrine 11, 195-202; Miyamoto et al. (1998) Proc. Natl. Acad. Sci. U.S.A. 95, 11083-11088). Briefly, 1-4×10⁵ cells were plated on 35-mm or 60-mm dishes 24 h before adding the precipitation mix containing a CAT or Luc reporter gene and a β-galactosidase expression plasmid (pCMV-β-gal) as an internal control for normalization of transfection efficiency. The medium was changed to phenol-red-free DMEM with 5% charcoal-stripped FCS 1 h before transfection. In each experiment, the total amount of transfected DNA per dish was maintained as a constant by addition of empty expression vector (pSG5 or pVP16, as appropriate). The medium was changed again 24 h after transfection, and the cells were treated with 1 nM of DHT or 1 μM of HF for 24 h. The cells were then harvested and whole cell extracts were used for CAT or Luc assay. The CAT activity was quantitated with a PhosphorImager (Molecular Dynamics). The Luc assay was determined using a Dual-Luciferase Reporter Assay System (Promega) and luminometer.

(5) Establishment of LNCaP Cell Lines Stably Transfected with the Plasmids Encoding the Mutant ARA54 Under the Inducible Promoter

395. The pBIG2i vector contains all of the elements required for tetracycline-responsive gene expression and a selective marker conferring resistance to hygromycin B for the generation of stable cell lines (Strathdee, C. A., McLeod, M. R., and Hall, J. R. (1999) Gene 229, (Moilanen et al. (1998) Mol. Cell. Biol. 18, 5128-5139; Di Croce et al. (1999) EMBO J. 18, 6201-6210; Yeh, S, and Chang, C, (1996) Proc. Natl. Acad. Sci. U.S.A. 93, 5517-5521; Yeh et al. (1998) Biochem. Biophys. Res. Commun. 248,361-367; Fujimoto et al. (1999) J. Biol. Chem. 274, 8316-8321; Kang et al. (1999) J. Biol. Chem. 274, 8570-8576; Hsiao et al. (1999) J. Biol. Chem. 274, 20229-20234; Hsiao, P.-W., and Chang, C. (1999) J. Biol. Chem. 274, 22373-22379; Yeh et al. (1999) Endocrine 11, 195-202). We first constructed pBIG2i-C′-ARA54, pBIG2i-mt-ARA54, and pBIG2i-fl-ARA54, and then transfected each plasmid into LNCaP or PC-3 cells using SuperFect transfection reagent (Qiagen). After transfection, cells were cultured in the presence of 100 μg/ml hygromycin B (GIBCO BRL) to select for stably transfected cells that had incorporated the pBIG2i-based construct. After growth for a further 2 weeks, individual clones were picked. Then, we confirmed stable expression of the mutant (C-terminal fragment) or wild-type (full-length) ARA54 induced by doxycycline using Northern blotting. Northern blotting was performed using total RNAs from the stable LNCaP or PC-3 cells and C-terminal fragment of ARA54 as a DNA probe, as described previously (Fujimoto et al. (1999) J. Biol. Chem. 274, 8316-8321; Kang et al. (1999) J. Biol. Chem. 274, 8570-8576)).

(6) Western Blot

396. Western blotting analysis was performed in the stable LNCaP cells, using NH27 polyclonal antibody for the AR and monoclonal prostate-specific antigen (PSA) antibody (DAKO), as described previously (Miyamoto et al. (1998) Proc. Natl. Acad. Sci. U.S.A. 95, 7379-7384). An antibody for β-actin (Santa Cruz Biotechnology) was used as the internal control.

(7) Mammalian Two-Hybrid Assay

397. DU145 cells were transiently cotransfected with a GAL4-hybrid expression plasmid, a VP16-hybrid expression plasmid, the reporter plasmid pG5-CAT, and the pCMV-O-gal internal control plasmid. Transfections and CAT assays were performed as described above.

b) Results

(1) Isolation of Dominant-Negative Mutant ARA54

398. An in vitro mutagenesis strategy combined with the yeast two-hybrid system was used to isolate dominant-negative forms of ARA54. ARA54 was initially isolated from a human prostate cDNA library as a C-terminal fragment that interacted with AR (Kang et al. (1999) J. Biol. Chem. 274, 8570-8576). This C-terminal region of ARA54 (amino acids 361-474) was cloned into pACT2 and mutagenized with 1M hydroxylamine to create the mutant library ARA54 C-terminal for yeast two-hybrid screening. This library was screened against pAS2-AR for the selection of clones that did not interact with AR. 11 colonies were selected that showed no interaction between pAS2-AR and the pACT2-ARA54 mutant from approximately 50,000 yeast colonies. The interactions with AR were confirmed by subcloning each clone into pACT2 and yeast two-hybrid assay with sequential transformation with PAS2-AR and pACT2-mutant clone. These 11 pACT2 constructs were then subcloned into pSG5 to assess their effect on AR-mediated transactivation in the prostate cancer cell lines LNCaP (AR- and ARA54-positive), PC-3 (AR-negative and ARA54-positive), and DU145 (AR- and ARA54-negative) (Kang et al. (1999) J. Biol. Chem. 274, 8570-8576), using a reporter gene assay. It has been shown that transcription activity of a mutant AR or wild-type AR could be induced in LNCaP or PC-3 cells in response to both androgen (DHT) and the antiandrogen, HF, and that fl-ARA54 can enhance the AR transactivation in DU145 cells (Kang et al. (1999) J. Biol. Chem. 274, 8570-8576; Yeh et al. (1999) Endocrine 11, 195-202; Miyamoto et al. (1998) Proc. Natl. Acad. Sci. U.S.A. 95, 11083-11088; Chang et al. (1999) Proc. Natl. Acad. Sci. U.S.A. 96, 11173-11177; Miyamoto et al. (2000) Int. J. Urol. 7, 32-34). FIG. 1 shows that C′-ARA54 suppresses DHT- or HF-mediated AR transcription activity. One mutant ARA54 clone (mt-ARA54) was found to have a stronger dominant-negative effect both for endogenous fl-ARA54 in LNCaP and PC-3 cells and for exogenous fl-ARA54 in DU145 cells. However, both mutants (C′-ARA54 or mt-ARA54) showed an only marginal effect on AR transactivation in the absence of fl-ARA54 in DU145 cells (FIG. 1E, 1F). The suppression of AR transactivation by either C′-ARA54 or mt-ARA54 was not the result of down-regulation of AR protein expression. LNCaP cells transfected with C′-ARA54 or mt-ARA54 showed little change in endogenous AR expression compared to non-transfected cells. These results suggest that a mutant ARA54 dominant-negatively suppresses endogenous AR- and exogenous AR-mediated transactivation. Sequencing analysis revealed that mt-ARA54 contained a single point mutation (a G to A transition) at the first position of codon 472, resulting in a glutamic acid to lysine substitution.

(2) Effect of the Dominant-Negative ARA54 Mutant on the Transactivation Mediated by Different Steroid Receptors

399. Previous studies demonstrated ARA54 had a marginal transcription effect on the glucocorticoid receptor (GR) but could enhance the transcription activity of the progesterone receptor (PR) by up to 4-fold (Kang et al. (1999) J. Biol. Chem. 274, 8570-8576). The effect of mt-ARA54 on PR and GR transactivation in the presence of endogenous or exogenous fl-ARA54 was examined. Both C′-ARA54 and mt-ARA54 had only a marginal effect on PR-mediated transactivation in the presence of P in the PC-3 cell line. Similarly, GR transactivation was only marginally repressed by either C′-ARA54 or mt-ARA54 (FIG. 2A). When fl-ARA54 was cotransfected with PR or GR into DU145 cells, fl-ARA54 induced PR transcription by 2.9-fold and GR transcription activity by 1.6-fold (FIG. 2B). In DU145 cells, mt-ARA54 suppressed fl-ARA54-induced PR transactivation by 43%, but only marginally suppressed GR transactivation. C′-ARA54 showed little effect on PR or GR transcription.

(3) Coregulator Specificity of the Dominant-Negative ARA54 Mutant

400. To determine whether C′-ARA54 and mt-ARA54 inhibited only wild-type ARA54-mediated transactivation, we examined their effect in DU145 cells in the presence of other AR coregulators. C′-ARA54 or mt-ARA54 was cotransfected with AR and ARA55, SRC-1, ARA70, Rb, or SRC-1 into DU145 cells. As shown in FIG. 3A, and consistent with previous reports (Yeh, S, and Chang, C, (1996) Proc. Natl. Acad. Sci. U.S.A. 93, 5517-5521; Yeh et al. (1998) Biochem. Biophys. Res. Commun. 248, 361-367; Fujimoto et al. (1999) J. Biol. Chem. 274, 8316-8321; Kang et al. (1999) J. Biol. Chem. 274, 8570-8576, 29), these coactivators alone enhanced AR transcription activity an additional 2.9- to 6.0-fold in the presence of DHT. C′-ARA54 and mt-ARA54 showed only marginal or slight suppressive effects on ARA55-, ARA70-, Rb-, or SRC-1-enhanced AR transactivation. Similar results were also obtained when a mutant AR (mtAR877, codon 877 mutation threonine to serine derived from a prostate cancer) (Taplin et al. (1995) N. Engl. J. Med. 332, 1393-1398), was substituted for wild-type AR (FIG. 3B). These results indicate that the suppressive effect of mt-ARA54 or C′-ARA54 is relatively specific for fl-ARA54-enhanced AR transactivation.

(4) Effect of the Dominant-Negative ARA54 Mutant on Growth of Prostate Cancer Cells and PSA Expression

401. Prostate cancer cell lines stably transfected with the plasmids encoding the mutant ARA54 (C′-ARA54 or mt-ARA54) or fl-ARA54 under the doxycycline (doxy)-inducible promoter were made to investigate the effect of the dominant-negative ARA54 mutant on cell proliferation. Stable expression of the ARA54 induced was confirmed by doxy using Northern blotting. The LNCaP or PC-3 cells express endogenous ARA54 (wild-type) bands appeared at 3 Kb, and strong shorter bands (2 Kb) suggestive of C-terminal fragment transcript (C′-ARA54 or mt-ARA54) were detected only in the presence of doxy. Similarly, a stronger 3 Kb band was detected in the LNCaP cells stably transfected with fl-ARA54 when treated with doxy, compared to no doxy treatment or transfection with vector (pBIG2i) alone.

402. As shown in FIG. 4A, expression of the mt-ARA54 (+doxy) resulted in significant decrease of cell growth indicating the dominant-negative mutants of ARA54 reduced cell proliferation of the stable LNCaP cells, which had endogenous AR and wild-type ARA54. As a control the effects of fl-ARA54 in LNCaP and mt-ARA54 in AR-negative PC-3 cells was also tested. The results showed that fl-ARA54 or mt-ARA54 without AR does not suppress prostate cancer cell growth. The Luc assay also demonstrated that, using transient transfection of a reporter gene into these stable cell lines, expression of the mt-ARA54 (+doxy) significantly decreased AR transcription activity in the presence of DHT (FIG. 4B). These results confirm and strengthen the transient transfection data described herein.

403. The PSA is an AR target gene and presently the most useful marker to monitor the progression of prostate cancer. It is therefore of interest to determine if overexpression of the mutant ARA as dominant-negative inhibitors of AR transcription suppresses PSA expression in prostate cancer cells. The Western blotting assay showed that endogenous PSA expression in the LNCaP cells was decreased to 60% and 87% when the mt-ARA54 and C′-ARA54 were expressed in the cells (+doxy), respectively (FIG. 4C). There were no differences in AR protein levels in the LNCaP cells cultured with or without doxy. These results indicate that a dominant-negative mutant ARA54 can inhibit AR-mediated prostate cancer progression.

(5) Effect of the Dominant-Negative ARA54 Mutant on AR-ARA54 and ARA54-ARA54 Interactions

404. A mammalian two-hybrid assay was used to show the mechanism through which mt-ARA54 suppresses ARA54-enhanced AR transactivation. DU145 cells were cotransfected with a GAL4 DBD and a VP16 AD fusion protein. Protein-protein interaction was assessed by measuring the activity of the pG5-CAT reporter gene. First, we tested the influence of mt-ARA54 on the interaction between AR and fl-ARA54. As shown in FIG. 5A, AR interacted with fl-ARA54 in an androgen-dependent manner (lanes 1-4), as previously reported (Kang et al. (1999) J. Biol. Chem. 274, 8570-8576). The addition of C′-ARA54 or mt-ARA54 resulted in very little change in AR-ARA54 interaction (lanes 5 and 6). Also, AR still interacted with C′-ARA54 but not with mt-ARA54 (lanes 7 and 8), consistent with the yeast two-hybrid screening results disclosed herein. As shown in FIG. 5B, GAL4-fl-ARA54 interacted with VP16-fl-ARA54 in the presence or absence of androgen (lanes 1-4), indicating fl-ARA54 can form homodimers in an androgen-independent manner. When cotransfected with C′-ARA54 or mt-ARA54, CAT activities returned to the basal levels (lanes 5 and 6). Interestingly, fl-ARA54 can still interact with C′-ARA54 or mt-ARA54 (lanes 7 and 8). These results indicate that C′-ARA54 and mt-ARA54 can function in a dominant-negative manner through blocking the homodimerization of fl-ARA54.

405. Disclosed herein is a dominant-negative mutant of an AR coactivator, ARA54, identified using in vitro mutagenesis and a yeast two-hybrid screening assay. A mutated C-terminal ARA54 library using hydroxylamine-mediated mutagenesis to induce random transition mutations was used (Narusaka et al. (1999) J. Biol. Chem. 274, 23270-23275). The mutant ARA54, mt-ARA54, carrying a glutamic acid to lysine substitution at codon 472 has lost its binding ability to AR and significantly suppressed the ability of endogenous or exogenous fl-ARA54 to enhance AR transcription in prostate cancer cells. The inhibitory effect was more pronounced for exogenously expressed fl-ARA54 in DU145 cells than for endogenously expressed ARA54 in PC-3 and LNCaP cells. C′-ARA54 was shown to have a weak dominant-negative effect, but the mutant derived from this C-terminal fragment had a stronger suppressive effect on AR transactivation as well as AR-mediated prostate cancer proliferation.

406. ARA54 has the ability to form homodimers, as determined by using a mammalian two-hybrid assay. Because C′-ARA54 or mt-ARA54 did not influence fl-ARA54-AR interaction but did influence the interaction between fl-ARA54 and fl-ARA54, the molecular mechanism of these dominant-negative mutants appears to involve the formation of inactive dimers with fl-ARA54. In FIG. 6, a working model for the repression of AR transcription activity by C′-ARA54 or mt-ARA54 is presented. AR transactivation is induced by androgen and further enhanced through the interaction of AR with ARA54. For ARA54 to enhance AR transactivation, it may need to form homodimers. When fl-ARA54 dimerizes with C′-ARA54 or with mt-ARA54, the capacity of ARA54 to enhance transcription is reduced, resulting in a decrease in the observed AR-mediated transactivation.

407. Both normal prostate development and prostate cancer growth are largely dependent on the presence of androgens. Consequently, androgen ablation and/or blockage of androgen action through AR produces a brief response in most prostate cancer patients. However, in some cases prostate tumors are induced to proliferate by antiandrogens exerting an agonistic effect (Miyamoto et al. (1998) Proc. Natl. Acad. Sci. U.S.A. 95, 7379-7384; Kelly et al. (1997) Urol. Clin. North Am. 24, 421-431), and androgen dependence is eventually lost during treatment (Goktas, S., and Crawford, D. (1999) Semin. Oncol. 26, 162-173). It has been suggested that, due to changing the activity, for example, altering ligand specificity by AR variations and abnormalities, the activation of the AR pathway likely remains important in most prostate cancer cells from patients with clinically defined androgen-independent disease (Jenster, G. (1999) Semin. Oncol. 26, 407-421). Thus, in addition to current endocrine therapy, new approaches leading to inhibition of AR-mediated prostate cancer growth are needed. Currently, several in vivo gene therapies involving the insertion of suicide genes, the replacement of mutated tumor suppressor genes, and antisense strategies are being evaluated in prostate cancer model systems as potential treatments (Hrouda et al. (1999) Semin. Oncol. 26, 455-471). Disclosed herein are the suppression of AR coactivator function can be targeted to reduce AR activity. Loss of the function of an AR coactivator resulted in a complete androgen-insensitivity syndrome patient in whom the AR gene was completely normal (Adachi et al. (2000) N. Engl. J. Med. 343, 856-862). Disclosed are mutant coactivators, such as ATA-54, such as mt-ARA54 that suppresses androgen- and antiandrogen-mediated AR transactivation and PSA expression in prostate cancer cells. Disclosed herein these molecules can be used in gene therapy approaches to treat AR androgen independent prostate cancers. These results can lead to the development of new types of gene therapy strategies using mutant ARA54 or other suppressive mutant coactivators.

408. Also disclosed are method for obtaining dominant negative mutants of other AR coactivators.

### 3. Example 3

### Functional Domain and Signature Motif Analyses of Androgen Receptor Coregulator ARA70 and Its Differential Expression in Prostate Cancer

409. Androgen receptor (AR) associated coregulator 70 (ARA70) was first isolated as an AR interaction protein that could enhance AR transactivation in prostate cancer DU145 cells. Here we show that ARA70 can interact with the AR in an androgen-enhanced manner via a region lacking the classical LXXLL motif. This region, located between amino acids 176-401 (named ARA70-N2), can also function as a dominant-negative repressor of endogenous AR target genes, such as PSA, in prostate cancer cells. Although our results suggest that LXXLL motif is not responsible for the interaction to AR, however, mutation of this motif on ARA70 differentially effects its interaction to PPARr and RXR. Furthermore, ARA70N, containing amino acids 1-401, has better coregulator activity than full length ARA70 (ARA70-FL), and can translocate with the AR in the presence of 10 nM dihydrotestosterone (DHT). Interestingly, while immunocytofluorescence suggests that full length ARA70 is located in the cytosol, semi-quantitative analysis indicates that the coexpression of ARA70 can significantly enhance AR nuclear staining (p<0.0005), presumably either by promoting nuclear translocation or by stabilization of nuclear AR protein. Pulse-chase labeling and western blot analysis further confirm that ARA70 may stabilize or increase newly synthesized AR. Furthermore, immunochemical staining results indicate that ARA70 increases in the later stages and hormone refractory prostate cancer tissues, which correlates the roles of ARA70 to AR activity and function. Together, our data suggest that ARA70 may go through multiple mechanisms using various functional domains to regulate AR function.

a) Materials and Methods

(1) Materials and Plasmids

410. DHT was obtained from Sigma, and the plasmids pSG5-AR and pSG5-ARA70N were constructed as previously described (Dynlacht et al. (1991) Cell 66, 563-576; Miyamoto et al. (1998) Proc Natl Acad Sci USA 95, 7379-7384). The plasmid construction junctions were verified by sequencing.

(2) Cell Culture and Transfections

411. Human prostate cancer DU145 and PC-3 cells were maintained in Dulbecco's Minimum Essential Medium (DMEM) containing penicillin (25 U/ml), streptomycin (25 μg/ml), and 5% fetal calf serum (FCS). Human LNCaP cells were maintained in RPMI containing penicillin (25 U/ml), streptomycin (25 μg/ml), and 10% FCS. Transfections were performed using the calcium phosphate precipitation method, as previously described (Dynlacht et al. (1991) Cell 66, 563-576). Briefly, 4×10⁵ cells were plated on 60-mm dishes 24 hours before transfection, and the medium was changed to DMEM with 5% charcoal-dextran stripped FCS (CS-FBS) one hour before transfection. Transfection medium contained a constant amount of reporter plasmid and indicated amounts of pSG5-receptor, ARA70, or pCMX-GAL fusion construct using pSG5 as a carrier to provide equal amounts of transfected DNA. Twenty-four hours after transfection, the medium was changed again, and the cells were treated with DHT or other treatments. After another 24 hours, the cells were harvested for chloramphenicol transferase (CAT) or luciferase assays. At least three independent experiments were carried out in each case. Superfect (Qiagen) was used for transfection in LNCaP cells. The transfection conditions followed the manufacturer's protocol. Cell extracts were prepared and assayed for CAT or luciferase activity (Promega) and normalized against β-galactosidase or Renilla luciferase activity as indicated. All data were the mean±SD results from three to six independent experiments.

(3) Glutathione S-Transferase (GST) Pull-Down Assay

412. GST-ARA70 fusion protein and GST control protein were purified as described by the manufacturer (Amersham Pharmacia). The purified GST proteins were then resuspended in 100 μl of interaction buffer (20 mM HEPES/pH 7.9, 150 mM KCl, 5 mM MgCl₂, 0.5 mM EDTA, 0.5 mM Dithiothreitol, 0.1% (v/v) NP-40, 0.1% (w/v) BSA and 1 mM PMSF) and mixed with 5 μl of [³⁵S]-labeled TNT AR protein in the presence or absence of 1 μM ligand at 4° C. for 3 hours. After several washes with NETN buffer, the bound proteins were separated by SDS/8% PAGE and visualized using autoradiography.

(4) Yeast Two-Hybrid Interaction Assay

413. A fusion protein (GAL4AR) containing the GAL4 DNA binding domain (GAL4DBD) and the C-terminus of the AR was used as bait to test the interaction with different regions of ARA70. The transformed yeast Y190 cells were selected for growth on plates with 20 mM 3-aminotriazole and serial concentrations of androgens but without histidine, leucine, or tryptophan. The liquid assay was performed as described (Dynlacht et al. (1991) Cell 66, 563-576).

(5) In Vitro Site-Directed Mutagenesis

414. a VP16-ARA70 LXXAA mutant was generated by using the following four primers: 5′-CCGGAATTCTCAGTCCACCCAAGGTCT-3′,5′-GCTCTACTCGGCAGCGGGCCAGTTCAATTG-3′, 5′GAACTGGCCCGCTGCCGAGTAGAGCGCTG-3′, and 5′-CGCGGATCCCTCTACCTTACATGGGTC-3′. Mutagenesis was carried out on the cDNA fragment encoding amino acids 1-401 or full length ARA70 by PCR. The mutated fragment was then reinserted in frame into the pCMX-VP16 and pSG5 expression plasmids.

(6) Immunocytofluorescence Detection of the AR and ARA70 in COS-1 Cells

415. COS-1 cells were seeded on two-well Labtek II slides (Nalge) 24 hours before transfection. Two micrograms of DNA per 10⁵ cell was transfected with the AR, with or without full length ARA70 (ARA70-FL) using FuGENE6 transfection reagent (Roche). Twelve hours after transfection, the cells were treated with 10 nM DHT or ethanol. Immunostaining was performed by incubation with anti-AR polyclonal antibody (NH27) or anti-ARA70 mouse monoclonal antibody (CC70), followed by incubation with either fluorescence-conjugated goat anti-rabbit or anti-mouse antibodies (ICN). The red signal represents the AR and the green signal represents ARA70. Blue DAP1 staining shows the location of the nucleus.

(7) Semi-Quantitative Analysis & Student's T-Test

416. Three hundred cells with normal morphologies and clear AR nuclear translocation were scored for AR staining using a fluorescence microscope. Cells were scored on a scale of one to five, with one representing the lowest AR staining intensity above the background level. The cells were then separated into two groups based on the presence or absence of ARA70.

417. The mean AR staining intensity and standard deviation were then calculated for the ARA70 negative and positive populations. Using STATAQUEST, a two sample t-test (assuming unequal variances) was then performed to determine if the difference in the mean AR staining intensities in the two populations was statistically significant (α=0.05).

(8) Pulse-Chase Labeling

418. COS-1 cells were seeded in 100-mm dishes and transfected with the AR, with or without ARA70 as indicated for 3 hours using Superfect (Qiagen) and then subjected to pulse-chase metabolic labeling with [³⁵S] methionine/cysteine for 30 minutes. After changing the medium, the cells were harvested at the times indicated in FIG. 13. Whole cell extracts were prepared by RIPA buffer (150 mM NaCl, 50 mM Tris, 10% SDS, 0.5% DOC (w/v) and 1% NP-40) and then immunoprecipitated with anti-AR antibody (NH27). The specificity of the immunoprecipitation was confirmed using preimmune serum as well as protein A-Sepharose beads alone (data not shown).

b) Results

(1) Interaction Domains of the AR and ARA70

419. ARA70-FL was cut into several fragments, which were ligated into pAS2 vectors for the yeast two-hybrid assay to determine which domain(s) of ARA70 can interact with the AR. As shown in FIG. 7A-B, ARA70N peptide (aa 1 to 401) and ARA70-N2 peptide (aa 176 to 401) can interact with the AR ligand binding domain (AR-LBD) in the presence of 10 nM DHT. In contrast, three other ARA70 peptides, ARA70 LXXLL (aa 90 to 99; L, leucine; X, any amino acid), ARA70-N1 (aa 1 to 175) and ARA70-C (aa 383-614) could not interact with the AR-LBD.

420. Using the mammalian two-hybrid system, the data from FIG. 7C, further confirmed that ARA70-N2, but not ARA70-N1 or ARA70-C, can interact with the AR in an androgen-dependent manner (FIG. 7C). Data from the yeast and mammalian systems together demonstrate that ARA70-N2, lacking the conserved LXXLL motif, is the essential domain for interaction with the AR-LBD in the presence of androgen.

(2) The LXXLL Motif of ARA70 is Dispensable for Interaction with the AR, but is Necessary for Interaction with the Nonclassical Nuclear Receptor PPARγ

421. The LXXLL motif in ARA70N was mutated to a LXXAA and tested whether this mutated ARA70N (mtARA70N) could still interact with the AR. As shown in FIG. 8A-B, data from the mammalian two-hybrid system clearly demonstrate that there is no difference in the interaction of VP16 fused wild-type ARA70N (ARA70N) or VP16 fused mtARA70N with the AR-LBD. The results of the site-directed mutagenesis assay confirm that the LXXLL motif is dispensable for AR-ARA70 interaction (FIG. 8B), but this mutation does affect the interaction of ARA70 with the LBD of PPARγ (FIG. 8C). Together, these data suggest distinct molecular mechanisms for ARA70 interaction with classical versus non-classical nuclear receptors.

(3) The Function of Different Domains of ARA70 in AR Transactivation

422. To delineate the functional domains of ARA70, the CAT assay was used to study the potential influence of various ARA70 peptides on AR transactivation in DU145 cells. As shown in FIG. 9, ARA70N and ARA70-FL, as well as their mutants, mtARA70N and mtARA70-FL, lacking the LXXLL domain, showed similar enhancement of AR transactivation. These results are consistent with the above mammalian two-hybrid data showing that mtARA70N, lacking the LXXLL domain, can still interact with the AR. The data in FIG. 9 also show that ARA70N has better AR enhancement activity than ARA70-FL, and that neither ARA70-N1 nor ARA70-N2 can enhance AR transactivation in DU145 cells (lanes 3 & 4).

(4) ARA70-N2 Functions as a Dominant-Negative Repressor of AR Transactivation

423. The data further indicate that ARA70-N2, the AR interaction motif lacking coactivational activity, can function as a dominant-negative repressor to inhibit ARA70N-enhanced AR transactivation (FIG. 10). ARA70-N2 only slightly represses other AR coregulators, however, such as ARA55 (Yeh, S., and Chang, C. (1996) Proc. Natl. Acad. Sci. USA 93, 5517-5521), ARA54 (Fujimoto et al. (1999) J. Biol. Chem. 274, 8316-8321), and SRC-1 (Hsiao et al. (1999) J. Biol. Chem. 274, 20229-20234) (FIG. 10A). Without exogenously transfected ARA70-FL and wtAR, ARA70-N2 can also suppress endogenous ARA70-FL-mediated mtAR (mtAR877) transactivation in LNCaP cells (FIG. 10B). These results, together with mammalian two-hybrid data showing that only ARA70-N2 can interact with the AR, strongly suggest that ARA70-N2 can function as a dominant-negative repressor of ARA70-enhanced AR transactivation.

424. ARA70-N2 can repress AR transactivation of the endogenous AR-target gene, PSA, in LNCaP cells. Instead of using transiently transfected ARE-CAT reporter, northern blotting and western blotting were applied to assay the influence of ARA70-N2 on endogenous AR-mediated PSA expression. As shown in FIG. 10, the addition of ARA70-N2 repressed PSA mRNA (FIG. 10C) and protein (FIG. 10D) expression in LNCaP cells. These results indicate that ARA70-N2 can serve as a dominant-negative repressor to inhibit in vivo AR transactivation.

(5) FXXLF Motif Within ARA70 N2 Domain in is Essential for the Interaction Between ARA70 and AR

425. Using E. coli. expressed AR-DBD-LBD protein as a bait to screen a 12-mer random peptide library expressed on the coat of M13 bateriophage, a unique motif FXXLF in at least 5 different peptides that can interact with AR, was identified. These individual peptides were tested and can still interact with AR in the mammalian two-hybrid system. After data analysis, a FXXLF motif in the ARA70 N2 was identified. The ARA70N FXXLF motif was mutated and tested its influence on the binding to AR. Results from the mammalian two-hybrid system show that wild-type ARA70N-FXXLF can interact well with AR. In contrast, mutants ARA70N-AXXLF or ARA70N-FXXAA have little capacity to interact with AR. These results indicated that the FXXLF motif within the ARA70 N2 domain is essential for the interaction between ARA70 and AR and consistent with the results in FIGS. 7 and 8 that ARA70 N2 is the AR interaction region.

(6) FXXLF Signature Motif Influences AR Transactivation.

426. The ARA70N which contains wild-type FXXLF, mutated AXXLF or FXXAA was constructed in pSG5 expression vectors and their influence on the AR transactivation was tested. As shown in FIG. 11B, in COS-1 cells, 10 nM T can induce AR transactivation 8 fold (lanes 1 vs 2). Addition of wild-type pSG5-ARA70N-FXXLF further enhances AR transactivation to 310 fold (lanes 2 vs 3). In contrast, addition of mutant pSG5-ARA70N-AXXLF or pSG5-ARA70N-FXXAA only shows marginal induction effect for AR transactivation (lanes 2 vs 4 and 5). Together, our results indicated that mutation of the FXXLF in ARA70 may cause the ARA70 lost interaction with AR, and this can be translated to influence AR transactivation.

(7) Immunostaining of the AR and ARA70

427. Immunocytofluorescence staining assays using specific antibodies against the AR (NH27) or ARA70 (CC70) were applied to further dissect the molecular mechanism of ARA70 coregulator activity. As shown in FIG. 12, the AR was mainly located in the cytoplasm in the absence of androgen (FIG. 12A) and moved to the nucleus after the addition of 10 nM DHT (FIG. 12B). ARA70 was located in the cytoplasm in the absence or presence of the AR and 10 nM DHT in COS-1 cells (FIG. 12C vs. D). Co-transfection of ARA70 with the AR in the presence of 10 nM DHT, however, enhanced the immunostaining intensity of nuclear AR (FIG. 12 E-H). Semi-quantitative analysis of nuclear AR staining intensity and Student's t-test, (STATAQUEST), indicate that ARA70 coexpression significantly enhances nuclear AR staining intensity (p<0.0005). These results suggest that ARA70 may enhance AR transactivation by promoting AR nuclear translocation or stabilization, and/or increasing the amount of nuclear AR protein.

(8) Co-Localization of the AR and ARA70N by Immunocytofluorescence Assay

428. As the data consistently show that ARA70N has better AR enhancement activity than ARA70-FL (FIG. 10 B), the cellular distribution of ARA70N was determined. Using the same immunocytofluorescence assay in COS-1 cells, our results indicate that ARA70N alone, without co-transfection of the AR, is homogeneously distributed in the cell in the absence or in the presence of 10 nM DHT (FIG. 121). Furthermore, ARA70N is also homogeneously distributed in the cell with co-transfection of the AR in the absence of DHT (FIG. 12J). In contrast, when co-transfected with the AR in the presence of 10 nM DHT, ARA70-N translocated into the nucleus (FIG. 12K), suggesting that liganded AR can interact with ARA70N and facilitate ARA70N nuclear translocation. The nuclear translocation of ARA70N in the presence of 10 nM DHT may account for the increased enhancement of AR transactivation compared to ARA70-FL.

(9) Full Length ARA70, but not Antisense ARA70, Enhances the Expression of AR

429. To confirm the results observed in the immunocytofluorescence experiments, a western blotting assay was applied to assay the AR protein level. As shown in FIG. 13, both ARA70N and ARA70-FL enhance the amount of AR protein, while antisense ARA70 does not influence AR protein levels. Furthermore, the expression of TR4, another AR interacting protein (Lee et al. (1999) Proc Natl Acad Sci USA. 96, 14724-14729), slightly decreases the amount of AR protein. The results from FIG. 13 indicate that the enhancement of AR protein levels by ARA70 is specific because: 1) both ARA70 and ARA70N increase AR protein levels, 2) expression of TR4 does not increase, but instead slightly decreases AR protein levels, and 3) antisense ARA70, which cannot potentiate AR transactivation, does not enhance the protein level of the AR.

(10) ARA70 may Enhance AR Transactivation by Stabilization and/or Increasing Newly Synthesized AR Protein

430. ARA70 can stabilize AR protein, as demonstrated by pulse-chase labeling using [³⁵S]-Methionine-AR to assay the amount of newly synthesized AR. As shown in FIG. 14A, the amount of newly synthesized AR within the first 2 hours was relatively higher in the presence of ARA70, which likely due to enhancing the metabolic stability or increasing the amount of newly synthesized AR. In contrast, the amount of newly synthesized AR after 2 hours was lower in the presence of TR4 (FIG. 14B). These results suggest that ARA70 may be able to enhance AR transactivation by metabolic stabilization and/or increasing the amount of newly synthesized AR. Together, data from immunostaining (FIG. 12), western blot analysis (FIG. 13), and pulse-chase labeling (FIG. 14), all indicate that ARA70 may enhance AR transactivation by metabolic stabilization or increasing newly synthesized AR, resulting in enhanced nuclear staining of the AR.

431. Using prostate cancer DU145 cells, it was found that among all classic steroid receptors, including the GR, progesterone receptor (PR), ER, and AR, co-transfection with ARA70 could enhance the transactivation of GR, PR, or ER only 2-3 fold. In contrast, AR transactivation would be enhanced by ARA70 from 1 fold up to 8-10 fold, depending on the ratio of AR to ARA70 in the cells. Using other cell lines, it was found that ARA70 could enhance AR transactivation 8-fold in CV-1 cells 6-fold in PC-3 cells, and 8-fold in COS-1 cells. Recently, when the analysis of ARA70 was extended to non-classical nuclear receptors, our results indicated that ARA70 could also enhance the transactivation of PPARγ and heterodimers of PPARγ-RXR. In CV-1 cells, it was reported that ARA70 functions as a relatively weak AR coactivator and only enhances AR activity 2-3 fold.

432. Considering that different cell lines may express a variety of different endogenous AR coactivators, the combination of different expression vectors, transfection methods, and cell lines may result in varying amounts of exogenous ARA70 to yield diverse squelching effects. Fluctuating ARA70 enhancement activity under these varying experimental conditions should be observed. The variation in ARA70 enhancement activity is not a unique phenomenon among SR coregulators.

433. The relevant domains in AR-ARA70 functional interaction are disclosed herein. The LXXLL motif has been identified as the signature motif for p160 coregulators to interact with SRs (Anzick et al. (1997) Science 277, 965-968; Heery et al. (1997) Nature 387, 733-736). It has been well documented that the removal of the LXXLL motif can abolish the interaction between p 160 coregulators and steroid receptors. Disclosed herein, however, this motif is not essential for ARA70 to interact with the AR. In addition, sequence analysis revealed that ARA70 is lacking other common coregulator motifs, such as the basic helix-loop-helix (bHLH) domain, and the Per-AhR-Sim (PAS), that are shared by the coregulator family of SRC-1, TIF2/GRIP1, and AIB1/P/CIP/RAC3/ACTR/SRC3 (Hsiao et al. (1999) J. Biol. Chem. 274, 20229-20234; Onate et al. (1995) Science 270, 1354-1357; Hong et al. (1996) Proc Natl Acad Sci USA 93, 4948-4952; Voegel et al. (1996) EMBO J. 15, 3667-3675; Li et al. (1997) Proc Natl Acad Sci USA 94, 8479-8484;

434. Chen et al. (1997) Cell 90, 569-580; Anzick et al. (1997) Science 277, 965-968). While the LXXLL motif is dispensable for the interaction with the AR, ARA70 utilizes this motif to interact with the non-classical nuclear receptor PPARγ.

435. SRs function as transcription factors to regulate the expression of their target genes in the nucleus. Before ligand binding, some SRs are located in the cytosol (McNally et al. (2000) Science 287, 1262-1265) and are associated with heat shock proteins. Heat shock proteins behave as protein chaperones in maintaining the proper conformation of SRs, thereby assisting in their consequent activation (Rajapandi et al. (2000) J. Biol. Chem. 275, 22597-22604; Pratt, W. B., and Toft, D. O. (1997) Endocr. Rev. 18, 306-360; Pratt et al. (1993) J. Steroid Biochem. Mol. Biol. 46, 269-279)). Cytosolic proteins may also be involved in the proper functioning of individual receptors, including cytosolic mediators of signal transduction phosphorylation cascades, transportation, anchoring, ubiquination, or degradation of steroid receptors. Overall, this cytosolic regulation may subsequently affect SR transactivation events in the nucleus.

436. Using immunocytofluorescence, disclosed herein, full length ARA70, an AR associated protein, is located in the cytosol, and yet still has the capacity to enhance AR transactivation. The results from pulse-chase labeling indicate that newly synthesized AR protein is stabilized and/or increased by the co-transfection of ARA70 during the first 4 hours. The difference, however, gradually reduces to insignificance, which is in agreement with our earlier report (Miyamoto et al. (1998) Proc Natl Acad Sci USA 95, 7379-7384) showing that AR protein was only slightly enhanced (12%) 48 hours after co-transfection with ARA70 in DU145 cells. The metabolic stabilization and/or increase in the amount of AR protein in the presence of ARA70 was also confirmed by western blot analysis of COS-1 cell extracts and semi-quantitation of nuclear AR immunostaining using fluorescence microscopy. Other reports have also demonstrated that cytosolic proteins or even membrane-bound proteins, such as β-catenin and caveolin, can behave as coactivators to enhance AR transactivation (Heery et al. (1997) Nature 387, 733-736; McNally et al. (2000) Science 287, 1262-1265), though the detail mechanism underlying this phenomenon remains to be elucidated.

437. It has been found that SR coregulators may exist as different isoforms to function as receptor coregulators. For example, SRC-1a and SRC-1e possess different capacities to regulate SR activity (Kalkhoven et al. (1998) EMBO J. 17, 232-243; Hayashi et al. (1997) Biochem. Biophys. Res. Commun. 236, 83-88).

438. The disclosed data also indicate that ARA70N, a peptide lacking the C-terminal domain of ARA70, has better coregulator activity. Furthermore, while the distribution of cytosolic ARA70 was not influenced by the addition of the AR and 10 nM DHT, ARA70N translocated to the nucleus with the AR in the presence of androgen.

### 4. Example 4

### Identification and Characterization of a Novel Androgen Receptor Coregulator ARA267 in Prostate Cancer Cells

a) Materials and Merthods

(1) Materials and Plasmids

439. 5α-dihydrotestosterone (DHT), dexamethasone (Dex), progesterone (P), 17β-estradiol (E2), Δ5-androstendiol and dehydoepiandrosterone (DHEA) were obtained from Sigma and hydroxyflutamide (HF) were obtained from Schering. pSG5AR, pSG5ARA55, pSG5ARA54 and pSG5ARA70N (ARA70 N-terminal) was constructed as described previously (Chang et al. (1995) Crit. Rev. Eukaryotic Gene Expression 5, 97-125; Fujimoto et al. (1999) J. Biol. Chem. 274, 8316-8321; Kang et al. (1999) 274, 8570-8576; Yeh et al. (1999) Proc Natl Acad Sci USA 96, 5458-5463). Expression plasmid of BRCA1 was from Michael R Erdos (Genetics and molecular Biology Bronch, National Human Genome Research Institute, National Institute of Health). Smad3 Expression plasmid was provided by Rik Derynck (Univ. of California, San Francisco). Expression plasmid of CBP was provided by Richard H. Goodman (Vollum Institute, Oregon Health Sciences University, Portland, Oreg.) and reconstructed into pCMV expression vector by ourself. pCMX-GAL4ARC (AR DBD+LBD) and pCMX-VP16ARN (AR activation domain) were constructed for mammalian two-hybrid assay (11C), pGEX-GST-ARA267N1, pGEX-GST-ARA267N2 and pGEX-GST-ARA267C were constructed for the Glutathione S-transferase (GST) pull-down assay.

(2) Cell Culture

440. Human cancer cell lines PC-3, U2OS, SAO2, DU145, and H1299 were grown in Dulbecco's minimal essential medium (DMEM) containing 10% fetal calf serum (FCS), penicillin (25 units/ml) and streptomycin (25 μg/ml). T47D, MCF-7 and LNCaP were maintained in RPMI 1640 with 10% FCS, penicillin (25 units/ml), and streptomycin (25 μg/ml).

(3) Yeast Two-Hybrid Screening

441. A MATCHMAKER yeast two-hybrid human brain cDNA library (CLONTECH) that consists of GAL4 activation domain, amino acid (aa) 768-881, fused with human brain cDNA was used in our yeast two-hybrid screening. The library was screened by co-transformation with a bait construct, GAL4-DBD fused with full-length testicular receptor 4 (TR4) protein, as previously described (Yeh et al. Proc. Natl. Acad. Sci. U.S.A. (1996) 93, 5517-5521). The transformed yeast Y190 cells were selected for growth on plates with 20 mM 3-aminotriazole and 1 μM 5α-DHT but without histidine, leucine, or trytophan. TR4 is a nuclear orphan receptor with an unknown ligand. Mating tests were used to further confirm the protein-protein interaction in yeast cell. One of the initial 31 potentially positive clones reacted firmly with TR4 and AR-LBD fusion protein (GAL4-DBD-AR-LBD, aa 595-918). This clone was designated as Y1600 and selected for the further evaluation.

(4) Polymerase Chain Reaction and Cloning Full-Length ARA267

442. Using the sequence of the clone we isolated from the library, we searched the GeneBank database. According to the sequence of the EST clones, several primers were designed with 5′ linker containing restriction enzyme site in order to amplify the full length of this clone. An ˜8.0 kb product was amplified, sequenced (BigDye Terminator Kit, Perkin-Elmer), and subcloned into pSG5 vector. The PCR template was Marathon human testis cDNA library (CLONTECH) and the program was 94° C. 1 min, 5 cycles of 94° C. for 5 sec, 72° C. for 12 min, 5 cycles of 94° C. for 5 sec, 70° C. for 12 min, 30 cycles of 94° C. for 5 sec, and 68° C. for 12 min. The 5′ start codon ATG was confirmed by 5′-RACE-PCR.

(5) Northern Blot and Dot Blot

443. Human cancer cell lines, PC-3, U2OS, SAO2, T47D, LNCaP, DU145, H1299, and MCF-7 were cultured following the method as previously described. Total RNA was isolated from each cell line using total RNA isolation reagent, TRIZOL Reagent (Gibco/BRL). We loaded 25 μg of total RNA from each cell line onto denaturing agarose gel, the RNA samples were separated by electrophoresis, and blotted onto a nylon membrane through a vacuum blotter. Y1600 clone containing a 1.6 kb fragment of ARA267 (911 bp-2542 bp) was used as the probe for the hybridization. A β-actin probe was used as a control for equivalent RNA loading. A human multiple tissue RNA dot-blot, purchased from CLONTECH (Catalog number 7775-1), was also hybridized with the same ARA267 (Y1600 clone) probe to evaluate tissue distributions of ARA267 in normal human tissues.

(6) Transfection and Report Gene Assay

444. Human prostate cancer cell line PC-3 and DU145, lung cancer cell line H1299, and hepatoma cell line HepG2 were grown in DMEM-10% FCS. For transfection the cells were plated in 60-mm dishes and experiments performed by modified calcium phosphate technique as previously described (Yeh et al. Proc. Natl. Acad. Sci. U.S.A. (1996) 93, 5517-5521). After incubation for 24 h, the cells were treated with steroid hormones for another 24 h, then harvested for the chloramphenicol acetyltransferase (CAT) assay. Mouse mammary tumor virus-(MMTV)-CAT reporter gene was used to measure AR transcription activity, and a β-Galactosidase expression gene (pCMV-O-gal) was incorporated into the experiments as an internal control (Yeh et al. Proc. Natl. Acad. Sci. U.S.A. (1996) 93, 5517-5521). CAT activity was visualized by a PhosphorImager (Molecular Dynamics) and quantitated by IMAGEQUANT software (molecular Dynamics). For Luciferase (LUC) assay, pG5-LUC, pMMTV-LUC or estrogen response element (ERE)-LUC plasmid was used as the reporter gene and SV40-PRL (promega) was used as an internal control. Dual-luciferase Reporter 1000 Assay System (promega) was employed to measure the luciferase activity.

(7) Glutathione S-transferase (GST) Pull-Down Assay

445. GST-ARA-267 N-terminal and C-terminal fusion proteins were expressed in E. coli strain BL21, and purified as described by manufacturer (Amersham Pharmacia). The purified fusion proteins were resuspended in 100 μl interaction buffer [20 mM HEPES/pH 7.9, 150 mM KCL, 5 mM MgCL₂, 0.5 mM EDTA, 0.5 mM DTT, 0.1% (vol/vol) Nonidet P-40, 0.1% (wt/vol) BSA, 1 mM PMSF and 10% glycerol] and mixed with 5 μl of [³⁵S]-labeled TNT expressed AR N-terminal, C-terminal, and full-length proteins (TNT coupled reticulocyte lysate system, Promega) in the presence or absence of 1 μM DHT and incubated at 4° C. for 5 h. After several washes with NETN buffer [20 mM Tris/pH 8.0, 100 mM NaCl, 6 mM MgCL₂, 1.0 mM EDTA, 1.0 mM DTT, 0.5% (vol/vol) Nonidet P-40, 1 mM PMSF, and 8% glycerol], the bound proteins were separated on SDS-PAGE gel and visualized by PhosphorImager (Molecular Dynamics).

(8) Mammalian Two-Hybrid Assay

446. For Luciferase assay, 3 μg pG5-LUC plasmid was used as the reporter gene and 10 ng SV40-PRL was used as an internal control. We transfected 4.0 μg ARA267 and 2.0 μg of each GAL4-ARC and VP16-ARN into PC-3 cells, with or without 1 nM DHT, using calcium phosphate method. Dual-luciferase Reporter 1000 Assay System (Promega) was employed to measure the luciferase activity.

(9) Western Blot Assay

447. LNCaP cells were transfected with pSG5ARA267 and pSG5 vector by Superfect (Qiagen) respectively. After transfection 2 hours, medium was changed, and ethanol and 10 nM DHT were applied for another 36 hours respectively. The cells were harvested and lysed following the protocol from Santa Cruz Biotechnology. In each sample, 50 μg whole-cell lysis proteins were separated on 10% SDS-polyacrylamide gel. After transfering, the membrane was blotted with polyclonal AR antibody (NH27), PSA antibody (Dako Corporation), and β-actin antibody (Santa Cruz Biotechnology). The bands were developed with an alkaline phosphatase detection kit (Bio-Rad).

b) Results

(1) Cloning and Sequence of ARA267

448. To further understand the function and mechanism of nuclear receptor action, LBDs of AR and TR4, an orphan receptor, were used as baits to fish out the interacting proteins from yeast two-hybrid system. ARA267 was isolated which can interact not only with TR4, but also with AR-LBD, in the presence of 1 μM DHT. RACE-PCR technology with the isolated DNA insert as template and several primers were then designed to amplify the full-length human ARA267 from the Marathon human testis cDNA library. Unexpectedly, the amplified DNA turns out to be an exceptionally long insert over 8 kb in size. The longest uninterrupted coding sequence within this 8 kb transcript has 2427 amino acids with a calculated molecular weight of 267 kD (FIG. 15). The sequence analysis indicates that ARA267 is a novel human gene, with no homology with previously identified AR coregulators, such as ARA24, ARA54, ARA55, SRC-1, ARA70, and ARA160. ARA267 contains several important functional domains shown boxed or underlined in FIG. 15. For example: ARA267 contains one SET domain (aa 1668-1795), two LXXLL motifs (aa 726-730 and aa 1283-1287), three nuclear translocation signals (NLS) (aa 243-260, aa 888-905, and aa 1202-1219), four plant homodomain (PHD) fingers (aa 1274-1320, aa 1321-1377, aa 1438-1482, and aa 1849-1896) and a proline-rich region. In the four PHD finger regions a Cysteine-rich region (aa 1277-1342), a ring finger (aa 1324-1369) and a Zinc-finger (aa 1884-1909) were also found.

(2) Northern Blot and Tissue Distribution

449. Northern blot analysis indicated that ARA267-is expressed as two mRNA transcripts of about 13 kb and 10 kb in many cell lines, such as PC-3, U2OS, SAO2, T47D, LNCaP, DU145, H1299, and MCF7 (FIG. 16A, lanes 1-7 and 9), but absent in HepG2 cell line (FIG. 16a, lane 8). Multiple tissues dot blot was used to determine the expression pattern of ARA267 in different tissues, using prostate as an indicator. Lung, placenta, uterus, kidney, thymus, lymph node, liver, pancreas and thyroid gland tissues have higher expression of ARA 267 than prostate tissue, with lymph node as the highest one. In contrast, tissues like bladder, testis, ovary, skeletal muscle, and mammary gland have relatively lower expression than prostate tissue (FIG. 16B).

(3) Interaction Between ARA267 and AR

450. To confirm the interaction between ARA267 and AR that was shown in the yeast two-hybrid system, GST pull-down assay was applied to confirm and further map the interaction domains between ARA267 and AR. Two ARA267 N-terminal domains, ARA267N1 (aa 1-382) and ARA267N2 (aa 1-984), and one C-terminal domain, ARA267C (aa 1716-2427), were constructed in GST fused vector (FIG. 17A). Each of these E. Coli-generated GST fusion proteins were then incubated with in vitro translated [³⁵S]-methionine-labeled AR-N (aa 36-553), AR-C (aa 553-918), or AR full length (FIG. 17A) for the GST pull-down assay. The results indicate that both GST-ARA267N1 and GST-ARA267N2 cannot interact with ARN (FIG. 17B, lanes 3 and 4), but can interact with AR-C (FIG. 17B, lanes 8-11) and AR full-length in the presence and absence of 1 μM DHT (FIG. 17B, lanes 15-18). FIG. 17C further demonstrates that ARA267C can interact with ARC peptide and full length AR in a DHT-enhanced manner (FIG. 17C, lanes 7-8 and 12-13). In contrast ARA267C cannot interact with ARN (FIG. 17C, lane 3). These data suggest that AR-C terminal (DBD+LBD domain), but not N-terminal, is responsible for the interaction between AR and ARA267.

451. As early data suggested that AR N-terminus can also interact with AR C-terminus (He et al. (1999) J Biol Chem 274, 37219-37225), ARA267 associatio with the AR C-terminus shows little influence on the interaction between AR N-terminus and C-terminus. Using the relative luciferase activity assay, we found while the coregulator CBP can enhance the interaction between AR N-terminal and C-terminal ARA267 is more like our previously identified coregulators, such as ARA70, ARA55, or ARA54 that show little influence on the AR N—C interaction (FIG. 18).

(4) Enhancement of AR Transactivation by ARA267

452. Human prostate cancer PC-3 cells which is AR negative cell line were transiently transfected with 3 μg of MMTV-CAT reporter, 1 μg of AR expression vector (pSG5AR), and with increasing amounts of full-length ARA267 (pSG5-ARA267) in 60-mm culture dishes. The total plasmid amount was adjusted to 11 μg with pSG5. As shown in FIG. 19A, ARA267 can enhance DHT-mediated AR transactivation in a dose-dependent manner. Similar results were also observed in human lung cancer H1299 cells (FIG. 19A). To further confirm ARA267 coregulator activity, western blot analysis was performed to see if ARA267 can also enhance AR endogenous target gene, prostate-specific antigen (PSA), expression in LNCaP cells. As shown in FIG. 19B, ARA267 can enhance DHT-induced PSA protein expression. In contrast, ARA267 showed little induction on the AR protein expression.

453. For the ligand specificity assay, the data show that DHT is the best ligand for the ARA267 coregulator activity. Unlike ARA70, which was able to enhance AR transactivation in the presence of other ligands, such as 1713-Estradiol (E2), Hydroxyflutamide (HF), A5-Androstenediol (Adiol), ARA267 only shows marginal effects on the AR transactivation in the presence of 10 nM E2 (FIG. 20).

454. To test the ARA267 receptor specificity, we replaced AR with other members of the SR family, such as glucocorticoid receptor (GR), progesterone receptor (PR), and estrogen receptor (ER), in luciferase assay with HepG2 cells that do not express endogenous ARA267. As shown in FIG. 21, ARA267 has better coregulator activity on AR as compared to PR. In contrast, ARA267 only has a marginal effect on the transactivation of GR and ER. Similar results also occurred when we replaced HepG2 cells with PC3 cells.

(5) ARA267 Additionally Enhances AR Transactivation with Other AR Coregulators

455. Since it has been demonstrated that several AR coregulators have the capacity to enhance AR transactivation (Yeh et al. Proc. Natl. Acad. Sci. U.S.A. (1996) 93, 5517-5521; Fujimoto et al. (1999) J. Biol. Chem. 274, 8316-8321; Kang et al. (1999) 274, 8570-8576; Hsiao et al. (1999) J. Biol. Chem. 274, 20229-20234; Hsiao et al. (1999) J. Biol. Chem. 274, 22373-22379; Yeh et al. Biochem. Biophys. Res Commun. (1998) 248, 361-367; Yeh et al. (2000) Proc. Natl. Acad. Sci. U.S.A. 97, 11256-11261; Kang et al. (2001) Proc. Natl. Acad. Sci. U.S.A. 98, 3018-3023; Yeh et al. Proc. Natl. Acad. Sci. U.S.A. (1998) 95, 5527-5532) it was determined if ARA267 has any additive or synergistic effects with other coregulators on AR transactivation. As shown in FIG. 22, it was found that ARA267 can additionally enhance AR transactivation with other AR coregulators, such as ARA24 (Hsiao et al. (1999) J. Biol. Chem. 274, 20229-20234) or PCAF, a coregulator with histone acetylase activity (Yeh et al. (1999) Endocrine 11, 195-202) in PC-3 cells. Together, the data demonstrated that the ARA267 functions as a coregulator to increase AR transcription activity in a ligand-dependent manner.

### 5. Example 5

### Identification of Gelsolin as an Antiandrogen-Potentiated Androgen Receptor Coregulator with Enhanced Expression in Prostate Cancers Following Androgen Ablation Therapy

a) Results

(1) Cloning of Gelsolin as an AR-Associated Protein

456. In order to determine if any AR-associated proteins are involved in antiandrogen withdrawal syndrome or progression of prostate cancer from androgen-dependent to androgen-independent stage, a yeast two-hybrid system was applied to screen AR interacting proteins in human prostate cDNA library using mtARt877s, point mutation at amino acid (aa) 877 from threonine to serine, as bait in the presence of 10 μM HF. The mtARt877s was identified from a patient with androgen-independent prostate cancer and its altered hormone specificity was demonstrated (Taplin et al. N Engl J Med 332, 1393-1398 (1995)). Since HF can activate this mtAR (Fenton et al. Clin Cancer Res 3, 1383-1388 (1997)), which was also confirmed in our laboratory (data not shown), we chose the ligand-binding domain (LBD) of mtARt877s as bait.

457. One of the positive cDNA clones, which can interact with mtARt877s, was further isolated and its cDNA sequence was identical with the C terminus (aa 281-731) of human gelsolin. The clone also interacted with wild type (wt) AR LBD in the presence of 100 nM DHT or 10 μM HF in yeast two-hybrid assays.

(2) Ligand-Dependent Interaction Between AR and Gelsolin

458. To determine whether AR interacts with gelsolin in a ligand-dependent manner, the yeast liquid β-galactosidase (β-gal) assay was first applied, which enables us to quantify interaction strength by measuring the β-gal activity. Y190 yeast cells were transformed with Gal4 DBD fused with the C-terminus (aa 595-918) of mtARt877s and Gal4AD fused with C terminus (aa 281-731) of gelsolin. Transformants were selected by their growth in medium with 10 μM HF, 100 nM DHT, 1 μM E2, 1 μM P, or ethanol (EtOH). HF, DHT, E2, and P promoted significant interaction between mtARt877s and gelsolin compared to EtOH (FIG. 23A). These results indicate a broad specific ligand-induced interaction between mtAR and gelsolin. The interactions between gelsolin and wtAR were next analyzed by mammalian two-hybrid assays, which are sensitive enough to detect relatively weak interactions. A Gal fusion protein containing wtAR (aa 36-918) and a VP16-gelsolin (aa 281-731) were co-expressed in COS-7 cells in the presence of T or HF (FIG. 23B). T promoted the significant interactions between wtAR and gelsolin in a dose-dependent manner at the concentration of 10 nM. Likewise, HF induced significant interaction of these proteins at 1 μM, a pharmaceutical concentration used in the treatment of prostate cancer. The ligand-dependent interaction of Gal4-gelsolin (aa 281-731) and VP16-AR (aa 36-918) were also confirmed in PC-3 cells.

(3) Interaction Domains are Located in Gelsolin C-Terminal and AR DBD-LBD

459. According to yeast and mammalian two-hybrid assays, gelsolin C-terminal interacts with AR. The nteraction domains between gelsolin and AR were determined by in vitro GST pull-down assay. A plasmid for expressing GST conjugated C-terminal fragment of gelsolin (aa 376-755) (GSNc), one of the products generated after caspase digestion (Sun et al. J Biol Chem 274, 33179-33182 (1999)), was constructed as well as an expression plasmid of GST conjugated full-length gelsolin (GSN). AR was truncated to several fragments according to the functional domain and expressed in vitro (FIG. 24A). The results from the GST pull-down assay indicate AR DBD and LBD but not N-terminus interact with both GSN and GSNc compared to GST protein along (FIG. 24B). The ligand effect is not obvious in this assay, possibly due to lacking chaperone proteins in this assay system.

(4) Gelsolin Enhances AR Activity in a Ligand-Dependent Manner

460. To address the functional significance of the interaction between AR and gelsolin, reporter gene assays by transient transfection of gelsolin and AR expression plasmids into human prostate cancer DU145 cells were performed. Transfection of full-length gelsolin enhanced AR transcription activity by 2-3 fold in the presence of 10 nM DHT, whereas transfection of full-length gelsolin had no significant effect on AR transcription activity in the absence of DHT. The results were confirmed by two additional reporter systems: the AR target genes (PSA and MMTV) promoter and one oligomer containing four repeats of AR response element (ARE). The results show that gelsolin can enhance the DHT induced AR transactivation in three different reporter gene assays (FIG. 25).

(5) AR Peptides Block Gelsolin from Enhancing AR Activity

461. Since the coactivator activity of gelsolin may depend on its association with AR, we designed AR peptides to disrupt the interaction between AR and gelsolin. Three of these AR peptides covering either whole or partial DBD domain are D, D1, and D2. The others designed by dissecting twelve helixes of AR LBD are H1-2, H3, H4-5, H6-7, H8-9, H10-11, and H12 (FIG. 26A). Gelsolin enhanced AR activity was demonstrated by reporter gene assay. Co-transfection of D, D1, or HI-2 peptides suppressed gelsolin enhanced AR activity (FIG. 26B lane 3, 4, 6). Several peptides in other regions of AR LBD also reduced AR activity but blocked gelsolin coactivator effect to a lesser degree. Together, these data suggest that D1 (aa 551-600) and H1-2 (aa 655-695) may represent the major sites to suppress gelsolin-enhanced AR transactivation via interruption of the interaction between AR and gelsolin.

(6) AR and Gelsolin Co-Exist in Prostate Cancer Cells and Tissue

462. Western blotting assays further confirmed that AR and gelsolin co-exist in the same cell. Gelsolin expression can be detected in CWR22 and LNCaP cells (FIG. 27A). As CWR22 and LNCaP cells were well documented as expressing mutated ARs (McDonald et al. Cancer Res 60, 2317-2322. (2000)), the data showed gelsolin expression in these two cell lines and demonstrated that AR and gelsolin coexist in the same cell. In addition to CWR22 and LNCaP cells, gelsolin is also expressed in two other prostate cancer cells, PC-3 and DU145, those are AR negative cells (FIG. 27A). Human prostate cancer specimens from patients treated with or without androgen ablation were then used to demonstrate the co-distribution of AR and gelsolin. Both gelsolin and AR were expressed heterogeneously in the nucleus of cancer cells (FIG. 27C-b, -d).

(7) Androgen Ablation Enhances Gelsolin Expression in Prostate Cancer Cells

463. To determine if androgens have any feedback mechanism to control gelsolin expression, LNCaP xenograft nude mice as an in vivo assay model were used first. LNCaP xenografts in castrated nude mice show growth arrest after castration and no apparent re-growth for six weeks before harvest. In contrast, xenografts in the control group continue to grow after sham operation. Those viable cancer cells that represent LNCaP xenografts are confirmed by hemotoxylin-enosin staining (FIG. 27B-a, b). Immunostaining of gelsolin in these LNCaP xenograft cells show gelsolin expression is much more intense in the xenografts of castrated nude mice (FIG. 27B-d) as compared to control group (FIG. 27B-c) indicating that androgens ablation by castration may increase gelsolin expression. This conclusion was further supported using human prostate cancer specimen from patients treated with and without androgen ablation therapy. Gelsolin expression is up-regulated in cancer cells after androgen ablation therapy (FIG. 27C-c and -d). Together, both results from LNCaP xenografted nude mice and human prostate cancer specimens demonstrate that withdrawal of androgen can enhance gelsolin expression, consistent with a feed back control mechanism between gelsolin and androgen-AR.

(8) Gelsolin Enhances the Androgenic Activity of HF and Reduces its Capacity to Suppress AR Activity

464. To examine any role of gelsolin for clinical “antiandrogen withdrawal syndrome”, the effect of gelsolin on AR activity in the presence of 100 nM HF (FIG. 28) was analyzed. For this experiment, medium containing normal 10% fetal calf serum (FCS), which contains low level of androgen, instead of charcoal-stripped FCS was used to mimic a condition after medical/surgical castration. The degree of AR transactivation in the presence of low levels is shown in lane 1 of FIG. 28. Addition of 100 nM HF can then inhibit 80% of AR transactivation (lane 2 vs. lane 1). Further addition of gelsolin can then enhance the androgenic activity of HF and reduce its capacity in inhibiting AR activity to 40% (lane 3-4 vs. lane 1).

b) Methods

(1) Yeast Two-Hybrid Screening.

465. The C-terminal fragments (aa 595-918) from mtARt877s, a gift from Dr. S. P. Balk (University of Massachusetts Medical Center), was inserted into pAS2 yeast expression plasmid (Clontech, Palo Alto, Calif.). The pAS2-mtARt877s was used as a bait, and expressed in yeast Y190, cultured on synthetic dropout medium (tryptophan was eliminated). Human prostate cDNA library, a gift from Dr. S. Ellege (Baylor College of Medicine), was sequentially transformed into the yeast Y190 expressing the bait plasmid. The screening protocol was as described in previous report (Ting et al. Proc Natl Acad Sci USA 99, 661-666. (2002)).

(2) Yeast Liquid β-gal Assays.

466. Y190 yeast cells were transformed with pAS2-mtARt877s (aa 595-918) and pATC2-gelsolin (aa 281-731). Transformants were selected by their growth in the presence of 100 nM 5α-dihydrotestosterone (DHT), 10 μM HF, 1 μM progesterone (P), 1 μM 17β-estradiol (E2), or EtOH vehicle, and assayed for liquid β-gal assays as described previously (Hsiao et al. J Biol Chem 274, 20229-20234 (1999)).

(3) Glutathione S-Transferase (GST) Pull-Down Assay.

467. The plasmids expressing GST-gelsolin (GSN) and GST-GSNc fusion proteins are constructed by inserting PCR amplified GSN and GSNc cDNA into pGEX-KG plasmid (Guan et al. Anal Biochem 192, 262-267 (1991)). GST-GSN, GST-GSNc fusion proteins, and GST control protein were purified as instructed by the manufacturer (Amersham Pharmacia, Piscataway, N.J.). AR, AR DBD-LBD (ARDL), AR LBD (ARL), AR DBD (ARD), or AR N-terminus (ARN) was expressed in vitro and ³⁵S-methionine-labeled by TNT coupled reticulocyte lysate system (Promega, Madison, Wis.). The assay was carried out as previous report (Ting et al. Proc Natl Acad Sci USA 99, 661-666 (2002)).

(4) Transfection Studies

468. A C-terminal fragment of gelsolin (aa 281-731) was isolated from pACT2 encoding gelsolin, and inserted into pSG5-Gal4 DNA-binding domain (DBD) (constructed by Dr. R. Nakao). AR fragment (aa 36-918) was inserted into pCMX-VP16 (a gift from Dr. D. Chen). For gelsolin expression vector, a full-length cDNA fragment of gelsolin from LKCG, a gift from Dr. D. Kwiatkowski (Northwestern University, Evanston, Ill.), was inserted into pSG5. Dr. M. L. Lu (Harvard Medical School, Boston, Mass.) provided the p (ARE) 4-luciferase (LUC) plasmid. Dr. A. Mizokami (University of Kanazawa, Kanazawa, Japan) provided the pGL3-PSA6.0LUC plasmid. The expression plasmids of AR peptides were constructed by inserting the PCR amplified cDNA fragment of AR DBD into pFlag-CMV (Sigma) and the fragments of AR LBD into pCDNA-flag plasmid. Transfection protocol and reporter gene assay were described in previous report (Ting et al. Proc Natl Acad Sci USA 99, 661-666 (2002)).

(5) Preparation of Cellular Protein and Western Blots

469. CWR22, LNCaP, DU145, PC-3, PC-3(AR2), C2C12, COS-1, and HTB-14 cells were collected, suspended in lysis buffer, and centrifuged. After determination of protein concentration, the supernatant was diluted in loading buffer and boiled for 3 min. Aliquots corresponding to 50 μg protein of each sample were loaded to a 10% SDS-PAGE. The protocol for Western Blotting was described in a previous report (Ting et al. Proc Natl Acad Sci USA 99, 661-666 (2002)).

(6) Animal Study.

470. LNCaP (3×10⁷) cells were inoculated into the dorsal region of nude mice. One group of mice (n=3) was castrated at 11 weeks after cell inoculation, while another group (n=3) underwent sham operation at the same time. A representative LNCaP xenograft of each group was harvested 6 weeks after castration or sham operation.

(7) Immunohistochemical Analysis.

471. Human prostate tumor or LNCaP mice xenograft tissues were fixed in 10% neutral buffered formalin, processed routinely, and embedded in paraffin. Localization of gelsolin protein expression was investigated on 5 μm serial sections of tumor specimen. Slides were deparaffinized, rehydrated, and incubated with 3% (v/v) hydrogen peroxide for 15 min to inhibit endogenous peroxidase activity. The sections were then blocked with bovine serum albumin for 15 min and incubated for 3 h at 37° C. with rabbit polyclonal anti-AR (SantaCruz, Santa Cruz, Calif.) or gelsolin antibody at a dilution of 1:500. Mouse immunoglobin was used as the negative control in place of the primary antibody. The bound primary antibody was visualized by avidin-biotin-peroxidase detection with the DAKO kit (DAKO, Carpinteria, Calif.) according to the manufacturer's instructions and nuclei were stained with hematoxylin.

### 6. Example 6

### Supervillin Associates with Androgen Receptor and Modulates its Transcription Activity

A) Materials and Methods

(1) Expression Plasmids.

472. pCMX-VP16-hSVn and pCMX-VP16-hSVc were constructed by releasing fragments from pACTII-hSV(558-1788) using restriction enzyme digestion and inserted to pCMX-VP16 vector. pEGFP-bSV, pEGFP-bSV(831-1792), pEGFP-bSV(1010-1792) and pEGFP-bSV(831-1286) were kindly provided by Dr. Elizabeth J. Luna. pSG5-bSV was constructed by inserting bSV cDNA, which was released from pEGFP-bSV, into the pSG5 vector. The p(ARE)-4-Luc plasmid is described in previous report (17E). The pGL3-PSA6.0Luc plasmid is kindly provided by Dr. Atsushi Mizokami (University of Kanazawa).

(2) Yeast Two-Hybrid Screening

473. A fusion protein (Gal4-AR) containing Gal4 DNA binding domain, Gal4(DBD) and carboxyl terminus of AR (a.a. 595-918) was used as bait to screen from 3×10⁶ transformants of MATCHMAKER human skeletal muscle library (Clontech). Transformants were selected for growth on nutrition selection plates containing −3SD media (synthetic dropout lacking histidine, leucine, and tryptophan) with 25 mM 3-aminotriazole and 10 nM T. The yeast were cultured in humidified 30° C. chamber for 3 days. Colonies were also filter-assay for β-galactosidase (β-gal) activity. Plasmids isolated from candidate clones were co-transformed into Y190 with bait and the ligand dependant interaction was then further confirmed by filter-assayed for β-gal activity with EtOH or 10 nM T treatment. The plasmid pACTII or pACTII-SV(558-1788) was co-transformed into yeast with bait and plated on −2SD plates (lacking leucine and tryptophan). The yeast colonies that grew on −2SD plates were selected and plated on −3SD plate with or without 10 nM DHT to test for growth ability.

(3) Cell Culture and Transfection

474. Mouse myoblast cell line (C2C12), human prostate cancer cell lines (PC-3 and DU145), and monkey kidney fibroblast cell line (COS-1) were maintained in Dulbecco's minimum essential medium (DMEM) containing penicillin (25 units/ml), streptomycin (25 mg/ml), and 10% fetal bovine serum (FBS). In mammalian two-hybrid assay, transfections were performed using the calcium phosphate precipitation method as described previously (15). Briefly, 1.5-3×10⁵ cells were plated on 35-mm dishes for 24 h, and the medium was changed to DMEM containing 10% charcol-dextran stripped FBS (CD-FBS) 2 h before transfection. Cells were transfected with 0.5 μg plasmids expressing Gal4(DBD) and VP-16 fusion proteins as indicated. Gal4 response element controlled Firefly luciferase expression plasmid, pG5-Luc, was used as reporter gene. A Renilla luciferase expression plasmid pRL-SV40 was used as an internal control for transfection efficiency. The total amount of DNA was adjusted to 5 μg with pCMX-VP16 vectors. After 16 h transfection, cells were treated with ligands as described for another 24 h.

475. In AR transactivation activity assays, transfections were performed using SuperFect (Qiagen, Chatsworth, Calif.) following protocols described in manual provided by Qiagen. Briefly, cells were plated on 35 mm dishes and after 24 h were transfected using the SuperFect kit. The total DNA amount was adjusted to 2 μg with pSG5 or pEGFP vectors. The medium was changed to DMEM with 10% CD-FBS 2 h after transfection. After 24 h, the DMEM with 10% CD-FBS was changed again, and the cells were treated with various steroids. Cells were harvested after 24 h for dual-luciferase assay as described in protocol provided by Promega. At least three independent experiments were carried out in each case.

(4) Glutathione S-Transferase (GST) Pull-Down Assay

476. GST-ARN, GST-AR-DBD-LBD (AR-DL) fusion proteins, and GST control protein were purified as instructed by the manufacturer (Amersham Pharmacia). Briefly, plasmids containing GST-fusion protein expressing cDNA were transformed into BL21(DE3)pLysS bacteria strain and selected for ampicillin and chloramphenicol resistant colonies. Selected colonies were grown in LB medium (bacteria expressing GST-AR-DL were cultured under 1 μM DHT treatment) at 30° C. until OD600 reached 0.6 to 1. Then add 0.4 mM IPTG into medium for 3 hours. Bacteria were lysed by 3 cycles of freezing-thawing in NETN buffer (20 mM Tris/pH 8.0, 100 mM NaCl, 6 mM MgCl₂, 1 mM EDTA, 0.5 mM NP-40, 1 mM DTT, 8% glycerol, and 1 mM PMSF). Lysed bacteria were spun down and the supernatants were collected. The GST fusion proteins were pulled down by glutathione (GSH)-beads in 4° C. for 1 h then washed three times with NETN buffer. The purified GST fusion proteins and beads were suspended in 100 μl NETN buffer. Resuspended GST-proteins and beads were incubated with 5 μl in vitro-translated (Leo, C. & Chen, J. D. (2000) Gene 245, 1-11) S-methionine-labeled VP16-hSVn or VP16-hSVc expressed from pCMX-VP16-hSVn or pCMX-VP16-hSVc by TNT coupled reticulocyte lysate system (Promega). After incubating for 1 h at 4° C. in the presence or absence of 1 μM DHT, GSH-beads were washed with NETN buffer four times then the protein complexes were loaded in SDS-PAGE and visualized using phosphorimager.

(5) Immunocytofluorescence and Confocal Microscopy

477. COS-1 cells were seeded on two-well Lab Tek Chamber slides (Nalge) in DMEM with 10% CD-FBS for 18 h before transfected with 2 μg DNA/10⁵ cells by the FuGENE6 transfection reagent (Boehringer-Mannheim). Transfected cells were treated with 10 nM DHT or vehicle for 16 h, then fixed in fixation solution (3% formaldehyde and 10% sucrose in PBS) for 15 min on ice and permeabilized by methanol. Immunostaining was performed by incubating slides with blocking solution (2% bovine serum albumin in PBS) for 15 min at room temperature, stained with 1:200 dilution of anti-AR polyclonal antibody (NH27) for 45 min, followed by Texas-red-conjugated goat anti-rabbit antibody (ICN) for 45 min at room temperature. Stained slides were washed and mounted (Vectashield; Vector Laboratories, Inc., Burlingame, Calif.). The slides were photographed under 40 fold magnification with a Leica TCS SP Spectral Confocal Microscope.

(6) Western Blotting

478. Protein samples extracted from the cell were separated on 15% SDS-PAGE and transferred to nitrocellulose membranes. Membranes were incubated 1 h with 5% non-fat milk in TBST at room temperature, followed by the antibodies against p27^{(KIP1)} (Santa Cruz), followed by AP conjugated goat-antimouse antibody. Blots were developed using the AP developing reagent from Bio-Rad. Band intensity was quantitated by Collage® image analysis software (Fotodyne Inc.).

b) Results

(1) Supervillin is an AR Associated Protein

479. The human AR ligand binding domain (LBD) was used as a bait to screen AR interaction proteins in a human skeletal muscle cDNA library in the presence of 10 nM T. Several positive clones were selected by nutrition deprivation and confirmed by the β-gal assay. Further analysis indicated that 5 clones containing cDNA inserts match well with various segments of SV cDNA. As shown in FIG. 29A, one of these clones, encoding a.a. 558-1788 of SV, interacted well with AR-peptide bait in the presence of 10 nM DHT. This SV cDNA was then truncated and fused with VP16 as indicated in FIG. 29B. Mammalian two-hybrid indicated that hSVn peptide (a.a. 594-1335), but not hSVc peptide (a.a. 1268-1788), could interact with the AR-DBD-LBD (AR-DL) in a DHT dependent manner (FIG. 29C). The hSVn can also interact with the AR N-terminal domain (ARN) (FIG. 29C, lane 14). GST pull-down assay further confirmed that VP16-hSVn but not VP16-hSVc can be pulled down by GST-AR-DL (FIG. 29D). Together, data from yeast two-hybrid, mammalian two-hybrid and GST pull-down assays all suggest that hSV peptide (a.a. 594-1268) can interact with the ARN as well as the AR-DL in a DHT enhanced manner.

(2) Nuclear Localization and Enhancement of AR Transactivation by SV Domain (a.a. 831-1281)

480. Results from FIG. 29 demonstrate that the SV peptide, a.a. 594-1268, can interact with AR. To further test if this interaction also influences AR transactivation, plasmids encoding various domains of bovine SV (bSV) along with AR expressing plasmid and mouse mammary tumor virus-luciferase (MMTV-Luc) reporter were co-transfected in COS-1 cells. The bSV contains 1792 amino acids sharing 92.7% homology with human SV (Pope et al. (1998) Genomics 52,342-51). Fragments of bSV were conjugated with EGFP, which emits fluorescence under light elicitation. As shown in FIG. 30A, addition of 10 nM DHT induced AR transactivation 25 fold (lane 1 vs. 2) when AR was co-expressed with EGFP. The full-length bSV (a.a. 1-1792) further enhanced AR transactivation to 132 fold (lane 2 vs. 8). A peptide containing a.a. 831-1281 of bSV, which is within the interaction domain, can further enhance AR transactivation to 248 fold (lane 6 vs. 8). In contrast, the other domain within SV (a.a. 1010-1792) had only a marginal effect on the AR transactivation (lanes 2 vs. 4). These data strongly suggest that bSV(831-1281) in the interaction domain is sufficient to enhance AR transactivation function. As shown in FIG. 30B, subcellular colocalization studies using confocal microscope further demonstrated that bSV(831-1281) is exclusively located in the nucleus and colocalizes with DHT-bound AR in nucleus. In contrast, bSV(1010-1792) is located mainly in the cytosol. Together, the results in FIG. 30 demonstrated that full length bSV as well as the domain (a.a. 831-1281) could enhance AR transactivation and colocalize with AR in the nucleus.

(3) Supervillin Enhances AR Transactivation

481. Co-transfection of the full length bSV and AR expression plasmids at 25:1 and 50:1 ratios enhanced AR transactivation 3-8 fold in C2C12 muscle cells in the presence of DHT. Similar results were also observed when we replaced C2C12 cells with COS-1, DU145, and PC-3 (FIG. 32A). In addition to MMTV-Luc, two other AR reporter genes, prostate specific antigen-Luc (PSA-Luc) and androgen response element-Luc [(ARE)-4-Luc], were applied to demonstrate the coactivation function of SV. All results demonstrate that regardless of different ARE containing promoters, SV can enhance AR transactivation function in PC-3 cells (FIG. 31B). To further rule out the possible artifact effect using reporter gene assays, we analyzed the effect of SV on AR endogenous target genes expression, such as p27^{KIP} (Ling et al. (2001) J Endocrinol. 170, 287-96), in the PC-3 cells stably transfected with AR expression plasmid, PC-3(AR2) cells (Yuan et al. (1993) Cancer Res. 53, 1304-11). As shown in FIG. 31C, 10 nM DHT induced p27^{(KIP1)} protein expression (lane 1 vs. 2). Addition of bSV further enhanced p27^{(KIP1)} protein expression (lane 2 vs. 4). These data clearly demonstrate that SV can function as an AR coregulator to enhance AR transactivation.

(4) The Specificity of SV Coregulator Activity

482. Using mammalian two-hybrid assay, the data indicated that SV could also interact with other steroid receptors such as glucocorticoid receptor (GR), estrogen receptor-α (ER-α), and peroxisome proliferating activation receptor-γ (PPAR-γ). The interaction of SV with these receptors was similar (ER-α) or relatively weaker (GR and PPAR-γ) as compared to the interaction with AR (FIG. 32A), which could be due to the different coregulator context in the cell. The activation function-2 domain of GR and PPARγ might be able to recruit more coactivators or have stronger affinity to certain coactivators that result in the lower coactivation activity of SV with these two receptors. SV modulated transcription activities of nuclear receptors were then assayed by using AR and GR reporter gene (MMTV-Luc), PPAR-γ reporter gene (PPRE-Luc) and ER-α reporter gene (ERE-Luc). The results show SV has less enhancement effect on the transactivation of GR as compared to AR, and has little effect on PPAR-γ, and ER-α (FIG. 32B).

(5) Comparison of Cooperative Effect and Ligand Enhancement Effect Between SV and Other ARAs

483. To compare the coregulator function of SV and other known AR coregulators, the cooperative effect between SV and two other AR coregulators, ARA55 and ARA70N (a.a. 1-401) was tested. The combination of SV and ARA55 or ARA70N show better than additive effect as compared to the enhancement of SV, ARA55 or ARA70N alone (FIG. 33A). This indicates these coactivators may modulate AR activity through multiple yet cooperative mechanisms to potentiate AR function.

484. It has been known that coregulators can enhance AR transactivation under various steroid treatments. For example, ARA70N could enhance AR transactivation in the presence of T and DHT, as well as 17β-estradiol (E2), hydroxyflutamide (HF), and androst-5-ene-3β,17β-diol (Adiol) (20, 21, 22). Here the effect of SV with ARA70N in the induction of AR function was compared under these steroids. The results show that SV significantly enhances T and DHT induced AR transactivation, slightly enhances Adiol induced AR transactivation, but shows marginal effect on E2- or HF-induced AR transactivation. These data therefore again demonstrated only selective AR coregulators were able to enhance AR transactivation induced by various steroids.

(6) The Interaction Between AR N-Terminus and C-Terminus is Suppressed by SV

485. Early reports suggested that interaction between ARN and C-terminus (ARC) may help to stabilize the dimer complexes of AR (23). Since SV can interact with both ARN and AR-DL (FIG. 31C, D), it is possible that SV may stabilize the dimer complexes by holding the ARN and ARC together. By using mammalian two-hybrid assays, we demonstrated AR N—C interaction in a DHT dependent manner (FIG. 34). Selective AR coregulators, such as SRC-1, could further enhance this N—C interaction. Surprisingly, addition of SV showed a mild suppressive effect on this N—C interaction. The contrasting effects between SV and SRC-1 strongly suggest that different AR coregulators may go through different mechanisms to enhance AR transactivation.

### G. REFERENCES

McNally, J. G., Müller, W. G, Walker, D., Wolford, R., and Hager, G. L. (2000) Science 287, 1262 1265
Aasland, R., Gibson, T. J, & Stewart, A. F. (1995) Trends Biochem Sci 20, 56-59.
Adachi, M., Takayanagi, R., Tomura, A., Imasaki, K., Kato, S., Goto, K., Yanase, T., Ikuyama, S., and Nawata, H. (2000) N. Engl. J. Med. 343, 856-862.
Agoulnik, I., Stenoien, D., Mancini, M., & Weigel, N. (2000) Abstract (#302) in Keystone Steroid Symposium, Colorado
Alen, P., Claessens, F., Schoenmakers, E., Swinnen, J. V., Verhoeven, G., Rombauts, W., Peeters, B. (1999) Mol. Endocrinol 12, 117-128
Alen, P., Claessens, F., Verhoeven, G., Rombauts, W. & Peeters, B. (1999) Mol. Cell. Biol. 19, 6085-97.
Anzick, S. L., Kononen, J., Walker, R. L., Azorsa, D. O., Tanner, M. M., Guan, X.-Y., Sauter, G., Kallioniemi, O.-P., Trent, J. M., and Meltzer, P. S. (1997) Science 277, 965-968
Asch, H. L. et al. Widespread loss of gelsolin in breast cancers of humans, mice, and rats. Cancer Res 56, 4841-4845. (1996).
Baniahmad, A., Ha, I., Reinberg, D., Tsai, S., Tsai, M.-J., and O'Malley, B. W. (1993) Proc. Natl. Acad. Sci. USA 90, 8832-8836
Berrevoets, C. A., Doesburg, P., Steketee, K., Trapman, J. & Brinkmann, A. O. Functional interactions of the AF-2 activation domain core region of the human androgen receptor with the amino-terminal domain and with the transcription coactivator TIF2 (transcription intermediary factor2). Mol Endocrinol 12, 1172-1183. (1998).
Bevan, C. L., Hoare, S., Claessens, F., Heery, D. M. & Parker, M. G. (1999) Mol. Cell. Biol. 19, 8383-92.
Brinkmann, A. O., Blok, L. J., de Ruiter, P. E., Doesburg, P., Steketee, K., Berrevoets, C. A., & Trapman, J. (1999) J Steroid Biochem Mol Biol 69, 307-313.
Cardoso, C., Timsit, S., Villard, L., Khrestchatisky, M., Fontes, M., & Colleaux, L. (1998) Hum Mol Genet 7, 679-684.
Cavailles, V., Dauvois, S., L'Horset, F., Lopez, G., Hoare, S., Kushner, P. J., and Parker, M. G. (1995) EMBO J. 14, 3741-3751
Chakravarti, D., LaMorte, V. J., Nelson, M. C., Nakajima, T., Schulman, I. G., Juguilon, H., Montminy, M., and Evans, R. M. (1996) Nature 383, 99-103
Chang, C. et al. Androgen receptor: an overview. Crit Rev Eukaryot Gene Expr 5, 97-125 (1995).
Chang, C., Kokontis, J., & Liao, S. T. (1988) Science 240, 324-326.
Chang, C., Kokotonis, J., and Liao, S. T. (1989) Proc. Natl. Acad. Sci. USA 85, 7211-7215
Chang, C., Saltzman, A., Yeh, S., Young, W., Keller, E., Lee, H. J., Wang, C. & Mizokami, A. (1995) Crit. Rev. Eukaryot. Gene Expr. 5, 97-125.
Chang, H.-C., Miyamoto, H., Marwah, P., Lardy, H., Yeh, S., Huang, K.-E., and Chang, C. (1999) Proc. Natl. Acad. Sci. U.S.A. 96, 11173-11177
Chen, H., Lin, R. J., Schiltz, R. L., Chakravarti, D., Nash, A., Nagy, L., Privalsky, M. L., Nakatani, Y., and Evans, R. M. (1997) Cell 90, 569-580
Chen, S., and Smith, D. F. (1998) J. Biol. Chem. 273, 35194-35200
Crawford, E. D. et al. A controlled trial of leuprolide with and without flutamide in prostatic carcinoma. N Engl J Med 321, 419-424. (1989).
Cui, X., De Vivo, I., Slany, R., Miyamoto, A., Firestein, R., & Cleary, M. L. (1998) Nat Genet 18, 331-337.
Dhanasekaran, S. M. et al. Delineation of prognostic biomarkers in prostate cancer. Nature 412, 822-826. (2001).
Di Croce, L., Okret, S., Kersten, S., Gustafsson, J.-A., Parker, M., Wahli, W., and Beato, M. (1999) EMBO J. 18, 6201-6210
Ding, X. F. et al. Nuclear receptor-binding sites of coactivators glucocorticoid receptor interacting protein 1 (GRIP1) and steroid receptor coactivator 1 (SRC-1): multiple motifs with different binding specificities. Mol Endocrinol 12, 302-313. (1998).
Dingwall, C., & Laskey, R. A. (1991) Trends Biochem Sci 16, 478-481
Dosaka-Akita, H. et al. Frequent loss of gelsolin expression in non-small cell lung cancers of heavy smokers. Cancer Res 58, 322-327. (1998).
Doumit, M. E., Cook, D. R. & Merkel, R. A. (1996) Endocrinology 137, 1385-94.
Dreicer, R. The evolving role of hormone therapy in advanced prostate cancer. Cleve Clin J
Dynlacht, B. D., Hoey, T., and Tjian, R. (1991) Cell 66, 563-576
Eisenberger, M. A. et al. Bilateral orchiectomy with or without flutamide for metastatic prostate cancer. N Engl J Med339, 1036-1042. (1998).
Evans, R. M. (1988) Science 240, 889-895
Fang, Y., Fliss, A. E., Robins, D. M., and Caplan, A. J. (1996) J. Biol. Chem. 271, 28697-28702
Fenton, M. A. et al. Functional characterization of mutant androgen receptors from androgen-independent prostate cancer. Clin Cancer Res 3, 1383-1388. (1997).
Firestein, R., Cui, X., Huie, P., & Cleary, M. L. (2000) Mol Cell Biol, 20, 4900-4909.
Fliss, A. E., Rao, J., Melville, M. W., Cheetham, M. E., and Caplan, A. J. (1999) J. Biol. Chem. 274, 34045-34052
Fraser, A. G., Kamath, R. S., Zipperlen, P., Martinez-Campos, M., Sohrmann, M. & Ahringer, J. (2000) Nature 408, 325-30.
Fujimoto, N. et al. Cloning and characterization of androgen receptor coactivator, ARA55, in human prostate. J Biol Chem 274, 8316-8321. (1999).
Fujita, H. et al. Induction of apoptosis by gelsolin truncates. Ann NY Acad Sci 886, 217-220 (1999).
Gao, T., Brantley, K., Bolu, E., and McPhaul, M. J. (1999) Mol. Endocrinol 13, 1645-1656
Glass, C. K. & Rosenfeld, M. G. (2000) Genes & Development. 14, 121-41.
Goktas, S., and Crawford, D. (1999) Semin. Oncol. 26, 162-173
Gould, A. (1997) Curr Opin Genet Dev 7(4), 488-494
Greenlee, R. T., Hill-Harrnon, M. B., Murray, T., and Thun, M. (2001) CA Cancer J. Clin. 51, 15-36
Gregory, C. W. et al. A mechanism for androgen receptor-mediated prostate cancer recurrence after androgen deprivation therapy. Cancer Res 61, 4315-4319. (2001).
Gregory, C. W., Hamil, K. G., Kim, D., Hall, S. H., Pretlow, T. G., Mohler, J. L., and French, F. S. (1998) Cancer Res. 58, 5718-5724
Gu, Y., Nakamura, T., Alder, H., Prasad, R., Canaani, O., Cimino, G., Croce, C. M., & Canaani, E. (1992) Cell 71, 701-708
Guan, K. L. & Dixon, J. E. Eukaryotic proteins expressed in Escherichia coli: an improved thrombin cleavage and purification procedure of fusion proteins with glutathione S-transferase. Anal Biochem 192, 262-267. (1991).
Hakimi, J. M., Rondinelli, R. H., Schoenberg, M. P. & Barrack, E. R. Androgen-receptor gene structure and function in prostate cancer. World J Urol 14, 329-337 (1996).
Han, G., Foster, B. A., Mistry, S., Buchanan, G., Harris, J. M., Tilley, W. D., & Greenberg, N. M. (2001) J Biol Chem 276, 11204-11213
Hayashi, Y., Ohmori, S., Ito, T., and Seo, H. (1997) Biochem. Biophys. Res. Commun. 236, 83-88
He, B., Kemppainen, J. A. & Wilson, E. M. (2000) J. Biol. Chem. 275, 22986-94.
He, B., Kemppainen, J. A., Voegel, J. J., Gronemeyer, H., & Wilson, E. M. (1999) J Biol Chem 274, 37219-37225
Heery, D. M., Kalkhoven, E., Hoare, S. & Parker, M. G. (1997) Nature 387, 733-6.
Heinlein, C. A., Ting, H., Yeh, S., and Chang, C. (1999) J. Biol. Chem. 274, 16147-16152
Heisler, L. E., Evangelou, A., Lew, A. M., Trachtenberg, J., Elsholtz, H. P. & Brown, T. J. (1997)
Hong, H., Kohli, K., Trived, A., Johnson, D. L., and Stallcup, M. R. (1996) Proc Natl Acad Sci USA 93, 4948-4952
Hrouda, D., Perry, M., and Dalgleish, A. G. (1999) Semin. Oncol. 26, 455-471
Hsiao, P. W. & Chang, C. Isolation and characterization of ARA 160 as the first androgen receptor N-terminal-associated coactivator in human prostate cells. J Biol Chem 274, 22373-22379. (1999).
Hsiao, P. W., Lin, D. L., Nakao, R. & Chang, C. The linkage of Kennedy's neuron disease to ARA24, the first identified androgen receptor polyglutamine region-associated coactivator. J Biol Chem 274, 20229-20234. (1999).
Hsing, A. W., Gao, Y. T., Wu, G., Wang, X., Deng, J., Chen, Y. L., Sesterhenn, I. A., Mostofi, F. K., Benichou, J. & Chang, C. (2000) Cancer. Res. 60, 5111-5116.
Hughes, I. A. Minireview: sex differentiation. Endocrinology 142, 3281-3287. (2001).
Jenster, G. (1999) Semin. Oncol. 26, 407-421
Jenuwein, T., Laible, G., Dom, R., & Reuter, G. (1998) Cell Mol Life Sci. 54, 80-93.
Kalkhoven, E., Valentine, J. E., Heery, D. M., and Parker, M. G. (1998) EMBO J. 17, 232-243
Kamei, Y., Xu, L., Heinzel, T., Torchia, J., Kurokawa, R., Gloss, B., Lin, S.-C., Heyman, R. A., Rose, D. W., Glass, C. K., and Rosenfeld, M. G. (1996) Cell 85,403-414
Kang, H. Y., Yeh, S., Fujimoto, N. & Chang, C. Cloning and characterization of human prostate coactivator ARA54, a novel protein that associates with the androgen receptor. J Biol Chem 274, 8570-8576. (1999).
Kang, H-Y., Lin, H-K., Hu, Y.-C., Yeh, S., Huang, K. E., & Chang, C. (2001) Proc. Natl. Acad. Sci. U.S.A. 98, 3018-3023.
Katzenellenbogen, J. A., O'Malley, B. W., and Katzenellenbogen, B. S. (1996) Mol. Endocrinol. 10, 119-131
Kelly, W. K., Slovin, S. & Scher, H. I. Steroid hormone withdrawal syndromes. Pathophysiology and clinical significance. Urol Clin North Am 24, 421-431. (1997).
Kemppainen, J. A., Lane, M. V., Sar, M., and Wilson, E. M. (1992) J. Biol. Chem. 267, 968-974
Kokontis, J., Ito, K., Hiipakka, R. A., and Laio, S. (1991) Receptor 1, 271-279 Truica
Kotaja, N., Aittomaki, S., Silvennoinen, O., Palvimo, J. J. & Janne, O. A. (2000) Mol. Endocrinol. 14, 1986-2000.
Koya, R. C. et al. Gelsolin inhibits apoptosis by blocking mitochondrial membrane potential loss and cytochrome c release. J Biol Chem 275, 15343-15349. (2000).
Kwiatkowski, D. J. Functions of gelsolin: motility, signaling, apoptosis, cancer. Curr Opin Cell Biol 11, 103-108. (1999).
Langeler, E. G., van Uffelen, C. J., Blankenstein, M. A., van Steenbrugge, G. J. & Mulder, E. (1993) Prostate 23, 213-23.
Langley, E., Zhou, Z. X., & Wilson, E. M. (1995) J Biol Chem 270, 29983-29990.
Le Douarin, B., Zechel, C., Garnier, J.-M., Lutz, Y., Tora, L., Pierrat, B., Heery, D., Gronemeyer, H., Chambon, P., and Losson, R. (1995) EMBO J. 14, 2020-2033
Lee, D. K., Duan, H. O., and Chang, C. (2000) J. Biol. Chem. 275, 9308-9313
Lee, H. K., Driscoll, D., Asch, H., Asch, B. & Zhang, P. J. Downregulated gelsolin expression in hyperplastic and neoplastic lesions of the prostate. Prostate 40, 14-19. (1999).
Lee, Y. F., Shyr, C. R., Thin, T. H., Lin, W. J., and Chang, C. (1999) Proc Natl Acad Sci USA. 96, 14724-14729
Leers, J., Treuter, E., & Gustafsson, J. A. (1998) Mol Cell Biol 18, 6001-6013.
Leo, C. & Chen, J. D. (2000) Gene 245, 1-1.
Li, H., Gomes, P. J., and Chen, J. D. (1997) Proc Natl Acad Sci USA 94, 8479-8484
Ling, M. T., Chan, K. W. & Choo, C. K. (2001) J. Endocrinol. 170, 287-96.
Loewith, R., Meijer, M., Lees-Miller, S. P., Riabowol, K., & Young, D. (2000) Mol Cell Biol 20, 3807-3816
Lu, M. L., Schneider, M. C., Zheng, Y., Zhang, X. & Richie, J. P. (2001) J. Biol. Chem. 276, 13442-51.
Magi-Galluzzi, C. et al. Heterogeneity of androgen receptor content in advanced prostate cancer. Mod Pathol 10, 839-845. (1997).
McDonald, S., Brive, L., Agus, D. B., Scher, H. I. & Ely, K. R. Ligand responsiveness in human prostate cancer: structural analysis of mutant androgen receptors from LNCaP and CWR22 tumors. Cancer Res 60, 2317-2322. (2000).
McEwan, I. J., and Gustafsson, J. (1997) Proc. Natl. Acad. Sci. USA 94, 8485-8490
McInerney, E. M., Tsai, M. J., O'Malley, B. W. & Katzenellenbogen, B. S. (1996) Proc. Natl Acad. Sci. USA 93, 10069-73.
McKenna, N. J., Xu, J., Nawaz, Z., Tsai, S. Y., Tsai, M.-J., and O'Malley, B. W. (1999) J. Steroid Biochem. Mol. Biol. 69, 3-12
Med 67, 720-722, 725-726. (2000).
Mitchell, S. H., Zhu, W., and Young, C. Y. (1999) Cancer Res. 59, 5892-5895
Miyamoto, H., and Chang, C. (2000) Int. J. Urol. 7, 32-34
Miyamoto, H., Yeh, S., Lardy, H., Messing, E. & Chang, C. (1998) Proc. Natl. Acad. Sci. USA 95, 11083-8.
Miyamoto, H., Yeh, S., Wilding, G., and Chang, C. (1998) Proc Natl Acad Sci USA 95, 7379-7384
Moilanen, A.-M., Poukka, H., Karvonen, U., Hakli, M., Janne, O. A., and Palvimo, J. J. (1998) Mol. Cell. Biol. 18, 5128-5139
Montie, J. E., and Pienta, K. J. (1994) Urology 43, 892-899
Mooradian, A. D., Morley, J. E. & Korenman, S. G. (1987) Endocr. Rev. 8, 1-28.
Muller, J. M., Isele, U., Metzger, E., Rempel, A., Moser, M., Pscherer, A., Breyer, T., Holubarsch, C., Buettner, R. & Schule, R. (2000) EMBO J. 19, 359-69.
Nakamura, T., Blechman, J., Tada, S., Rozovskaia, T., Itoyama, T., Bullrich, F., Mazo, A., Croce, C. M., Geiger, B., & Canaani, E. (2000) Proc Natl Acad Sci USA 97, 7284-7289
Narusaka, Y., Narusaka, M., Satoh, K., and Kobayashi, H. (1999) J. Biol. Chem. 274, 23270-23275
Ogryzko, V. V., Schiltz, R. L., Russanova, V., Howard, V. H., and Nakatani, Y. (1996) Cell 87,953-959
Onate, S. A., Tsa, S. Y., Tsai, M. J., and O'Malley, B. W. (1995) Science 270, 1354-1357
Onda, H., Lueck, A., Marks, P. W., Warren, H. B. & Kwiatkowski, D. J. Tsc2(+/−) mice develop tumors in multiple sites that express gelsolin and are influenced by genetic background. J Clin Invest 104, 687-695. (1999).
Ozanne, D. M. et al. Androgen receptor nuclear translocation is facilitated by the f-actin cross-linking protein filamin. Mol Endocrinol 14, 1618-1626. (2000).
Pan, H. J., Uno, H., Inui, S., Fulmer, N. O. & Chang, C. (1999) Endocrine 11, 321-7.
Pestonjamasp, K. N., Pope, R. K., Wulfkuhle, J. D. & Luna, E. J. (1997) J. Cell Biol. 139, 1255-69.
Pope, R. K., Pestonjamasp, K. N., Smith, K. P., Wulfkuhle, J. D., Strassel, C. P., Lawrence, J. B. & Luna, E. J. (1998) Genomics 52, 342-51.
Poukka, H., Aamisalo, P., Karvonen, U., Palvimo, J. J. & Janne, O. A. (1999) J. Biol. Chem. 274, 19441-6.
Poukka, H., Karvonen, U., Janne, O. A. & Palvimo, J. J. (2000) Proc. Natl. Acad. Sci. USA 97, 14145-50.
Poukka, H., Karvonen, U., Yoshikawa, N., Tanaka, H., Palvimo, J. J. & Janne, O. A. (2000) J. Cell Sc. 113, 2991-3001.
Prasad, R., Zhadanov, A. B., Sedkov, Y., Bullrich, F., Druck, T., Rallapalli, R., Yano, T., Alder, H., Croce, C. M., Huebner, K., Mazo, A., & Canaani, E. (1997) Oncogene 15, 549-560
Pratt, W. B., and Toft, D. O. (1997) Endocr. Rev. 18, 306-360
Pratt, W. B., Czar. M. J., Stancato, L. F., and Owens, J. K. (1993) J. Steroid Biochem. Mol. Biol. 46, 269-279
Prendergast, G. C. & Ziff, E. B. Mbh 1: a novel gelsolin/severin-related protein which binds actin in vitro and exhibits nuclear localization in vivo. Embo J 10, 757-766. (1991).
Prins, G. S., Sklarew, R. J. & Pertschuk, L. P. Image analysis of androgen receptor immunostaining in prostate cancer accurately predicts response to hormonal therapy. J Urol 159, 641-649. (1998).
Ptashne, M., and Gann, A. A. F. (1990) Nature 346, 329-331
Pugh, B. F., and Tjian, R. (1990) Cell 61, 1187-1197
Puigserver, P., Wu, Z., Park, C. W., Graves, R., Wright, M., and Spiegelman, B. M. (1998) A cold-inducible coactivator of nuclear receptors linked to adaptive thermogenesis. Cell 92, 829-839
Rajapandi, T., Greene, L. E., and Eisenberg, E. (2000) J. Biol. Chem. 275, 22597-22604
Rozovskaia, T., Rozenblatt-Rosen, O., Sedkov, Y., Burakov, D., Yano, T., Nakamura, T., Petruck, S., Ben-Simchon, L., Croce, C. M., Mazo, A., & Canaani, E. (2000) Oncogene 20, 351-357
Ruckle, H. C., and Oesterling, J. E. (1993) World J. Urol. 11, 227-232
Ruijter, E., van de Kaa, C., Miller, G., Ruiter, D., Debruyne, F., and Schalken, J. (1999) Endocr. Rev. 20, 22-45
Sadovsky, Y., Webb, P., Lopez, G., Baxter, J. D., Fitzpatrick, P. M., Gizang-Ginsberg, E., Cavailles, V., Parker, M. G., and Kushner, P. J. (1995) Mol. Cell. Biol. 15, 1554-1563
Salazar, R., Bell, S. E. & Davis, G. E. Coordinate induction of the actin cytoskeletal regulatory proteins gelsolin, vasodilator-stimulated phosphoprotein, and profilin during capillary morphogenesis in vitro. Exp Cell Res 249, 22-32. (1999).
Scher, H. I. & Kelly, W. K. Flutamide withdrawal syndrome: its impact on clinical trials in hormone-refractory prostate cancer. J Clin Oncol 11, 1566-1572. (1993).
Sengupta, S., Vonesch, J. L., Waltzinger, C., Zheng, H. & Wasylyk, B. (2000) EMBO J. 19, 6051-64.
Shibata, H., Spencer, T. E., Onate, S. A., Jenster, G., Tsai, S. Y., Tsai, M. J., and O'Malley, B. W. (1997). Recent Prog. Horm. Res. 52, 141-164
Shieh, D. B. et al. Cell motility as a prognostic factor in Stage I nonsmall cell lung carcinoma: the role of gelsolin expression. Cancer 85, 47-57. (1999).
Shim, W. S., DiRenzo, J., DeCaprio, J. A., Santen, R. J., Brown, M. & Jeng, M. H. (1999) Proc. Natl. Acad. Sci USA 96, 208-13.
Smith, C. L., Onate, S. A., Tsai, M.-J., and O'Malley, B. W. (1996) Proc Natl Acad Sci USA 93, 8884-8888
Sonnenschein, C., Olea, N., Pasanen, M. E. & Soto, A. M. (1989) Cancer Res. 49, 3474-81.
Sotiropoulos, A., Gineitis, D., Copeland, J. & Treisman, R. (1999) Cell 98, 159-69.
Spencer, T. E., Jenster, G., Burcin, M. M., Allis, C. D., Zhou, J., Mizzen, C. A., Mckenna, N. J., Onate, S. A., Tsai, S. Y., Tsai, M. J., and O'Malley, B. W. (1997) Nature 389, 194-198
Strathdee, C. A., McLeod, M. R., and Hall, J. R. (1999) Gene 229, 21-29
Sun, H. Q., Yamamoto, M., Mejillano, M. & Yin, H. L. Gelsolin, a multifunctional actin regulatory protein. J Biol Chem 274, 33179-33182. (1999).
Takeshita, A., Cardona, G. R., Koibuchi, N., Suen, C. S. & Chin, W. W. (1997) J. Biol. Chem. 272, 27629-34.
Tan, J. A., Hall, S. H., Petrusz, P. & French, F. S. Thyroid receptor activator molecule, TRAM-1, is an androgen receptor coactivator. Endocrinology 141, 3440-3450. (2000).
Tanaka, M. et al. Gelsolin: a candidate for suppressor of human bladder cancer. Cancer Res 55, 3228-3232. (1995).
Taplin, M. E. et al. Mutation of the androgen-receptor gene in metastatic androgen-independent prostate cancer. N Engl J Med 332, 1393-1398. (1995).
Ting, H. J., Yeh, S., Nishimura, K. & Chang, C. Supervillin associates with androgen receptor and modulates its transcription activity. Proc Natl Acad Sci USA 99, 661-666. (2002).
Torchia, J., Glass, C., and Rosenfeld, M. G. (1998) Curr. Opin. Cell Biol. 10, 373-383
Torchia, J., Rose, D. W., Inostroza, J., Kamei, Y., Westin, S., Glass, C. K., and Rosenfeld, M. G. (1997) Nature 387, 677-684
Truica, C. I., Byers, S. and Gelmann, E. P. (2000) Cancer Res. 1, 4709-4713.
Tsai, M.-J., and O'Malley, B. W. (1994) Annu. Rev. Biochem. 63, 451-486
Verrier, C. S., Roodi, N., Yee, C. J., Bailey, L. R., Jensen, R. A., Bustin, M., and Parl, F. F. (1997) Mol. Endocrinol. 11, 1009-1019
Voegel, J. J., Heine, M. J., Zechel, C., Chambon, P., and Gronemeyer, H. (1996) EMBO J. 15, 3667-3675
Wang, X. et al. Identification and characterization of a novel androgen receptor coregulator ARA267-alpha in prostate cancer cells. J Biol Chem 276, 40417-40423. (2001).
Wingo, P. A., Tong, T. & Bolden, S. Cancer statistics, 1995. CA Cancer J Clin 45, 8-30. (1995).
Wulfkuhle, J. D. et al Domain analysis of supervillin, an F-actin bundling plasma membrane protein with functional nuclear localization signals. J Cell Sci 112, 2125-2136. (1999).
Yeh, .S, Hu, Y. Rahman, M., Lin, H., Ting, H., Kang, H.-Y., and Chang, C. (2000) Proc. Natl. Acad. Sci. USA 97, 11256-11261
Yeh, S. & Chang, C. Cloning and characterization of a specific coactivator, ARA70, for the androgen receptor in human prostate cells. Proc Natl Acad Sci USA 93, 5517-5521. (1996).
Yeh, S. et al. Differential induction of androgen receptor transactivation by different androgen receptor coactivators in human prostate cancer DU145 cells. Endocrine 11, 195-202. (1999).
Yeh, S. et al. From HER2/Neu signal cascade to androgen receptor and its coactivators: a novel pathway by induction of androgen target genes through MAP kinase in prostate cancer cells. Proc Natl Acad Sci USA 96, 5458-5463. (1999).
Yeh, S. et al. Retinoblastoma, a tumor suppressor, is a coactivator for the androgen receptor in human prostate cancer DU145 cells. Biochem Biophys Res Commun 248, 361-367. (1998).
Yeh, S., Miyamoto, H., and Chang, C. (1996) Lancet 349, 852-853
Yeh, S., Miyamoto, H., Nishimura, K., Kang, H., Ludlow, J., Hsiao, P., Wang, C., Su, C., and Chang, C. (1998) Biochem. Biophys. Res. Commun. 248, 361-367
Yeh, S., Miyamoto, H., Shima, H. & Chang, C. (1998) Proc. Natl. Acad. Sci. USA 95, 5527-32.
Yeh, S., Sampson, E. R., Lee, D. K., Kim, E., Hsu, C. L., Chen, Y. L., Chang, H. C., Altuwaijri, S., Huang, K. E., & Chang, C. J. Formos. Med. Assoc. (2000) 99, 885-894.
Yong, E. L., Lim, J., Qi, W., Ong, V. & Mifsud, A. Molecular basis of androgen receptor diseases. Ann Med 32, 15-22. (2000).
Yuan, S., Trachtenberg, J., Mills, G. B., Brown, T. J., Xu, F. & Keating, A. (1993) Cancer Res. 53, 1304-11.
Zhou Z X, He B, Hall S H, Wilson E M, and French F S. (2001) Mol. Endocrino 16, 287-300
Zhou, Z. X., Lane, M. V., Kemppainen, J. A., French, F. S. & Wilson, E. M. (1995) Mol. Endocrinol. 9, 208-18.

### H. SEQUENCES

1. SEQ ID NO:13 Genbank Accession No. X80172. M. musculus gene for androgen-receptor 5′ untranslated region.
2. SEQ ID NO:14 Genbank Accession No. X59591. Mouse gene for androgen receptor promoter region.
3. SEQ ID NO:15 Genbank Accession No. X59590. Mouse gene for androgen receptor, 3′ UTR.
4. SEQ ID NO:16 Genbank Accession No. X59592. Mouse protein for androgen receptor.
5. SEQ ID NO:17 Genbank Accession No. X59592. Mouse mRNA for androgen receptor
6. SEQ ID NO:18 Genbank Accession No. X59592. Mouse protein for androgen receptor
7. SEQ ID NO:19 Genbank Accession No. X59592. Mouse mRNA for androgen receptor.
8. SEQ ID NO:20 Genbank Accession No. M37890. Mouse androgen receptor protein, complete cds.
9. SEQ ID NO:21 Genbank Accession No. M37890. Mouse androgen receptor mRNA, complete cds
10. SEQ ID NO:22 Genbank Accession No. NM_{—}000044 Human AR mRNA
11. SEQ ID NO:23 Genbank Accession No. NM_{—}000044 Human AR protein sequence
12. SEQ ID NO:24 Genbank accession number X03635. for Human protein sequence of an estrogen receptor
13. SEQ ID NO:25 Genbank accession number X03635. for Human mRNA sequence of an estrogen receptor
14. SEQ ID NO:26 Human ARA70 mRNA, complete protein. ACCESSION L49399.
15. SEQ ID NO:27 Human ARA70 mRNA, complete cds. ACCESSION L49399 Homo sapiens prostate cDNA to mRNA.
16. SEQ ID NO:28 Homo sapiens androgen receptor associated protein 54 (ARA54) protein, complete protein ACCESSION AF060544
17. SEQ ID NO:29 Homo sapiens androgen receptor associated cDNA 54 (ARA54) mRNA, complete cds ACCESSION AF060544
18. SEQ ID NO:30 Homo sapiens androgen receptor coactivator ARA55 mRNA, complete protein ACCESSION AF116343
19. SEQ ID NO:31 Homo sapiens androgen receptor coactivator ARA55 mRNA, complete cds. ACCESSION AF116343
20. SEQ ID NO:32 Homo sapiens androgen receptor associated protein 24 (ARA2 mRNA, complete protein ACCESSION AF052578
21. SEQ ID NO:33 Homo sapiens androgen receptor associated protein 24 (ARA24) mRNA, complete cds. ACCESSION AF052578
22. SEQ ID NO:34 Homo sapiens androgen receptor-associated coregulator 267-a mRNA, complete protein. ACCESSION AF380302
23. SEQ ID NO:35 Homo sapiens androgen receptor-associated coregulator 267-a mRNA, complete cds. ACCESSION AF380302
24. SEQ ID NO:36 Homo sapiens androgen receptor associated coregulator 267-b(ARA267b) protein, complete cds. SEQ ID NO:20 ACCESSION AY049721
25. SEQ ID NO:37 Homo sapiens androgen receptor associated coregulator 267-b(ARA267b) mRNA, complete cds. ACCESSION AY049721
26. SEQ ID NO:38 Homo sapiens supervillin protein, complete cds. ACCESSION AF051850
27. SEQ ID NO:39 Homo sapiens supervillin mRNA, complete cds. ACCESSION AF051850
28. SEQ ID NO:40 Mouse gelsolin gene, complete protein ACCESSION J04953
29. SEQ ID NO:41 Mouse gelsolin gene, complete cDNA ACCESSION J04953
30. SEQ ID NO:42 Human retinoblastoma susceptibility protein complete cds. ACCESSION M28419
31. SEQ ID NO:43 Human retinoblastoma susceptibility mRNA, complete cds. ACCESSION M28419
32. SEQ ID NO:44 Human Gelsolin Genbank Accession No. BC026033. Homo sapiens, gelsolin (amyloidosis, Finnish type), clone MGC:39262
33. SEQ ID NO:45 Human Gelsolin Genbank Accession No. BC026033. Homo sapiens, gelsolin (amyloidosis, Finnish type), clone MGC:39262
34. SEQ ID NO:46 SRC-1 protein Genbank Accession No. U90661. Human steroid receptor coactivator-1 mRNA, complete protein.
35. SEQ ID NO:47 SRC-1 protein Genbank Accession No. U90661. Human steroid receptor coactivator-1 mRNA, complete cds.

1. A composition comprising an isolated mutant of an ARA54 peptide comprising a peptide having at least 80% identity to SEQ ID NO:1, wherein the peptide prevents homodimerization of ARA54.

2. The composition of claim 1, wherein the mutant ARA further comprises a substitution at position 472 of SEQ ID NO:1.

3. The composition of claim 2, wherein the mutant ARA comprises a lysine substitution at position 472 of SEQ ID NO:1.

4. A composition comprising a nucleic acid encoding the mutant ARA of claim 1.

5. The composition of claim 4, wherein the nucleic acid further comprises a promoter sequence operably linked to the sequence encoding the mutant ARA.

6. A composition comprising a cell comprising the nucleic acid of claim 5.

7. An animal comprising the cell of claim 6.

8. (canceled)

9. A method of identifying a molecule that modulates the activity of androgen receptor comprising administering the molecule to a system comprising androgen receptor and the composition of claim 1, assaying the activity of androgen receptor, and selecting molecules that modulate the activity of androgen receptor.

10. The method of claim 9, wherein the system further comprises ARA54, ARA55, SRC-1, ARA24, Rb, ARA70, RB, ARA24, ARA267, gelsolin, or supervillin, or variant comprising androgen receptor modulating activity, in any combination.

11. The method of claim 9, wherein the system further comprises a nucleic acid encoding the ARA54, ARA55, SRC-1, ARA24, Rb, ARA70, RB, ARA24, ARA267, gelsolin, or supervillin, or variant comprising androgen receptor modulating activity.

12. (canceled)

13. The method of claim 9, wherein the system further comprises three molecules wherein the molecules are ARA54, ARA55, SRC-1, ARA24, Rb, ARA70, ARA267, gelsolin, or supervillin, or variant comprising androgen receptor modulating activity, in any combination.

14-19. (canceled)

20. A method of identifying a dominant negative inhibitor of androgen receptor comprising administering a mutagen to a nucleic acid encoding an ARA interacting protein forming a nucleic acid encoding a mutated ARA interacting protein, performing a screening system, wherein the system comprises the mutated ARA interacting protein and androgen receptor, assaying the activity of the androgen receptor, and identifying those mutated ARA interacting proteins that reduce androgen receptor activity.

21. The method of claim 20, wherein the mutagen comprises hydroxylamine.

22. A composition comprising an ARA267 peptide comprising a peptide having at least 80% identity to SEQ ID NO:34, wherein the peptide enhances androgen receptor transactivation of androgen receptor.

23. The composition of claim 22, wherein the mutant ARA wherein the mutant ARA further comprises an LXXLL motif, a set motif, a praline rich region, a ring finger motif, or a zinc finger motif.

24-27. (canceled)

28. A composition comprising an ARA267 peptide comprising amino acids 1668-1795 of SEQ ID NO: 34, amino acids 726-730 of SEQ ID NO:34, and amino acids 1283-1287 of SEQ ID NO:34, amino acids 1324-1369 of SEQ ID NO:34 and amino acids 1884-1909 of SEQ ID NO:34.

29. A nucleic acid encoding the ARA267 of claims 22.

30. (canceled)

31. A cell comprising the nucleic acid of claim 30.

32. An animal comprising the cell of claim 30.

33. A method of enhancing androgen receptor transactivation comprising administering the composition of claims 22.

34. A method of inhibiting androgen receptor transactivation comprising administering the nucleic acid of claims 30.

35. A method of identifying a molecule that modulates the activity of androgen receptor comprising administering the molecule to a system comprising androgen receptor and the composition of claims 22, assaying the activity of androgen receptor, and selecting molecules that modulate the activity of androgen receptor.

36. The method of claim 35, wherein the system further comprises ARA54, ARA55, SRC-1, SRC-1, ARA24, Rb, ARA70, RB, ARA24, ARA267, gelsolin, or supervillin, or variant comprising androgen receptor modulating activity, in any combination.

37. The method of claim 35, wherein the system further comprises a nucleic acid encoding the ARA54, ARA55, SRC-1, SRC-1, ARA24, Rb, ARA70, RB, ARA24, ARA267, gelsolin, or supervillin, or variant comprising androgen receptor modulating activity.

38. (canceled)

39. The method of claim 35, wherein the system further comprises three molecules wherein the molecules are ARA54, ARA55, SRC-1, ARA24, Rb, ARA70, ARA267, gelsolin, or supervillin, or variant comprising androgen receptor modulating activity, in any combination.

40-45. (canceled)

46. A composition comprising an isolated mutant of an ARA70 peptide comprising a peptide having at least 80% identity to SEQ ID NO:26, wherein the peptide prevents androgen receptor transactivation of androgen receptor.

47-49. (canceled)

50. An isolated peptide comprising FXXLF, wherein the peptide interacts with androgen receptor, and wherein the peptide is not ARA54, ARA55, SRC-1, SRC-1, ARA24, Rb, ARA70, RB, ARA24, ARA267, gelsolin, and supervillin.

51. (canceled)

52. A nucleic acid encoding the mutant ARA of claims 46.

53. The nucleic acid of claims 52, wherein the nucleic acid further comprises a promoter sequence operably linked to the sequence encoding the mutant ARA.

54. A cell comprising the nucleic acid of claim 52.

55. An animal comprising the cell of claim 54.

56. A method of inhibiting androgen receptor transactivation comprising administering the composition of claims 46.

57. A method of inhibiting androgen receptor transactivation comprising administering the nucleic acid of claim 53.

58. A method of identifying a molecule that modulates the activity of androgen receptor comprising administering the molecule to a system comprising androgen receptor and the composition of claim 46, assaying the activity of androgen receptor, and selecting molecules that modulate the activity of androgen receptor.

59. The method of claim 58, wherein the system further comprises ARA54, ARA55, SRC-1, SRC-1, ARA24, Rb, ARA70, RB, ARA24, ARA267, gelsolin, or supervillin, or variant comprising androgen receptor modulating activity, in any combination.

60. The method of claim 58, wherein the system further comprises a nucleic acid encoding the ARA54, ARA55, SRC-1, SRC-1, ARA24, Rb, ARA70, RB, ARA24, ARA267, gelsolin, or supervillin, or variant comprising androgen receptor modulating activity.

61. (canceled)

62. The method of claim 58, wherein the system further comprises three molecules wherein the molecules are ARA54, ARA55, SRC-1, ARA24, Rb, ARA70, ARA267, gelsolin, or supervillin, or variant comprising androgen receptor modulating activity, in any combination.

63-68. (canceled)

69. A method of inhibiting androgen receptor activity comprising, administering a molecule that blocks an interaction between the androgen receptor and gelsolin.

70-73. (canceled)

74. A method of identifying an androgen receptor activity inhibiting molecule, comprising administering a molecule or set of molecules to a system, wherein the system comprises androgen receptor and gelsolin, and assaying whether the molecule reduces the interaction between androgen receptor and gelsolin.

75-76. (canceled)

77. A method of identifying an mutant androgen receptor activity inhibiting molecule, comprising administering a molecule or set of molecules to a system, wherein the system comprises the mutant androgen receptor and gelsolin, and assaying whether the molecule reduces the interaction between the mutant androgen receptor and gelsolin.

78-79. (canceled)

80. A method of making a composition, the method comprising synthesizing a molecule, wherein the molecule inhibits androgen receptor activity, and wherein the molecule inhibits an interaction between androgen receptor and gelsolin.

81. A system comprising ARA267 or a peptide or protein comprising FXXLF.

82-85. (canceled)

86. The system of claim 81, wherein the system further comprises three of ARA54, ARA55, SRC-1, ARA24, Rb, ARA70, ARA267, gelsolin, or supervillin, or fragment or variant thereof.

87-92. (canceled)

93. A method of inhibiting androgen receptor activity comprising, administering a molecule that blocks an interaction between the androgen receptor and Supervillin.

94-95. (canceled)

96. A method of inhibiting activity of a mutant androgen receptor comprising, administering a molecule that blocks an interaction between the mutant androgen receptor and supervillin.

97-98. (canceled)

99. A method of identifying an androgen receptor activity inhibiting molecule, comprising administering a molecule or set of molecules to a system, wherein the system comprises androgen receptor and supervillin, and assaying whether the molecule reduces the interaction between androgen receptor and supervillin.

100-102. (canceled)


SEQUENCE LISTING (47 sequences)

SEQ ID NO: 1 - DNA, 1721 bp, Homo sapien
  CDS (40)...(1464)
  misc_feature (1120)...(1452): Coding sequence and polypeptide region for the C-terminal domain
ggtctctggtctcccctctctgagcactctgaggtccttatgtcgtcagaagatcgagaagctcaggaggatgaattgctggccctggcaagtatttacgatggagatgaatttagaaaagcagagtctgtccaaggtggagaaaccaggatctatttggatttgccacagaatttcaagatatttgtgagcggcaattcaaatgagtgtctccagaatagtggctttgaatacaccatttgctttctgcctccacttgtgctgaactttgaactgccaccagattatccatcctcttccccaccttcattcacacttagtggcaaatggctgtcaccaactcagctatctgctctatgcaagcacttagacaacctatgggaagaacaccgtggcagcgtggtcctgtttgcctggatgcaatttcttaaggaagagaccctagcatacttgaatattgtctctccttttgagctcaagattggttctcagaaaaaagtgcagagaaggacagctcaagcttctcccaacacagagctagattttggaggagctgctggatctgatgtagaccaagaggaaattgtggatgagagagcagtgcaggatgtggaatcactgtcaaatctgatccaggaaatcttggactttgatcaagctcagcagataaaatgctttaatagtaaattgttcctgtgcagtatctgtttctgtgagaagctgggtagtgaatgcatgtacttcttggagtgcaggcatgtgtactgcaaagcctgtctgaaggactactttgaaatccagatcagagatggccaggttcaatgcctcaactgcccagaaccaaagtgcccttcggtggccactcctggtcaggtcaaagagttagtggaagcagagttatttgcccgttatgaccgccttctcctccagtcctccttggacctgatggcagatgtggtgtactgcccccggccgtgctgccagctgcctgtgatgcaggaacctggctgcaccatgggtatctgctccagctgcaattttgccttctgtactttgtgcaggttgacctaccatggggtctccccatgtaaggtgactgcagagaaattaatggacttacgaaatgaatacctgcaagcggatgaggctaataaaagacttttggatcaaaggtatggtaagagagtgattcagaaggcactggaagagatggaaagtaaggagtggctagagaagaactcaaagagctgcccatgttgtggaactcccatagagaaattagacggatgtaacaagatgacatgtactggctgtatgcaatatttctgttggatttgcatgggttctctctctagagcaaacccttacaaacatttcaatgaccctggttcaccatgttttaaccggctgttttatgctgtggatgttgacgacgatatttgggaagatgaggtagaagactagttaactactgctcaagatatttaactactgctcaagatatggaagtggattgtttttccctaatcttccgtcaagtacacaaagtaactttgcgggatatttagggtactattcattcactcttcctgcgtagaagatatggaagaacgaggtttatattttcatgtggtactactgaagaaggtgcattgatacatttttaaatgtaagttgagaaaaatttataagccaaaggttcagaaaattaaactacagaa

SEQ ID NO: 2 - protein, 474 aa, Homo sapien
MSSEDREAQEDELLALASIYDGDEFRKAESVQGGETRIYLDLPQNFKIFVSGNSNECLQNSGFEYTICFLPPLVLNFELPPDYPSSSPPSFTLSGKWLSPTQLSALCKHLDNLWEEHRGSVVLFAWMQFLKEETLAYLNIVSPFELKIGSQKKVQRRTAQASPNTELDFGGAAGSDVDQEEIVDERAVQDVESLSNLIQEILDFDQAQQIKCFNSKLFLCSICFCEKLGSECMYFLECRHVYCKACLKDYFEIQIRDGQVQCLNCPEPKCPSVATPGQVKELVEAELFARYDRLLLQSSLDLMADVVYCPRPCCQLPVMQEPGCTMGICSSCNFAFCTLCRLTYHGVSPCKVTAEKLMDLRNEYLQADEANKRLLDQRYGKRVIQKALEEMESKEWLEKNSKSCPCCGTPIEKLDGCNKMTCTGCMQYFCWICMGSLSRANPYKHFNDPGSPCFNRLFYAVDVDDDIWEDEVED

SEQ ID NO: 3 - DNA, 1335 bp, Homo sapien
  CDS (1)...(1335)
  misc_feature (750)...(1332): Coding sequence and polypeptide region for the C-terminal binding domain
atgccaaggtcaggggctcccaaagagcgccctgcggagcctctcacccctcccccatcctatggccaccagccaacagggcagtctggggagtcttcaggagcctcgggggacaaggaccacctgtacagcacggtatgcaagcctcggtccccaaagcctgcagccccggccgcccctccattctcctcttccagcggtgtcttgggtaccgggctctgtgagctagatcggttgcttcaggaacttaatgccactcagttcaacatcacagatgaaatcatgtctcagttcccatctagcaaggtggcttcaggagagcagaaggaggaccagtctgaagataagaaaagacccagcctcccttccagcccgtctcctggcctcccaaaggcttctgccacctcagccactctggagctggatagactgatggcctcactccctgacttccgcgttcaaaaccatcttccagcctctgggccaactcagccaccggtggtgagctccacaaatgagggctccccatccccaccagagccgactgcaaagggcagcctagacaccatgctggggctgctgcagtccgacctcagccgccggggtgttcccacccaggccaaaggcctctgtggctcctgcaataaacctattgctgggcaagtggtgacggctctgggccgcgcctggcaccccgagcacttcgtttgcggaggctgttccaccgccctgggaggcagcagcttcttcgagaaggatggagcccccttctgccccgagtgctactttgagcgcttctcgccaagatgtggcttctgcaaccagcccatccgacacaagatggtgaccgccttgggcactcactggcacccagagcatttctgctgcgtcagttgcggggagcccttcggagatgagggtttccacgagcgcgagggccgcccctactgccgccgggacttcctgcagctgttcgccccgcgctgccagggctgccagggccccatcctggataactacatctcggcgctcagcctgctctggcacccggactgtttcgtctgcagggaatgcttcgcgcccttctcgggaggcagctttttcgagcacgagggccgcccgttgtgcgagaaccacttccacgcacgacgcggctcgctgtgccccacgtgtggcctccctgtgaccggccgctgcgtgtcggccctgggtcgccgcttccacccggaccacttcgcatgcaccttctgcctgcgcccgctcaccaaggggtccttccaggagcgcgccggcaagccctactgccagccctgcttcctgaagctcttcggctga

SEQ ID NO: 4 - protein, 444 aa, Homo sapien
MPRSGAPKERPAEPLTPPPSYGHQPTGQSGESSGASGDKDHLYSTVCKPRSPKPAAPAAPPFSSSSGVLGTGLCELDRLLQELNATQFNITDEIMSQFPSSKVASGEQKEDQSEDKKRPSLPSSPSPGLPKASATSATLELDRLMASLPDFRVQNHLPASGPTQPPVVSSTNEGSPSPPEPTAKGSLDTMLGLLQSDLSRRGVPTQAKGLCGSCNKPIAGQVVTALGRAWHPEHFVCGGCSTALGGSSFFEKDGAPFCPECYFERFSPRCGFCNQPIRHKMVTALGTHWHPEHFCCVSCGEPFGDEGFHEREGRPYCRRDFLQLFAPRCQGCQGPILDNYISALSLLWHPDCFVCRECFAPFSGGSFFEHEGRPLCENHFHARRGSLCPTCGLPVTGRCVSALGRRFHPDHFACTFCLRPLTKGSFQERAGKPYCQPCFLKLFG

SEQ ID NO: 5 - DNA, 1566 bp, Homo sapien
  CDS (25)...(675)
  3′UTR (676)...(1566)
  5′UTR (1)...(24)
ggcgcttctggaaggaacgccgcgatggctgcgcagggagagccccaggtccagttcaaacttgtattggttggtgatggtggtactggaaaaacgaccttcgtgaaacgtcatttgactggtgaatttgagaagaagtatgtagccaccttgggtgttgaggttcatcccctagtgttccacaccaacagaggacctattaagttcaatgtatgggacacagccggccaggagaaattcggtggactgagagatggctattatatccaagcccagtgtgccatcataatgtttgatgtaacatcgagagttacttacaagaatgtgcctaactggcatagagatctggtacgagtgtgtgaaaacatccccattgtgttgtgtggcaacaaagtggatattaaggacaggaaagtgaaggcgaaatccattgtcttccaccgaaagaagaatcttcagtactacgacatttctgccaaaagtaactacaactttgaaaagcccttcctctggcttgctaggaagctcattggagaccctaacttggaatttgttgccatgcctgctctcgccccaccagaagttgtcatggacccagctttggcagcacagtatgagcacgacttagaggttgctcagacaactgctctcccggatgaggatgatgacctgtgagaatgaagctggagcccagcgtcagaagtctagttttataggcagctgtcctgtgatgtcagcggtgcagcgtgtgtgccacctcattattatctagctaagcggaacatgtgctttatctgtgggatgctgaaggagatgagtgggcttcggagtgaatgtggcagtttaaaaaataacttcattgtttggacctgcatatttagctgtttggacgcagttgattccttgagtttcatatataagactgctgcagtcacatcacaatattcagtggtgaaatcttgtttgttactgtcattcccattccttttctttagaatcagaataaagttgtatttcaaatatctaagcaagtgaactcatcccttgtttataaatagcatttggaaaccactaaagtagggaagttttatgccatgttaatatttgaattgccttgcttttatcacttaatttgaaatctattgggttaatttctccctatgtttatttttgtacatttgagccatgtcacacaaactgatgatgacaggtcagcagtattctatttggttagaagggttacatggtgtaaatattagtgcagttaagctaaagcagtgtttgctccaccttcatattggctaggtagggtcacctagggaagcacttgctcaaaatctgtgacctgtcagaataaaaatgtggtttgtacatatcaaatagatattttaagggtaatattttcttttatggcaaaagtaatcatgttttaatgtagaacctcaaacaggatggaacatcagtggatggcaggaggttgggaattcttgctgttaaaaataattacaaattttgcactttttgtttgaatgttagatgcttagtgtgaagttgatacgcaagccg

SEQ ID NO: 6 - protein, 216 aa, Homo sapien
MAAQGEPQVQFKLVLVGDGGTGKTTFVKRHLTGEFEKKYVATLGVEVHPLVFHTNRGPIKFNVWDTAGQEKFGGLRDGYYIQAQCAIIMFDVTSRVTYKNVPNWHRDLVRVCENIPIVLCGNKVDIKDRKVKAKSIVFHRKKNLQYYDISAKSNYNFEKPFLWLARKLIGDPNLEFVAMPALAPPEVVMDPALAAQYEHDLEVAQTTALPDEDDDL

SEQ ID NO: 7 - DNA, 4839 bp, Homo sapien
  CDS (138)...(2924)
tccggtttttctcaggggacgttgaaattatttttgtaacgggagtcgggagaggacggggcgtgccccgcgtgcgcgcgcgtcgtcctccccggcgctcctccacagctcgctggctcccgccgcggaaaggcgtcatgccgcccaaaaccccccgaaaaacggccgccaccgccgccgctgccgccgcggaacccccggcaccgccgccgccgccccctcctgaggaggacccagagcaggacagcggcccggaggacctgcctctcgtcaggcttgagtttgaagaaacagaagaacctgattttactgcattatgtcagaaattaaagataccagatcatgtcagagagagagcttggttaacttgggagaaagtttcatctgtggatggagtattgggaggttatattcaaaagaaaaaggaactgtggggaatctgtatctttattgcagcagttgacctagatgagatgtcgttcacttttactgagctacagaaaaacatagaaatcagtgtccataaattctttaacttactaaaagaaattgataccagtaccaaagttgataatgctatgtcaagactgttgaagaagtatgatgtattgtttgcactcttcagcaaattggaaaggacatgtgaacttatatatttgacacaacccagcagttcgatatctactgaaataaattctgcattggtgctaaaagtttcttggatcacatttttattagctaaaggggaagtattacaaatggaagatgatctggtgatttcatttcagttaatgctatgtgtccttgactattttattaaactctcacctcccatgttgctcaaagaaccatataaaacagctgttatacccattaatggttcacctcgaacacccaggcgaggtcagaacaggagtgcacggatagcaaaacaactagaaaatgatacaagaattattgaagttctctgtaaagaacatgaatgtaatatagatgaggtgaaaaatgtttatttcaaaaattttataccttttatgaattctcttggacttgtaacatctaatggacttccagaggttgaaaatctttctaaacgatacgaagaaatttatcttaaaaataaagatctagatgcaagattatttttggatcatgataaaactcttcagactgattctatagacagttttgaaacacagagaacaccacgaaaaagtaaccttgatgaagaggtgaatgtaattcctccacacactccagttaggactgttatgaacactatccaacaattaatgatgattttaaattcagcaagtgatcaaccttcagaaaatctgatttcctattttaacaactgcacagtgaatccaaaagaaagtatactgaaaagagtgaaggatataggatacatctttaaagagaaatttgctaaagctgtgggacagggttgtgtcgaaattggatcacagcgatacaaacttggagttcgcttgtattaccgagtaatggaatccatgcttaaatcagaagaagaacgattatccattcaaaattttagcaaacttctgaatgacaacatttttcatatgtctttattggcgtgcgctcttgaggttgtaatggccacatatagcagaagtacatctcagaatcttgattctggaacagatttgtctttcccatggattctgaatgtgcttaatttaaaagcctttgatttttacaaagtgatcgaaagttttatcaaagcagaaggcaacttgacaagagaaatgataaaacatttagaacgatgtgaacatcgaatcatggaatcccttgcatggctctcagattcacctttatttgatcttattaaacaatcaaaggaccgagaaggaccaactgatcaccttgaatctgcttgtcctcttaatcttcctctccagaataatcacactgcagcagatatgtatctttctcctgtaagatctccaaagaaaaaaggttcaactacgcgtgtaaattctactgcaaatgcagagacacaagcaacctcagccttccagacccagaagccattgaaatctacctctctttcactgttttataaaaaagtgtatcggctagcctatctccggctaaatacactttgtgaacgccttctgtctgagcacccagaattagaacatatcatctggacccttttccagcacaccctgcagaatgagtatgaactcatgagagacaggcatttggaccaaattatgatgtgttccatgtatggcatatgcaaagtgaagaatatagaccttaaattcaaaatcattgtaacagcatacaaggatcttcctcatgctgttcaggagacattcaaacgtgttttgatcaaagaagaggagtatgattctattatagtattctataactcggtcttcatgcagagactgaaaacaaatattttgcagtatgcttccaccaggccccctaccttgtcaccaatacctcacattcctcgaagcccttacaagtttcctagttcacccttacggattcctggagggaacatctatatttcacccctgaagagtccatataaaatttcagaaggtctgccaacaccaacaaaaatgactccaagatcaagaatcttagtatcaattggtgaatcattcgggacttctgagaagttccagaaaataaatcagatggtatgtaacagcgaccgtgtgctcaaaagaagtgctgaaggaagcaaccctcctaaaccactgaaaaaactacgctttgatattgaaggatcagatgaagcagatggaagtaaacatctcccaggagagtccaaatttcagcagaaactggcagaaatgacttctactcgaacacgaatgcaaaagcagaaaatgaatgatagcatggatacctcaaacaaggaagagaaatgaggatctcaggaccttggtggacactgtgtacacctctggattcattgtctctcacagatgtgactgtataactttcccaggttctgtttatggccacatttaatatcttcagctctttttgtggatataaaatgtgcagatgcaattgtttgggtgattcctaagccacttgaaatgttagtcattgttatttatacaagattgaaaatcttgtgtaaatcctgccatttaaaaagttgtagcagattgtttcctcttccaaagtaaaattgctgtgctttatggatagtaagaatggccctagagtgggagtcctgataacccaggcctgtctgactactttgccttcttttgtagcatataggtgatgtttgctcttgtttttattaatttatatgtatatttttttaatttaacatgaacacccttagaaaatgtgtcctatctatcttccaaatgcaatttgattgactgcccattcaccaaaattatcctgaactcttctgcaaaaatggatattattagaaattagaaaaaaattactaattttacacattagattttattttactattggaatctgatatactgtgtgcttgttttataaaattttgcttttaattaaataaaagctggaagcaaagtataaccatatgatactatcatactactgaaacagatttcatacctcagaatgtaaaagaacttactgattattttcttcatccaacttatgtttttaaatgaggattattgatagtactcttggtttttataccattcagatcactgaatttataaagtacccatctagtacttgaaaaagtaaagtgttctgccagatcttaggtatagaggaccctaacacagtatatcccaagtgcactttctaatgtttctgggtcctgaagaattaagatacaaattaattttactccataaacagactgttaattataggagccttaatttttttttcatagagatttgtctaattgcatctcaaaattattctgccctccttaatttgggaaggtttgtgttttctctggaatggtacatgtcttccatgtatcttttgaactggcaattgtctatttatcttttatttttttaagtcagtatggtctaacactggcatgttcaaagccacattatttctagtccaaaattacaagtaatcaagggtcattatgggttaggcattaatgtttctatctgattttgtgcaaaagcttcaaattaaaacagctgcattagaaaaagaggcgcttctcccctcccctacacctaaaggtgtatttaaactatcttgtgtgattaacttatttagagatgctgtaacttaaaataggggatatttaaggtagcttcagctagcttttaggaaaatcactttgtctaactcagaattatttttaaaaagaaatctggtcttgttagaaaacaaaattttattttgtgctcatttaagtttcaaacttactattttgacagttattttgataacaatgacactagaaaacttgactccatttcatcattgtttctgcatgaatatcatacaaatcagttagtttttaggtcaagggcttactatttctgggtcttttgctactaagttcacattagaattagtgccagaattttaggaacttcagagatcgtgtattgagatttcttaaataatgcttcagatattattgctttattgcttttttgtattggttaaaactgtacatttaaaattgctatgttactattttctacaattaatagtttgtctattttaaaataaattagttgttaagagtcttaatggtctgatgttgtgttctttgtattaagtacactaatgttctcttttctgtctaggagaagatagatagaagataactctcctagtatctcatcc

SEQ ID NO: 8 - protein, 928 aa, Homo sapien
MPPKTPRKTAATAAAAAAEPPAPPPPPPPEEDPEQDSGPEDLPLVRLEFEETEEPDFTALCQKLKIPDHVRERAWLTWEKVSSVDGVLGGYIQKKKELWGICIFIAAVDLDEMSFTFTELQKNIEISVHKFFNLLKEIDTSTKVDNAMSRLLKKYDVLFALFSKLERTCELIYLTQPSSSISTEINSALVLKVSWITFLLAKGEVLQMEDDLVISFQLMLCVLDYFIKLSPPMLLKEPYKTAVIPINGSPRTPRRGQNRSARIAKQLENDTRIIEVLCKEHECNIDEVKNVYFKNFIPFMNSLGLVTSNGLPEVENLSKRYEEIYLKNKDLDARLFLDHDKTLQTDSIDSFETQRTPRKSNLDEEVNVIPPHTPVRTVMNTIQQLMMILNSASDQPSENLISYFNNCTVNPKESILKRVKDIGYIFKEKFAKAVGQGCVEIGSQRYKLGVRLYYRVMESMLKSEEERLSIQNFSKLLNDNIFHMSLLACALEVVMATYSRSTSQNLDSGTDLSFPWILNVLNLKAFDFYKVIESFIKAEGNLTREMIKHLERCEHRIMESLAWLSDSPLFDLIKQSKDREGPTDHLESACPLNLPLQNNHTAADMYLSPVRSPKKKGSTTRVNSTANAETQATSAFQTQKPLKSTSLSLFYKKVYRLAYLRLNTLCERLLSEHPELEHIIWTLFQHTLQNEYELMRDRHLDQIMMCSMYGICKVKNIDLKFKIIVTAYKDLPHAVQETFKRVLIKEEEYDSIIVFYNSVFMQRLKTNILQYASTRPPTLSPIPHIPRSPYKFPSSPLRIPGGNIYISPLKSPYKISEGLPTPTKMTPRSRILVSIGESFGTSEKFQKINQMVCNSDRVLKRSAEGSNPPKPLKKLRFDIEGSDEADGSKHLPGESKFQQKLAEMTSTRTRMQKQKMNDSMDTSNKEEK

SEQ ID NO: 9 - DNA, 30 bp, Artificial Sequence
  Description of Artificial Sequence note = Oligonucleotide
ttctgtagtttaattttctgaacctttggc

SEQ ID NO: 10 - DNA, 27 bp, Artificial Sequence
  Description of Artificial Sequence note = synthetic construct
tcagccgaagagcttcaggaagcaggg

SEQ ID NO: 11 - protein, 32 aa, Homo sapien
  VARIANT 2-3, 5-13, 15, 17-18, 20-21, 23-28, 30-31: Xaa can be any amino acid
CXXCXXXXXXXXXCXHXXCXXCXXXXXXCXXC

SEQ ID NO: 12 - protein, 50 aa, Homo sapien
  VARIANT 2-3, 5-20, 22-23, 25-26, 28-29, 31-46, 48-49: Xaa can be any amino acid
CXXCXXXXXXXXXXXXXXXXHXXCXXCXXCXXXXXXXXXXXXXXXXCXXC

SEQ ID NO: 13 - DNA, 1497 bp, Artificial Sequence
  Description of Artificial Sequence; note = synthetic construct
ctgcagcttgttctttaatgtcaggagactctcccttctgcttgtcctggtgggccctggggggagcggggagggaatacctaagagcaattggtagctggtacttctaatgcctcttcctcctccaacctccaagagtctgttttgggattgggttcaggaatgaaattctgcctgtgctaacctcctggggagccggtagacttgtctgttaaaaatcgcttctgcttttggagcctaaagcccggttccgaaaaacaagtggtatttaggggaaagaggggtcttcaaaggctacagtgagtcattccagccttcaaccatactacgccagcactacgttctctaaagccactctgcgctagcttgcggtgaggggaggggagaaaaggaaaggggaggggaggggaggggagggagaaaggaggtgggaaggcagagaggccggctgcgggggcgggaccgactcacaaactgttcgatttcgtttccacctcccagcgccccctcggagatccctaggagccagcctgctgggagaaccagagggtccggagcaaacctggaggctgagagggcatcagaggggaaaagactgagctagccactccagtgccatacagaagcttaagggacgcaccacgccagccccagcccagcgacagccaacgcctgttgcagagcggcggcttcgaagccgccgcccaggagctgccctttcctcttcggtgaagtttctaaaagctgcgggagactcagaggaagcaaggaaagtgtccggtaggactacggctgcctttgtcctcttcccctctacccttaccccctcctgggtcccctctccaggagctgactaggcaggctttctggccaaccctctcccctacacccccagctctgccagccagtttgcacagaggtaaactccctttggctgagagtaggggagcttgttgcacattgcaaggaaggcttttgggagcccagagactgaggagcaacagcacgcccaggagagtccctggttccaggttctcgcccctgcacctcctcctgcccgcccctcaccctgtgtgtggtgttagaaatgaaaagatgaaaaggcagctagggtttcagtagtcgaaagcaaaacaaaagctaaaagaaaacaaaaagaaaatagcccagttcttatttgcacctgcttcagtggactttgaatttggaaggcagaggatttccccttttccctcccgtcaaggtttgagcatcttttaatctgttcttcaagtatttagagacaaactgtgtaagtagcagggcagatcctgtcttgcgcgtgccttcctttactggagactttgaggttatctgggcactccccccacccaccccccctcctgcaagttttcttccccggagcttcccgcaggtgggcagctagctgcagatactacatcatcagtcaggagaactcttcagagcaagagacgaggaggcaggataagggaattc

SEQ ID NO: 14 - DNA, 600 bp, Artificial Sequence
  Description of Artificial Sequence; note = synthetic construct
ctgcagcttgttctttaatgtcaggagactctcccttctgcttgtcctggtgggccctggggggagcggggagggaatacctaagagcaattggtagctggtacttctaatgcctcttcctcctccaacctccaagagtctgttttgggattgggttcaggaatgaaattctgcctgtgctaacctcctggggagccggtagacttgtctgttaaaaatcgcttctgcttttggagcctaaagcccggttccgaaaaacaagtggtatttaggggaaagaggggtcttcaaaggctacagtgagtcattccagccttcaaccatactacgccagcactacgttctctaaagccactctgcgctagcttgcggtgaggggaggggagaaaaggaaaggggaggggaggggaggggagggagaaaggaggtgggaaggcagagaggccggctgcgggggcgggaccgactcacaaactgttcgatttcgtttccacctcccagcgccccctcggagatccctaggagccagcctgctgggagaaccagagggtccggagcaaacctggaggctgagagggcatcagaggggaaaagactgag

SEQ ID NO: 15 - DNA, 359 bp, Artificial Sequence
  Description of Artificial Sequence; note = synthetic construct
cccaagcgctagtgttctgttctctttttgtaatcttggaatcttttgttgctctaaatacaattaaaaatggcagaaacttgtttgttggaatacatgtgtgactcttggtttgtctctgcgtctggctttagaaatgtcatccattgtgtaaaatactggcttgttggtctgccagctaaaacttgccacagcccctgttgtgactgcaggctcaagttattgttaacaaagagccccaagaaaagctgctaatgtcctcttatcaccattgttaatttgttaaaacataaaacaatctaaaatttcagatgaatgtcatcagagttcttttcattagctctttttattggctgtct

SEQ ID NO: 16 - protein, 899 aa, Artificial Sequence
  Description of Artificial Sequence; note = synthetic construct
MEVQLGLGRVYPRPPSKTYRGAFQNLFQSVREAIQNPGPRHPEAANIAPPGACLQQRQETSPRRRRRQQHTEDGSPQAHIRGPTGYLALEEEQQPSQQQAASEGHPESSCLPEPGAATAPGKGLPQQPPAPPDQDDSAAPSTLSLLGPTFPGLSSCSADIKDILNEAGTMQLLQQQQQQQQHQQQHQQHQQQQEVISEGSSARAREATGAPSSSKDSYLGGNSTISDSAKELCKAVSVSMGLGVEALEHLSPGEQLRGDCMYASLLGGPPAVRPTPCAPLPECKGLPLDEGPGKSTEETAEYSSFKGGYAKGLEGESLGCSGSSEAGSSGTLEIPSSLSLYKSGALDEAAAYQNRDYYNFPLALSGPPHPPPPTHPHARIKLENPLDYGSAWAAAAAQCRYGDLGSLHGGSVAGPSTGSPPATTSSSWHTLFTAEEGQLYGPGGGGGSSSPSDAGPVAPYGYTRPPQGLTSQESDYSASEVWYPGGVVNRVPYPSPNCVKSEMGPWMENYSGPYGDMRLDSTRDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGENSNAGSPTEDPSQKMTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 17 - DNA, 2988 bp, Artificial Sequence
  Description of Artificial Sequence; note = synthetic construct
gcttcccgcaggtgggcagctagctgcagatactacatcatcagtcaggagaactcttcagagcaagagacgaggaggcaggataagggaattcggtggaagctacagacaagctcaaggatggaggtgcagttagggctgggaagggtctacccacggcccccatccaagacctatcgaggagcgttccagaatctgttccagagcgtgcgcgaagcgatccagaacccgggccccaggcaccctgaggccgctaacatagcacctcccggcgcctgtttacagcagaggcaggagactagcccccggcggcggcggcggcagcagcacactgaggatggttctcctcaagcccacatcagaggccccacaggctacctggccctggaggaggaacagcagccttcacagcagcaggcagcctccgagggccaccctgagagcagctgcctccccgagcctggggcggccaccgctcctggcaaggggctgccgcagcagccaccagctcctccagatcaggatgactcagctgccccatccacgttgtccctgctgggccccactttcccaggcttaagcagctgctccgccgacattaaagacattttgaacgaggccggcaccatgcaacttcttcagcagcagcaacaacagcagcagcaccaacagcagcaccaacagcaccaacagcagcaggaggtaatctccgaaggcagcagcgcaagagccagggaggccacgggggctccctcttcctccaaggatagttacctagggggcaattcaaccatatctgacagtgccaaggagttgtgtaaagcagtgtctgtgtccatgggattgggtgtggaagcattggaacatctgagtccaggggaacagcttcggggagactgcatgtacgcgtcgctcctgggaggtccacccgcggtgcgtcccactccttgtgcgccgctgcccgaatgcaaaggtcttcccctggacgaaggcccaggcaaaagcactgaagagactgctgagtattcctctttcaagggaggttacgccaaaggattggaaggtgagagcttggggtgctctggcagcagtgaagcaggtagctctgggacacttgagatcccgtcctctctgtctctgtataaatctggagcactagacgaggcagcagcataccagaatcgcgactactacaactttccgctggctctgtccgggccgccgcaccccccgccccctacccatccacacgcccgtatcaagctggagaacccattggactacggcagcgcctgggctgcggcggcagcgcaatgccgctatggggacttgggtagtctacatggagggagtgtagccgggcccagcactggatcgcccccagccaccacctcttcttcctggcatactctcttcacagctgaagaaggccaattatatgggccaggaggcgggggcggcagcagcagcccaagcgatgccgggcctgtagccccctatggctacactcggccccctcaggggctgacaagccaggagagtgactactctgcctccgaagtgtggtatcctggtggagttgtgaacagagtaccctatcccagtcccaattgtgtcaaaagtgaaatgggaccttggatggagaactactccggaccttatggggacatgcgtttggacagtaccagggaccatgttttacccatcgactattactttccaccccagaagacctgcctgatctgtggagatgaagcttctggctgtcactacggagctctcacttgtggcagctgcaaggtcttcttcaaaagagccgctgaagggaaacagaagtatctatgtgccagcagaaacgattgtaccattgataaatttcggaggaaaaattgcccatcttgtcgtctccggaaatgttatgaagcagggatgactctgggagctcgtaagctgaagaaacttggaaatctaaaactacaggaggaaggagaaaactccaatgctggcagccccactgaggacccatcccagaagatgactgtatcacacattgaaggctatgaatgtcagcctatctttcttaacgtcctggaagccattgagccaggagtggtgtgtgccggacatgacaacaaccaaccagattcctttgctgccttgttatctagcctcaatgagcttggagagaggcagcttgtgcatgtggtcaagtgggccaaggccttgcctggcttccgcaacttgcatgtggatgaccagatggcggtcattcagtattcctggatgggactgatggtatttgccatgggttggcggtccttcactaatgtcaactccaggatgctctactttgcacctgacttggttttcaatgagtaccgcatgcacaagtctcggatgtacagccagtgtgtgaggatgaggcacctgtctcaagagtttggatggctccaaataaccccccaggaattcctgtgcatgaaagcactgctgctcttcagcattattccagtggatgggctgaaaaatcaaaaattctttgatgaacttcgaatgaactacatcaaggaactcgatcgcatcattgcatgcaaaagaaagaatcccacatcctgctcaaggcgcttctaccagctcaccaagctcctggattctgtgcagcctattgcaagagagctgcatcagttcacttttgacctgctaatcaagtcccatatggtgagcgtggactttcctgaaatgatggcagagatcatctctgtgcaagtgcccaagatcctttctgggaaagtcaagcccatctatttccacacacagtgaagatttggaaaccctaatacccaaaacccaccttgttccctttccagatgtcttctgcctgttatataactctgcactacttctctgcagtgccttgggggaaattcctctactgatgtacagtcagacgtgaacaggttcctcagttctatttcctgggcttctcct

SEQ ID NO: 18 - protein, 899 aa, Artificial Sequence; identical to SEQ ID NO: 16
  Description of Artificial Sequence; note = synthetic construct

SEQ ID NO: 19 - DNA, 2988 bp, Artificial Sequence; identical to SEQ ID NO: 17
  Description of Artificial Sequence; note = synthetic construct

SEQ ID NO: 20 - protein, 899 aa, Artificial Sequence; identical to SEQ ID NO: 16
  Description of Artificial Sequence; note = synthetic construct

SEQ ID NO: 21 - DNA, 2700 bp, Artificial Sequence
  Description of Artificial Sequence; note = synthetic construct
atggaggtgcagttagggctgggaagggtctacccacggcccccatccaagacctatcgaggagcgttccagaatctgttccagagcgtgcgcgaagcgatccagaacccgggccccaggcaccctgaggccgctaacatagcacctcccggcgcctgtttacagcagaggcaggagactagcccccggcggcggcggcggcagcagcacactgaggatggttctcctcaagcccacatcagaggccccacaggctacctggccctggaggaggaacagcagccttcacagcagcaggcagcctccgagggccaccctgagagcagctgcctccccgagcctggggcggccaccgctcctggcaaggggctgccgcagcagccaccagctcctccagatcaggatgactcagctgccccatccacgttgtccctgctgggccccactttcccaggcttaagcagctgctccgccgacattaaagacattttgaacgaggccggcaccatgcaacttcttcagcagcagcaacaacagcagcagcaccaacagcagcaccaacagcaccaacagcagcaggaggtaatctccgaaggcagcagcgcaagagccagggaggccacgggggctccctcttcctccaaggatagttacctagggggcaattcaaccatatctgacagtgccaaggagttgtgtaaagcagtgtctgtgtccatgggattgggtgtggaagcattggaacatctgagtccaggggaacagcttcggggagactgcatgtacgcgtcgctcctgggaggtccacccgcggtgcgtcccactccttgtgcgccgctgcccgaatgcaaaggtcttcccctggacgaaggcccaggcaaaagcactgaagagactgctgagtattcctctttcaagggaggttacgccaaaggattggaaggtgagagcttggggtgctctggcagcagtgaagcaggtagctctgggacacttgagatcccgtcctctctgtctctgtataaatctggagcactagacgaggcagcagcataccagaatcgcgactactacaactttccgctggctctgtccgggccgccgcaccccccgccccctacccatccacacgcccgtatcaagctggagaacccattggactacggcagcgcctgggctgcggcggcagcgcaatgccgctatggggacttgggtagtctacatggagggagtgtagccgggcccagcactggatcgcccccagccaccacctcttcttcctggcatactctcttcacagctgaagaaggccaattatatgggccaggaggcgggggcggcagcagcagcccaagcgatgccgggcctgtagccccctatggctacactcggccccctcaggggctgacaagccaggagagtgactactctgcctccgaagtgtggtaccctggtggagttgtgaacagagtaccctatcccagtcccaattgtgtcaaaagtgaaatgggaccttggatggagaactactccggaccttatggggacatgcgtttggacagtaccagggaccatgttttacccatcgactattactttccaccccagaagacctgcctgatctgtggagatgaagcttctggctgtcactacggagctctcacttgtggcagctgcaaggtcttcttcaaaagagccgctgaagggaaacagaagtatctatgtgccagcagaaacgattgtaccattgataaatttcggaggaaaaattgcccatcttgtcgtctccggaaatgttatgaagcagggatgactctgggagctcgtaagctgaagaaacttggaaatctaaaactacaggaggaaggagaaaactccaatgctggcagccccactgaggacccatcccagaagatgactgtatcacacattgaaggctatgaatgtcagcctatctttcttaacgtcctggaagccattgagccaggagtggtgtgtgccggacatgacaacaaccaaccagattcctttgctgccttgttatctagcctcaatgagcttggagagaggcagcttgtgcatgtggtcaagtgggccaaggccttgcctggcttccgcaacttgcatgtggatgaccagatggcggtcattcagtattcctggatgggactgatggtatttgccatgggttggcggtccttcactaatgtcaactccaggatgctctactttgcacctgacttggttttcaatgagtaccgcatgcacaagtctcggatgtacagccagtgtgtgaggatgaggcacctgtctcaagagtttggatggctccaaataaccccccaggaattcctgtgcatgaaagcactgctgctcttcagcattattccagtggatgggctgaaaaatcaaaaattctttgatgaacttcgaatgaactacatcaaggaactcgatcgcatcattgcatgcaaaagaaagaatcccacatcctgctcaaggcgcttctaccagctcaccaagctcctggattctgtgcagcctattgcaagagagctgcatcagttcacttttgacctgctaatcaagtcccatatggtgagcgtggactttcctgaaatgatggcagagatcatctctgtgcaagtgcccaagatcctttctgggaaagtcaagcccatctatttccacacacagtga

SEQ ID NO: 22 - DNA, 4321 bp, Artificial Sequence
  Description of Artificial Sequence; note = synthetic construct
cgagatcccggggagccagcttgctgggagagcgggacggtccggagcaagcccacaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcggcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttagaattccggcggagagaaccctctgttttcccccactctctctccacctcctcctgccttccccaccccgagtgcggagcagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgaacatgttcctgaattctatttgctgggctttttttttctctttctctcctttctttttcttcttccctccctatctaaccctcccatggcaccttcagactttgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaagattatctggggaaatcaaaacaaaaaacaagcaaacaaaaaaaaaa

SEQ ID NO: 23 - protein, 919 aa, Artificial Sequence
  Description of Artificial Sequence; note = synthetic construct
MEVQLGLGRVYPRPPSKTYRGAFQNLFQSVREVIQNPGPRHPEAASAAPPGASLLLLQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 24 - protein, 595 aa, Artificial Sequence
  Description of Artificial Sequence; note = synthetic construct
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 25 - DNA, 6450 bp, Artificial Sequence
  Description of Artificial Sequence; note = synthetic construct
gagttgtgcctggagtgatgtttaagccaatgtcagggcaaggcaacagtccctggccgtcctccagcacctttgtaatgcatatgagctcgggagaccagtacttaaagttggaggcccgggagcccaggagctggcggagggcgttcgtcctgggagctgcacttgctccgtcgggtcgccggcttcaccggaccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgagagctccctggctcccacacggttcagataatccctgctgcattttaccctcatcatgcaccactttagccaaattctgtctcctgcatacactccggcatgcatccaacaccaatggctttctagatgagtggccattcatttgcttgctcagttcttagtggcacatcttctgtcttctgttgggaacagccaaagggattccaaggctaaatctttgtaacagctctctttcccccttgctatgttactaagcgtgaggattcccgtagctcttcacagctgaactcagtctatgggttggggctcagataactctgtgcatttaagctacttgtagagacccaggcctggagagtagacattttgcctctgataagcactttttaaatggctctaagaataagccacagcaaagaatttaaagtggctcctttaattggtgacttggagaaagctaggtcaagggtttattatagcaccctcttgtattcctatggcaatgcatccttttatgaaagtggtacaccttaaagcttttatatgactgtagcagagtatctggtgattgtcaattcacttccccctataggaatacaaggggccacacagggaaggcagatcccctagttggccaagacttattttaacttgatacactgcagattcagagtgtcctgaagctctgcctctggctttccggtcatgggttccagttaattcatgcctcccatggacctatggagagcaacaagttgatcttagttaagtctccctatatgagggataagttcctgatttttgtttttatttttgtgttacaaaagaaagccctccctccctgaacttgcagtaaggtcagcttcaggacctgttccagtgggcactgtacttggatcttcccggcgtgtgtgtgccttacacaggggtgaactgttcactgtggtgatgcatgatgagggtaaatggtagttgaaaggagcaggggccctggtgttgcatttagccctggggcatggagctgaacagtacttgtgcaggattgttgtggctactagagaacaagagggaaagtagggcagaaactggatacagttctgagcacagccagacttgctcaggtggccctgcacaggctgcagctacctaggaacattccttgcagaccccgcattgcctttgggggtgccctgggatccctggggtagtccagctcttattcatttcccagcgtggccctggttggaagaagcagctgtcaagttgtagacagctgtgttcctacaattggcccagcaccctggggcacgggagaagggtggggaccgttgctgtcactactcaggctgactggggcctggtcagattacgtatgcccttggtggtttagagataatccaaaatcagggtttggtttggggaagaaaatcctcccccttcctcccccgccccgttccctaccgcctccactcctgccagctcatttccttcaatttcctttgacctataggctaaaaaagaaaggctcattccagccacagggcagccttccctgggcctttgcttctctagcacaattatgggttacttcctttttcttaacaaaaaagaatgtttgatttcctctgggtgaccttattgtctgtaattgaaaccctattgagaggtgatgtctgtgttagccaatgacccaggtagctgctcgggcttctcttggtatgtcttgtttggaaaagtggatttcattcatttctgattgtccagttaagtgatcaccaaaggactgagaatctgggagggcaaaaaaaaaaaaaaaagtttttatgtgcacttaaatttggggacaattttatgtatctgtgttaaggatatgcttaagaacataattcttttgttgctgtttgtttaagaagcaccttagtttgtttaagaagcaccttatatagtataatatatatttttttgaaattacattgcttgtttatcagacaattgaatgtagtaattctgttctggatttaatttgactgggttaacatgcaaaaaccaaggaaaaatatttagtttttttttttttttttgtatacttttcaagctaccttgtcatgtatacagtcatttatgcctaaagcctggtgattattcatttaaatgaagatcacatttcatatcaacttttgtatccacagtagacaaaatagcactaatccagatgcctattgttggatattgaatgacagacaatcttatgtagcaaagattatgcctgaaaaggaaaattattcagggcagctaattttgcttttaccaaaatatcagtagtaatatttttggacagtagctaatgggtcagtgggttctttttaatgtttatacttagattttcttttaaaaaaattaaaataaaacaaaaaaaatttctaggactagacgatgtaataccagctaaagccaaacaattatacagtggaaggttttacattattcatccaatgtgtttctattcatgttaagatactactacatttgaagtgggcagagaacatcagatgattgaaatgttcgcccaggggtctccagcaactttggaaatctctttgtatttttacttgaagtgccactaatggacagcagatattttctggctgatgttggtattgggtgtaggaacatgatttaaaaaaaaaactcttgcctctgctttcccccactctgaggcaagttaaaatgtaaaagatgtgatttatctggggggctcaggtatggtggggaagtggattcaggaatctggggaatggcaaatatattaagaagagtattgaaagtatttggaggaaaatggttaattctgggtgtgcaccaaggttcagtagagtccacttctgccctggagaccacaaatcaactagctccatttacagccatttctaaaatggcagcttcagttctagagaagaaagaacaacatcagcagtaaagtccatggaatagctagtggtctgtgtttcttttcgccattgcctagcttgccgtaatgattctataatgccatcatgcagcaattatgagaggctaggtcatccaaagagaagaccctatcaatgtaggttgcaaaatctaacccctaaggaagtgcagtctttgatttgatttccctagtaaccttgcagatatgtttaaccaagccatagcccatgccttttgagggctgaacaaataagggacttactgataatttacttttgatcacattaaggtgttctcaccttgaaatcttatacactgaaatggccattgatttaggccactggcttagagtactccttcccctgcatgacactgattacaaatactttcctattcatactttccaattatgagatggactgtgggtactgggagtgatcactaacaccatagtaatgtctaatattcacaggcagatctgcttggggaagctagttatgtgaaaggcaaataaagtcatacagtagctcaaaaggcaaccataattctctttggtgcaagtcttgggagcgtgatctagattacactgcaccattcccaagttaatcccctgaaaacttactctcaactggagcaaatgaactttggtcccaaatatccatcttttcagtagcgttaattatgctctgtttccaactgcatttcctttccaattgaattaaagtgtggcctcgtttttagtcatttaaaattgttttctaagtaattgctgcctctattatggcacttcaattttgcactgtcttttgagattcaagaaaaatttctattcatttttttgcatccaattgtgcctgaacttttaaaatatgtaaatgctgccatgttccaaacccatcgtcagtgtgtgtgtttagagctgtgcaccctagaaacaacatacttgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcgggatgctcgagcacctgtaaacaattttctcaacctatttgatgttcaaataaagaattaaact

SEQ ID NO: 26 - protein, 614 aa, Artificial Sequence
  Description of Artificial Sequence; note = synthetic construct
MNTFQDQSGSSSNREPLLRCSDARRDLELAIGGVLRAEQQIKDNLREVKAQIHSCISRHLECLRSREVWLYEQVDLIYQLKEETLQQQAQQLYSLLGQFNCLTHQLECTQNKDLANQVSVCLERLGSLTLKPEDSTVLLFEADTITLRQTITTFGSLKTIQIPEHLMAHASSANIGPFLEKRGCISMPEQKSASGIVAVPFSEWLLGSKPASGYQAPYIPSTDPQDWLTQKQTLENSQTSSRACNFFNNVGGNLKGLENWLLKSEKSSYQKCNSHSTTSSFSIEMEKVGDQELPDQDEMDLSDWLVTPQESHKLRKPENGSRETSEKFKLLFQSYNVNDWLVKTDSCTNCQGNQPKGVEIENLGNLKCLNDHLEAKKPLSTPSMVTEDWLVQNHQDPCKVEEVCRANEPCTSFAECVCDENCEKEALYKWLLKKEGKDKNGMPVEPKPEPEKHKDSLNMWLCPRKEVIEQTKAPKAMTPSRIADSFQVIKNSPLSEWLIRPPYKEGSPKEVPGTEDRAGKQKFKSPMNTSWCSFNTADWVLPGKKMGNLSQLSSGEDKWLLRKKAQEVLLNSPLQEEHNFPPDHYGLPAVCDLFACMQLKVDKEKWLYRTPLQM

SEQ ID NO: 27 - DNA, 1845 bp, Artificial Sequence
  Description of Artificial Sequence; note = synthetic construct
atgaataccttccaagaccagagtggcagctccagtaatagagaaccccttttgaggtgtagtgatgcacggagggacttggagcttgctattggtggagttctccgggctgaacagcaaattaaagataacttgcgagaggtcaaagctcagattcacagttgcataagccgtcacctggaatgtcttagaagccgtgaggtatggctgtatgaacaggtggaccttatttatcagcttaaagaggagacacttcaacagcaggctcagcagctctactcgttattgggccagttcaattgtcttactcatcaactggagtgtacccaaaacaaagatctagccaatcaagtctctgtgtgcctggagagactgggcagtttgacccttaagcctgaagattcaactgtcctgctctttgaagctgacacaattactctgcgccagaccatcaccacatttgggtctctcaaaaccattcaaattcctgagcacttgatggctcatgctagttcagcaaatattgggcccttcctggagaagagaggctgtatctccatgccagagcagaagtcagcatccggtattgtagctgtccctttcagcgaatggctccttggaagcaaacctgccagtggttatcaagctccttacatacccagcaccgacccccaggactggcttacccaaaagcagaccttggagaacagtcagacttcttccagagcctgcaatttcttcaataatgtcgggggaaacctaaagggcttagaaaactggctcctcaagagtgaaaaatcaagttatcaaaagtgtaacagccattccactactagttctttctccattgaaatggaaaaggttggagatcaagagcttcctgatcaagatgagatggacctatcagattggctagtgactccccaggaatcccataagctgcggaagcctgagaatggcagtcgtgaaaccagtgagaagtttaagctcttattccagtcctataatgtgaatgattggcttgtcaagactgactcctgtaccaactgtcagggaaaccagcccaaaggtgtggagattgaaaacctgggcaatctgaagtgcctgaatgaccacttggaggccaagaaaccattgtccacccccagcatggttacagaggattggcttgtccagaaccatcaggacccatgtaaggtagaggaggtgtgcagagccaatgagccctgcacaagctttgcagagtgtgtgtgtgatgagaattgtgagaaggaggctctgtataagtggcttctgaagaaagaaggaaaggataaaaatgggatgcctgtggaacccaaacctgagcctgagaagcataaagattccctgaatatgtggctctgtcctagaaaagaagtaatagaacaaactaaagcaccaaaggcaatgactccttctagaattgctgattccttccaagtcataaagaacagccccttgtcggagtggcttatcaggcccccatacaaagaaggaagtcccaaggaagtgcctggtactgaagacagagctggcaaacagaagtttaaaagccccatgaatacttcctggtgttcctttaacacagctgactgggtcctgccaggaaagaagatgggcaacctcagccagttatcttctggagaagacaagtggctgcttcgaaagaaggcccaggaagtattacttaattcacctctacaggaggaacataacttccccccagaccattatggcctccctgcagtttgtgatctctttgcctgtatgcagcttaaagttgataaagagaagtggttatatcgaactcctctacagatgtga

SEQ ID NO: 28 - protein, 474 aa, Artificial Sequence; identical to SEQ ID NO: 2
  Description of Artificial Sequence; note = synthetic construct

SEQ ID NO: 29 - DNA, 1701 bp, Artificial Sequence
  Description of Artificial Sequence; note = synthetic construct
ggtctctggtctcccctctctgagcactctgaggtccttatgtcgtcagaagatcgagaagctcaggaggatgaattgctggccctggcaagtatttacgatggagatgaatttagaaaagcagagtctgtccaaggtggagaaaccaggatctatttggatttgccacagaatttcaagatatttgtgagcggcaattcaaatgagtgtctccagaatagtggctttgaatacaccatttgctttctgcctccacttgtgctgaactttgaactgccaccagattatccatcctcttccccaccttcattcacacttagtggcaaatggctgtcaccaactcagctatctgctctatgcaagcacttagacaacctatgggaagaacaccgtggcagcgtggtcctgtttgcctggatgcaatttcttaaggaagagaccctagcatacttgaatattgtctctccttttgagctcaagattggttctcagaaaaaagtgcagagaaggacagctcaagcttctcccaacacagagctagattttggaggagctgctggatctgatgtagaccaagaggaaattgtggatgagagagcagtgcaggatgtggaatcactgtcaaatctgatccaggaaatcttggactttgatcaagctcagcagataaaatgctttaatagtaaattgttcctgtgcagtatctgtttctgtgagaagctgggtagtgaatgcatgtacttcttggagtgcaggcatgtgtactgcaaagcctgtctgaaggactactttgaaatccagatcagagatggccaggttcaatgcctcaactgcccagaaccaaagtgcccttcggtggccactcctggtcaggtcaaagagttagtggaagcagagttatttgcccgttatgaccgccttctcctccagtcctccttggacctgatggcagatgtggtgtactgcccccggccgtgctgccagctgcctgtgatgcaggaacctggctgcaccatgggtatctgctccagctgcaattttgccttctgtactttgtgcaggttgacctaccatggggtctccccatgtaaggtgactgcagagaaattaatggacttacgaaatgaatacctgcaagcggatgaggctaataaaagacttttggatcaaaggtatggtaagagagtgattcagaaggcactggaagagatggaaagtaaggagtggctagagaagaactcaaagagctgcccatgttgtggaactcccatagagaaattagacggatgtaacaagatgacatgtactggctgtatgcaatatttctgttggatttgcatgggttctctctctagagcaaacccttacaaacatttcaatgaccctggttcaccatgttttaaccggctgttttatgctgtggatgttgacgacgatatttgggaagatgaggtagaagactagttaactactgctcaagatatggaagtggattgtttttccctaatcttccgtcaagtacacaaagtaactttgcgggatatttagggtactattcattcactcttcctgcgtagaagatatggaagaacgaggtttatattttcatgtggtactactgaagaaggtgcattgatacatttttaaatgtaagttgagaaaaatttataagccaaaggttcagaaaattaaactacagaa

SEQ ID NO: 30 - protein, 444 aa, Artificial Sequence
  Description of Artificial Sequence; note = synthetic construct
MPRSGAPKERPAEPLTPPPSYGHQPQTGSGESSGASGDKDHLYSTVCKPRSPKPAAPAAPPFSSSSGVLGTGLCELDRLLQELNATQFNITDEIMSQFPSSKVASGEQKEDQSEDKKRPSLPSSPSPGLPKASATSATLELDRLMASLPDFRVQNHLPASGPTQPPVVSSTNEGSPSPPEPTAKGSLDTMLGLLQSDLSRRGVPTQAKGLCGSCNKPIAGQVVTALGRAWHPEHFVCGGCSTALGGSSFFEKDGAPFCPECYFERFSPRCGFCNQPIRHKMVTALGTHWHPEHFCCVSCGEPFGDEGFHEREGRPYCRRDFLQLFAPRCQGCQGPILDNYISALSLLWHPDCFVCRECFAPFSGGSFFEHEGRPLCENHFHARRGSLWPTCGLPVTGRCVSALGRRFHPDHFACTFCLRPLTKGSFQERAGKPYCQPCFLKLFG

SEQ ID NO: 31 - DNA, 1335 bp, Artificial Sequence
  Description of Artificial Sequence; note = synthetic construct
atgccaaggtcaggggctcccaaagagcgccctgcggagcctctcacccctcccccatcctatggccaccagccacagacagggtctggggagtcttcaggagcctcgggggacaaggaccacctgtacagcacggtatgcaagcctcggtccccaaagcctgcagccccggccgcccctccattctcctcttccagcggtgtcttgggtaccgggctctgtgagctagatcggttgcttcaggaacttaatgccactcagttcaacatcacagatgaaatcatgtctcagttcccatctagcaaggtggcttcaggagagcagaaggaggaccagtctgaagataagaaaagacccagcctcccttccagcccgtctcctggcctcccaaaggcttctgccacctcagccactctggagctggatagactgatggcctcactccctgacttccgcgttcaaaaccatcttccagcctctgggccaactcagccaccggtggtgagctccacaaatgagggctccccatccccaccagagccgactgcaaagggcagcctagacaccatgctggggctgctgcagtccgacctcagccgccggggtgttcccacccaggccaaaggcctctgtggctcctgcaataaacctattgctgggcaagtggtgacggctctgggccgcgcctggcaccccgagcacttcgtttgcggaggctgttccaccgccctgggaggcagcagcttcttcgagaaggatggagcccccttctgccccgagtgctactttgagcgcttctcgccaagatgtggcttctgcaaccagcccatccgacacaagatggtgaccgccttgggcactcactggcacccagagcatttctgctgcgtcagttgcggggagcccttcggagatgagggtttccacgagcgcgagggccgcccctactgccgccgggacttcctgcagctgttcgccccgcgctgccagggctgccagggccccatcctggataactacatctcggcgctcagcctgctctggcacccggactgtttcgtctgcagggaatgcttcgcgcccttctcgggaggcagctttttcgagcacgagggccgcccgttgtgcgagaaccacttccacgcacgacgcggctcgctgtggcccacgtgtggcctccctgtgaccggccgctgcgtgtcggccctgggtcgccgcttccacccggaccacttcgcatgcaccttctgcctgcgcccgctcaccaaggggtccttccaggagcgcgccggcaagccctactgccagccctgcttcctgaagctcttcggctga

SEQ ID NO: 32 - protein, 216 aa, Artificial Sequence; identical to SEQ ID NO: 6
  Description of Artificial Sequence; note = synthetic construct

SEQ ID NO: 33 - DNA, 1566 bp, Artificial Sequence; identical to SEQ ID NO: 5
  Description of Artificial Sequence; note = synthetic construct

SEQ ID NO: 34 - protein, 2427 aa, Artificial Sequence
  Description of Artificial Sequence; note = synthetic construct
MPLKTRTALSDDPDSSTSTLGNMLELPGTSSSSTSQELPFCQPKKKSTPLKYEVGDLIWAKFKRRPWWPCRICSDPLINTHSKMKVSNRRPYRQYYVEAFGDPSERAWVAGKAIVMFEGRHQFEELPVLRRRGKQKEKGYRHKVPQKILSKWEASVGLAEQYDVPKGSKNRKCIPGSIKLDSEEDMPFEDCTNDPESEHDLLLNGCLKSLAFDSEHSADEKEKPCAKSRARKSSDNPKRTSVKKGHIQFEAHKDERRGKIPENLGLNFISGDISDTQASNELSRIANSLTGSNTAPGSFLFSSCGKNTAKKEFETSNGDSLLGLPEGALISKCSREKNKPQRSLVCGSKVKLCYIGAGDEEKRSDSISICTTSDDGSSDLDPIEHSSESDNSVLEIPDAFDRTENMLSMQKNEKIKYSRFAATNTRVKAKQKPLISNSHTDHLMGCTKSAEPGTETSQVNLSDLKASTLVHKPQSDFTNDALSPKFNLSSSISSENSLIKGGAANQALLHSKSKQPKFRSIKCKHKENPVMAEPPVINEECSLKCCSSDTKGSPLASISKSGKVDGLKLLNNMHEKTRDSSDIETAVVKHVLSELKELSYRSLGEDVSDSGTSKPSKPLLFSSASSQNHIPIEPDYKFSTLLMMLKDMHDSKTKEQRLMTAQNLVSYRSPGRGDCSTNSPVGVSKVLVSGGSTHNSEKKGDGTQNSANPSPSGGDSALSGELSASLPGLLSDKRDLPASGKSRSDCVTRRNCGRSKPSSKLRDAFSAQMVKNTVNRKALKTERKRKLNQLPSVTLDAVLQGDRERGGSLRGGAEDPSKEDPLQIMGHLTSEDGDHFSDVHFDSKVKQSDPGKISEKGLSFENGKGPELDSVMNSENDELNGVNQVVPKKRWQRLNQRRTKPRKRMNRFKEKENSECAFRVLLPSDPVQEGRDEFPEHRTPSASILEEPLTEQNHADCLDSAGPRLNVCDKSSASIGDMEKEPGIPSLTPQAELPEPAVRSEKKRLRKPSKWLLEYTEEYDQIFAPKKKQKKVQEQVHKVSSRCEEESLLARGRSSAQNKQVDENSLISTKEEPPVLEREAPFLEGPLAQSELGGGHAELPQLTLSVPVAPEVSPRPALESEELLVKTPGNYESKRQRKPTKKLLESNDLDPGFMPKKGDLGLSKKCYEAGHLENGITESCATSYSKDFGGGTTKIFDKPRKRKRQRHAAAKMQCKKVKNDDSSKEIPGSEGELMPHRTATSPKETVEEGVEHDPGMPASKKMQGERGGGAALKENVCQNCEKLGELLLCEAQCCGAFHLECLGLTEMPRGKFICNECRTGIHTCFVCKQSGEDVKRCLLPLCGKFYHEECVQKYPPTVMQNKGFRCSLHICITCHAANPANVSASKGRLMRCVRCPVAYHANDFCLAAGSKILASNSIICPNHFTPRRGCRNHEHVNVSWCFVCSEGGSLLCCDSCPAAFHRECLNIDIPEGNWYCNDCKAGKKPHYREIVWVKVGRYRWWPAEICHPRAVPSNIDKMRHDVGEFPVLFFGSNDYLWTHQARVFPYMEGDVSSKDKMGKGVDGTYKKALQEAAARFEELKAQKELRQLQEDRKNDKKPPPYKHIKVNRPIGRVQIFTADLSEIPRCNCKATDENPCGIDSECINRMLLYECHPTVCPAGGRCQNQCFSKRQYPEVEIFRTLQRGWGLRTKTDIKKGEFVNEYVGELIDEEECRARIRYAQEHDITNFYMLTLDKDRIIDAGPKGNYARFMNHCCQPNCETQKWSVNGDTRVGLFALSDIKAGTELTFNYNLECLGNGKTVCKCGAPNCSGFLGVRPKNQPIATEEKSKKFKKKQQGKRRTQGEITKEREDECFSCGDAGQLVSCKKPGCPKVYHADCLNLTKRPAGKWECPWHQCDICGKEAASFCEMCPSSFCKQHREGMLFISKLDGRLSCTEHDPCGPNPLEPGEIREYVPPPVPLPPGPSTHLAEQSTGMAAQAPKMSDKPPADTNQMLSLSKKALAGTCQRPLLPERPLERTDSRPQPLDKVRDLAGSGTKSQSLVSSQRPLDRPPAVAGPRPQLSDKPSPVTSPSSSPSVRSQPLERPLGTADPRLDKSIGAASPRPQSLEKTSVPTGLRLPPPDRLLITSSPKPQTSDRPTDKPHASLSQRLPPPEKVLSAVVQTLVAKEKALRPVDQNTQSKNRAALVMDLIDLTPRQKERAASPHQVTPQADEKMPVLESSSWPASKGLGHMPRAVEKGCVSDPLQTSGKAAAPSEDPWQAVKSLTQARLLSQPPAKAFLYEPTTQASGRASAGAEQTPGPLSQSPGLVKQAKQMVGGQQLPALAAKSGQSFRSLGKAPASLPTEEKKLVTTEQSPWALGKASSRAGLWPIVAGQTLAQSCWSAGSTQTLAQTCWSLGRGQDPKPEQNTLPALNQAPSSHKCAESEQK

SEQ ID NO: 35 - DNA, 7707 bp, Artificial Sequence
  Description of Artificial Sequence; note = synthetic construct
cctccgcctcccctcaggttgatgccggcccaggatggatcagacctgtgaactacccagaagaaattgtctgctgcccttttccaatccagtgaatttagatgcccctgaagacaaggacagccctttcggatgatccagattccagtaccagtacattaggaaacatgctagaattacctggaacttcatcatcatctacttcacaggaattgccattttgtcaacctaagaaaaagtctacgccactgaagtatgaagttggagatctcatctgggcaaaattcaagagacgcccatggtggccctgcaggatttgttctgatccgttgattaacacacattcaaaaatgaaagtttccaaccggaggccctatcggcagtactacgtggaggcttttggagatccttctgagagagcctgggtggctggaaaagcaatcgtcatgtttgaaggcagacatcaattcgaagagctacctgtccttaggagaagagggaaacagaaagaaaaaggatataggcataaggttcctcagaaaattttgagtaaatgggaagccagtgttggacttgcagaacagtatgatgttcccaaggggtcaaagaaccgaaaatgtattcctggttcaatcaagttggacagtgaagaagatatgccatttgaagactgcacaaatgatcctgagtcagaacatgacctgttgcttaatggctgtttgaaatcactggcttttgattctgaacattctgcagatgagaaggaaaagccttgtgctaaatctcgagccagaaagagctctgataatccaaaaaggactagtgtgaaaaagggccacatacaatttgaagcacataaagatgaacggaggggaaagattccagagaaccttggcctaaactttatctctggggatatatctgatacgcaggcctctaatgaactttccaggatagcaaatagcctcacagggtccaacactgccccaggaagttttctgttttcttcctgtggaaaaaacactgcaaagaaagaatttgagacttcaaatggtgactctttattgggcttgcctgagggtgctttgatctcaaagtgttctcgagagaagaataaaccccaacgaagcctggtgtgtggttcaaaagtgaagctctgctatattggagcaggtgatgaggaaaagcgaagtgattccattagtatctgtaccacttctgatgatggaagcagtgacctggatcccatagaacacagctcagagtctgataacagtgtccttgaaattccagatgctttcgatagaacagagaacatgttatctatgcagaaaaatgaaaagataaagtattctaggtttgctgccacaaacactagggtaaaagcaaaacagaagcctctcattagtaactcacatacagaccacttaatgggttgtactaagagtgcagagcctggaaccgagacgtctcaggttaatctctctgatctgaaggcatctactcttgttcacaaaccccagtcagattttacaaatgatgctctctctccaaaattcaacctgtcatcaagcatatccagtgagaactcgttaataaagggtggggcagcaaatcaagctctattacattcgaaaagcaaacagcccaagttccgaagtataaagtgcaaacacaaagaaaatccagttatggcagaacccccagttataaatgaggagtgcagtttgaaatgctgctcttctgataccaaaggctctcctttggccagcatttctaaaagtgggaaagtggatggtctaaaactactgaacaatatgcatgagaaaaccagggattcaagtgacatagaaacagcagtggtgaaacatgttttatccgagttgaaggaactctcttacagatccttaggtgaggatgtcagtgactctggaacatcaaagccatcaaaaccattacttttctcttctgcttctagtcagaatcacatacctattgaaccagactacaaattcagtacattgctaatgatgttgaaagatatgcatgatagtaagacgaaggagcagcggttgatgactgctcaaaacctggtctcttaccggagtcctggtcgtggggactgttctactaatagtcctgtaggagtctctaaggttttggtttcaggaggctccacacacaattcagagaaaaagggagatggcactcagaactccgccaatcctagccctagtgggggtgactctgcattatctggcgagttgtctgcttccctacctggcttactgtccgacaagagagacctccctgcttctggtaaaagtcgttcagactgtgttactaggcgcaactgtggacgatcaaagccttcatccaaattgcgagatgctttttcagcccaaatggtaaagaacacagtgaaccgtaaagccttaaagaccgagcgcaaaagaaaactgaatcagcttccaagtgtgactcttgatgctgtactgcagggagaccgagaacgtggaggttcattgagaggtggggcagaagatcctagtaaagaggatccccttcagataatgggccacttaacaagtgaagatggtgaccatttttctgatgtgcatttcgatagcaaggttaagcaatctgatcctggtaaaatttctgaaaaaggactctcttttgaaaacggaaaaggcccagagctggactctgtaatgaacagtgagaatgatgaactcaatggtgtaaatcaagtggtgcctaaaaagcggtggcagcgtttaaaccaaaggcgcactaaacctcgtaagcgcatgaacagatttaaagagaaagaaaactctgagtgtgcctttagggtcttacttcctagtgaccctgtgcaggaggggcgggatgagtttccagagcatagaactccttcagcaagcatacttgaggaaccactgacagagcaaaatcatgctgactgcttagattcagctgggccacggttaaatgtttgtgataaatccagtgccagcattggtgacatggaaaaggagccaggaattcccagtttgacaccacaggctgagctccctgaaccagctgtgcggtcagagaagaaacgccttaggaagccaagcaagtggcttttggaatatacagaagaatatgatcagatatttgctcctaagaaaaaacaaaagaaggtacaggagcaggtgcacaaggtaagttcccgctgtgaagaggaaagccttctagcccgaggtcgatctagtgctcagaacaagcaggtggacgagaattctttgatttcaaccaaagaagagcctccagttcttgaaagggaggctccgtttttggagggccccttggctcagtcagaacttggaggtggacatgctgagttgccgcagctgaccttgtctgtgcctgtggctccggaagtctctccacggcctgcccttgagtctgaggaattgctagttaaaacgccaggaaattatgaaagtaaacgtcaaagaaaaccaactaagaaacttcttgaatccaatgatttagaccctggatttatgcccaagaagggggaccttggcctttctaaaaagtgctatgaagctggtcacctggagaatggcataactgaatcttgtgccacatcttattcaaaagattttggtggaggcactaccaagatatttgacaagccaaggaagcgaaaacgacagaggcatgctgcagccaagatgcagtgtaaaaaagtgaaaaatgatgactcgtcaaaagagattccaggctcagagggagaactaatgcctcacaggacggccacaagccccaaggagactgttgaggaaggtgtagaacacgatcccgggatgcctgcctctaaaaaaatgcagggtgaacgcggtggaggagctgcactcaaggagaatgtctgtcagaattgtgaaaaattgggtgagctgctgttatgtgaggctcagtgctgtggggctttccacctggagtgccttggattgactgagatgccaagaggaaaatttatctgcaatgaatgtcgcacaggaatccatacctgttttgtatgtaagcagagtggggaagatgttaaaaggtgccttctacccttgtgtggaaagttttaccatgaagagtgtgtccagaagtacccacccactgttatgcagaacaagggcttccggtgctccctccacatctgtataacctgtcatgctgctaatccagccaatgtttctgcatctaaaggtcggttgatgcgctgtgtccgctgtcctgtggcataccacgccaatgacttttgcctggctgctgggtcaaagatccttgcatctaatagtatcatctgccctaatcactttacccctaggcggggctgccgaaatcatgagcatgttaatgttagctggtgctttgtgtgctcagaaggaggcagccttctgtgctgtgattcttgccctgctgcttttcatcgtgaatgcctgaacattgatatccctgaaggaaactggtattgcaatgactgtaaagcaggcaaaaagccacactacagggagattgtctgggtaaaagttggacgatacaggtggtggccagctgagatctgccatcctcgagctgttccttccaacattgataagatgagacatgatgtgggagagttcccagtcctcttttttggatctaatgactatttgtggactcaccaggcccgagtcttcccttacatggagggtgacgtgagcagcaaggataagatgggcaaaggagtggatgggacatataaaaaagctcttcaggaagctgcagcaaggtttgaggaattaaaggcccaaaaagagctaagacagctgcaggaagaccgaaagaatgacaagaagccaccaccttataaacatataaaggtaaaccgtcctattggcagggtacagatcttcactgcagacttatctgaaataccccgttgcaactgtaaagctactgatgagaacccctgtgggatagactctgaatgcatcaaccgcatgctgctctatgagtgccaccccacagtgtgtcctgccggagggcgctgtcaaaaccagtgcttttccaagcgccaatatccagaggttgaaattttccgcacattacagcggggttggggtctacggacaaaaacagatattaaaaagggtgaatttgtgaatgagtatgtgggtgagcttatagatgaagaagaatgcagagctcgaattcgctatgctcaagaacatgatatcactaatttctatatgctcaccctagacaaagaccgaatcattgatgctggtcccaaaggaaactatgctcggttcatgaatcattgctgccagcccaactgtgaaacacagaagtggtctgtgaatggagatacccgtgtaggcctttttgcactaagtgacattaaagcaggcactgaacttaccttcaactacaacctagaatgtcttgggaatggaaagactgtttgcaaatgtggagccccgaactgcagtggcttcttgggtgtaaggccaaagaatcaacccattgccacggaagaaaagtcaaagaaattcaagaagaagcaacagggaaagcgcaggacccagggtgaaatcacaaaggagcgagaagatgagtgttttagttgtggggatgctggccagctcgtctcctgcaagaaaccaggctgcccaaaagtttaccacgcagactgtctcaatctgaccaagcgaccagcagggaaatgggaatgtccgtggcatcagtgtgacatctgcgggaaggaagcagcctccttctgtgagatgtgccccagctccttttgtaagcagcatcgagaagggatgcttttcatttccaaactggatgggcgtctgtcttgtactgagcatgacccctgtgggcccaatcctctggaacctggggagatccgtgagtatgtgcctcccccagtaccgctgcctccagggccaagcactcacctggcagagcaatcaacaggaatggctgctcaggcacccaaaatgtcagataaacctcctgctgacaccaaccagatgctgtcgctctccaaaaaagctctggcagggacttgtcagaggccactgctacctgaaagacctcttgagagaactgactccaggccccagcctttagataaggtcagagacctcgctggctcagggaccaaatcccaatccttggtttccagccagaggccactggacaggccaccagcagtggcaggaccaagaccccagctaagcgacaaaccctctccagtgaccagcccaagctcctcaccctcagtcaggtcccaaccactggaaagacctctggggacggctgacccaaggctggataaatccataggtgctgccagcccaaggccccagtcactggagaaaacctcagttcccactggcctgagacttccgccgccagacagactgctcattactagcagtcccaaaccccagacttcagacaggcctactgacaaaccccatgcctctttgtcccagagactcccacctcctgagaaagtactatcagctgtggtccagacccttgtagctaaagaaaaagcactgaggcctgtggaccagaatactcagtcaaaaaatagagctgctttggtgatggatctcatagacctaactcctcgccagaaggagcgggcagcttcacctcatcaggtcacaccacaggctgatgagaagatgccagtgttggagtcaagttcatggcctgccagcaaaggtctggggcatatgccgagagctgttgagaaaggctgtgtgtcagatcctcttcagacatctgggaaagcagcagccccttcagaggacccctggcaagctgttaaatcactcacccaggccagacttctttctcagcctcctgccaaggcctttttatatgagccaacaactcaggcctcaggaagagcttctgcaggggctgagcagaccccagggcctcttagccaatccccgggcctggtgaagcaggcgaagcagatggtcggaggccagcaactacctgcacttgccgccaagagtgggcaatcttttaggtctctcgggaaggccccagcctccctccccactgaagaaaagaagttggtaaccacagagcaaagtccctgggccctgggaaaagcctcatcacgggcagggctctggcccatagtggctggacagacactggcacagtcttgctggtctgctgggagcacacagacattggcacagacttgctggtctcttggaagagggcaagaccccaaaccagagcaaaatacacttccagctcttaaccaggctccttccagtcacaagtgtgcagaatcagaacagaagtagtaccaatcaatgtcacatgaacaaacaagctgcccccagggtaccatttggggaggggaaatcttttctttctttcccccttaaaaaaaaacacatctgccccgaacactttcccactggtattctttcctcatatcccaacactcagaactcttgtgacattagccagtgggggcttatggttgtgtgaaccatgtatgaaaatccagtgggccccaaccaaggagacagacagacttgggtctctttcccccaacttttccacatggtcatcgtgaaataaaaagtccactctggagtcaaaaaaaaaaaaaaaaaaaaa

SEQ ID NO: 36 - protein, 2696 aa, Artificial Sequence
  Description of Artificial Sequence; note = synthetic construct
MDQTCELPRRNCLLPFSNPVNLDAPEDKDSPFGNGQSNFSEPLNGCTMQLSTVSGTSQNAYGQDSPSCYIPLRRLQDLASMINVEYLNGSADGSESFQDPEKSDSRAQTPIVCTSLSPGGPTALAMKQEPSCNNSPELQVKVTKTIKNGFLHFENFTCVDDADVDSEMDPEQPVTEDESIEEIFEETQTNATCNYETKSENGVKVAMGSEQDSTPESRHGAVKSPFLPLAPQTETQKNKQRNEVDGSNEKAALLPAPFSLGDTNITIEEQLNSINLSFQDDPDSSTSTLGNMLELPGTSSSSTSQELPFCQPKKKSTPLKYEVGDLIWAKFKRRPWWPCRICSDPLINTHSKMKVSNRRPYRQYYVEAFGDPSERAWVAGKAIVMFEGRHQFEELPVLRRRGKQKEKGYRHKVPQKILSKWEASVGLAEQYDVPKGSKNRKCIPGSIKLDSEEDMPFEDCTNDPESEHDLLLNGCLKSLAFDSEHSADEKEKPCAKSRARKSSDNPKRTSVKKGHIQFEAHKDERRGKIPENLGLNFISGDISDTQASNELSRIANSLTGSNTAPGSFLFSSCGKNTAKKEFETSNGDSLLGLPEGALISKCSREKNKPQRSLVCGSKVKLCYIGAGDEEKRSDSISICTTSDDGSSDLDPIEHSSESDNSVLEIPDAFDRTENMLSMQKNEKIKYSRFAATNTRVKAKQKPLISNSHTDHLMGCTKSAEPGTETSQVNLSDLKASTLVHKPQSDFTNDALSPKFNLSSSISSENSLIKGGAANQALLHSKSKQPKFRSIKCKHKENPVMAEPPVINEECSLKCCSSDTKGSPLASISKSGKVDGLKLLNNMHEKTRDSSDIETAVVKHVLSELKELSYRSLGEDVSDSGTSKPSKPLLFSSASSQNHIPIEPDYKFSTLLMMLKDMHDSKTKEQRLMTAQNLVSYRSPGRGDCSTNSPVGVSKVLVSGGSTHNSEKKGDGTQNSANPSPSGGDSALSGELSASLPGLLSDKRDLPASGKSRSDCVTRRNCGRSKPSSKLRDAFSAQMVKNTVNRKALKTERKRKLNQLPSVTLDAVLQGDRERGGSLRGGAEDPSKEDPLQIMGHLTSEDGDHFSDVHFDSKVKQSDPGKISEKGLSFENGKGPELDSVMNSENDELNGVNQVVPKKRWQRLNQRRTKPRKRMNRFKEKENSECAFRVLLPSDPVQEGRDEFPEHRTPSASILEEPLTEQNHADCLDSAGPRLNVCDKSSASIGDMEKEPGIPSLTPQAELPEPAVRSEKKRLRKPSKWLLEYTEEYDQIFAPKKKQKKVQEQVHKVSSRCEEESLLARGRSSAQNKQVDENSLISTKEEPPVLEREAPFLEGPLAQSELGGGHAELPQLTLSVPVAPEVSPRPALESEELLVKTPGNYESKRQRKPTKKLLESNDLDPGFMPKKGDLGLSKKCYEAGHLENGITESCATSYSKDFGGGTTKIFDKPRKRKRQRHAAAKMQCKKVKNDDSSKEIPGSEGELMPHRTATSPKETVEEGVEHDPGMPASKKMQGERGGGAALKENVCQNCEKLGELLLCEAQCCGAFHLECLGLTEMPRGKFICNECRTGIHTCFVCKQSGEDVKRCLLPLCGKFYHEECVQKYPPTVMQNKGFRCSLHICITCHAANPANVSASKGRLMRCVRCPVAYHANDFCLAAGSKILASNSIICPNHFTPRRGCRNHEHVNVSWCFVCSEGGSLLCCDSCPAAFHRECLNIDIPEGNWYCNDCKAGKKPHYREIVWVKVGRYRWWPAEICHPRAVPSNIDKMRHDVGEFPVLFFGSNDYLWTHQARVFPYMEGDVSSKDKMGKGVDGTYKKALQEAAARFEELKAQKELRQLQEDRKNDKKPPPYKHIKVNRPIGRVQIFTADLSEIPRCNCKATDENPCGIDSECINRMLLYECHPTVCPAGGRCQNQCFSKRQYPEVEIFRTLQRGWGLRTKTDIKKGEFVNEYVGELIDEEECRARIRYAQEHDITNFYMLTLDKDRIIDAGPKGNYARFMNHCCQPNCETQKWSVNGDTRVGLFALSDIKAGTELTFNYNLECLGNGKTVCKCGAPNCSGFLGVRPKNQPIATEEKSKKFKKKQQGKRRTQGEITKEREDECFSCGDAGQLVSCKKPGCPKVYHADCLNLTKRPAGKWECPWHQCDICGKEAASFCEMCPSSFCKQHREGMLFISKLDGRLSCTEHDPCGPNPLEPGEIREYVPPPVPLPPGPSTHLAEQSTGMAAQAPKMSDKPPADTNQMLSLSKKALAGTCQRPLLPERPLERTDSRPQPLDKVRDLAGSGTKSQSLVSSQRPLDRPPAVAGPRPQLSDKPSPVTSPSSSPSVRSQPLERPLGTADPRLDKSIGAASPRPQSLEKTSVPTGLRLPPPDRLLITSSPKPQTSDRPTDKPHASLSQRLPPPEKVLSAVVQTLVAKEKALRPVDQNTQSKNRAALVMDLIDLTPRQKERAASPHQVTPQADEKMPVLESSSWPASKGLGHMPRAVEKGCVSDPLQTSGKAAAPSEDPWQAVKSLTQARLLSQPPAKAFLYEPTTQASGRASAGAEQTPGPLSQSPGLVKQAKQMVGGQQLPALAAKSGQSFRSLGKAPASLPTEEKKLVTTEQSPWALGKASSRAGLWPIVAGQTLAQSCWSAGSTQTLAQTCWSLGRGQDPKPEQNTLPALNQAPSSHKCAESEQK

SEQ ID NO: 37 - DNA, 8431 bp, Artificial Sequence
  Description of Artificial Sequence; note = synthetic construct
ggttgatgccggcccaggatggatcagacctgtgaactacccagaagaaattgtctgctgcccttttccaatccagtgaatttagatgcccctgaagacaaggacagccctttcggtaatggtcaatccaatttttctgagccacttaatgggtgtactatgcagttatcgactgtcagtggaacatcccaaaatgcttatggacaagattctccatcttgttacattccactgcggagactacaggatttggcctccatgatcaatgtagagtatttaaatgggtctgctgatggatcagaatcctttcaagaccctgaaaaaagtgattcaagagctcagacgccaattgtttgcacttccttgagtcctggtggtcctacagcacttgctatgaaacaggaaccctcttgtaataactcccctgaactccaggtaaaagtaacaaagactatcaagaatggctttctgcactttgagaattttacttgtgtggacgatgcagatgtagattctgaaatggacccagaacagccagtcacagaggatgagagtatagaggagatctttgaggaaactcagaccaatgccacctgcaattatgagactaaatcagagaatggtgtaaaagtggccatgggaagtgaacaagacagcacaccagagagtagacacggtgcagtcaaatcgccattcttgccattagctcctcagactgaaacacagaaaaataagcaaagaaatgaagtggacggcagcaatgaaaaagcagcccttctcccagcccccttttcactaggagacacaaacattacaatagaagagcaattaaactcaataaatttatcttttcaggatgatccagattccagtaccagtacattaggaaacatgctagaattacctggaacttcatcatcatctacttcacaggaattgccattttgtcaacctaagaaaaagtctacgccactgaagtatgaagttggagatctcatctgggcaaaattcaagagacgcccatggtggccctgcaggatttgttctgatccgttgattaacacacattcaaaaatgaaagtttccaaccggaggccctatcggcagtactacgtggaggcttttggagatccttctgagagagcctgggtggctggaaaagcaatcgtcatgtttgaaggcagacatcaattcgaagagctacctgtccttaggagaagagggaaacagaaagaaaaaggatataggcataaggttcctcagaaaattttgagtaaatgggaagccagtgttggacttgcagaacagtatgatgttcccaaggggtcaaagaaccgaaaatgtattcctggttcaatcaagttggacagtgaagaagatatgccatttgaagactgcacaaatgatcctgagtcagaacatgacctgttgcttaatggctgtttgaaatcactggcttttgattctgaacattctgcagatgagaaggaaaagccttgtgctaaatctcgagccagaaagagctctgataatccaaaaaggactagtgtgaaaaagggccacatacaatttgaagcacataaagatgaacggaggggaaagattccagagaaccttggcctaaactttatctctggggatatatctgatacgcaggcctctaatgaactttccaggatagcaaatagcctcacagggtccaacactgccccaggaagttttctgttttcttcctgtggaaaaaacactgcaaagaaagaatttgagacttcaaatggtgactctttattgggcttgcctgagggtgctttgatctcaaagtgttctcgagagaagaataaaccccaacgaagcctggtgtgtggttcaaaagtgaagctctgctatattggagcaggtgatgaggaaaagcgaagtgattccattagtatctgtaccacttctgatgatggaagcagtgacctggatcccatagaacacagctcagagtctgataacagtgtccttgaaattccagatgctttcgatagaacagagaacatgttatctatgcagaaaaatgaaaagataaagtattctaggtttgctgccacaaacactagggtaaaagcaaaacagaagcctctcattagtaactcacatacagaccacttaatgggttgtactaagagtgcagagcctggaaccgagacgtctcaggttaatctctctgatctgaaggcatctactcttgttcacaaaccccagtcagattttacaaatgatgctctctctccaaaattcaacctgtcatcaagcatatccagtgagaactcgttaataaagggtggggcagcaaatcaagctctattacattcgaaaagcaaacagcccaagttccgaagtataaagtgcaaacacaaagaaaatccagttatggcagaacccccagttataaatgaggagtgcagtttgaaatgctgctcttctgataccaaaggctctcctttggccagcatttctaaaagtgggaaagtggatggtctaaaactactgaacaatatgcatgagaaaaccagggattcaagtgacatagaaacagcagtggtgaaacatgttttatccgagttgaaggaactctcttacagatccttaggtgaggatgtcagtgactctggaacatcaaagccatcaaaaccattacttttctcttctgcttctagtcagaatcacatacctattgaaccagactacaaattcagtacattgctaatgatgttgaaagatatgcatgatagtaagacgaaggagcagcggttgatgactgctcaaaacctggtctcttaccggagtcctggtcgtggggactgttctactaatagtcctgtaggagtctctaaggttttggtttcaggaggctccacacacaattcagagaaaaagggagatggcactcagaactccgccaatcctagccctagtgggggtgactctgcattatctggcgagttgtctgcttccctacctggcttactgtccgacaagagagacctccctgcttctggtaaaagtcgttcagactgtgttactaggcgcaactgtggacgatcaaagccttcatccaaattgcgagatgctttttcagcccaaatggtaaagaacacagtgaaccgtaaagccttaaagaccgagcgcaaaagaaaactgaatcagcttccaagtgtgactcttgatgctgtactgcagggagaccgagaacgtggaggttcattgagaggtggggcagaagatcctagtaaagaggatccccttcagataatgggccacttaacaagtgaagatggtgaccatttttctgatgtgcatttcgatagcaaggttaagcaatctgatcctggtaaaatttctgaaaaaggactctcttttgaaaacggaaaaggcccagagctggactctgtaatgaacagtgagaatgatgaactcaatggtgtaaatcaagtggtgcctaaaaagcggtggcagcgtttaaaccaaaggcgcactaaacctcgtaagcgcatgaacagatttaaagagaaagaaaactctgagtgtgcctttagggtcttacttcctagtgaccctgtgcaggaggggcgggatgagtttccagagcatagaactccttcagcaagcatacttgaggaaccactgacagagcaaaatcatgctgactgcttagattcagctgggccacggttaaatgtttgtgataaatccagtgccagcattggtgacatggaaaaggagccaggaattcccagtttgacaccacaggctgagctccctgaaccagctgtgcggtcagagaagaaacgccttaggaagccaagcaagtggcttttggaatatacagaagaatatgatcagatatttgctcctaagaaaaaacaaaagaaggtacaggagcaggtgcacaaggtaagttcccgctgtgaagaggaaagccttctagcccgaggtcgatctagtgctcagaacaagcaggtggacgagaattctttgatttcaaccaaagaagagcctccagttcttgaaagggaggctccgtttttggagggccccttggctcagtcagaacttggaggtggacatgctgagttgccgcagctgaccttgtctgtgcctgtggctccggaagtctctccacggcctgcccttgagtctgaggaattgctagttaaaacgccaggaaattatgaaagtaaacgtcaaagaaaaccaactaagaaacttcttgaatccaatgatttagaccctggatttatgcccaagaagggggaccttggcctttctaaaaagtgctatgaagctggtcacctggagaatggcataactgaatcttgtgccacatcttattcaaaagattttggtggaggcactaccaagatatttgacaagccaaggaagcgaaaacgacagaggcatgctgcagccaagatgcagtgtaaaaaagtgaaaaatgatgactcgtcaaaagagattccaggctcagagggagaactaatgcctcacaggacggccacaagccccaaggagactgttgaggaaggtgtagaacacgatcccgggatgcctgcctctaaaaaaatgcagggtgaacgcggtggaggagctgcactcaaggagaatgtctgtcagaattgtgaaaaattgggtgagctgctgttatgtgaggctcagtgctgtggggctttccacctggagtgccttggattgactgagatgccaagaggaaaatttatctgcaatgaatgtcgcacaggaatccatacctgttttgtatgtaagcagagtggggaagatgttaaaaggtgccttctacccttgtgtggaaagttttaccatgaagagtgtgtccagaagtacccacccactgttatgcagaacaagggcttccggtgctccctccacatctgtataacctgtcatgctgctaatccagccaatgtttctgcatctaaaggtcggttgatgcgctgtgtccgctgtcctgtggcataccacgccaatgacttttgcctggctgctgggtcaaagatccttgcatctaatagtatcatctgccctaatcactttacccctaggcggggctgccgaaatcatgagcatgttaatgttagctggtgctttgtgtgctcagaaggaggcagccttctgtgctgtgattcttgccctgctgcttttcatcgtgaatgcctgaacattgatatccctgaaggaaactggtattgcaatgactgtaaagcaggcaaaaagccacactacagggagattgtctgggtaaaagttggacgatacaggtggtggccagctgagatctgccatcctcgagctgttccttccaacattgataagatgagacatgatgtgggagagttcccagtcctcttttttggatctaatgactatttgtggactcaccaggcccgagtcttcccttacatggagggtgacgtgagcagcaaggataagatgggcaaaggagtggatgggacatataaaaaagctcttcaggaagctgcagcaaggtttgaggaattaaaggcccaaaaagagctaagacagctgcaggaagaccgaaagaatgacaagaagccaccaccttataaacatataaaggtaaaccgtcctattggcagggtacagatcttcactgcagacttatctgaaataccccgttgcaactgtaaagctactgatgagaacccctgtgggatagactctgaatgcatcaaccgcatgctgctctatgagtgccaccccacagtgtgtcctgccggagggcgctgtcaaaaccagtgcttttccaagcgccaatatccagaggttgaaattttccgcacattacagcggggttggggtctacggacaaaaacagatattaaaaagggtgaatttgtgaatgagtatgtgggtgagcttatagatgaagaagaatgcagagctcgaattcgctatgctcaagaacatgatatcactaatttctatatgctcaccctagacaaagaccgaatcattgatgctggtcccaaaggaaactatgctcggttcatgaatcattgctgccagcccaactgtgaaacacagaagtggtctgtgaatggagatacccgtgtaggcctttttgcactaagtgacattaaagcaggcactgaacttaccttcaactacaacctagaatgtcttgggaatggaaagactgtttgcaaatgtggagccccgaactgcagtggcttcttgggtgtaaggccaaagaatcaacccattgccacggaagaaaagtcaaagaaattcaagaagaagcaacagggaaagcgcaggacccagggtgaaatcacaaaggagcgagaagatgagtgttttagttgtggggatgctggccagctcgtctcctgcaagaaaccaggctgcccaaaagtttaccacgcagactgtctcaatctgaccaagcgaccagcagggaaatgggaatgtccgtggcatcagtgtgacatctgcgggaaggaagcagcctccttctgtgagatgtgccccagctccttttgtaagcagcatcgagaagggatgcttttcatttccaaactggatgggcgtctgtcttgtactgagcatgacccctgtgggcccaatcctctggaacctggggagatccgtgagtatgtgcctcccccagtaccgctgcctccagggccaagcactcacctggcagagcaatcaacaggaatggctgctcaggcacccaaaatgtcagataaacctcctgctgacaccaaccagatgctgtcgctctccaaaaaagctctggcagggacttgtcagaggccactgctacctgaaagacctcttgagagaactgactccaggccccagcctttagataaggtcagagacctcgctggctcagggaccaaatcccaatccttggtttccagccagaggccactggacaggccaccagcagtggcaggaccaagaccccagctaagcgacaaaccctctccagtgaccagcccaagctcctcaccctcagtcaggtcccaaccactggaaagacctctggggacggctgacccaaggctggataaatccataggtgctgccagcccaaggccccagtcactggagaaaacctcagttcccactggcctgagacttccgccgccagacagactgctcattactagcagtcccaaaccccagacttcagacaggcctactgacaaaccccatgcctctttgtcccagagactcccacctcctgagaaagtactatcagctgtggtccagacccttgtagctaaagaaaaagcactgaggcctgtggaccagaatactcagtcaaaaaatagagctgctttggtgatggatctcatagacctaactcctcgccagaaggagcgggcagcttcacctcatcaggtcacaccacaggctgatgagaagatgccagtgttggagtcaagttcatggcctgccagcaaaggtctggggcatatgccgagagctgttgagaaaggctgtgtgtcagatcctcttcagacatctgggaaagcagcagccccttcagaggacccctggcaagctgttaaatcactcacccaggccagacttctttctcagcctcctgccaaggcctttttatatgagccaacaactcaggcctcaggaagagcttctgcaggggctgagcagaccccagggcctcttagccaatccccgggcctggtgaagcaggcgaagcagatggtcggaggccagcaactacctgcacttgccgccaagagtgggcaatcttttaggtctctcgggaaggccccagcctccctccccactgaagaaaagaagttggtaaccacagagcaaagtccctgggccctgggaaaagcctcatcacgggcagggctctggcccatagtggctggacagacactggcacagtcttgctggtctgctgggagcacacagacattggcacagacttgctggtctcttggaagagggcaagaccccaaaccagagcaaaatacacttccagctcttaaccaggctccttccagtcacaagtgtgcagaatcagaacagaagtagtaccaatcaatgtcacatgaacaaacaagctgcccccagggtaccatttggggaggggaaatcttttctttctttcccccttaaaaaaaaacacatctgccccgaacactttcccactggtattctttcctcatatcccaacactcagaactcttgtgacattagccagtgggggcttatggttgtgtgaaccatgtatgaaaatccagtgggccccaaccaaggagacagacagacttgggtctctttcccccaacttttccacatggtcatcgtgaaataaaaagtccactctggagtcaaaaaaaaaaaaaaaaaaaaa

SEQ ID NO: 38 - protein, 1784 aa, Artificial Sequence
  Description of Artificial Sequence; note = synthetic construct
MKRKERIARRLEGIENDTQPILLQSCTGLVTHRLLEEDTPRYMRASDPASPHIGRSNEEEETSDSSLEKQTRSKYCTETSGVHGDSPYGSGTMDTHSLESKAERIARYKAERRRQLAEKYGLTLDPEADSEYLSRYTKSRKEPDAVEKRGGKSDKQEESSRDASSLYPGTETMGLRTCAGESKDYALHAGDGSSDPEVLLNIENQRRGQELSATRQAHDLSPAAESSSTFSFSGRDSSFTEVPRSPKHAHSSSLQQAASRSPSFGDPQLSPEARPRCTSHSETPTVDDEEKVDERAKLSVAAKRLLFREMEKSFDEQNVPKRRSRNTAVEQRLRRLQDRSLTQPITTEEVVIAATLQASAHQKALAKDQTNEGKELAEQGEPDSSTLSLAEKLALFNKLSQPVSKAISTRNRIDTRQRRMNARYQTQPVTLGEVEQVQSGKLIPFSPAVNTSVSTVASTVAPMYAGDLRTKPPLDHNASATDYKFSSSIENSDSPVRSILKSQAWQPLVEGSENKGMLREYGETESKRALTGRDSGMEKYGSFEEAEASYPILNRAREGDSHKESKYAVPRRGSLERANPPITHLGDEPKEFSMAKMNAQGNLDLRDRLPFEEKVEVENVMKRKFSLRAAEFGEPTSEQTGTAAGKTIAQTTAPVSWKPQDSSEQPQEKLCKNPCAMFAAGEIKTPTGEGLLDSPSKTMSIKERLALLKKSGEEDWRNRLSRRQEGGKAPASSLHTQEAGRSLIKKRVTESRESQMTIEERKQLITVREEAWKTRGRGAANDSTQFTVAGRMVKKGLASPTAITPVASAICGKTRGTTPVSKPLEDIEARPDMQLESDLKLDRLETFLRRLNNKVGGMHETVLTVTGKSVKEVMKPDDDETFAKFYRSVDYNMPRSPVEMDEDFDVIFDPYAPKLTSSVAEHKRAVRPKRRVQASKNPLKMLAAREDLLQEYTEQRLNVAFMESKRMKVEKMSSNSNFSEVTLAGLASKENFSNVSLRSVNLTEQNSNNSAVPYKRLMLLQIKGRRHVQTRLVEPRASALNSGDCFLLLSPHCCFLWVGEFANVIEKAKASELATLIQTKRELGCRATYIQTIEEGINTHTHAAKDFWKLLGGQTSYQSAGDPKEDELYEAAIIETNCIYRLMDDKLVPDDDYWGKIPKCSLLQPKEVLVFDFGSEVYVWHGKEVTLAQRKIAFQLAKHLWNGTFDYENCDINPLDPGECNPLIPRKGQGRPDWAIFGRLTEHNETILFKEKFLDWTELKRSNEKNPGELAQHKEDPRTDVKAYDVTRMVSMPQTTAGTILDGVNVGRGYGLVEGHDRRQFEITSVSVDVWHILEFDYSRLPKQSIGQFHEGDAYVVKWKFMVSTAVGSRQKGEHSVRAAGKEKCVYFFWQGRHSTVSEKGTSALMTVELDEERGAQVQVLQGKEPPCFLQCFQGGMVVHSGRREEEEENVQSEWRLYCVRGEVPVEGNLLEVACHCSSLRSRTSMVVLNVNKALIYLWHGCKAQAHTKEVGRTAANKIKEQCPLEAGLHSSSKVTIHECDEGSEPLGFWDALGRRDRKAYDCMLQDPGSFNFAPRLFILSSSSGDFAATEFVYPARAPSVVSSMPFLQEDLYSAPQPALFLVDNHHEVYLWQGWWPIENKITGSARIRWASDRKSAMETVLQYCKGKNLKKPAPKSYLIHAGLEPLTFTNMFPSWEHREDIAEITEMDTEVSNQITLVEDVLAKLCKTIYPLADLLARPLPEGVDPLKLEIYLTDEDFEFALDMTRDEYNALPAWKQVNLKKA

SEQ ID NO: 39 - DNA, 6719 bp, Artificial Sequence
  Description of Artificial Sequence; note = synthetic construct
tcggcgggaagcggcgatcctgccaccgggaggtgtggaagagccgggtagattctggctacattggagattggttgctttctaaaactgaaggagaagcccatgaagagatggtggattctcactgagttttgactagcggaagaaaagagagagttcaagtggatggccttgaggacttgaaaagctgagatatgatgattttgaagtcatttcacatcgaagccatgatttaaatatcggcgttaagatttcaacaagaaaaacttaagcttccttggattcccacgtcaaaggaaagtttcaagctttcagaaggagttctcactcgaagataaagaacagctcgctaaccacgaaagaggaatcgatgctcagcttttagttgcacttcctaaagttgcagaattaagacaaatctttgaaccaaagaagaaagaattcttagaaatgaaaagaaaagaaagaattgccaggcgcctggaagggattgaaaatgacactcagcccatcctcttgcagagctgcacaggattggtgactcaccgcctgctggaggaagacacccctcgatacatgagagccagcgaccctgccagcccccacatcggccgatcaaatgaagaggaggaaacttctgattcttctctagaaaagcaaactcgatccaaatactgcacagaaacctccggtgtccacggtgactcaccctatggttcgggtaccatggacacccacagtctggagtccaaagccgaaagaattgcaaggtacaaagcagaaagaaggcgacagctggcagagaagtatgggctgactctggatcccgaggccgactccgagtatttatcccgctataccaagtccaggaaggagcctgatgctgtcgagaagcggggaggaaaaagtgacaaacaggaagagtcaagcagagatgcgagttctctgtaccccgggaccgagacgatggggctcaggacctgtgccggtgaatccaaggactatgccctccatgcgggtgacggctcttccgacccggaggtgctgctgaacatagaaaaccaaagacgaggtcaagagctgagtgccacccggcaggcccatgacctgtccccagcagccgagagttcctcgaccttctctttctctgggcgagactcctccttcactgaagtgccacggtcccccaagcacgcccacagctcctccctgcagcaggcagcctcccggagcccctcctttggtgacccacagctatcccctgaggcccgacccaggtgcacttcacattcagaaacgccaactgtcgatgatgaagaaaaggtggatgaacgagccaagctgagcgtcgccgccaagaggttgcttttcagggagatggaaaaatcttttgatgaacaaaatgttccaaagcgacgctcaagaaacacagctgtggagcagaggctacgccgtctgcaggacaggtccctcacccagcccatcaccactgaagaggtggtcatcgcagccacattgcaggcctctgctcaccaaaaggccttagccaaggaccagacaaatgagggcaaagagcttgctgagcaaggagaacctgattcctccactctaagcttggccgaaaagttggccttgtttaacaaattgtcccagccagtctcaaaagcgatttctacccggaacagaatagacacgagacagaggagaatgaacgctcgctatcaaactcagccagtcacactgggagaggtggagcaggtgcagagtggaaagctcattcctttctcacctgccgtgaacacatcagtgtctaccgtagcatccacggttgctccaatgtatgccggagatcttcgcacaaagccacctcttgaccacaatgcaagtgccactgactataagttttcttcttcaatagaaaattcggactctccagttagaagcattctgaaatcgcaagcttggcagcctttggtagagggtagcgagaacaagggaatgttgagagaatatggagagacagaaagcaagagagctttgacaggtcgagacagtgggatggagaagtatgggtcctttgaggaagcagaagcatcctaccccatcctgaacagagccagggaaggagacagccataaggaatctaaatatgctgttcccagaagaggaagcctggaacgggcgaaccctcccatcacccacctcggggatgaaccgaaggaattttccatggctaaaatgaatgcacaaggaaacttggacttgagggacaggctgccctttgaagagaaggtggaggtggagaatgttatgaaaaggaagttttcactaagagcggcagagttcggggagcccacttccgagcagacggggacagctgctgggaaaactattgctcaaaccacagcccccgtgtcctggaagccccaggattcttcggaacagccacaggagaagctctgcaagaatccatgtgcgatgtttgctgctggagagatcaaaacgccgacaggggagggccttcttgactcacccagcaaaaccatgtctattaaagaaagattggcactgttgaagaaaagcggggaggaagattggagaaacagactcagcaggaggcaggagggcggcaaggcgccggccagcagcctgcacacccaggaagcagggcggtccctcatcaagaagcgggtcacagaaagtcgagagagccaaatgacgattgaggagaggaagcagctcatcactgtgagagaggaggcctggaagacgagaggcagaggagcggccaacgactcgacccagttcactgtggctggcaggatggtgaagaaaggtttggcgtcacctactgccataaccccagtagcctcagccatttgcggtaaaacaagaggcaccacacccgtttccaaacccctggaagatatcgaagccagaccagatatgcagttagaatcggacctgaagttggacaggctggaaacctttctaagaaggctgaataacaaagttggcgggatgcacgaaacggtgctcactgtcaccggcaaatctgtgaaggaggtgatgaagccagatgatgatgaaacctttgccaaattttaccgcagcgtggattataatatgccaagaagtcctgtggagatggatgaggacttcgatgtcattttcgatccttatgcacccaaattgacgtcttccgtggccgagcacaagcgggcagttaggcccaagcgccgggttcaggcctccaaaaaccccctgaaaatgctggcggcaagagaagatctccttcaggaatacactgagcagagattaaacgttgccttcatggagtcaaagcggatgaaagtagaaaagatgtcttccaactccaacttctcagaagtcaccctggcgggtttagccagtaaagaaaacttcagcaacgtcagcctgcggagcgtcaacctgacggaacagaactctaacaacagcgccgtgccctacaagaggctgatgctgttgcagattaaaggaagaagacatgtgcagaccaggctggtggaacctcgagcttcggcgctcaacagtggggactgcttcctcctgctctctccccactgctgcttcctgtgggtaggagagtttgcaaacgtcatagaaaaggcgaaggcctcagaacttgcaactttaattcagacaaagagggaacttggttgtagagctacttatatccaaaccattgaagaaggaattaatacacacactcatgcagccaaagacttctggaagcttctgggtggccaaaccagttaccaatctgctggagacccaaaagaagatgaactctatgaagcagccataatagaaactaactgcatttaccgtctcatggatgacaaacttgttcctgatgacgactactgggggaaaattccgaagtgctcccttctgcaacccaaagaggtactggtgtttgattttggtagtgaagtttacgtatggcatgggaaagaagtcacattagcacaacgaaaaatagcatttcagctggcaaagcacttatggaatggaacctttgactatgagaactgtgacatcaatcccctggatcctggagaatgcaatccgcttatccccagaaaaggacaggggcggcccgactgggcgatatttgggagacttactgaacacaatgagacgattttgttcaaagagaagtttctggattggacggaactgaagagatcgaatgagaagaaccccggggaacttgcccagcacaaggaagaccccaggactgatgtcaaggcatacgatgtgacacggatggtgtccatgccccagacgacagcaggcaccatcctggacggagtgaacgtcggccgtggctatggcctggtggaaggacacgacaggaggcagtttgagatcaccagcgtttccgtggatgtctggcacatcctggaattcgactatagcaggctccccaaacaaagcatcgggcagttccatgagggggatgcctatgtggtcaagtggaagttcatggtgagcacggcagtgggaagtcgccagaagggagagcactcggtgagggcagccggcaaagagaagtgcgtctacttcttctggcaaggccggcactccaccgtgagtgagaagggcacgtcggcgctgatgacggtggagctggacgaggaaaggggggcccaggtccaggttctccagggaaaggagcccccctgtttcctgcagtgtttccagggggggatggtggtgcactcggggaggcgggaagaggaagaagaaaatgtgcaaagtgagtggcggctgtactgcgtgcgtggagaggtgcccgtggaagggaatttgctggaagtggcctgtcactgtagcagcctgaggtccagaacttccatggtggtgcttaacgtcaacaaggccctcatctacctgtggcacggatgcaaagcccaggcccacacgaaggaggtcggaaggaccgctgcgaacaagatcaaggaacaatgtcccctggaagcaggactgcatagtagcagcaaagtcacaatacacgagtgtgatgaaggctccgagccactcggattctgggatgccttaggaaggagagacaggaaagcctacgattgcatgcttcaagatcctggaagttttaacttcgcgccccgcctgttcatcctcagcagctcctctggggattttgcagccacagagtttgtgtaccctgcccgagccccctctgtggtcagttccatgcccttcctgcaggaagatctgtacagcgcgccccagccagcacttttccttgttgacaatcaccacgaggtgtacctctggcaaggctggtggcccatcgagaacaagatcactggttccgcccgcatccgctgggcctccgaccggaagagtgcgatggagactgtgctccagtactgcaaaggaaaaaatctcaagaaaccagcccccaagtcttaccttatccacgctggtctggagcccctgacattcaccaatatgtttcccagctgggagcacagagaggacatcgctgagatcacagagatggacacggaagtttccaatcagatcaccctcgtggaagacgtcttagccaagctctgtaaaaccatttacccgctggccgacctcctggccaggccactcccggagggggtcgatcctctgaagcttgagatctatctcaccgacgaagacttcgagtttgcactagacatgacgagggatgaatacaacgccctgcccgcctggaagcaggtgaacctgaagaaagcaaaaggcctgttctgagtggggagacgccagaggagcctcacggtcacgtccaacaacaccactgcaccagggaaatggatatatatttttggactggtgtttttcacaaagtatttttcaatcagagttttcagaacctgacattgttaaagatactgcttgtcccggagttgtgtattttgtaaatgttcaagggaactgtttggaaacttctttccaccattcaggaggttatcagaattaataaaagtatctgttatgtgcacttaagccgcagctgctatagatagcactgccttcttgttccagctaggcaatgcctttttttttttttttgaagcagttctctttataaagtgttattttgatagtttgtggattctaaaatatatatatatttatataaacaccatataagtcaaatatgtatttaacaaagcaatatgtattcattcactttcaagatttgttttggtgtcaaaataacatgaaaaggtagatggagttgcttctgttgaattagctctgccaccaatatgtatcttcatacacgtttggaaatgtttcctgcagcattaggtatgacttgttctgagtactgcttccggtgctaaaatgaacaaagaatttgtacttaatggcatggactctggagaatctatgcgaatcaacctttctaccttaatatctccccaaaaatgtatagtgccttgtttttatgtacagtttatatacagaaaagtttgctctgcatttttgatgatggtttggaacattatctacaattttactctcaaatagtcaaaataaaaacatctcaatttctaataccggttgtaaacaaacagtacacatgtcattttgtgatataggactcccaaataaaagtatcagaataaacacaacaattaactggt

SEQ ID NO: 40 - protein, 731 aa, Artificial Sequence
  Description of Artificial Sequence; note = synthetic construct
MVVEHPEFLKAGKEPGLQIWRVEKFDLVPVPPNLYGDFFTGDAYVILKTVQLRNGNLQYDLHYWLGNECSQDESGAAAIFTVQLDDYLNGRAVQHREVQGFESSTFSGYFKSGLKYKKGGVASGFKHVVPNEVVVQRLFQVKGRRVVRATEVPVSWDSFNNGDCFILDLGNNIYQWCGSGSNKFERLKATQVSKGIRDNERSGRAQVHVSEEETEPEAMLQVLGPKPALPEGTEDTAKEDAANRKLAKLYKVSNGAGSMSVSLVADENPFAQGPLRSEDCFILDHGRDGKIFVWKGKQANMEERKAALKTASDFISKMQYPRQTQVSVLPEGGETPLFKQFFKNWRDPDQTDGPGLGYLSSHIANVERVPFDAGTLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSRDGGQTAPASIRLFQVRASSSGATRAVEVMPKSGALNSNDAFVLKTPSAAYLWVGAGASEAEKTAAQELLKVLRSQHVQVEEGSEPDGFWEALGGKTSYRTSPRLKDKKMDAHPPRLFACSNRIGRFVIEEVPGELMQEDLATDDVMLLDTWDQVFVWVGKDSQEEEKTEALTSAKRYIETDPANRDRRTPITVVRQGFEPPSFVGWFLGWDNNYWSVDPLDRALAELAA

SEQ ID NO: 41 - DNA, 2447 bp, Artificial Sequence
  Description of Artificial Sequence; note = synthetic construct
tgagcgcggcccagcactatggtggtggagcaccccgaattcctgaaggcagggaaggagcctggcctgcagatctggcgtgtggagaagtttgacctggtgcctgtgccccccaacctctatggagacttcttcacgggtgatgcctatgtcatcctgaagactgtgcagctgaggaatgggaatctgcagtatgacctccactattggctgggcaatgaatgcagccaggatgagagcggggctgctgccatctttactgtgcaactggatgactacctgaacggccgggctgtgcagcaccgtgaggttcagggctttgagtcgtccaccttctccggctacttcaagtctggacttaagtacaagaaaggaggtgtggcatctggattcaaacacgtggtacccaatgaggtggtggtccagaggctcttccaggtcaaaggacgccgtgtagtccgtgctactgaggtacctgtgtcctgggacagtttcaacaatggcgactgcttcattctggacctgggaaacaatatctatcagtggtgtggctctggcagcaacaaatttgaaaggctgaaggccacacaggtgtccaagggcatccgggacaacgagaggagtggccgtgctcaagtacacgtgtctgaagaggagactgagcccgaggcgatgctgcaggtgctgggccccaagccggctctgcctgaaggtaccgaggacacagccaaggaagatgcagccaaccgcaagctggccaagctctacaaggtctccaacggtgcaggtagcatgtcagtctccctagtggctgatgagaaccccttcgcccaggggcccctgagatctgaggactgcttcatcctggaccatggcagagatgggaaaatctttgtttggaaaggcaagcaggccaacatggaggagcggaaggctgccctcaaaacagcctctgacttcatctccaagatgcagtaccccaggcagacccaggtttcagttctcccagagggcggtgagacccctctctttaagcagttcttcaagaactggcgggacccagaccagacagatggccccggcctgggctacctctccagccacattgccaacgtggagcgcgtacctttcgatgccggcacgctgcacacctccaccgccatggccgctcagcacggcatggatgatgatggaactggccagaaacagatctggagaattgaaggttccaacaaggtgccagtggaccctgccacatacggacagttctatggaggcgacagctacatcattctgtacaactaccgccacggtggccgccagggacagatcatctacaactggcagggtgctcagtctacccaggatgaggttgctgcttctgccatcctgactgcccagctggatgaggagctgggaggaactcctgtccagagccgagtggtccaaggcaaagagcctgcacacctcatgagcttgtttggcgggaagcccatgatcatctacaagggtggcacctcccgtgatggtgggcagacagctcctgccagtatccgcctcttccaagtgcgtgccagcagctctggagccaccagggctgtggaggtgatgcctaagtctggtgctctgaactccaacgatgcctttgtgctgaaaaccccctccgctgcctacctgtgggtgggcgcaggagccagtgaggcagagaagacggcggcccaggagcttctgaaggtccttcggtcccagcatgtgcaggtggaagaaggcagtgagccagatggcttctgggaggctctgggcgggaagacgtcctaccgcacatcccccaggcttaaggacaagaagatggatgcccatcctcctcgactctttgcctgctccaacaggatcggacgctttgtgatcgaagaggttcctggcgagcttatgcaggaagacctggctactgatgacgtcatgctcctggacacctgggaccaggtctttgtctgggttggaaaagactcccaggaagaagaaaagacggaagccttgacttctgctaagcggtacatcgagacagatccagcaaatcgggacaggcggacccccatcacagtcgttaggcagggctttgagcctccttccttcgtgggctggttcctcggctgggacaacaactactggtcggtggatcctttggaccgggccttggctgagctggctgcctgagtaaggaccaagccatcaatgtcaccaatcagtgcctttgagggttgtccatctcccaaagacatcatatggcaagcaggaaaactatgatgtgtgcgcgcgtgtttttgtttttgttttttacggtagccaaaacaagcccttgtggaaactcagggtctttacagaattgcttcaaatgtctgtactttggaaatgaaagccaataaaagctttttgaagtgaaaaaaaaa

SEQ ID NO: 42 - protein, 928 aa, Artificial Sequence; identical to SEQ ID NO: 8
  Description of Artificial Sequence; note = synthetic construct

SEQ ID NO: 43 - DNA, 2994 bp, Artificial Sequence
  Description of Artificial Sequence; note = synthetic construct
ttccggtttttctcaggggacgttgaaattatttttgtaacgggagtcgggagaggacggggcgtgccccgcgtgcgcgcgcgtcgtcctccccggcgctcctccacagctcgctggctcccgccgcggaaaggcgtcatgccgcccaaaaccccccgaaaaacggccgccaccgccgccgctgccgccgcggaacccccggcaccgccgccgccgccccctcctgaggaggacccagagcaggacagcggcccggaggacctgcctctcgtcaggcttgagtttgaagaaacagaagaacctgattttactgcattatgtcagaaattaaagataccagatcatgtcagagagagagcttggttaacttgggagaaagtttcatctgtggatggagtattgggaggttatattcaaaagaaaaaggaactgtggggaatctgtatctttattgcagcagttgacctagatgagatgtcgttcacttttactgagctacagaaaaacatagaaatcagtgtccataaattctttaacttactaaaagaaattgataccagtaccaaagttgataatgctatgtcaagactgttgaagaagtatgatgtattgtttgcactcttcagcaaattggaaaggacatgtgaacttatatatttgacacaacccagcagttcgatatctactgaaataaattctgcattggtgctaaaagtttcttggatcacatttttattagctaaaggggaagtattacaaatggaagatgatctggtgatttcatttcagttaatgctatgtgtccttgactattttattaaactctcacctcccatgttgctcaaagaaccatataaaacagctgttatacccattaatggttcacctcgaacacccaggcgaggtcagaacaggagtgcacggatagcaaaacaactagaaaatgatacaagaattattgaagttctctgtaaagaacatgaatgtaatatagatgaggtgaaaaatgtttatttcaaaaattttataccttttatgaattctcttggacttgtaacatctaatggacttccagaggttgaaaatctttctaaacgatacgaagaaatttatcttaaaaataaagatctagatgcaagattatttttggatcatgataaaactcttcagactgattctatagacagttttgaaacacagagaacaccacgaaaaagtaaccttgatgaagaggtgaatgtaattcctccacacactccagttaggactgttatgaacactatccaacaattaatgatgattttaaattcagcaagtgatcaaccttcagaaaatctgatttcctattttaacaactgcacagtgaatccaaaagaaagtatactgaaaagagtgaaggatataggatacatctttaaagagaaatttgctaaagctgtgggacagggttgtgtcgaaattggatcacagcgatacaaacttggagttcgcttgtattaccgagtaatggaatccatgcttaaatcagaagaagaacgattatccattcaaaattttagcaaacttctgaatgacaacatttttcatatgtctttattggcgtgcgctcttgaggttgtaatggccacatatagcagaagtacatctcagaatcttgattctggaacagatttgtctttcccatggattctgaatgtgcttaatttaaaagcctttgatttttacaaagtgatcgaaagttttatcaaagcagaaggcaacttgacaagagaaatgataaaacatttagaacgatgtgaacatcgaatcatggaatcccttgcatggctctcagattcacctttatttgatcttattaaacaatcaaaggaccgagaaggaccaactgatcaccttgaatctgcttgtcctcttaatcttcctctccagaataatcacactgcagcagatatgtatctttctcctgtaagatctccaaagaaaaaaggttcaactacgcgtgtaaattctactgcaaatgcagagacacaagcaacctcagccttccagacccagaagccattgaaatctacctctctttcactgttttataaaaaagtgtatcggctagcctatctccggctaaatacactttgtgaacgccttctgtctgagcacccagaattagaacatatcatctggacccttttccagcacaccctgcagaatgagtatgaactcatgagagacaggcatttggaccaaattatgatgtgttccatgtatggcatatgcaaagtgaagaatatagaccttaaattcaaaatcattgtaacagcatacaaggatcttcctcatgctgttcaggagacattcaaacgtgttttgatcaaagaagaggagtatgattctattatagtattctataactcggtcttcatgcagagactgaaaacaaatattttgcagtatgcttccaccaggccccctaccttgtcaccaatacctcacattcctcgaagcccttacaagtttcctagttcacccttacggattcctggagggaacatctatatttcacccctgaagagtccatataaaatttcagaaggtctgccaacaccaacaaaaatgactccaagatcaagaatcttagtatcaattggtgaatcattcgggacttctgagaagttccagaaaataaatcagatggtatgtaacagcgaccgtgtgctcaaaagaagtgctgaaggaagcaaccctcctaaaccactgaaaaaactacgctttgatattgaaggatcagatgaagcagatggaagtaaacatctcccaggagagtccaaatttcagcagaaactggcagaaatgacttctactcgaacacgaatgcaaaagcagaaaatgaatgatagcatggatacctcaaacaaggaagagaaatgaggatctcaggaccttggtggacactgtgtacacctctggattcattgtctctcacagatgtgactgtat

SEQ ID NO: 44 - protein, 782 aa, Artificial Sequence
  Description of Artificial Sequence; note = synthetic construct
MAPHRPAPALLCALSLALCALSLPVRAATASRGASQAGAPQGRVPEARPNSMVVEHPEFLKAGKEPGLQIWRVEKFDLVPVPTNLYGDFFTGDAYVILKTVQLRNGNLQYDLHYWLGNECSQDESGAAAIFTVQLDDYLNGRAVQHREVQGFESATFLGYFKSGLKYKKGGVASGFKHVVPNEVVVQRLFQVKGRRVVRATEVPVSWESFNNGDCFILDLGNNIHQWCGSNSNRYERLKATQVSKGIRDNERSGRARVHVSEEGTEPEAMLQVLGPKPALPAGTEDTAKEDAANRKLAKLYKVSNGAGTMSVSLVADENPFAQGALKSEDCFILDHGKDGKIFVWKGKQANTEERKAALKTASDFITKMDYPKQTQVSVLPEGGETPLFKQFFKNWRDPDQTDGLGLSYLSSHIANVERVPFDAATLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSREGGQTAPASTRLFQVRANSAGATRAVEVLPKAGALNSNDAFVLKTPSAAYLWVGTGASEAEKTGAQELLRVLRAQPVQVAEGSEPDGFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDDYWSVDPLDRAMAELAA

SEQ ID NO: 45 - DNA, 2663 bp, Artificial Sequence
  Description of Artificial Sequence; note = synthetic construct
ccaccatggctccgcaccgccccgcgcccgcgctgctttgcgcgctgtccctggcgctgtgcgcgctgtcgctgcccgtccgcgcggccactgcgtcgcggggggcgtcccaggcgggggcgccccaggggcgggtgcccgaggcgcggcccaacagcatggtggtggaacaccccgagttcctcaaggcagggaaggagcctggcctgcagatctggcgtgtggagaagttcgatctggtgcccgtgcccaccaacctttatggagacttcttcacgggcgacgcctacgtcatcctgaagacagtgcagctgaggaacggaaatctgcagtatgacctccactactggctgggcaatgagtgcagccaggatgagagcggggcggccgccatctttaccgtgcagctggatgactacctgaacggccgggccgtgcagcaccgtgaggtccagggcttcgagtcggccaccttcctaggctacttcaagtctggcctgaagtacaagaaaggaggtgtggcatcaggattcaagcacgtggtacccaacgaggtggtggtgcagagactcttccaggtcaaagggcggcgtgtggtccgtgccaccgaggtacctgtgtcctgggagagcttcaacaatggcgactgcttcatcctggacctgggcaacaacatccaccagtggtgtggttccaacagcaatcggtatgaaagactgaaggccacacaggtgtccaagggcatccgggacaacgagcggagtggccgggcccgagtgcacgtgtctgaggagggcactgagcccgaggcgatgctccaggtgctgggccccaagccggctctgcctgcaggtaccgaggacaccgccaaggaggatgcggccaaccgcaagctggccaagctctacaaggtctccaatggtgcagggaccatgtccgtctccctcgtggctgatgagaaccccttcgcccagggggccctgaagtcagaggactgcttcatcctggaccacggcaaagatgggaaaatctttgtctggaaaggcaagcaggcaaacacggaggagaggaaggctgccctcaaaacagcctctgacttcatcaccaagatggactaccccaagcagactcaggtctcggtccttcctgagggcggtgagaccccactgttcaagcagttcttcaagaactggcgggacccagaccagacagatggcctgggcttgtcctacctttccagccatatcgccaacgtggagcgggtgcccttcgacgccgccaccctgcacacctccactgccatggccgcccagcacggcatggatgacgatggcacaggccagaaacagatctggagaatcgaaggttccaacaaggtgcccgtggaccctgccacatatggacagttctatggaggcgacagctacatcattctgtacaactaccgccatggtggccgccaggggcagataatctataactggcagggtgcccagtctacccaggatgaggtcgctgcatctgccatcctgactgctcagctggatgaggagctgggaggtacccctgtccagagccgtgtggtccaaggcaaggagcccgcccacctcatgagcctgtttggtgggaagcccatgatcatctacaagggcggcacctcccgcgagggcgggcagacagcccctgccagcacccgcctcttccaggtccgcgccaacagcgctggagccacccgggctgttgaggtattgcctaaggctggtgcactgaactccaacgatgcctttgttctgaaaaccccctcagccgcctacctgtgggtgggtacaggagccagcgaggcagagaagacgggggcccaggagctgctcagggtgctgcgggcccaacctgtgcaggtggcagaaggcagcgagccagatggcttctgggaggccctgggcgggaaggctgcctaccgcacatccccacggctgaaggacaagaagatggatgcccatcctcctcgcctctttgcctgctccaacaagattggacgttttgtgatcgaagaggttcctggtgagctcatgcaggaagacctggcaacggatgacgtcatgcttctggacacctgggaccaggtctttgtctgggttggaaaggattctcaagaagaagaaaagacagaagccttgacttctgctaagcggtacatcgagacggacccagccaatcgggatcggcggacgcccatcaccgtggtgaagcaaggctttgagcctccctcctttgtgggctggttccttggctgggatgatgattactggtctgtggaccccttggacagggccatggctgagctggctgcctgaggaggggcagggcccacccatgtcaccggtcagtgccttttggaactgtccttccctcaaagaggccttagagcgagcagagcagctctgctatgagtgtgtgtgtgtgtgtgtgttgtttcttttttttttttttacagtatccaaaaatagccctgcaaaaattcagagtccttgcaaaattgtctaaaatgtcagtgtttgggaaattaaatccaataaaaacattttgaagtgtgaaaaaaaaaaaaaaaaaaaaaaaaaaaaaaaaaaaaaaaaaaaaaaaaaaaaaaaaaaaaaaaaaaaaaa

SEQ ID NO: 46 - protein, 1441 aa, Artificial Sequence [sequence omitted]

SEQ ID NO: 47 - DNA, 4547 bp, Artificial Sequence
  Description of Artificial Sequence; note = synthetic construct
cggatccactagtccagtgtggtggaattcggcttcatcatcatgagtggccttggggacagttcatccgaccctgctaacccagactcacataagaggaaaggatcgccatgtgacacactggcatcaagcacggaaaagaggcgcagggagcaagaaaataaatatttagaagaactagctgagttactgtctgccaacattagtgacattgacagcttgagtgtaaaaccagacaaatgcaagattttgaagaaaacagtcgatcagatacagctaatgaagagaatggaacaagagaaatcaacaactgatgacgatgtacagaaatcagacatctcatcaagtagtcaaggagtgatagaaaaggaatccttgggacctcttcttttggaggctttggatggatttttctttgttgtgaactgtgaagggagaattgtatttgtgtcagagaatgtaaccagctacttaggttacaatcaggaggaattaatgaatacgagcgtctacagcatactgcacgtgggggatcatgcagaatttgtgaagaatctgctaccaaaatcactagtaaatggagttccttggcctcaagaggcaacacgacgaaatagccatacctttaactgcaggatgctaattcaccctccagatgagccagggaccgagaaccaagaagcttgccagcgttatgaagtaatgcagtgtttcactgtgtcacagccaaaatcaattcaagaggatggagaagatttccagtcatgtctgatttgtattgcacggcgattacctcggcctccagctattacgggtgtagaatcctttatgaccaagcaagatactacaggtaaaatcatctctattgatactagttccctgagagctgctggcagaactggttgggaagatttagtgaggaagtgcatttatgcttttttccaacctcagggcagagaaccatcttatgccagacagctgttccaagaagtgatgactcgtggcactgcctccagcccctcctatagattcatattgaatgatgggacaatgcttagcgcccacaccaagtgtaaactttgctaccctcaaagtccagacatgcaacctttcatcatgggaattcatatcatcgacagggagcacagtgggctttctcctcaagatgacactaattctggaatgtcaattccccgagtaaatccctcggtcaatcctagtatctctccagctcatggtgtggctcgttcatccacattgccaccatccaacagcaacatggtatccaccagaataaaccgccagcagagctcagaccttcatagcagcagtcatagtaattctagcaacagccaaggaagtttcggatgctcacccggaagtcagattgtagccaatgttgccttaaacaaaggacaggccagttcacagagcagtaaaccctctttaaacctcaataatcctcctatggaaggtacaggaatatccctagcacagttcatgtctccaaggagacaggttacttctggattggcaacaaggcccaggatgccaaacaattcctttcctcctaatatttcgacattaagctctcccgttggcatgacaagtagtgcctgtaataataataaccgatcttattcaaacatcccagtaacatctttacagggtatgaatgaaggacccaataactccgttggcttctctgccagttctccagtcctcaggcagatgagctcacagaattcacctagcagattaaatatacaaccagcaaaagctgagtccaaagataacaaagagattgcctcaactttaaatgaaatgattcaatctgacaacagctctagtgatggcaaacctctggattcagggcttctgcataacaatgacagactttcagatggagacagtaaatactctcaaaccagtcacaaactagtgcagcttttgacaacaactgccgaacagcagttacggcatgctgatatagacacaagctgcaaagatgtcctgtcttgcacaggcacttccaactctgcctctgctaactcttcaggaggttcttgtccctcttctcatagctcattgacagcacggcataaaattctacaccggctcttacaggagggtagcccctcagatatcaccactttgtctgtcgagcctgataaaaaggacagtgcatctacttctgtgtcagtgactggacaggtacaaggaaactccagtataaaactagaactggatgcttcaaagaaaaaagaatcaaaagaccatcagctcctacgctatcttttagataaagatgagaaagatttaagatcaactccaaacctgagcctggatgatgtaaaggtgaaagtggaaaagaaagaacagatggatccatgtaatacaaacccaaccccaatgaccaaacccactcctgaggaaataaaactggaggcccagagccagtttacagctgaccttgaccagtttgatcagttactgcccacgctggagaaggcagcacagttgccaggcttatgtgagacagacaggatggatggtgcggtcaccagtgtaaccatcaaatcggagatcctgccagcttcacttcagtccgccactgccagacccacttccaggctgaatagattacctgagctggaattggaagcaattgataaccaatttggacaaccaggaacaggcgatcagattccatggacaaataatacagtgacagctataaatcagagtaaatcagaagaccagtgtattagctcacaattagatgagcttctctgtccacccacaacagtagaagggagaaatgatgagaaggctcttcttgaacagctggtatccttccttagtggcaaagatgaaactgagctagctgaactagacagagctctgggaattgacaaacttgttcaggggggtggattagatgtattatcagagagatttccaccacaacaagcaacgccacctttgatcatggaagaaagacccaacctttattcccagccttactcttctccttttcctactgccaatctccctagccctttccaaggcatggtcaggcaaaaaccttcactggggacgatgcctgttcaagtaacacctccccgaggtgctttttcacctggcatgggcatgcagcccaggcaaactctaaacagacctccggctgcacctaaccagcttcgacttcaactacagcagcgattacagggacaacagcagttgatacaccaaaatcggcaagctatcttaaaccagtttgcagcaactgctcctgttggcatcaatatgagatcaggcatgcaacagcaaattacacctcagccacccctgaatgctcaaatgttggcacaacgtcagcgggaactgtacagtcaacagcaccgacagaggcagctaatacagcagcaaagagccatgcttatgaggcagcaaagctttgggaacaacctccctccctcatctggactaccagttcaaacggggaacccccgtcttcctcagggtgctccacagcaattcccctatccaccaaactatggtacaaatccaggaaccccacctgcttctaccagcccgttttcacaactagcagcaaatcctgaagcatccttggccaaccgcaacagcatggtgagcagaggcatgacaggaaacataggaggacagtttggcactggaatcaatcctcagatgcagcagaatgtcttccagtatccaggagcaggaatggttccccaaggtgaggccaactttgctccatctctaagccctgggagctccatggtgccgatgccaatccctcctcctcagagttctctgctccagcaaactccacctgcctccgggtatcagtcaccagacatgaaggcctggcagcaaggagcgataggaaacaacaatgtgttcagtcaagctgtccagaaccagcccacgcctgcacagccaggagtatacaacaacatgagcatcaccgtttccatggcaggtggaaatacgaatgttcagaacatgaacccaatgatggcccagatgcagatgagctctttgcagatgccaggaatgaacactgtgtgccctgagcagataaatgatcccgcactgagacacacaggcctctactgcaaccagctctcatccactgaccttctcaaaacagaagcagatggaacccagcaggtgcaacaggttcaggtgtttgctgacgtccagtgtacagtgaatctggtaggcggggacccttacctgaaccagcctggtccactgggaactcaaaagcccacgtcaggaccacagaccccccaggcccagcagaagagcctccgtcagcagctactgactgaataaccacttttaaaggaatgtgaaatttaaataatagacatacagagatatacaaatatattatatatttttctgagatttttgatatctcaatctgcagccattcttcaggtcgtagcatttggagcaaaaaaaaaaaaaaaaatcgatgtcgagagtacttctagagggcccgtttaaac